(19) United States
(12) Patent Application Publication
(10) Pub. No.: US 2023/0183218 A1
(43) Pub. Date: Jun. 15, 2023

(54) CONDENSED SUBSTITUTED HYDROPYRROLES AS ANTAGONISTS OF THE MUSCARINIC ACETYLCHOLINE RECEPTOR M4

(71) Applicant: Vanderbilt University, Nashville, TN (US)

(72) Inventors: Craig W. Lindsley, Brentwood, TN (US); P. Jeffrey Conn, Nashville, TN (US); Darren W. Engers, Brentwood, TN (US); Aaron M. Bender, Spring Hill, TN (US); Changho Han, Nashville, TN (US)

(21) Appl. No.: 17/920,899

(22) PCT Filed: Apr. 23, 2021
(86) PCT No.: PCT/US2021/028763

§ 371 Date: Oct. 24, 2022

Related U.S. Application Data
(60) Provisional application No. 63/015,248, filed on Apr. 24, 2020.

(51) Int. Cl. C07D 405/14; C07D 403/12; C07D 409/14; C07D 417/14; C07D 471/04; C07D 403/14; C07D 401/14; C07D 487/04

(52) U.S. Cl. CPC C07D 405/14; C07D 403/12; C07D 409/14; C07D 417/14; C07D 471/04; C07D 403/14; C07D 401/14; C07D 487/04

(57) ABSTRACT

Disclosed herein are substituted hexahydro-1H-cyclopenta[c]pyrrole compounds, which may be useful as antagonists of the muscarinic acetylcholine receptor M₄ (mAChR M₄). Also disclosed herein are methods of making the compounds, pharmaceutical compositions comprising the compounds, and methods of treating disorders using the compounds and compositions.

### RELATED APPLICATIONS

This application claims priority to U.S. Provisional Application No. 63/015,248, filed Apr. 24, 2020, which is hereby incorporated by reference in its entirety.

### TECHNICAL FIELD

The present disclosure relates to compounds, compositions, and methods for treating disorders associated with muscarinic acetylcholine receptor dysfunction.

### BACKGROUND

Parkinson's disease (PD) is the second most common neurodegenerative disease with an increasing prevalence as a function of age. Moreover, early-onset PD is also increasing. A hallmark of PD is the progressive degeneration and loss of dopaminergic neurons in the substantia nigra (SN) and basal ganglia (BG), leading to pronounced motor symptoms including bradykinesia, tremor, rigidity, gait dysfunction and postural instability. At present, levodopa (L-DOPA) is the standard of care for treating the motor symptoms, but it is not curative, and prolonged use can engender L-DOPA induced dyskinesia (LID).

Prior to L-DOPA, compounds with anticholinergic activity represented the preferred mode of PD treatment. Cholinergic neurons provide important neuromodulatory control of the BG motor circuit. While the actions of cholinergic pathways on basal ganglia pathways are complex, activation of muscarinic acetylcholine receptors (mAChRs) generally have actions that oppose dopamine (DA) signaling. For instance, mAChR agonists inhibit DA release, and inhibit multiple behavioral effects of drugs that increase DA levels and signaling. Interestingly, muscarinic acetylcholine receptor (mAChR) antagonists were the first available treatments for PD and are still widely used for treatment of this disorder. While many studies of the actions of mAChR antagonists were carried out before randomized controlled trials were introduced, recent well controlled double-blind cross-over design studies demonstrate significant improvement in multiple aspects of motor function in patients receiving mAChR antagonists. Unfortunately, mAChR antagonists have a number of dose-limiting adverse effects that severely limit their clinical utility, including multiple peripheral adverse effects, as well as confusion and severe cognitive disturbances.

Because adverse effects associated with mAChR antagonists limit the doses that can be tolerated, previous clinical studies may underestimate the efficacy that could be achieved if doses of mAChR antagonists could be increased to achieve more complete blockade of specific mAChR subtypes responsible for the antiparkinsonian effects of these agents. The mAChRs include five subtypes, termed M₁-M₅. Available mAChR antagonists, such as scopolamine, are nonselective across these subtypes, and many of their adverse effects are likely mediated by mAChR subtypes that are not involved in the antiparkinsonian activity. Thus, compounds possessing a more selective profile for individual mAChRs may offer an advantage in PD, as well as related disorders such as dystonia. For example, some studies indicate that the M₄ mAChR subtype may play a dominant role in mAChR regulation of basal ganglia motor function.

### SUMMARY

In one aspect, disclosed are compounds of formula (I),

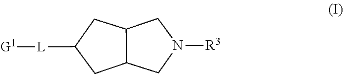

or a pharmaceutically acceptable salt thereof, wherein:
G¹ is
   a) a 5- to 6-membered monocyclic heteroaryl having 1, 2, or 3 heteroatoms independently selected from N, O, and S, the monocyclic heteroaryl being substituted with R^{1a} and 0-2 R^{1b};
   b) a phenyl substituted with R^{1a} and 0-2 R^{1b}; or
   c) an 8- to 12-membered fused bicyclic heteroaryl optionally substituted with 1-5 R²;
R^{1a} is G^{1a}, —O-G^{1a}, —SO₂-G^{1a}, —S(O)-G^{1a}, —C(O)NR^{1c}R^{1d} or halogen;
G^{1a} is a 6- to 12 membered aryl, a 5- to 12-membered heteroaryl, a 4- to 12-membered heterocyclyl, or a C₃₋₁₂carbocyclyl, wherein G^{1a} is optionally substituted with 1-5 substituents independently selected from the group consisting of halogen, cyano, C₁₋₄alkyl, C₁₋₄haloalkyl, —OR¹⁰, —N(R¹⁰)₂, and —NR¹⁰C(O)R¹⁰;
R^{1b}, at each occurrence, is independently halogen, cyano, C₁₋₄alkyl, C₁₋₄haloalkyl, —OR¹¹, or —N(R¹¹)₂;
R^{1c} is hydrogen, C₁₋₄alkyl, C₁₋₄haloalkyl, G^{1a}, or —C₁₋₃alkylene-G^{1a};
R^{1d} is hydrogen, C₁₋₄alkyl, C₁₋₄haloalkyl, C₃₋₄cycloalkyl, or —C₁₋₃alkylene-C₃₋₄cycloalkyl, or R^{1c} and R^{1d}, together with a nitrogen atom to which they attach form a 4- to 8-membered heterocyclic ring optionally substituted with 1-4 substituents independently selected from the group consisting of halogen and C₁₋₄alkyl;
R¹⁰ and R¹¹, at each occurrence, are independently hydrogen, C₁₋₄alkyl, C₁₋₄haloalkyl, C₃₋₄cycloalkyl, or C₁₋₃alkylene-C₃₋₄cycloalkyl, wherein alternatively two R¹⁰ and/or two R¹¹, together with a nitrogen to which the two R¹⁰ or two R¹¹ attach form a 4- to 6-membered heterocyclic ring optionally substituted with 1-4 substituents independently selected from the group consisting of halogen and C₁₋₄alkyl;
R², at each occurrence, is independently halogen, cyano, oxo, C₁₋₄alkyl, C₁₋₄haloalkyl, C₂₋₄alkenyl, C₃₋₆cycloalkyl, or C₁₋₃alkylene-C₃₋₄cycloalkyl;
L is NR, O, —NR—C(O)—; —NR—C₁₋₃alkylene-, or —O—C₁₋₃alkylene-;
R is hydrogen, C₁₋₄alkyl, C₃₋₄cycloalkyl, or —C₁₋₃alkylene-C₃₋₄cycloalkyl;
R³ is G², -L¹-G², -L²-G², -L²-L¹-G², —C₂₋₆alkylene-R^{3a}, or C₃₋₇alkyl;
L¹ is C₁₋₃alkylene;
L² is 1,1-cyclopropylene;
G² is a 6- to 12 membered aryl, a 5- to 12-membered heteroaryl, a 4- to 12-membered heterocyclyl, or a C₃₋₁₂carbocyclyl optionally fused to a phenyl, wherein G² is optionally substituted with 1-5 substituents independently selected from the group consisting of halogen, cyano, C₁₋₄alkyl, C₁₋₄haloalkyl, —OR¹³, —N(R¹³)₂, —C₁₋₃alkylene-OR¹³, and —C₁₋₃alkylene-N(R¹³)₂;
R^{3a} is —OR¹⁴ or —N(R¹⁴)₂; and
R¹³ and R¹⁴, at each occurrence, are independently hydrogen, C₁₋₄alkyl, C₁₋₄haloalkyl, C₃₋₄cycloalkyl, or C₁₋₃alkylene-C₃₋₄cycloalkyl, wherein alternatively two R¹³ or two R¹⁴ together with a nitrogen to which the two R¹³ or two R¹⁴ attach form a 4- to 6-membered heterocyclic ring optionally substituted with 1-4 substituents independently selected from the group consisting of halogen and C₁₋₄alkyl;
provided that R³ is G², -L²-G², -L²-L¹-G², or —C₂₋₆alkylene-R^{3a}, when R^{1a} is G^{1a}-O-G^{1a}, or halogen.

In another aspect, the invention provides a pharmaceutical composition comprising a compound of formula (I), or a pharmaceutically acceptable salt thereof, and a pharmaceutically acceptable carrier.

In another aspect, the invention provides a method of treating a disorder in a subject, wherein the subject would benefit from antagonism of mAChR M₄, comprising administering to the subject a therapeutically effective amount of a compound of formula (I), or a pharmaceutically acceptable salt or composition thereof.

In another aspect, the invention provides a method for antagonizing mAChR M₄ in a subject, comprising administering to the subject a therapeutically effective amount of a compound of formula (I), or a pharmaceutically acceptable salt or composition thereof.

In another aspect, the invention provides a method for the treatment of a neurodegenerative disorder, a movement disorder, or a brain disorder comprising administering to a subject in need thereof a therapeutically effective amount of a compound of formula (I), or a pharmaceutically acceptable salt or composition thereof.

In another aspect, the invention provides a compound of formula (I), or a pharmaceutically acceptable salt or composition thereof, for use in the treatment of a neurodegenerative disorder, a movement disorder, or a brain disorder.

In another aspect, the invention provides a compound of formula (I), or a pharmaceutically acceptable salt or composition thereof, for use in antagonizing mAChR M₄ in a subject.

In another aspect, the invention provides the use of a compound of formula (I), or a pharmaceutically acceptable salt or composition thereof, in the manufacture of a medicament for the treatment of a neurodegenerative disorder, a movement disorder, or a brain disorder.

In another aspect, the invention provides the use of a compound of formula (I), or a pharmaceutically acceptable salt or composition thereof, in the manufacture of a medicament for antagonizing mAChR M₄ in a subject.

In another aspect, the invention provides a kit comprising a compound of formula (I), or a pharmaceutically acceptable salt or composition thereof, and instructions for use.

Also disclosed are pharmaceutical compositions comprising the compounds, methods of making the compounds, kits comprising the compounds, and methods of using the compounds, compositions and kits for treatment of disorders, such as neurological and/or psychiatric disorders, associated with muscarinic acetylcholine receptor dysfunction in a mammal.

### DETAILED DESCRIPTION

Disclosed herein are compounds that are antagonists of the muscarinic acetylcholine receptor M₄ (mAChR M₄), methods of making the compounds, pharmaceutical compositions comprising the compounds, and methods of treating disorders using the compounds and pharmaceutical compositions. The compounds include substituted hexahydro-1H-cyclopenta[c]pyrrole compounds.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to a group —O-alkyl. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkoxyalkyl,” as used herein, refers to an alkoxy group, as defined herein, appended to the parent molecular moiety through an alkyl group, as defined herein.

The term “alkoxyfluoroalkyl,” as used herein, refers to an alkoxy group, as defined herein, appended to the parent molecular moiety through a fluoroalkyl group, as defined herein.

The term “alkylene,” as used herein, refers to a divalent group derived from a straight or branched chain hydrocarbon, for example, of 1 to 3 carbon atoms. Representative examples of alkylene include, but are not limited to, —CH₂—, —CH(CH₃)—, —C(CH₃)₂—, —CH₂CH₂—, —CH(CH₃)CH₂—, —C(CH₃)₂CH₂—, —CH₂CH₂CH₂—, —CH(CH₃)CH₂CH₂—, —C(CH₃)₂CH₂CH₂—, —CH₂C(CH₃)₂CH₂—, —CH₂CH₂CH₂CH₂—, and —CH₂CH₂CH₂CH₂CH₂—.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl,” as used herein, means at least one amino group, as defined herein, is appended to the parent molecular moiety through an alkylene group, as defined herein.

The term “amino,” as used herein, means —NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cyanoalkyl,” as used herein, means at least one —CN group, is appended to the parent molecular moiety through an alkylene group, as defined herein.

The term “cyanofluoroalkyl,” as used herein, means at least one —CN group, is appended to the parent molecular moiety through a fluoroalkyl group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl). Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The term “1,1-carbocyclylene” means a geminal divalent group derived from a cycloalkyl. A representative example is 1,1-C₃₋₆cycloalkylene

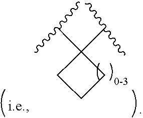

A further example is 1,1-cyclopropylene

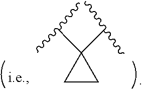

The term “fluoroalkyl,” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “fluoroalkoxy,” as used herein, means at least one fluoroalkyl group, as defined herein, is appended to the parent molecular moiety through an oxygen atom. Representative examples of fluoroalkoxy include, but are not limited to, difluoromethoxy, trifluoromethoxy and 2,2,2-trifluoroethoxy.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered aromatic monocyclic rings have three double bonds. The bicyclic heteroaryl is an 8- to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, and 3-oxaspiro[5.5]undecanyl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

The term “hydroxyfluoroalkyl,” as used herein, means at least one —OH group, is appended to the parent molecular moiety through a fluoroalkyl group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

The term “mAChR M₄ receptor antagonist” as used herein refers to any exogenously administered compound or agent that directly or indirectly antagonizes mAChR M₄, for example in an animal, in particular a mammal (e.g., a human).

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. COMPOUNDS

In one aspect, the invention provides compounds of formula (I), wherein L, G¹ and R³ are as defined herein.

Unsubstituted or substituted rings (i.e., optionally substituted) such as aryl, heteroaryl, etc. are composed of both a ring system and the ring system's optional substituents. Accordingly, the ring system may be defined independently of its substituents, such that redefining only the ring system leaves any previous optional substituents present. For example, a 5- to 12-membered heteroaryl with optional substituents may be further defined by specifying the ring system of the 5- to 12-membered heteroaryl is a 5- to 6-membered heteroaryl (i.e., 5- to 6-membered heteroaryl ring system), in which case the optional substituents of the 5- to 12-membered heteroaryl are still present on the 5- to 6-membered heteroaryl, unless otherwise expressly indicated.

In a first embodiment are compounds of formula (I), wherein G¹ is a 5- to 6-membered monocyclic heteroaryl having 1, 2, or 3 heteroatoms independently selected from N, O, and S, or G¹ is a phenyl, wherein G¹ is substituted with R^{1a} and 0-2 R^{1b}; R^{1a} is G^{1a}, —O-G^{1a}, or halogen; G^{1a} is a 6- to 12-membered aryl, a 5- to 12-membered heteroaryl, a 4- to 12-membered heterocyclyl, or a C₃₋₁₂carbocyclyl, wherein G^{1a} is optionally substituted with 1-5 substituents independently selected from the group consisting of halogen, cyano, C₁₋₄alkyl, C₁₋₄haloalkyl, —OR¹⁰, —N(R¹⁰)₂, and —NR¹⁰C(O)R¹⁰; R^{1b}, at each occurrence, is independently halogen, cyano, C₁₋₄alkyl, C₁₋₄haloalkyl, —OR¹¹, or —N(R¹¹)₂; R¹⁰ and R¹¹, at each occurrence, are independently hydrogen, C₁₋₄alkyl, C₁₋₄haloalkyl, C₃₋₄cycloalkyl, or C₁₋₃alkylene-C₃₋₄cycloalkyl, wherein alternatively two R¹⁰ and/or two R¹¹, together with a nitrogen to which the two R¹⁰ or two R¹¹ attach form a 4- to 6-membered heterocyclic ring optionally substituted with 1-4 substituents independently selected from the group consisting of halogen and C₁₋₄alkyl; L is NR, O, —NR—C(O)—; —NR—C₁₋₃alkylene-, or —O—C₁₋₃alkylene-; R is hydrogen, C₁₋₄alkyl, C₃₋₄cycloalkyl, —C₁₋₃alkylene-C₃₋₄cycloalkyl; R³ is G², -L²-G², -L²-L¹-G², or —C₂₋₆alkylene-R^{3a}; L¹ is C₁₋₃alkylene; L² is 1,1-cyclopropylene; G² is a 6- to 12-membered aryl, a 5- to 12-membered heteroaryl, a 4- to 12-membered heterocyclyl, or a C₃₋₁₂carbocyclyl optionally fused to a phenyl, wherein G² is optionally substituted with 1-5 substituents independently selected from the group consisting of halogen, cyano, C₁₋₄alkyl, C₁₋₄haloalkyl, —OR¹³, —N(R¹³)₂, —C₁₋₃alkylene-OR¹³, and —C₁₋₃alkylene-N(R¹³)₂; R^{3a} is —OR¹⁴ or —N(R¹⁴)₂; and R¹³ and R¹⁴, at each occurrence, are independently hydrogen, C₁₋₄alkyl, C₁₋₄haloalkyl, C₃₋₄cycloalkyl, or C₁₋₃alkylene-C₃₋₄cycloalkyl, wherein alternatively two R¹³ or two R¹⁴, together with a nitrogen to which the two R¹³ or two R¹⁴ attach form a 4- to 6-membered heterocyclic ring optionally substituted with 1-4 substituents independently selected from the group consisting of halogen and C₁₋₄alkyl, optionally wherein G¹-L- is not

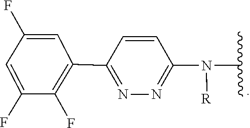

In a second embodiment are compounds of formula (I), wherein G¹ is a) a 5- to 6-membered monocyclic heteroaryl having 1, 2, or 3 heteroatoms independently selected from N, O, and S, the monocyclic heteroaryl being substituted with R^{1a} and 0-2 R^{1b}; b) a phenyl substituted with R^{1a} and 0-2 R^{1b}; or c) an 8- to 12-membered fused bicyclic heteroaryl optionally substituted with 1-5 R²; R^{1a} is —SO₂-G^{1a}, —S(O)-G^{1a}, or —C(O)NR^{1c}R^{1d}; G^{1a} is a 6- to 12-membered aryl, a 5- to 12-membered heteroaryl, a 4- to 12-membered heterocyclyl, or a C₃₋₁₂carbocyclyl, wherein G^{1a} is optionally substituted with 1-5 substituents independently selected from the group consisting of halogen, cyano, C₁₋₄alkyl, C₁₋₄haloalkyl, —OR¹⁰, —N(R¹⁰)₂, and —NR¹⁰C(O)R¹⁰; R^{1b}, at each occurrence, is independently halogen, cyano, C₁₋₄alkyl, C₁₋₄haloalkyl, —OR¹¹, or —N(R¹¹)₂; R¹¹ is hydrogen, C₁₋₄alkyl, C₁₋₄haloalkyl, G^{1a}, or —C₁₋₃alkylene-G^{1a}; R^{1d} is hydrogen, C₁₋₄alkyl, C₁₋₄haloalkyl, C₃₋₄cycloalkyl, or —C₁₋₃alkylene-C₃₋₄cycloalkyl, or R^{1c} and R^{1d}, together with a nitrogen atom to which they attach form a 4- to 8-membered heterocyclic ring optionally substituted with 1-4 substituents independently selected from the group consisting of halogen and C₁₋₄alkyl; R¹⁰ and R¹¹, at each occurrence, are independently hydrogen, C₁₋₄alkyl, C₁₋₄haloalkyl, C₃₋₄cycloalkyl, or C₁₋₃alkylene-C₃₋₄cycloalkyl, wherein alternatively two R¹⁰ and/or two R¹¹, together with a nitrogen to which the two R¹⁰ or two R¹¹ attach form a 4- to 6-membered heterocyclic ring optionally substituted with 1-4 substituents independently selected from the group consisting of halogen and C₁₋₄alkyl; R², at each occurrence, is independently halogen, cyano, oxo, C₁₋₄alkyl, C₁₋₄haloalkyl, C₂₋₄alkenyl, C₃₋₆cycloalkyl, or C₁₋₃alkylene-C₃₋₄cycloalkyl; L is NR, O, —NR—C(O)—; —NR—C₁₋₃alkylene-, or —O—C₁₋₃alkylene-; R is hydrogen, C₁₋₄alkyl, C₃₋₄cycloalkyl, —C₁₋₃alkylene-C₃₋₄cycloalkyl; R³ is G², -L¹-G², -L²-G², -L²-L¹-G², —C₂₋₆alkylene-R^{3a}, or C₃₋₇alkyl; L¹ is C₁₋₃alkylene; L² is 1,1-cyclopropylene; G² is a 6- to 12 membered aryl, a 5- to 12-membered heteroaryl, a 4- to 12-membered heterocyclyl, or a C₃₋₁₂carbocyclyl optionally fused to a phenyl, wherein G² is optionally substituted with 1-5 substituents independently selected from the group consisting of halogen, cyano, C₁₋₄alkyl, C₁₋₄haloalkyl, —OR¹³, —N(R¹³)₂, —C₁₋₃alkylene-OR¹³, and —C₁₋₃alkylene-N(R¹³)₂; R^{3a} is —OR¹⁴ or —N(R¹⁴)₂; and R¹³ and R¹⁴, at each occurrence, are independently hydrogen, C₁₋₄alkyl, C₁₋₄haloalkyl, C₃₋₄cycloalkyl, or C₁₋₃alkylene-C₃₋₄cycloalkyl, wherein alternatively two R¹³ or two R¹⁴, together with a nitrogen to which the two R¹³ or two R¹⁴ attach form a 4- to 6-membered heterocyclic ring optionally substituted with 1-4 substituents independently selected from the group consisting of halogen and C₁₋₄alkyl.

In formula (I) and according to the embodiments herein, G¹ may be a 5- to 6-membered monocyclic heteroaryl having 1, 2, or 3 heteroatoms independently selected from N, O, and S, the monocyclic heteroaryl being substituted with R^{1a} and 0-2 R^{1b}, wherein R^{1a} and R^{1b} are as defined herein. The 5- to 6-membered monocyclic heteroaryl may have 1 or 2 heteroatoms independently selected from the group consisting of N and S. The ring system of the 5- to 6-membered monocyclic heteroaryl may be a pyridazinyl or a thiazolyl. The 5- to 6-membered monocyclic heteroaryl at G¹ may be

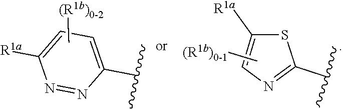

The 5- to 6-membered monocyclic heteroaryl at G¹ may be substituted with zero R^{1b}, such as

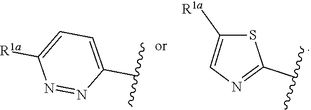

The 5- to 6-membered monocyclic heteroaryl at G¹ may be substituted with one R^{1b}, such as

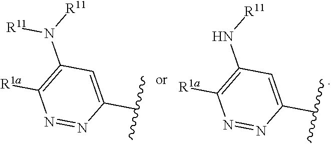

In the foregoing, R¹¹ may be C₃₋₄cycloalkyl. For example, G¹ may be

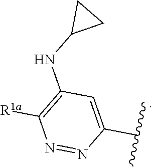

When G¹ is the 5- to 6-membered monocyclic heteroaryl, R^{1a} may be G^{1a}. G^{1a} may be a 6- to 12-membered aryl, optionally substituted as defined herein. The optionally substituted 6- to 12-membered aryl may be an optionally substituted phenyl. The optionally substituted phenyl may be phenyl,

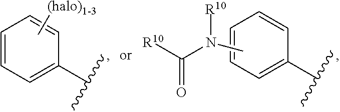

optionally wherein G^{1a} is not

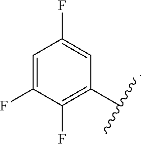 [SMILES: *c1cc(F)cc(F)c1F]

The optionally substituted phenyl may be phenyl,

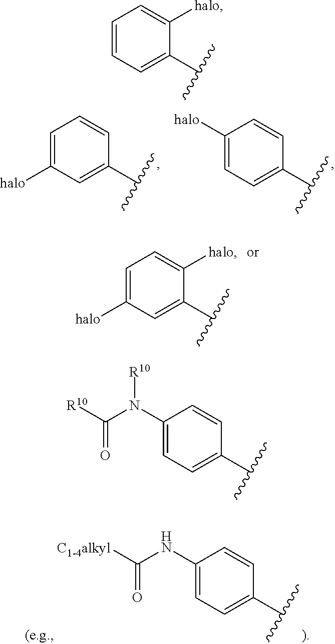

The halo in the optionally substituted phenyl may be fluoro or chloro. The optionally substituted phenyl may be phenyl,

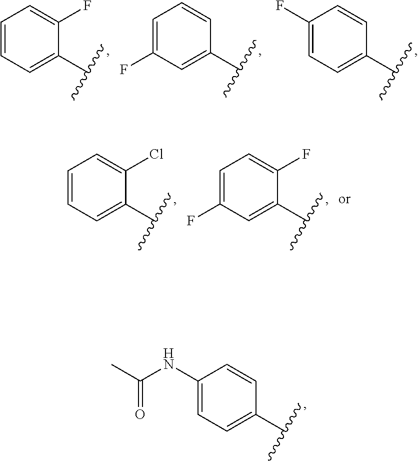

When R^{1a} is G^{1a}, G^{1a} may also be a 5- to 12-membered heteroaryl, optionally substituted as defined herein. The optionally substituted 5- to 12-membered heteroaryl may be an optionally substituted pyridinyl, pyrazolyl, indazolyl, or imidazopyridinyl. The optionally substituted 5- to 12-membered heteroaryl may be

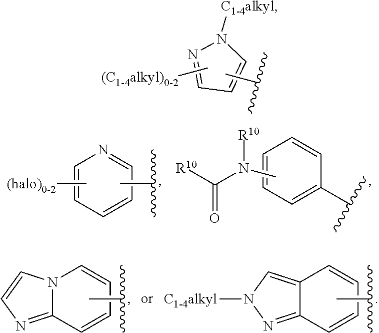

The halo in the optionally substituted heteroaryl may be fluoro or chloro. The optionally substituted 5- to 12-membered heteroaryl may be

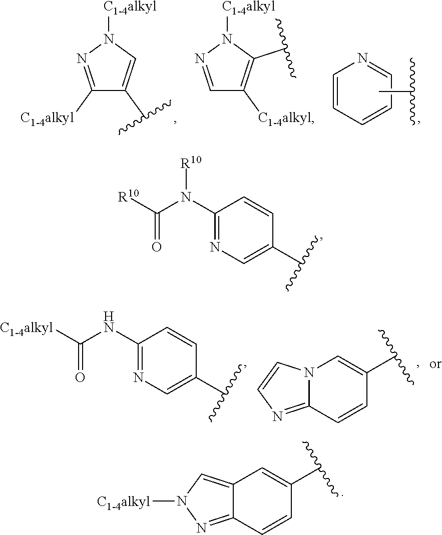

The optionally substituted 5- to 12-membered heteroaryl may be

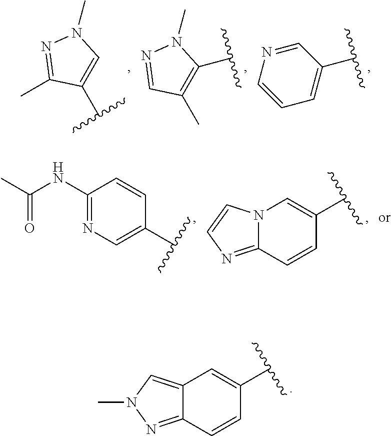

When R^{1a} is G^{1a}, G^{1a} may also be a 4- to 12-membered heterocyclyl, optionally substituted as defined herein. The optionally substituted 4- to 12-membered heterocyclyl may be an optionally substituted 4- to 8-membered monocyclic heterocyclyl containing 1-2 heteroatoms independently selected from the group consisting of N and O. The optionally substituted 4- to 8-membered monocyclic heterocyclyl containing 1-2 heteroatoms independently selected from the group consisting of N and O may be an optionally substituted morpholine or piperidine. The optionally substituted 4- to 8-membered monocyclic heterocyclyl containing 1-2 heteroatoms independently selected from the group consisting of N and O may be

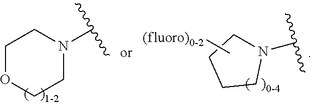

The optionally substituted 4- to 8-membered monocyclic heterocyclyl containing 1-2 heteroatoms independently selected from the group consisting of N and O may be

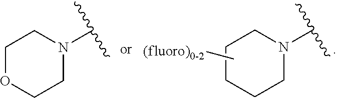

The optionally substituted 4- to 8-membered monocyclic heterocyclyl containing 1-2 heteroatoms independently selected from the group consisting of N and O may be

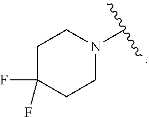 [SMILES: *N1CCC(F)(F)CC1]

When G¹ is the 5- to 6-membered monocyclic heteroaryl, R^{1a} may be —SO₂-G^{1a}, —S(O)-G^{1a}, or —C(O)NG^{1a}R^{1d}, wherein G^{1a} may be a 6- to 12-membered aryl, optionally substituted as defined herein. The optionally substituted 6- to 12-membered aryl may be an optionally substituted phenyl. The optionally substituted phenyl may be phenyl or

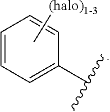

The optionally substituted phenyl may be phenyl,

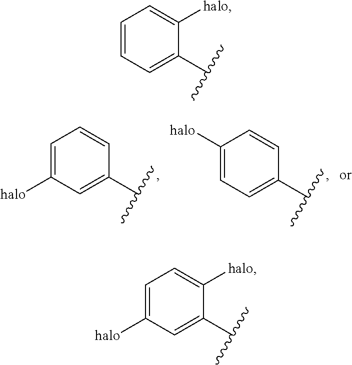

The halo in the optionally substituted phenyl may be fluoro or chloro. The optionally substituted phenyl may be phenyl,

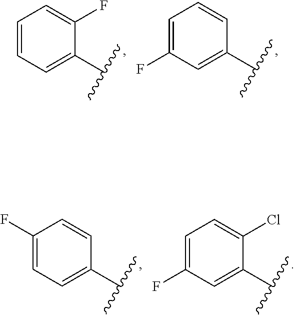

When R^{1a} is —SO₂-G^{1a} or —S(O)-G^{1a}, the optionally substituted phenyl at G^{1a} may be phenyl. When R^{1a} is —C(O)NG^{1a}R^{1d}, the optionally substituted phenyl at G^{1a} may be phenyl or

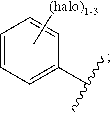

the optionally substituted phenyl at G^{1a} may be phenyl

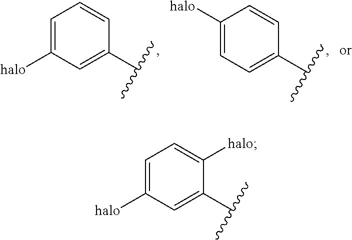

or the halo may be fluoro or chloro; or the optionally substituted phenyl at G^{1a} may be phenyl,

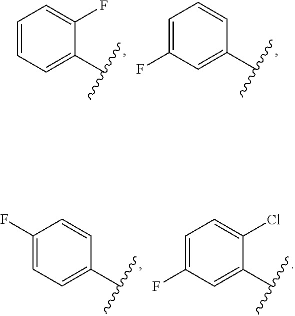

In the foregoing description of R^{1a}, R^{1d} may be hydrogen. In some embodiments, R^{1d} is preferably hydrogen.

When R^{1a} is —SO₂-G^{1a}, —S(O)-G^{1a}, or —C(O)NG^{1a}R^{1d}, G^{1a} may be a C₃₋₁₂carbocyclyl, optionally substituted as defined herein. The optionally substituted C₃₋₁₂carbocyclyl may be an optionally substituted C₃₋₈cycloalkyl. The optionally substituted C₃₋₈cycloalkyl may be a cyclopropyl or cyclohexyl. When R^{1a} is —SO₂-G^{1a} or —S(O)-G^{1a}, G^{1a} may be an optionally substituted C₃₋₈cycloalkyl; or G^{1a} may be cyclohexyl. When R^{1a} is —C(O)NG^{1a}R^{1d}, G^{1a} may be an optionally substituted C₃₋₈cycloalkyl; or G^{1a} may be cyclopropyl or cyclohexyl. In the foregoing description of R^{1a}, R^{1d} may be hydrogen. In some embodiments, R^{1d} is preferably hydrogen.

In formula (I) and according to the embodiments herein, G¹ may be a phenyl substituted with R^{1a} and 0-2 R^{1b}, wherein R^{1a} and R^{1b} are as defined herein.

When G¹ is the 5- to 6-membered monocyclic heteroaryl, R^{1a} may be halogen (e.g., chloro).

In compounds of formula (I), R^{1a} may be —O-G^{1a}.

When G¹ is the 5- to 6-membered monocyclic heteroaryl, R^{1a} may be —C(O)NR^{1c}R^{1d}. R^{1c} and R^{1d}, together with a nitrogen atom to which they attach may form a 4- to 8-membered heterocyclic ring optionally substituted with 1-4 substituents independently selected from the group consisting of halogen and C₁₋₄alkyl. The optionally substituted heterocycle may be optionally substituted morpholine or a piperidine. The optionally substituted heterocycle may be morpholine or a piperidine.

The 5- to 6-membered monocyclic heteroaryl and phenyl at G¹ are substituted with 0-2 R^{1b}, where R^{1b} is as defined herein. In some embodiments, the compounds of formula (I) have zero R^{b} substituents. In some embodiments, the compounds of formula (I) have one R^{b} substituent (e.g., CF₃, CN, —N(R¹¹)₂).

In compounds of formula (I), L may be NR. In compounds of formula (I), L may be —NR—C₁₋₃alkylene-. In compounds of formula (I), L may be —NR—C(O)—. In compounds of formula (I), R may be hydrogen.

In compounds of formula (I), L may be O. In compounds of formula (I), L may be —O—C₁₋₃alkylene-.

Compounds of formula (I) may have formula (I-A), (I-B), or (I-C).

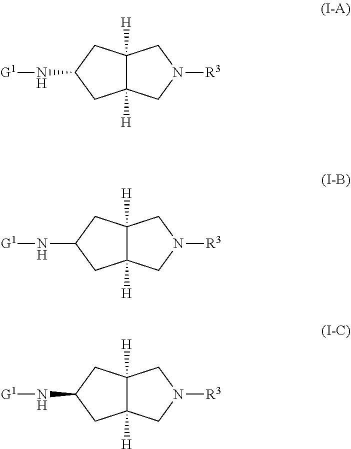

When G¹ is the 5- to 6-membered monocyclic heteroaryl, compounds of formula (I) may have formula (I-a), (I-b), (I-c), (I-d), (I-e), (I-f), (I-g), or (I-h), wherein R, G^{1a}, R^{b}, R^{1d}, and R³ are as defined herein.

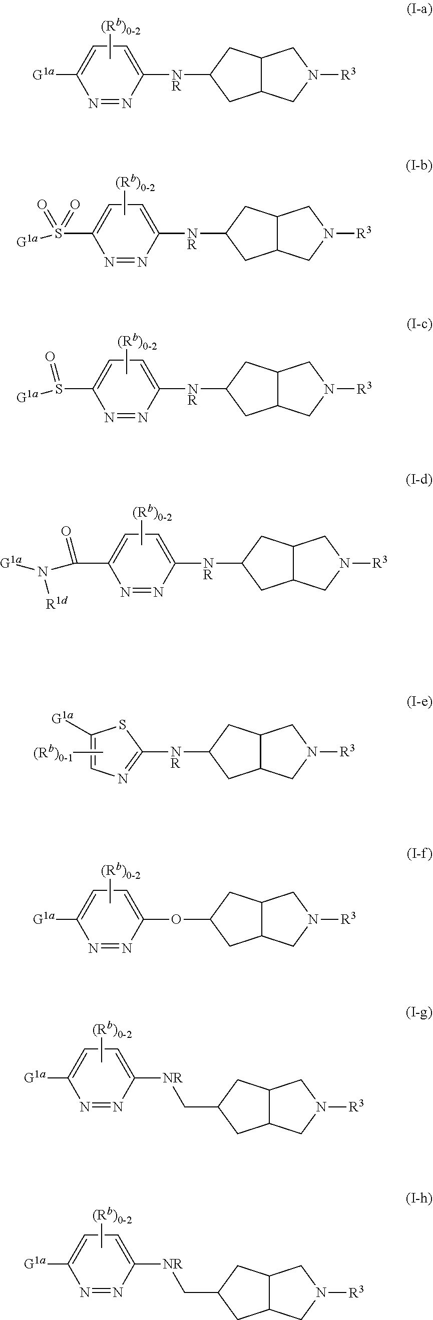

When G¹ is the 5- to 6-membered monocyclic heteroaryl, compounds of formula (I) may have formula (I-a1), (I-b1), (I-c1), (I-d1), or (I-e1), wherein R, G^{1a}, R^{b}, R^{1d}, and R³ are as defined herein.

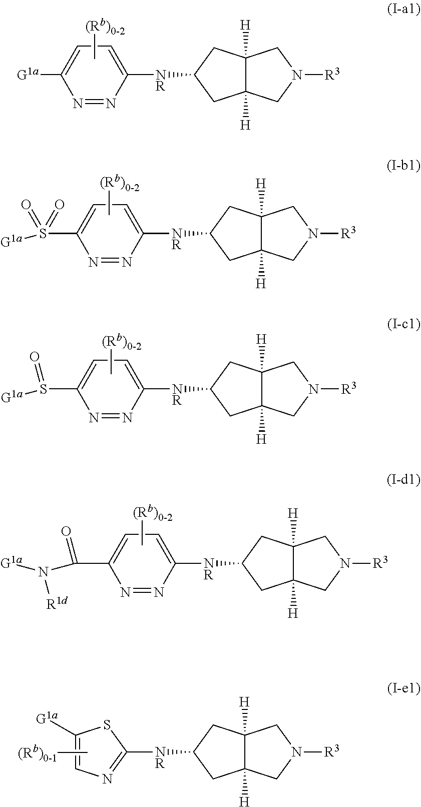

In some embodiments, the compounds of any of formulas (I-a), (I-b), (I-c), (I-d), (I-e), (I-f), (I-g), (I-h), (I-a1), (I-b1), (I-c1), (I-d1), or (I-e1) have zero R^{b} substituents, e.g.,

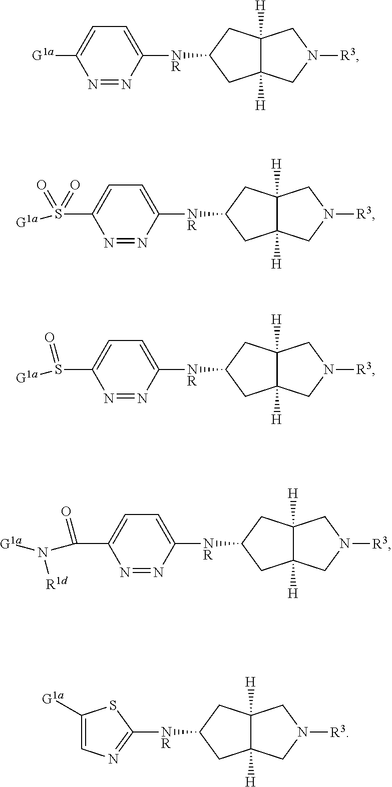

In some embodiments, the compounds of any of formulas (I-a), (I-b), (I-c), (I-d), (I-e), (I-f), (I-g), (I-h), (I-a1), (I-b1), (I-c1), (I-d1), or (I-e1) have one R^{b} substituent (e.g., CF₃, CN). In an embodiment, formula (I-b)/(I-b1) has one R^{b} substituent, wherein R^{b} is —N(R¹¹)₂ and R¹¹ is as defined herein

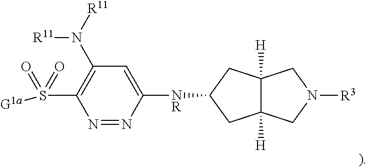

In a further embodiment R^{b} is —NHR¹¹, wherein R¹¹ is C₃₋₄cycloalkyl

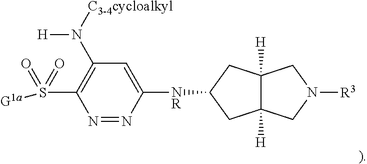

In formula (I) and according to the embodiments herein, G¹ may be an 8- to 12-membered fused bicyclic heteroaryl optionally substituted with 1-5 R². The 8- to 12-membered fused bicyclic heteroaryl may be a 9-membered fused bicyclic aromatic ring system having four double bonds and a nitrogen atom at the ring junction. The 9-membered fused bicyclic aromatic ring system having four double bonds and a nitrogen atom at the ring junction may be a [1,2,4]triazolo[4,3-b]pyridazinyl. For example, G¹ may be

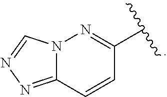 [SMILES: *c1ccc2nncn2n1]

The 8- to 12-membered fused bicyclic heteroaryl may be a pyridazin-3-yl fused to a pyrrolidine. The pyridazin-3-yl fused to a pyrrolidine may be a pyrrolo[2,3-c]pyridazin-3-yl. The pyrrolo[2,3-c]pyridazin-3-yl may be substituted with 1-4 R² substituents independently selected from the group consisting of oxo, C₁₋₄alkyl, C₂₋₄alkenyl, and C₃₋₆cycloalkyl. For example, G¹ may be

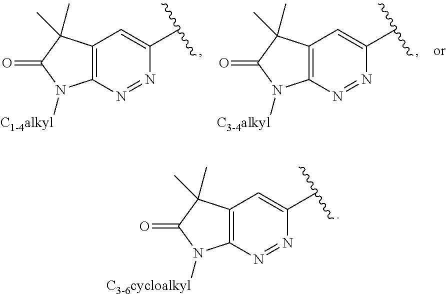

When G¹ is the 8- to 12-membered fused bicyclic heteroaryl, compounds of formula (I) may have formula (I-l), (I-m), (I-n), or (I-o), wherein R and R³ are as defined herein.

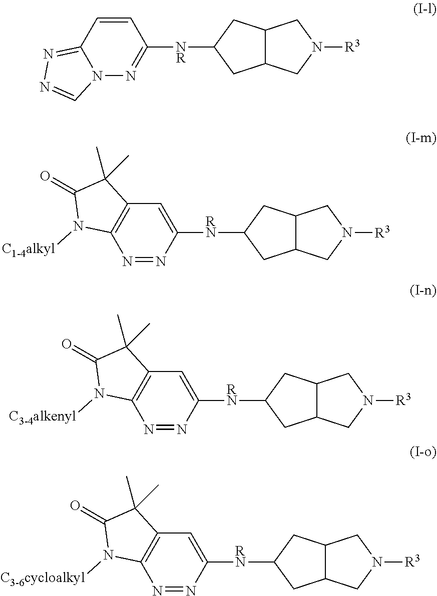

When G¹ is the 8- to 12-membered fused bicyclic heteroaryl, compounds of formula (I) may have formula (I-l1), (I-m1), (I-n1), or (I-o1), wherein R and R³ are as defined herein.

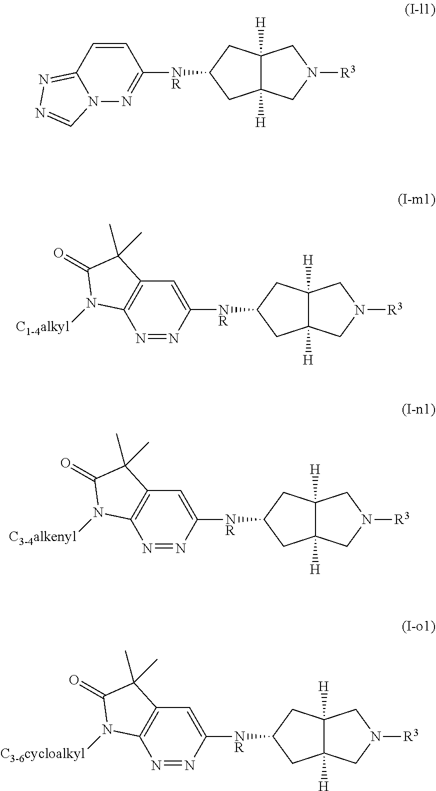

In formula (I) and according to the embodiments herein, R³ may be G², wherein G² is an optionally substituted 4- to 12-membered heterocyclyl. The optionally substituted 4- to 12-membered heterocyclyl may be an optionally substituted 4- to 8-membered monocyclic heterocyclyl or 7- to 12-membered spiro heterocyclyl, wherein the heterocyclyls contain one heteroatom selected from O and S. The optionally substituted 4- to 12-membered heterocyclyl may be an optionally substituted oxetanyl, tetrahydropyranyl, tetrahydrothiopyranyl, 2-oxaspiro[3.3]heptanyl, or a 3-oxaspiro[5.5]undecanyl. The optionally substituted 4- to 12-membered heterocyclyl may be

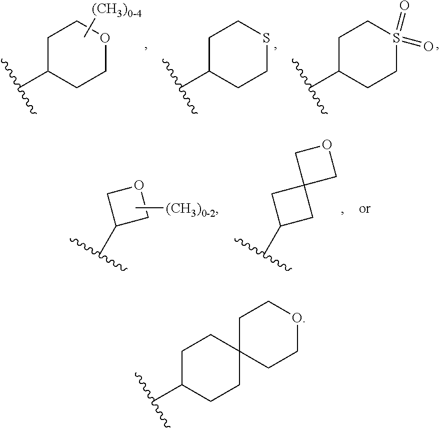

The optionally substituted 4- to 12-membered heterocyclyl may be

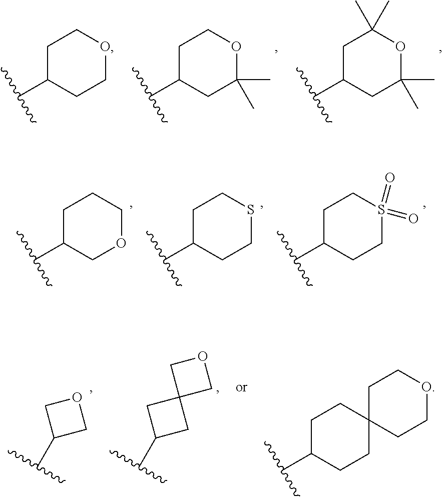

In formula (I) and according to the embodiments herein, R³ may be G², wherein G² is an optionally substituted 6- to 12-membered aryl. The optionally substituted 6- to 12-membered aryl may be an optionally substituted phenyl. The optionally substituted phenyl may be

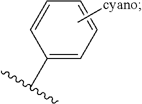

or the optionally substituted phenyl may be

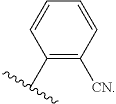 [SMILES: *c1ccccc1C#N]

In formula (I) and according to the embodiments herein, R³ may be G², wherein G² is an optionally substituted C₃₋₁₂carbocyclyl optionally fused to a phenyl. The optionally substituted C₃₋₁₂carbocyclyl optionally fused to a phenyl may be an optionally substituted C₃₋₈cycloalkyl optionally fused to a phenyl. The optionally substituted C₃₋₁₂carbocyclyl optionally fused to a phenyl may be a spiro[5.5]undecanyl. The optionally substituted C₃₋₁₂carbocyclyl optionally fused to a phenyl may be

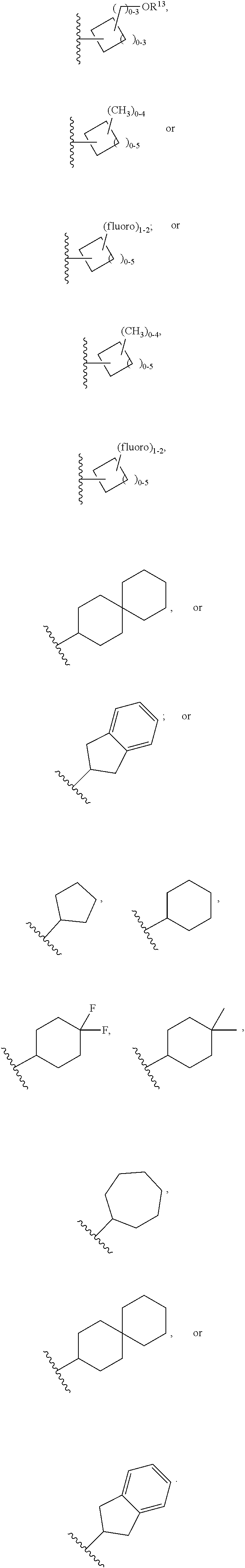

In formula (I) and according to the embodiments herein, R³ may be -L¹-G², wherein L¹ is as defined herein, and G² is an optionally substituted 4- to 12-membered heterocyclyl. The optionally substituted 4- to 12-membered heterocyclyl may be an optionally substituted 4- to 8-membered monocyclic heterocyclyl containing one oxygen atom. The optionally substituted 4- to 8-membered monocyclic heterocyclyl may be an optionally substituted tetrahydropyranyl. The optionally substituted 4- to 8-membered monocyclic heterocyclyl may be

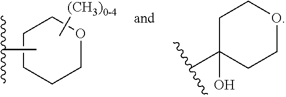

The optionally substituted 4- to 8-membered monocyclic heterocyclyl may be

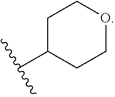 [SMILES: *C1CCOCC1]

In formula (I) and according to the embodiments herein, R³ may be -L¹-G², wherein L¹ is as defined herein, and G² is an optionally substituted 6- to 12-membered aryl. The optionally substituted 6- to 12-membered aryl may be an optionally substituted phenyl or a phenyl bonded to the parent molecule and fused to a 5- to 7-membered heterocycle containing 1-2 oxygen atoms. The optionally substituted 6- to 12-membered aryl may be

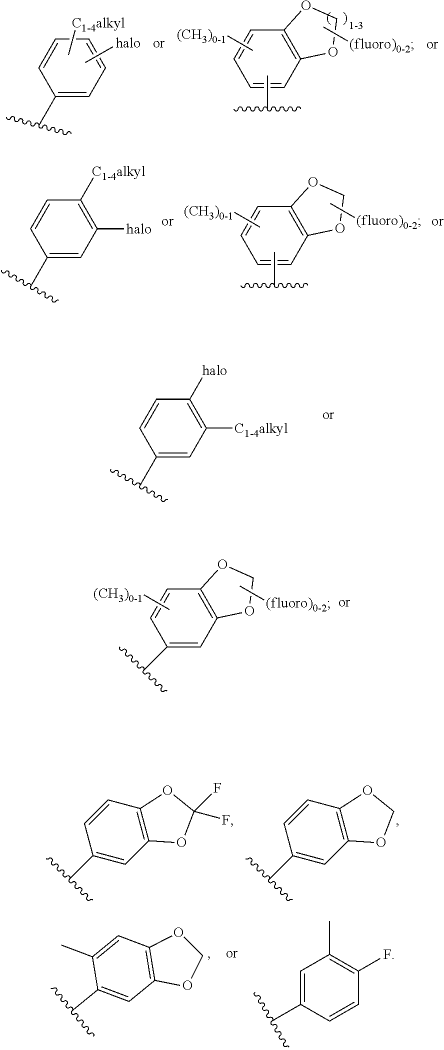

In formula (I) and according to the embodiments herein, R³ may be -L¹-G², wherein L¹ is as defined herein, and G² is an optionally substituted 5- to 12-membered heteroaryl. The optionally substituted 5- to 12-membered heteroaryl may be an optionally substituted pyrazolyl, pyridinyl, or indolyl. The optionally substituted 5- to 12-membered heteroaryl may be

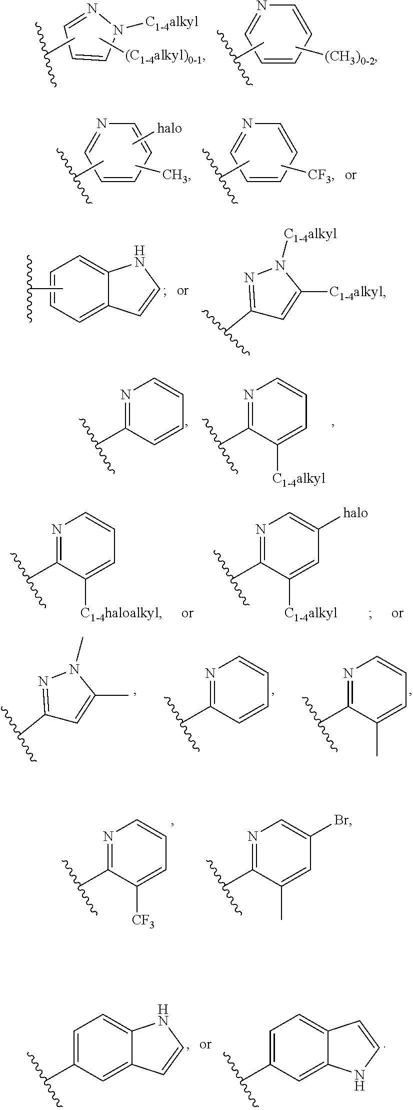

In formula (I) and according to the embodiments herein, R³ may be -L¹-G², wherein G² is as defined herein, and L¹ is CH₂.

In formula (I) and according to the embodiments herein, R³ may be -L²-G². When R³ is -L²-G², G² may be an optionally substituted 4- to 8-membered monocyclic heterocyclyl containing one oxygen atom. The optionally substituted 4- to 8-membered monocyclic heterocyclyl may be an optionally substituted tetrahydropyranyl. The optionally substituted 4- to 8-membered monocyclic heterocyclyl may be

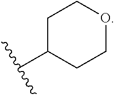 [SMILES: *C1CCOCC1]

In formula (I) and according to the embodiments herein, R³ may be C₃₋₇alkyl.

In formula (I) and according to the embodiments herein, R³ may be —C₂₋₆alkylene-OR¹⁴. R¹⁴ may be C₁₋₄alkyl. R¹⁴ may be hydrogen. R³ may be —(CH₂)₃—OCH₃ or —(CH₂)C(CH₃)₂OH.

Throughout the embodiments and description of the compounds of the invention, all instances of haloalkyl may be fluoroalkyl (e.g., any C₁₋₄haloalkyl may be C₁₋₄fluoroalkyl).

Representative compounds of formula (I) include, but are not limited to:
(3aR,5s,6aS)—N-(6-(2-chloro-5-fluorophenyl)pyridazin-3-yl)-2-(3-methoxypropyl)octahydrocyclopenta[c]pyrrol-5-amine;
((3aR,5s,6aS)—N-(6-(2-chloro-5-fluorophenyl)pyridazin-3-yl)-2-(tetrahydro-2H-pyran-4-yl)octahydrocyclopenta[c]pyrrol-5-amine;
N-cyclopropyl-6-(((3aR,5s,6aS)-2-((tetrahydro-2H-pyran-4-yl)methyl)octahydrocyclopenta[c]pyrrol-5-yl)amino)pyridazine-3-carboxamide;
N-cyclohexyl-6-(((3aR,5s,6aS)-2-((tetrahydro-2H-pyran-4-yl)methyl)octahydrocyclopenta[c]pyrrol-5-yl)amino)pyridazine-3-carboxamide;
piperidin-1-yl(6-(((3aR,5s,6aS)-2-((tetrahydro-2H-pyran-4-yl)methyl)octahydrocyclopenta[c]pyrrol-5-yl)amino)pyridazin-3-yl)methanone;
morpholino(6-(((3aR,5s,6aS)-2-((tetrahydro-2H-pyran-4-yl)methyl)octahydrocyclopenta[c]pyrrol-5-yl)amino)pyridazin-3-yl)methanone;
N-phenyl-6-(((3aR,5s,6aS)-2-((tetrahydro-2H-pyran-4-yl)methyl)octahydrocyclopenta[c]pyrrol-5-yl)amino)pyridazine-3-carboxamide;
N-(2-fluorophenyl)-6-(((3aR,5s,6aS)-2-((tetrahydro-2H-pyran-4-yl)methyl)octahydrocyclopenta[c]pyrrol-5-yl)amino)pyridazine-3-carboxamide;
N-(3-fluorophenyl)-6-(((3aR,5s,6aS)-2-((tetrahydro-2H-pyran-4-yl)methyl)octahydrocyclopenta[c]pyrrol-5-yl)amino)pyridazine-3-carboxamide;
N-(4-fluorophenyl)-6-(((3aR,5s,6aS)-2-((tetrahydro-2H-pyran-4-yl)methyl)octahydrocyclopenta[c]pyrrol-5-yl)amino)pyridazine-3-carboxamide;
N-(2-chloro-5-fluorophenyl)-6-(((3aR,5s,6aS)-2-((tetrahydro-2H-pyran-4-yl)methyl)octahydrocyclopenta[c]pyrrol-5-yl)amino)pyridazine-3-carboxamide;
(3aR,5s,6aS)—N-(6-(2-chloro-5-fluorophenyl)pyridazin-3-yl)-2-cyclohexyloctahydrocyclopenta[c]pyrrol-5-amine;
(3aR,5s,6aS)—N-(6-(2-chloro-5-fluorophenyl)pyridazin-3-yl)-2-(tetrahydro-2H-pyran-3-yl)octahydrocyclopenta[c]pyrrol-5-amine;
(3aR,5s,6aS)—N-(6-(2-chloro-5-fluorophenyl)pyridazin-3-yl)-2-(oxetan-3-yl)octahydrocyclopenta[c]pyrrol-5-amine;
(3aR,5s,6aS)—N-(6-(2-chloro-5-fluorophenyl)pyridazin-3-yl)-2-(tetrahydro-2H-thiopyran-4-yl)octahydrocyclopenta[c]pyrrol-5-amine;
(3aR,5s,6aS)—N-(6-(2-chloro-5-fluorophenyl)pyridazin-3-yl)-2-(2,2-dimethyltetrahydro-2H-pyran-4-yl)octahydrocyclopenta[c]pyrrol-5-amine;
2-((3aR,5s,6aS)-5-((6-(2-chloro-5-fluorophenyl)pyridazin-3-yl)amino)hexahydrocyclopenta[c]pyrrol-2(1H)-yl)benzonitrile;
(3aR,5s,6aS)—N-(6-(2-chloro-5-fluorophenyl)pyridazin-3-yl)-2-(2,2,6,6-tetramethyltetrahydro-2H-pyran-4-yl)octahydrocyclopenta[c]pyrrol-5-amine;
N-((3aR,5s,6aS)-2-(2,2-dimethyltetrahydro-2H-pyran-4-yl)octahydrocyclopenta[c]pyrrol-5-yl)-5-phenylthiazol-2-amine;
N-(4-(6-(((3aR,5s,6aS)-2-(2,2-dimethyltetrahydro-2H-pyran-4-yl)octahydrocyclopenta[c]pyrrol-5-yl)amino)pyridazin-3-yl)phenyl)acetamide;
(3aR,5s,6aS)-2-(2,2-dimethyltetrahydro-2H-pyran-4-yl)-N-(6-(imidazo[1,2-a]pyridin-6-yl)pyridazin-3-yl)octahydrocyclopenta[c]pyrrol-5-amine;
(3aR,5s,6aS)—N-(6-(1,3-dimethyl-1H-pyrazol-4-yl)pyridazin-3-yl)-2-(2,2-dimethyltetrahydro-2H-pyran-4-yl)octahydrocyclopenta[c]pyrrol-5-amine;
(3aR,5s,6aS)—N-(6-(phenylsulfonyl)pyridazin-3-yl)-2-((tetrahydro-2H-pyran-4-yl)methyl)octahydrocyclopenta[c]pyrrol-5-amine;
(3aR,5s,6aS)-2-((1,5-dimethyl-1H-pyrazol-3-yl)methyl)-N-(6-(phenylsulfonyl)pyridazin-3-yl)octahydrocyclopenta[c]pyrrol-5-amine;
(3aR,5s,6aS)—N-(6-(phenylsulfonyl)pyridazin-3-yl)-2-(pyridin-2-ylmethyl)octahydrocyclopenta[c]pyrrol-5-amine;
(3aR,5s,6aS)-2-((2,2-difluorobenzo[d][1,3]dioxol-5-yl)methyl)-N-(6-(phenylsulfonyl)pyridazin-3-yl)octahydrocyclopenta[c]pyrrol-5-amine;
(3aR,5s,6aS)-2-(benzo[d][1,3]dioxol-5-ylmethyl)-N-(6-(phenylsulfonyl)pyridazin-3-yl)octahydrocyclopenta[c]pyrrol-5-amine;
(3aR,5s,6aS)—N-(6-(phenylsulfinyl)pyridazin-3-yl)-2-((tetrahydro-2H-pyran-4-yl)methyl)octahydrocyclopenta[c]pyrrol-5-amine;
(3aR,5s,6aS)-2-(2,2-dimethyltetrahydro-2H-pyran-4-yl)-N-(6-(phenylsulfinyl)pyridazin-3-yl)octahydrocyclopenta[c]pyrrol-5-amine;
(3aR,5s,6aS)-2-(benzo[d][1,3]dioxol-5-ylmethyl)-N-(6-(phenylsulfinyl)pyridazin-3-yl)octahydrocyclopenta[c]pyrrol-5-amine;
(3aR,5s,6aS)-2-(4-fluoro-3-methylbenzyl)-N-(6-(phenylsulfonyl)pyridazin-3-yl)octahydrocyclopenta[c]pyrrol-5-amine;
(3aR,5s,6aS)-2-(4-fluoro-3-methylbenzyl)-N-(6-(phenylsulfinyl)pyridazin-3-yl)octahydrocyclopenta[c]pyrrol-5-amine;
(3aR,5s,6aS)—N-(6-(cyclohexylsulfonyl)pyridazin-3-yl)-2-(pyridin-2-ylmethyl)octahydrocyclopenta[c]pyrrol-5-amine;
(3aR,5s,6aS)—N-(6-(cyclohexylsulfonyl)pyridazin-3-yl)-2-((tetrahydro-2H-pyran-4-yl)methyl)octahydrocyclopenta[c]pyrrol-5-amine;
(3aR,5s,6aS)—N-(6-(cyclohexylsulfonyl)pyridazin-3-yl)-2-((1,5-dimethyl-1H-pyrazol-3-yl)methyl)octahydrocyclopenta[c]pyrrol-5-amine;
(3aR,5s,6aS)—N-(6-(cyclohexylsulfonyl)pyridazin-3-yl)-2-(2,2-dimethyltetrahydro-2H-pyran-4-yl)octahydrocyclopenta[c]pyrrol-5-amine;
(3aR,5s,6aS)-2-(benzo[d][1,3]dioxol-5-ylmethyl)-N-(6-(cyclohexylsulfonyl)pyridazin-3-yl)octahydrocyclopenta[c]pyrrol-5-amine;
(3aR,5s,6aS)—N-(6-(cyclohexylsulfonyl)pyridazin-3-yl)-2-(4-fluoro-3-methylbenzyl)octahydrocyclopenta[c]pyrrol-5-amine;
N⁵-cyclopropyl-6-(phenylsulfonyl)-N³-((3aR,5s,6aS)-2-((tetrahydro-2H-pyran-4-yl)methyl)octahydrocyclopenta[c]pyrrol-5-yl)pyridazine-3,5-diamine;
(3aR,5s,6aS)-2-((3-methylpyridin-2-yl)methyl)-N-(6-(phenylsulfonyl)pyridazin-3-yl)octahydrocyclopenta[c]pyrrol-5-amine;
(3aR,5s,6aS)—N-(6-(phenylsulfonyl)pyridazin-3-yl)-2-((3-(trifluoromethyl)pyridin-2-yl)methyl)octahydrocyclopenta[c]pyrrol-5-amine;
(3aR,5s,6aS)-2-((6-methylbenzo[d][1,3]dioxol-5-yl)methyl)-N-(6-(phenylsulfonyl)pyridazin-3-yl)octahydrocyclopenta[c]pyrrol-5-amine;
(3aR,5s,6aS)—N-(6-(cyclohexylsulfonyl)pyridazin-3-yl)-2-((3-methylpyridin-2-yl)methyl)octahydrocyclopenta[c]pyrrol-5-amine;
(3aR,5s,6aS)—N-(6-(cyclohexylsulfonyl)pyridazin-3-yl)-2-((6-methylbenzo[d][1,3]dioxol-5-yl)methyl)octahydrocyclopenta[c]pyrrol-5-amine;
(3aR,5s,6aS)-2-((5-bromo-3-methylpyridin-2-yl)methyl)-N-(6-(cyclohexylsulfonyl)pyridazin-3-yl)octahydrocyclopenta[c]pyrrol-5-amine;
4-(((3aR,5s,6aS)-5-((6-(cyclohexylsulfonyl)pyridazin-3-yl)amino)hexahydrocyclopenta[c]pyrrol-2(1H)-yl)methyl)tetrahydro-2H-pyran-4-ol;
(3aR,5s,6aS)-2-((1H-indol-5-yl)methyl)-N-(6-(cyclohexylsulfonyl)pyridazin-3-yl)octahydrocyclopenta[c]pyrrol-5-amine;
(3aR,5s,6aS)-2-((1H-indol-6-yl)methyl)-N-(6-(cyclohexylsulfonyl)pyridazin-3-yl)octahydrocyclopenta[c]pyrrol-5-amine;
(3aR,5s,6aS)—N-(6-(cyclohexylsulfonyl)pyridazin-3-yl)-2-(3,3-dimethylbutyl)octahydrocyclopenta[c]pyrrol-5-amine;
(3aR,5s,6aS)-2-(3,3-dimethylbutyl)-N-(6-(phenylsulfonyl)pyridazin-3-yl)octahydrocyclopenta[c]pyrrol-5-amine;
(3aR,5s,6aS)—N-(6-chloropyridazin-3-yl)-2-(2,2-dimethyltetrahydro-2H-pyran-4-yl)octahydrocyclopenta[c]pyrrol-5-amine;
(3aR,5s,6aS)-2-(2,2-dimethyltetrahydro-2H-pyran-4-yl)-N-(6-(2-fluorophenyl)pyridazin-3-yl)octahydrocyclopenta[c]pyrrol-5-amine;
(3aR,5s,6aS)-2-(2,2-dimethyltetrahydro-2H-pyran-4-yl)-N-(6-(3-fluorophenyl)pyridazin-3-yl)octahydrocyclopenta[c]pyrrol-5-amine;
(3aR,5s,6aS)-2-(2,2-dimethyltetrahydro-2H-pyran-4-yl)-N-(6-(4-fluorophenyl)pyridazin-3-yl)octahydrocyclopenta[c]pyrrol-5-amine;
(3aR,5s,6aS)—N-(6-(2,5-difluorophenyl)pyridazin-3-yl)-2-(2,2-dimethyltetrahydro-2H-pyran-4-yl)octahydrocyclopenta[c]pyrrol-5-amine;
(3aR,5s,6aS)-2-(2,2-dimethyltetrahydro-2H-pyran-4-yl)-N-(6-(pyridin-3-yl)pyridazin-3-yl)octahydrocyclopenta[c]pyrrol-5-amine;
(3aR,5s,6aS)—N-(6-(1,4-dimethyl-1H-pyrazol-5-yl)pyridazin-3-yl)-2-(2,2-dimethyltetrahydro-2H-pyran-4-yl)octahydrocyclopenta[c]pyrrol-5-amine;
(3aR,5s,6aS)—N-(6-(4,4-difluoropiperidin-1-yl)pyridazin-3-yl)-2-(2,2-dimethyltetrahydro-2H-pyran-4-yl)octahydrocyclopenta[c]pyrrol-5-amine;
N-(5-(6-(((3aR,5s,6aS)-2-(2,2-dimethyltetrahydro-2H-pyran-4-yl)octahydrocyclopenta[c]pyrrol-5-yl)amino)pyridazin-3-yl)pyridin-2-yl)acetamide;
(3aR,5s,6aS)-2-(2,2-dimethyltetrahydro-2H-pyran-4-yl)-N-(6-morpholinopyridazin-3-yl)octahydrocyclopenta[c]pyrrol-5-amine;
N-((3aR,5s,6aS)-2-(2,2-dimethyltetrahydro-2H-pyran-4-yl)octahydrocyclopenta[c]pyrrol-5-yl)-[1,2,4]triazolo[4,3-b]pyridazin-6-amine;
(3aR,5s,6aS)—N-(6-(2-methyl-2H-indazol-5-yl)pyridazin-3-yl)-2-(tetrahydro-2H-pyran-4-yl)octahydrocyclopenta[c]pyrrol-5-amine;
(3aR,5s,6aS)-2-(2,2-dimethyltetrahydro-2H-pyran-4-yl)-N-(6-(2-methyl-2H-indazol-5-yl)pyridazin-3-yl)octahydrocyclopenta[c]pyrrol-5-amine;
(3aR,5s,6aS)—N-(6-(2-chloro-5-fluorophenyl)pyridazin-3-yl)-2-(1-(tetrahydro-2H-pyran-4-yl)cyclopropyl)octahydrocyclopenta[c]pyrrol-5-amine;
(3aR,5s,6aS)-2-(2,2-dimethyltetrahydro-2H-pyran-4-yl)-N-(6-(phenylsulfinyl)pyridazin-3-yl)octahydrocyclopenta[c]pyrrol-5-amine;
(3aR,5s,6aS)—N-(6-(cyclohexylsulfonyl)pyridazin-3-yl)-2-(2,2-dimethyltetrahydro-2H-pyran-4-yl)octahydrocyclopenta[c]pyrrol-5-amine;
(3aR,5s,6aS)—N-(6-(2-chloro-5-fluorophenyl)pyridazin-3-yl)-2-(4,4-difluorocyclohexyl)octahydrocyclopenta[c]pyrrol-5-amine;
(3aR,5s,6aS)—N-(6-(2-chloro-5-fluorophenyl)pyridazin-3-yl)-2-(4,4-dimethylcyclohexyl)octahydrocyclopenta[c]pyrrol-5-amine;
(3aR,5s,6aS)—N-(6-(2-chloro-5-fluorophenyl)pyridazin-3-yl)-2-(2-oxaspiro[3.3]heptan-6-yl)octahydrocyclopenta[c]pyrrol-5-amine;
7-cyclopropyl-5,5-dimethyl-3-(((3aR,5s,6aS)-2-((tetrahydro-2H-pyran-4-yl)methyl)octahydrocyclopenta[c]pyrrol-5-yl)amino)-5,7-dihydro-6H-pyrrolo[2,3-c]pyridazin-6-one;
7-allyl-3-(((3aR,5s,6aS)-2-(3,3-dimethylbutyl)octahydrocyclopenta[c]pyrrol-5-yl)amino)-5,5-dimethyl-5,7-dihydro-6H-pyrrolo[2,3-c]pyridazin-6-one;
3-(((3aR,5s,6aS)-2-(3,3-dimethylbutyl)octahydrocyclopenta[c]pyrrol-5-yl)amino)-5,5-dimethyl-7-propyl-5,7-dihydro-6H-pyrrolo[2,3-c]pyridazin-6-one;
1-((3aR,5s,6aS)-5-((6-(2-Chloro-5-fluorophenyl)pyridazin-3-yl)amino)hexahydrocyclopenta[c]pyrrol-2(1H)-yl)-2-methylpropan-2-ol;
(3aR,5s,6aS)—N-(6-(2-chloro-5-fluorophenyl)pyridazin-3-yl)-2-cyclopentyloctahydrocyclopenta[c]pyrrol-5-amine;
(3aR,5s,6aS)—N-(6-(2-chloro-5-fluorophenyl)pyridazin-3-yl)-2-cycloheptyloctahydrocyclopenta[c]pyrrol-5-amine;
(3aR,5s,6aS)—N-(6-(2-chloro-5-fluorophenyl)pyridazin-3-yl)-2-(2,3-dihydro-1H-inden-2-yl)octahydrocyclopenta[c]pyrrol-5-amine;
(3aR,5s,6aS)—N-(6-(2-chloro-5-fluorophenyl)pyridazin-3-yl)-2-(spiro[5.5]undecan-3-yl)octahydrocyclopenta[c]pyrrol-5-amine;
(3aR,5s,6aS)—N-(6-(2-chloro-5-fluorophenyl)pyridazin-3-yl)-2-(3-oxaspiro[5.5]undecan-9-yl)octahydrocyclopenta[c]pyrrol-5-amine; and
4-((3aR,5s,6aS)-5-((6-(2-chloro-5-fluorophenyl)pyridazin-3-yl)amino)hexahydrocyclopenta[c]pyrrol-2(1H)-yl)tetrahydro-2H-thiopyran 1,1-dioxide;

or a pharmaceutically acceptable salt thereof.

Compound names and/or structures can be assigned/determined by using the Struct=Name naming algorithm as part of CHEMDRAW® ULTRA.

The compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry,” 5th edition (1989), Longman Scientific & Technical, Essex CM20 2JE, England, or (2) direct separation of the mixture of optical enantiomers on chiral chromatographic columns, or (3) fractional recrystallization methods.

Compounds have a 3,3a,4,5,6,6a-hexahydro-1H-cyclopenta[c]pyrrole core structure that has a plane of symmetry as in the following two representative structures.

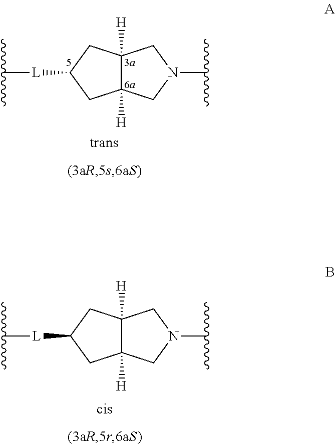

These structures are considered meso since A and B are superimposable with their respective mirror images. The 3a, 5, and 6a stereochemical designations are used herein for symmetrical structures of type A and B to designate relative stereochemistry between the ring fusion and the 5-position. Thus, when drawn in the orientation depicted above 3aR,5s,6aS refers to trans relative stereochemistry between the 5-position substituent and the ring fusion, and 3aR,5r,6aS refers to cis relative stereochemistry between the 5-position substituent and the ring fusion. The lower case s and r designations at the 5-position refer to pseudo assymetry as described by G. P. Moss in “Basic terminology of stereochemistry (IUPAC Recommendations)” in Pure and Applied Chemistry (1996), 68 (12) 2193-2222. The person skilled in the art will understand that when structures A and B are drawn as the respective mirror images, chemical naming programs may, depending on the program, reverse the stereochemical designation for 3a and 6 positions from R to S and S to R, respectively, but that the pseudo asymmetry at the 5-position remains invariant, due to R having priority over S according to priority rules and the reversal of the carbons having R and S designations. Compounds of formula (I) may have a 5-position substituent in a trans configuration or a cis configuration, or may be prepared as a mixture of trans and cis.

It should be understood that the compound may possess tautomeric forms, as well as geometric isomers, and that these also constitute embodiments of the disclosure.

In the compounds of formula (I), and any subformulas, any “hydrogen” or “H,” whether explicitly recited or implicit in the structure, encompasses hydrogen isotopes ¹H (protium) and ²H (deuterium).

The present disclosure also includes an isotopically-labeled compound, which is identical to those recited in formula (I), but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, i.e. ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I) are ¹¹C, ¹³N, ¹⁵O, and ¹⁸F. Isotopically-labeled compounds of formula (I) can generally be prepared by conventional techniques known to those skilled in the art or by processes analogous to those described in the accompanying Examples using appropriate isotopically-labeled reagent in place of non-isotopically-labeled reagent.

a. Pharmaceutically Acceptable Salts

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

b. General Synthesis

Compounds of formula (I) may be prepared by synthetic processes or by metabolic processes. Preparation of the compounds by metabolic processes includes those occurring in the human or animal body (in vivo) or processes occurring in vitro.

Abbreviations: AcOH is acetic acid; BMS is borane dimethyl sulfide complex; Boc is tert-butyloxycarbonyl; BrettPhos-Pd-G3 is [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate (CAS Number 1470372-59-8); t-BuXPhos is 2-di-tert-butylphosphino-2′,4′,6′-triisopropylbiphenyl; DAST is diethylaminosulfur trifluoride; DCE is 1,2-dichloroethane; DCM is dichloromethane; DIBAL is diisobutylaluminum hydride; DIEA and DIPEA both refer to N,N-diisopropylethylamine; DMF is N,N-dimethylformamide; HATU is 2-(7-aza-1H-benzotriazole-1-yl)-1,1,3,3-tetramethyluronium hexafluorophosphate; m-CPBA is meta-chloroperoxybenzoic acid; MeOH is methanol; MsCl is methanesulfonyl chloride; NaBH(OAc)₃ and STAB both refer to sodium triacetoxyborohydride; rt or r.t. is room temperature; NMP is N-methyl-2-pyrrolidone; Pd(dppf)Cl₂ is [1,1′-Bis(diphenylphosphino)ferrocene]dichloropalladium(II); Pd₂(dba)₃ is tris(dibenzylideneacetone)dipalladium(0); RuPhos-Pd-G³ is (2-dicyclohexylphosphino-2′,6′-diisopropoxy-1,1′-biphenyl)[2-(2′-amino-1,1′-biphenyl)]palladium(II) methanesulfonate (CAS Number 1445085-77-7); t-BuOH is tert-butyl alcohol; t-BuOK is potassium tert-butoxide; TBAI is tetrabutylammonium iodide; THF is tetrahydrofuran; and TosMIC is toluenesulfonylmethyl isocyanide.

Compounds of formula (I) may be synthesized as shown in the following schemes.

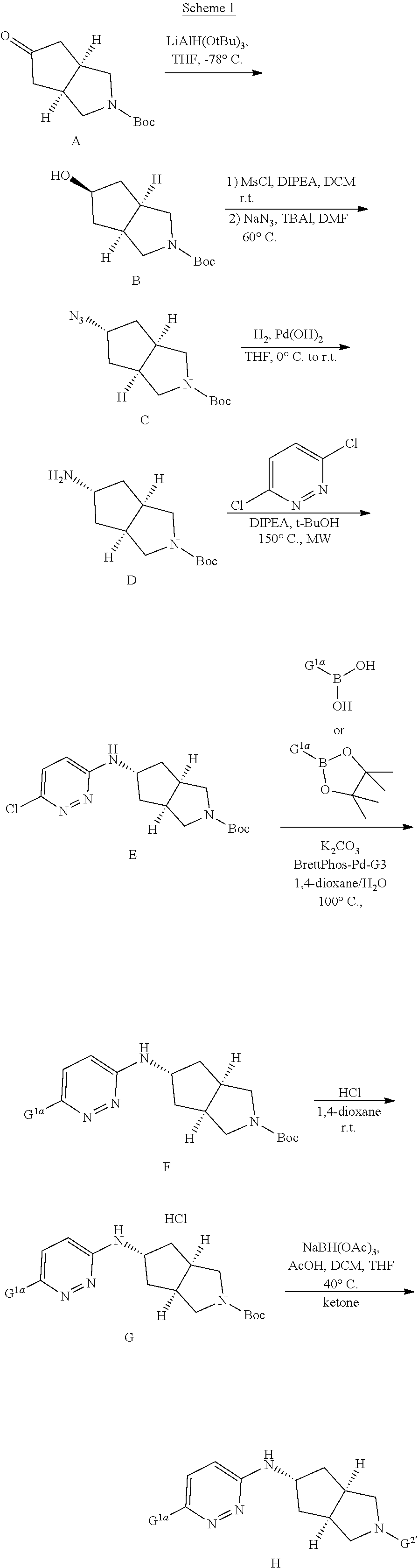

As shown in Scheme 1, cis-tert-butyl 5-oxohexahydrocyclopenta[c]pyrrole-2(1H)-carboxylate (compound A; CAS #146231-54-1, Synthonix, Catalog #B8253) can be reduced (e.g., lithium tri-t-butoxy aluminum hydride) to form compound B, which can then be converted to the corresponding azide compound C. Reduction to the amine provides compound D, which can be reacted with 3,6-dichloropyridazine to generate compound E. Coupling with a suitable boronic acid or ester provides compound F, which can be deprotected (e.g., with hydrochloric acid) to generate compound G. Compound G may be reacted with suitable carbocyclic or heterocyclic ketones corresponding to ring G² by reductive amination to provide H, wherein G^{2′} is a carbocycle or heterocycle.

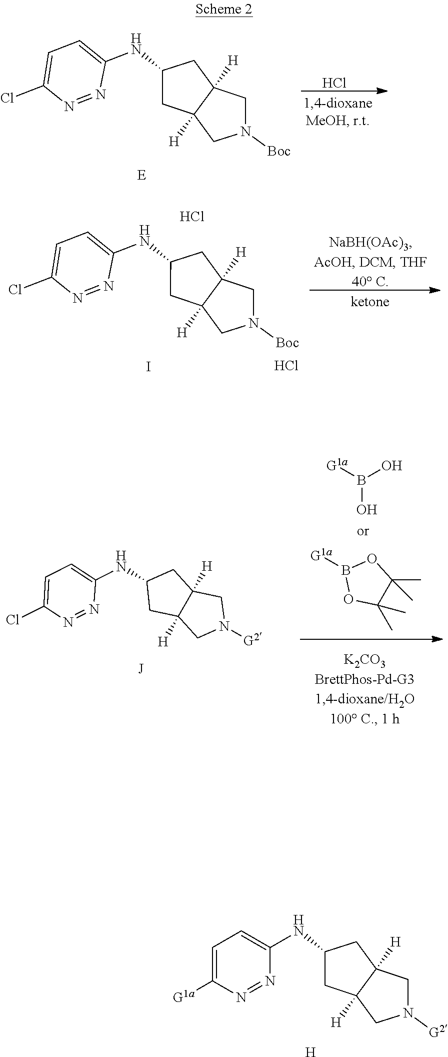

Scheme 2 illustrates an alternate synthesis route to compounds of formula H, wherein the reductive amination and boronic acid coupling steps are reversed. Deprotection of compound E under acid conditions provides compound I, which may be reacted with suitable carbocyclic or heterocyclic ketones corresponding to G² by reductive amination to provide compounds J, wherein G^{2′} is a carbocycle or heterocycle. In turn, reaction of compounds J with suitable boronic acids or esters may provide compounds H.

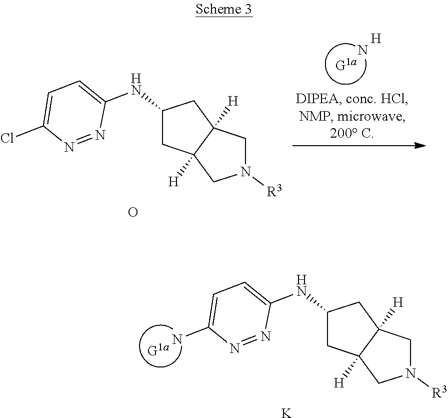

As provided in Scheme 3, reaction of compounds O with a cyclic secondary amine corresponding to a heterocyclic G^{1a} (e.g., morpholine, piperidine) provides compounds of formula K.

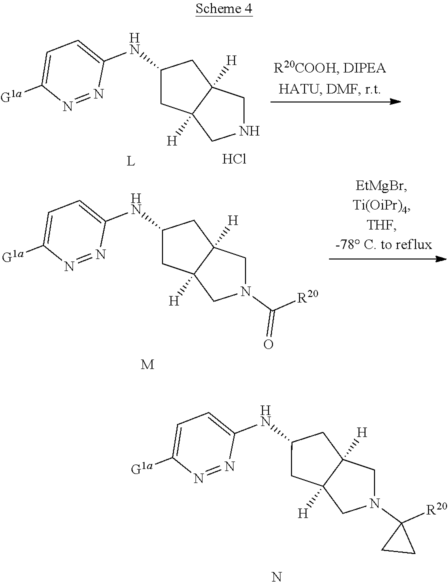

As shown in Scheme 4, reaction of compounds L with a carboxylic acid R²⁰CO₂H under standard amide bond forming conditions provides amides M, which may react with a titanacyclopropane generated in situ from an ethyl Grignard and Ti(OiPr)₄ (Kulinkovich-de Meijere reaction) to provide cyclopropyl compounds of formula N. In Scheme 4, R²⁰ is G², -L¹-G², an alkyl group (e.g., C₁₋₄alkyl), —C₁₋₃alkylene-OR¹³, or —C₁₋₃alkylene-N(R¹³)₂, wherein G², L¹, and R¹³ are as defined herein.

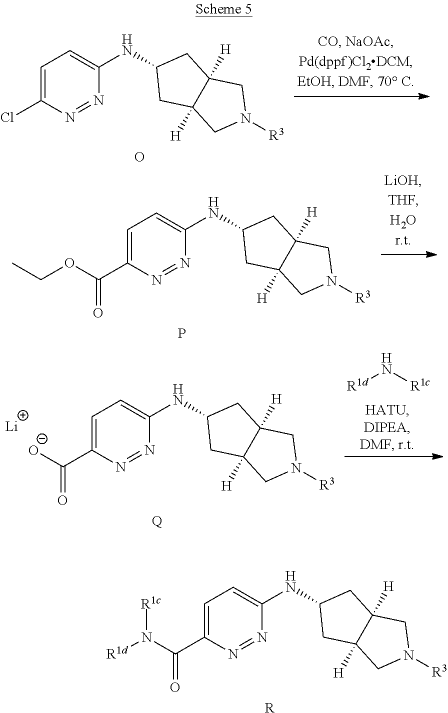

As shown in Scheme 5, carbonylation of compounds O may provide compounds P, which in turn may by hydrolyzed under basic conditions and the resultant acid (or its salt) converted to the corresponding amide R using standard amide bond forming reaction conditions.

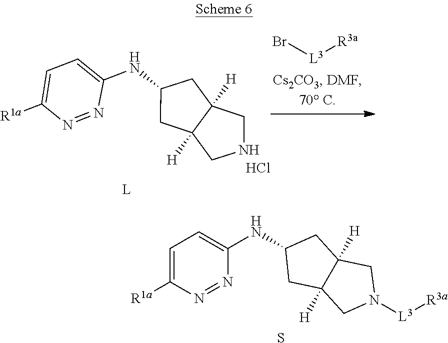

As shown in Scheme 6, compounds of formula L may be alkylated using standard secondary amine alkylation conditions to provide tertiary amines S, wherein L³ is an alkylene group and R^{3a} is as defined herein.

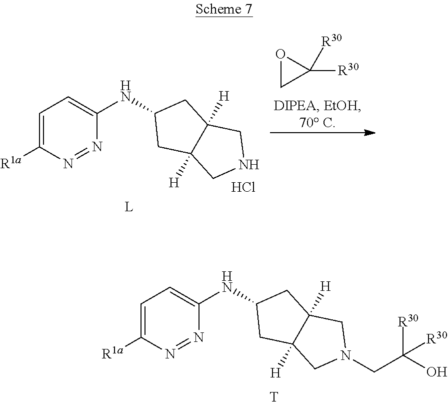

As shown in Scheme 7, secondary amine compounds L may be reacted with epoxides under basic conditions to provide hydroxy compounds T, wherein R³⁰ are alkyl groups, together having 2-4 carbons, or two R³⁰, together with the carbon to which they attach form the carbocyclyl or heterocyclyl of G² (e.g., tetrahydropyranyl, cyclohexyl).

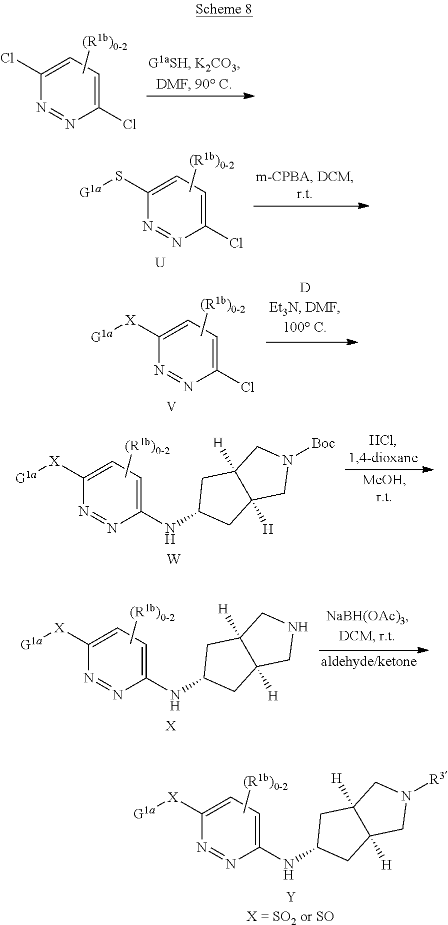

Compounds of formula Y, wherein X is SO₂ or S(O) may be prepared in a multistep sequence as shown in Scheme 8. Reaction of an optionally substituted 2,6-dichloropyridazine with an appropriate thiol under basic conditions may provide compounds U. Oxidation of U may provide sulfone or sulfoxide compounds V, which in turn may be coupled with primary amine D to provide compounds W. Deprotection of W under acidic conditions followed by reductive amination with an aldehyde or ketone may provide compounds Y, wherein R^{3′} is G^{2′} (as defined above), -L¹-G², —C₂₋₆alkylene-R^{3a}, or C₃₋₇alkyl, wherein L¹, G², and R^{3a} are as defined herein.

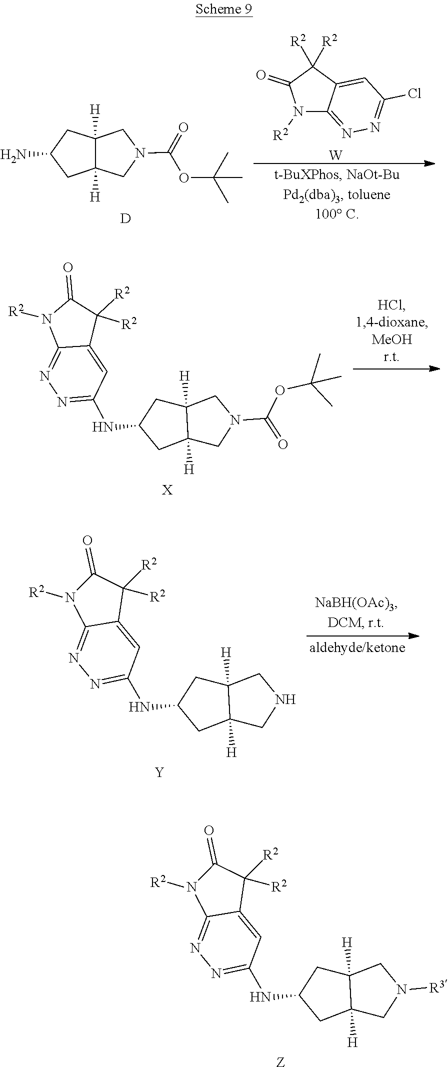

As shown in Scheme 9, compound D may be reacted with substituted 3-chloro-5,7-dihydro-6H-pyrrolo[2,3-c]pyridazin-6-ones W to provide compounds X, which may be deprotected under acidic conditions to provide Y. Reductive amination of Y with an aldehyde or ketone may provide compounds Z, wherein R^{3′} is G^{2′} (as defined above), -L¹-G², —C₂₋₆alkylene-R^{3a}, or C₃₋₇alkyl, wherein L¹, G², and R^{3a} are as defined herein.

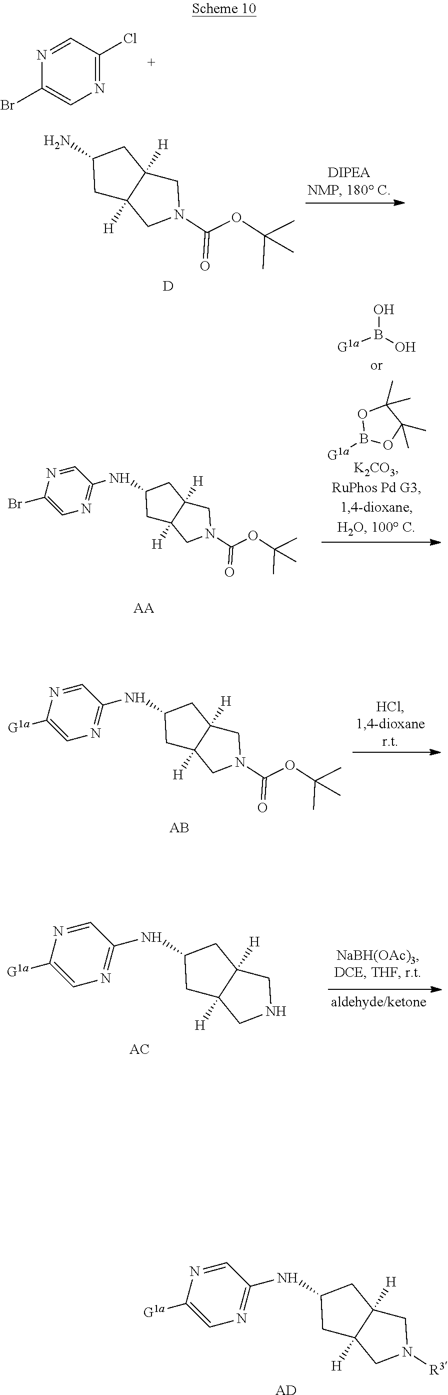

As shown in Scheme 10, compound D may be reacted with 2-bromo-5-chloropyrazine under basic conditions to provide compound AA, which may be coupled with suitable boronic acids or esters to provide intermediates AB. Deprotection of AB under acid conditions may form compounds AC, which may be reacted with suitable aldehydes or ketones under reductive amination conditions to provide compounds AD, wherein R^{3′} is G^{2′} (as defined above), -L¹-G², —C₂₋₆alkylene-R^{3a}, or C₃₋₇alkyl, wherein L¹, G², and R^{3a} are as defined herein.

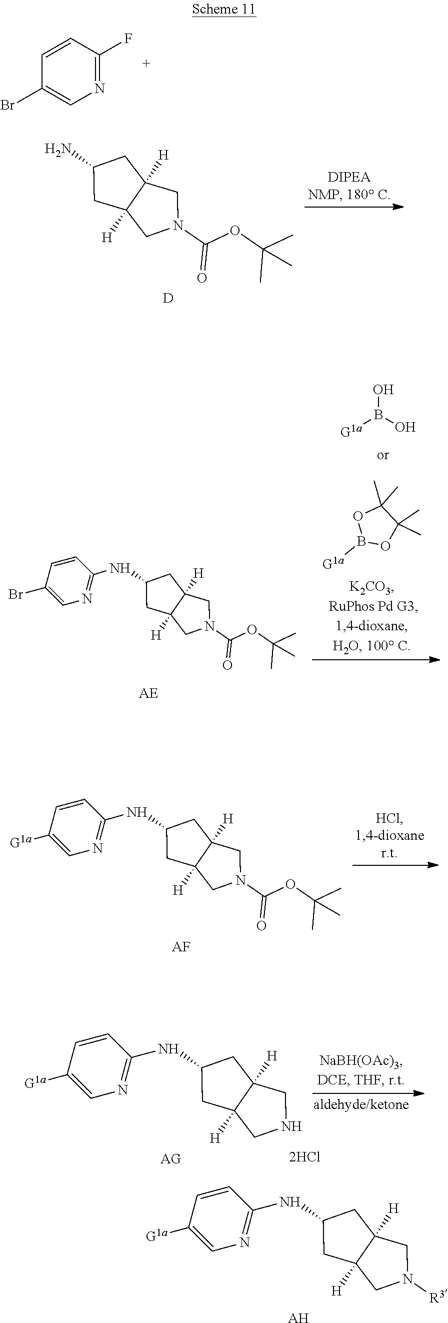

As shown in Scheme 11, compound D may be reacted with 3-bromo-6-fluoropyridine under basic conditions to form compounds AE, which may be coupled with suitable boronic acids or esters to provide compounds AF. Deprotection of AF under acid conditions may form compounds AG, which may be reacted with suitable aldehydes or ketones under reductive amination conditions to provide compounds AH, wherein R^{3′} is G^{2′} (as defined above), -L¹-G², —C₂₋₆alkylene-R^{3a}, or C₃₋₇alkyl, wherein L¹, G², and R^{3a} are as defined herein.

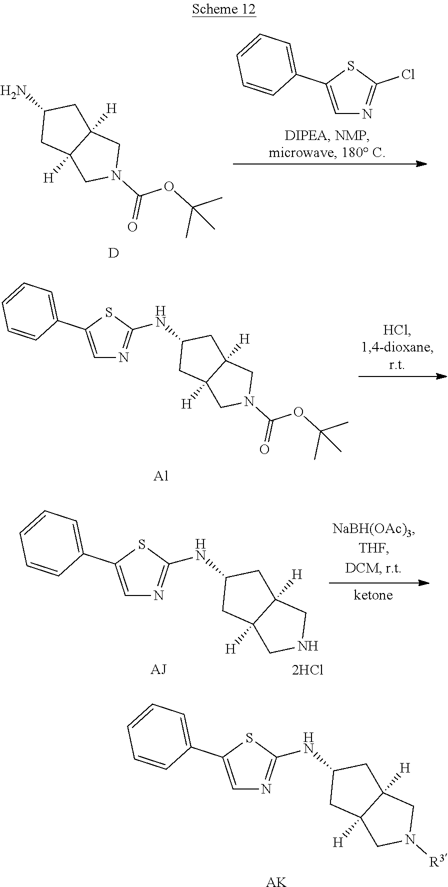

As shown in Scheme 12, compound D may be reacted with 2-chloro-5-phenylthiazole under basic conditions to form compound AI, which may be deprotected under acid conditions to form AJ and subjected to reductive amination to provide compounds AK, wherein R^{3′} is G^{2′} (as defined above), -L¹-G², —C₂₋₆alkylene-R^{3a}, or C₃₋₇alkyl, wherein L¹, G², and R^{3a} are as defined herein.

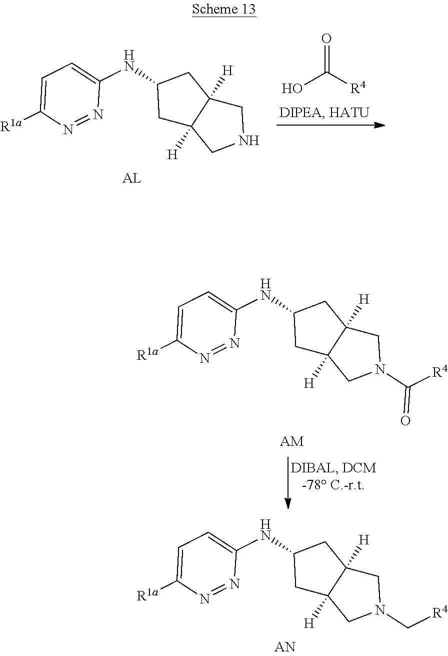

As shown in Scheme 13, compound AL may be reacted with an appropriate carboxylic acid to form amide compound AM, which may be reduced to generate compound AN, wherein R⁴ is G², —C₁₋₂alkylene-G², —C₁₋₅alkylene-R^{3a}, or C₂₋₆alkyl, wherein G² and R^{3a} are as defined herein.

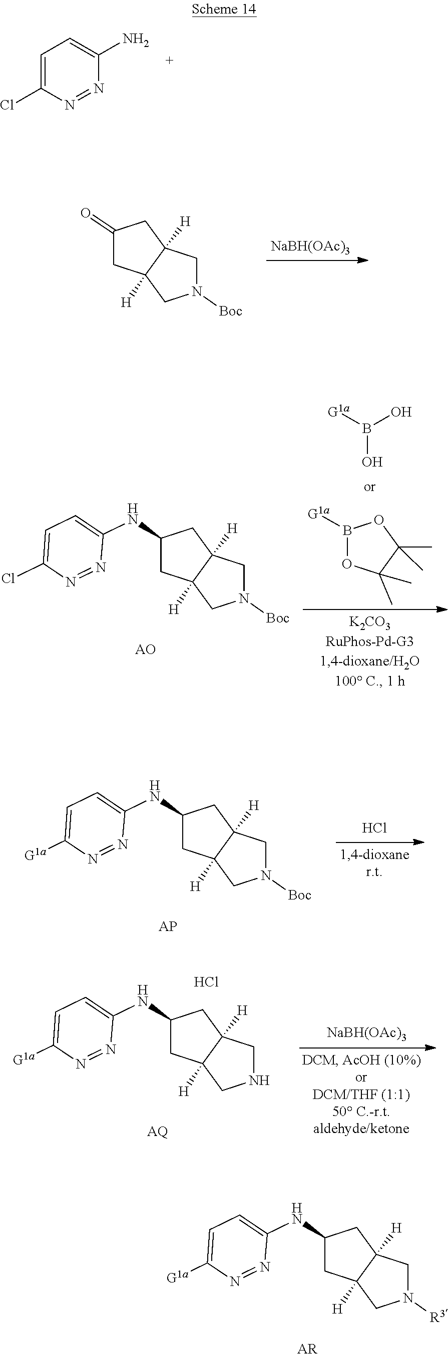

As shown in Scheme 14, 3-amino-6-chloropyridazine can be reacted with cis-N-Boc-5-oxo-octahydrocyclopenta[c]pyrrole to generate compound AO, which may be coupled with an appropriate boronic acid or ester to form compound AP. Deprotection (e.g., with hydrochloric acid) generates compounds AQ, and reaction with a suitable aldehyde or ketone generates compound AR, wherein R^{3′} is G^{2′} (as defined above), -L¹-G², —C₂₋₆alkylene-R^{3a}, or C₃₋₂alkyl, wherein L¹, G², and R^{3a} are as defined herein

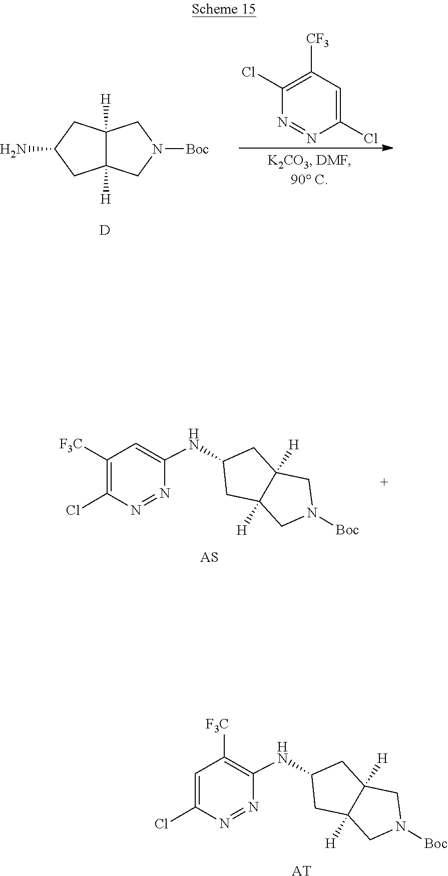

As shown in Scheme 15, reaction of D with 3,6-dichloro-4-PGP-90 (trifluoromethyl)pyridazine under basic conditions provides a mixture of regioisomeric substituted trifluoromethylpyridazines AS and AT that may be separated by standard chromatographic methods.

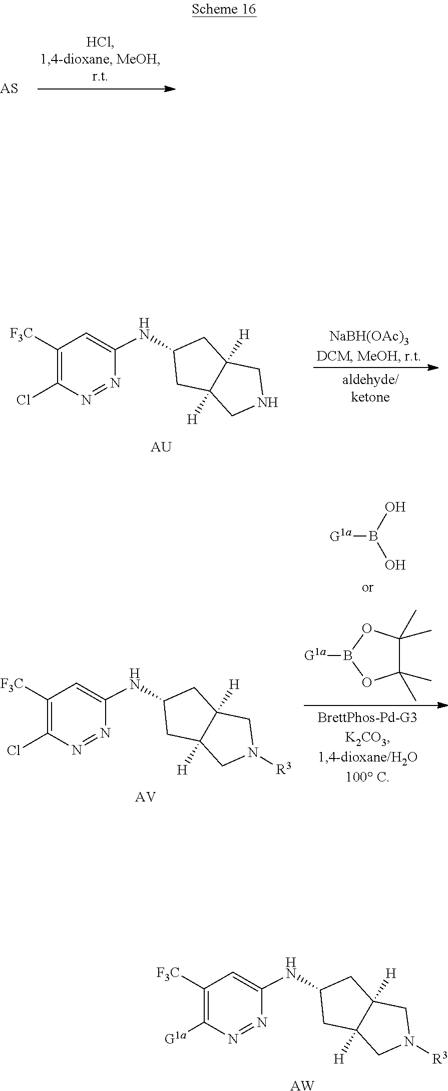

Deprotection of compound AS under acid conditions provides compound AU, which may be reacted with suitable aldehydes or ketones corresponding to R³ by reductive amination to provide compounds AV, wherein R³ is as defined herein. In turn, reaction of compounds AV with suitable boronic acids or esters may provide compounds AW, wherein G^{1a} and R³ are as defined herein.

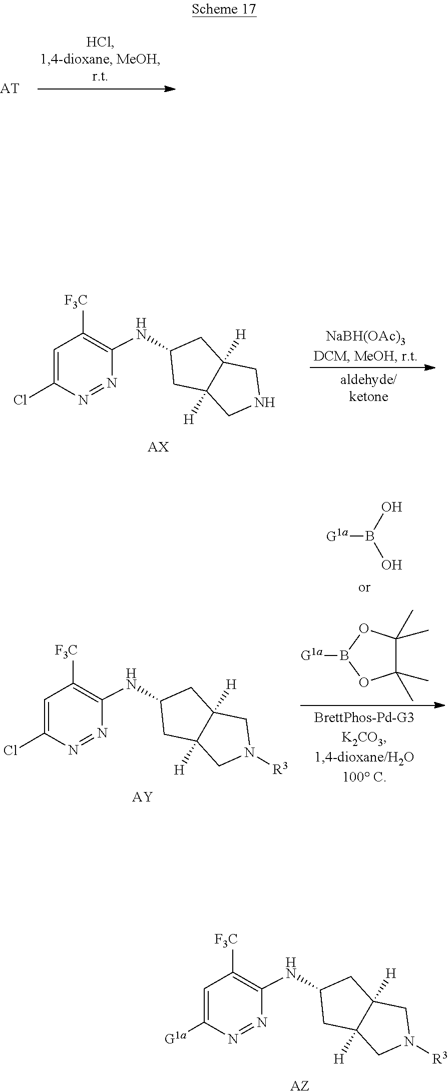

Deprotection of compound AT under acid conditions provides compound AX, which may be reacted with suitable aldehydes or ketones corresponding to R³ by reductive amination to provide compounds AY, wherein R³ is as defined herein. In turn, reaction of compounds AY with suitable boronic acids or esters may provide compounds AZ, wherein G^{1a} and R³ are as defined herein.

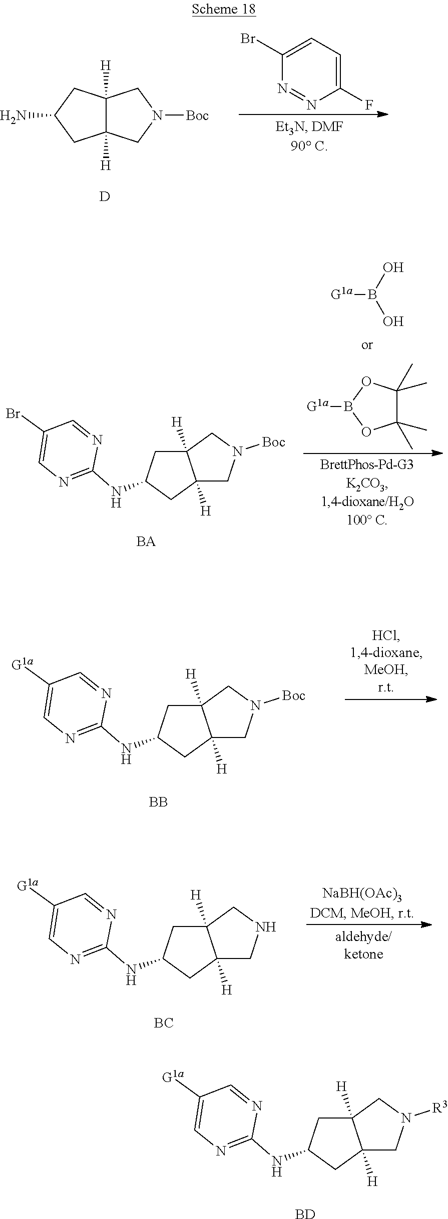

As shown in Scheme 18, reaction of D with 5-bromo-2-fluoropyrimidine under basic conditions provides compound BA. Coupling with a suitable boronic acid or ester provides compounds BB, which may be deprotected (e.g., with hydrochloric acid) to generate compounds BC. Compounds BC may be reacted with suitable aldehydes or ketones corresponding to R³ by reductive amination to provide BD, wherein G^{1a} and R³ are as defined herein.

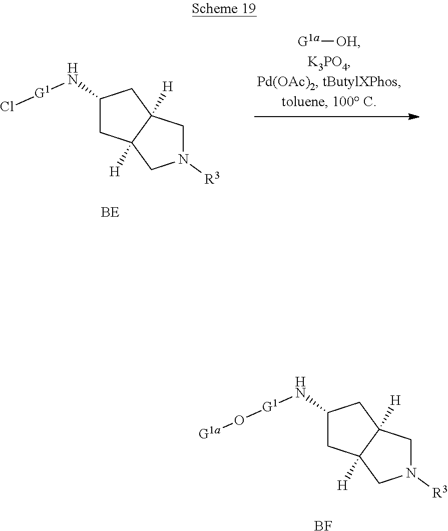

As shown in Scheme 19, compounds BE may provide compounds BF upon reaction with suitable alcohols G^{1a}OH under Buchwald coupling conditions.

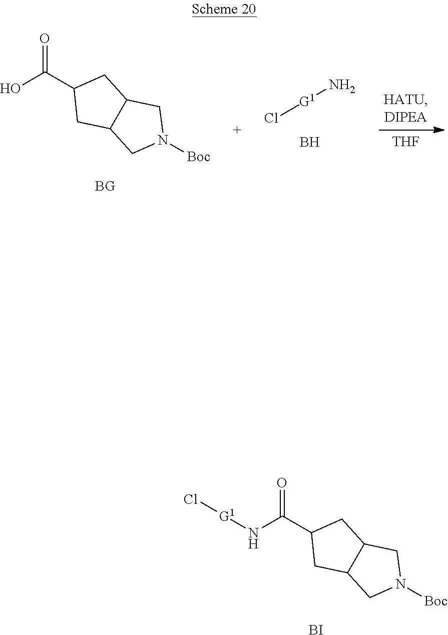

Reaction of 2-(tert-butoxycarbonyl)octahydrocyclopenta[c]pyrrole-5-carboxylic acid (CAS #1177319-91-3, Pharmablock, Catalog #PBN2011986) with amines BH under standard amide bond forming conditions may provide compounds BI. Compounds BI may be elaborated to compounds of the invention using synthetic methods analogous to those depicted in the Schemes and Examples herein. Amines BH include 3-amino-6-chloropyridazine, 2-AMINO-5-CHLOROPYRIMIDINE (CAS #5428-89-7, Matrix Scientific), 6-chloro-4-(trifluoromethyl)pyridazin-3-amine (CAS #1610008-47-3, PharmaBlock Sciences, Inc., WO 2014072261), and 6-Chloro-5-(trifluoromethyl)pyridazin-3-amine (CAS #2254670-55-6, WO 2018226150).

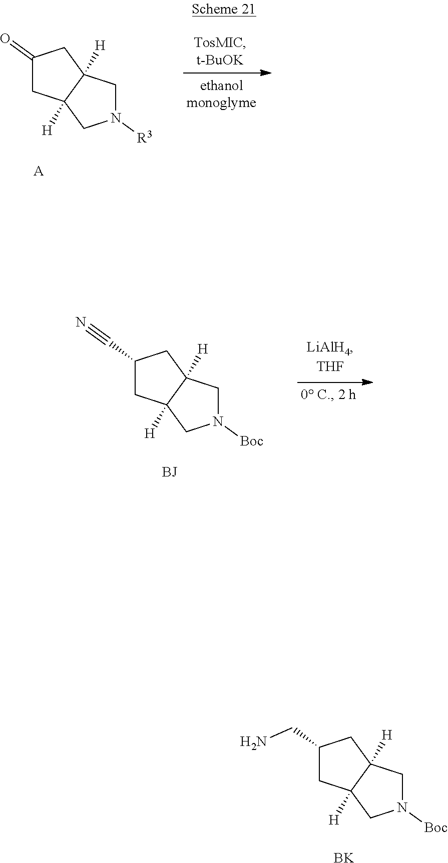

As shown in Scheme 21, compound A can be converted to the corresponding nitrile using TosMIC to generate compound BJ, which can be reduced to the corresponding amine compound BK. Compound BK may be further elaborated to compounds of the invention using synthetic methods analogous to those depicted in the Schemes and Examples herein.

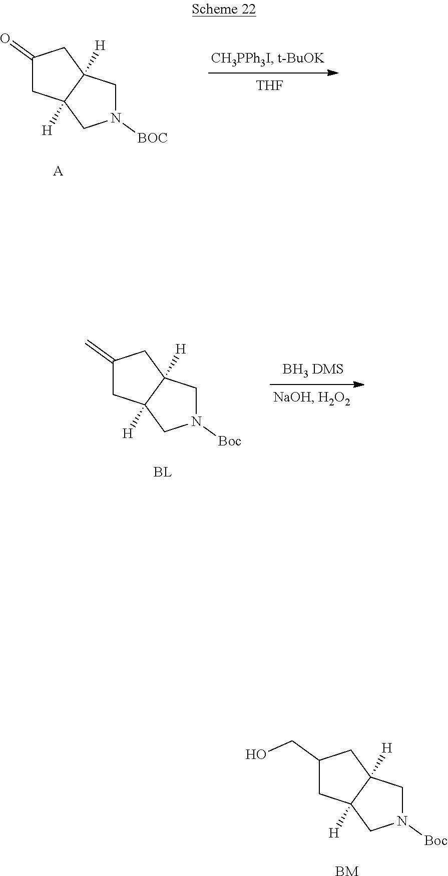

As shown in Scheme 22, compound A can be converted to the corresponding alkene using methyl(triphenyl)phosphonium iodide to generate compound BL, which can be subjected to hydroboration-oxidation to generate the corresponding alcohol compound BM. Compound BM may be elaborated to compounds of the invention by reaction with 3,6-dichloropyridazine, 3,6-dichloro-4-(trifluoromethyl)pyridazine, or 5-bromo-2-fluoropyrimidine under basic conditions (e.g., NaH, THF, r.t.), followed by further synthetic processing according to the methods of the Schemes and Examples herein.

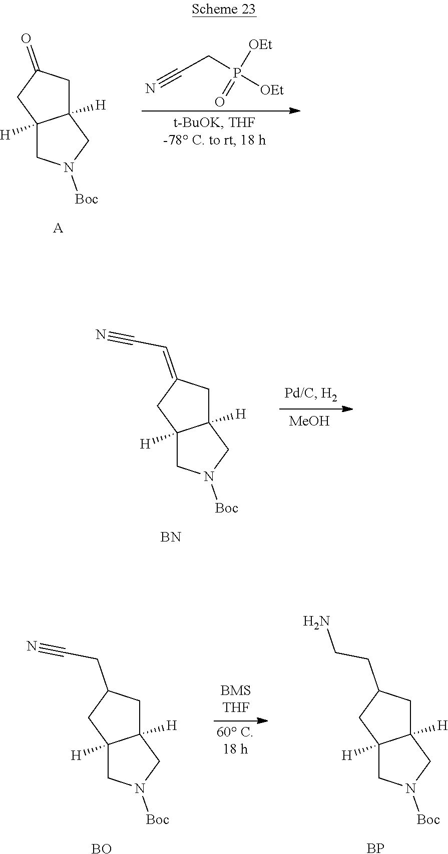

As shown in Scheme 23, compound A can be converted to compound BN using diethyl cyanomethylphosphonate, followed by hydrogenation to form compound BO. Reduction of nitrile compound BO with borane provides compound BP, which may be further elaborated to compounds of the invention using synthetic methods analogous to those depicted in the Schemes and Examples herein.

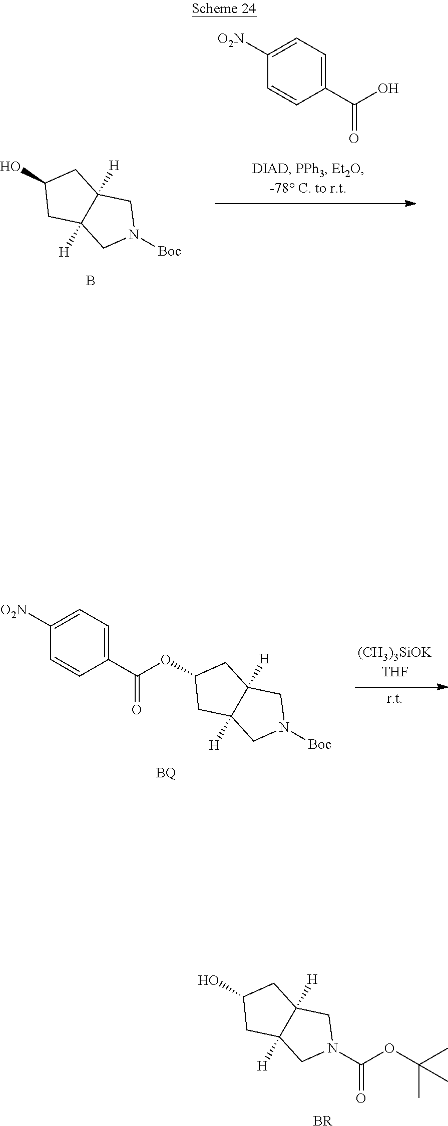

As shown in Scheme 24, compound B may be converted to compound BQ, using a Mitsunobu reaction, and cleaved to BR. Compounds B and BR may be elaborated to compounds of the invention by reaction with reagents including, but not limited to, 3,6-dichloropyridazine, 3,6-dichloro-4-(trifluoromethyl)pyridazine or 5-bromo-2-fluoropyrimidine under basic conditions (e.g., NaH, THF, r.t.), followed by further synthetic processing according to the Schemes and Examples herein.

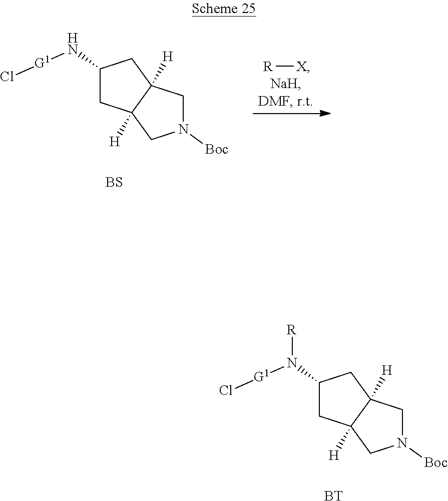

Scheme 25 illustrates a route to intermediates BT by N-alkylation of intermediates BS under basic conditions in the presence of a suitable alkylating agent (e.g., X is Cl, Br, I, OMs, OTs, OTf). Compounds BT may be useful to prepare further compounds of the invention according to the synthetic methods of the Schemes and Example herein.

The intermediates AC, AG, AJ, and AQ may also be reacted according to procedures shown in Schemes 4, 6, 7, and 13 to provide additional compounds of the invention and/or useful intermediates.

Routes to compounds wherein R^{1b} is CN may begin with 3,6-dichloropyridazine-4-carbonitrile replacing 3,6-dichloro-pyridazine or 3,6-dichloro-4-(trifluoromethyl)pyridazine in the Schemes and Examples described herein.

Reductive amination conditions suitable for use in the processes described herein are well known in the art. Representative reaction conditions for aldehyde reductive amination include treating the reactants with NaBH(OAc)₃ in solvents such as DCM, THF, and MeOH, and mixtures thereof, optionally in the presence of a base (e.g., DIPEA). Aldehyde reductive amination may also be effected by treatment with NaBH₃CN in EtOH with heating (e.g., to about 80° C.). Ketone reductive amination may be facilitated by addition of an acid like acetic acid to the solvent mixture (e.g., DCM-THF) and heating to 40° C. for about an hour. A representative solvent ratio of DCM:THF:AcOH is (3:3:0.5). Ketone reductive amination may also be effected by treatment with Ti(OiPr)₄ and NaBH₃CN or NaBH₄ in EtOH from room temperature to about 80° C. NaBD₃CN may be used instead of NaBH₃CN to incorporate deuterium and provide compounds enriched in deuterium over protium.

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry,” 5th edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in P G M Wuts and T W Greene, in Greene's book titled Protective Groups in Organic Synthesis (4^{th}, ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

c. Muscarinic Acetylcholine Receptor M₄ Activity

M₄ is the most highly expressed mAChR subtype in the striatum and its expression is similar in rodents and primates. Due to a lack of selective M₄ antagonists, mechanistic understanding of the role of M₄ has been guided by biochemical and genetic studies, as well as the use of highly selective M₄ positive allosteric modulators (PAMs). Highly selective M₄ PAMs induce robust decreases in behavioral responses to psychomotor stimulants that act by increasing striatal DA levels. Furthermore, genetic deletion of M₄ increases exploratory locomotor activity, potentiates locomotor responses to amphetamine and other stimulants, and eliminates effects of M₄ PAMs on locomotor activity and these effects are also observed with selective deletion of M₄ from striatal spiny projection neurons that express the D1 subtype of DA receptor (D1-SPNs). In vivo microdialysis studies reveal that administration of M₄ PAMs reduces amphetamine-induced DA release in the dorsal and ventral striatum and fMRI studies show that M₄ PAMs reverse amphetamine-induced increases in cerebral blood flow (CBV) in striatum and other basal ganglia nuclei. More recently, fast-scanning cyclic voltammetry (FSCV) and genetic studies, demonstrated that M₄ PAMs act, at least in part, by inhibition of DA release from presynaptic DA terminals in the striatum through release of an endocannabinoid from striatal spiny projection neurons (SPNs) and activation of CB2 cannabinoid receptors on DA terminals.

M₄ is heavily expressed in a subset of SPNs that also express the D₁ subtype of DA receptor (D₁DR), which form the direct pathway (D1-SPNs) sending inhibitory projections to the substantia nigra pars reticulata (SNr). Interestingly, D₁DRs activate a unique GTP-binding protein in D1-SPNs, termed G_{αoif} that couples D₁Rs to activation of adenylyl cyclase, formation of cAMP, and activation of protein kinase A (PKA). This signaling pathway is critical for many of the behavioral actions of DA-mediated activation of motor activity Interestingly, M₄ couples to Gα_{i/o} G proteins, which inhibit adenylyl cyclase and have the potential to directly counteract inhibit D₁ receptor signaling and effects on motor function. These studies raise the possibility that, in addition to inhibition of DA release, M₄ PAMs may directly inhibit D1R-mediated signaling in D₁-SPNs by direct inhibition of cAMP formation and this could also contribute to the powerful inhibitory effect of selective M₄ activation of DA signaling in the basal ganglia. Consistent with this, M₄ PAMs inhibit locomotor-stimulating effects of a direct acting D₁ agonist. Furthermore, a series of pharmacological, genetic, and molecular/cellular studies reveal that this response is mediated by inhibition of D₁DR signaling in D1-SPNs. Thus, the primary action of M₄ PAMs on D₁DR signaling is not in the striatum, but on GABAergic terminals of D₁-SPNs in the SNr, where activation of D₁DRs induces a robust increase in GABA release. This challenges the widespread view that cholinergic regulation of striatal function is almost exclusively mediated through ACh released from tonically active, striatal cholinergic interneurons (ChIs) and raises the possibility that cholinergic innervation of the SNr from cholinergic projections from the pedunculopontine nucleus may also play a critical role in regulating motor activity and other functions of the basal ganglia direct pathway. Together, these data suggest that in addition to inhibiting DA release, M₄ activation also acts postsynaptically in D₁-expressing SPNs to inhibit motor function.

Consistent with a prominent role of M₄ as the primary mAChR subtype involved in regulating motor function, multiple reports indicate that the locomotor-activating effects of the mAChR antagonist scopolamine are dramatically reduced in M₄ knockout mice, but not the other four mAChR subtypes (M_{1-3,5}). Furthermore, haloperidol-induced catalepsy, a model of parkinsonian motor disability, is reduced in M₄ knockout mice as compared to wild-type controls. Evaluation of the anti-parkinsonian effects of scopolamine, by assessing effects of this compound on catalepsy induced by the DA receptor antagonist haloperidol, display robust catalepsy that was completely reversed by scopolamine in WT mice. The reversal by scopolamine was uncommonly robust and more pronounced than we observe with agents targeting a number of other targets being evaluated for potential antiparkinsonian effects, including metabotropic glutamate (mGlu) receptors mGlu₄ or mGlu₅, A₂A adenosine receptors, and NMDA receptors. Importantly, scopolamine was ineffective in reducing catalepsy in M₄KO mice, suggesting that the anti-cataleptic effect of scopolamine requires actions on mAChR M₄. Taken together with the extensive studies of M₄ modulation of basal ganglia and motor function, these studies provide compelling evidence that M₄ is the dominant mAChR subtype involved in the antiparkinsonian effects of non-selective mAChR antagonists and provide support for discovery and development of selective M₄ antagonists for treatment of neurodegenerative disease such as PD, dystonia, tardive dyskinesia and other movement disorders.

Despite advances in mAChR research, there is still a scarcity of compounds that are potent, efficacious and selective antagonists of the M₄ mAChR. Highly selective M₄ antagonists represent a new therapeutic approach for the treatment of neurodegenerative diseases including PD, dystonia, tardive dyskinesia and other movement disorders and may offer the clinical benefit of scopolamine, without the adverse effects mediated by pan-mAChR inhibition.

In some embodiments, the disclosed compounds are antagonists of mAChR M₄. Such activity can be demonstrated by methodology known in the art. For example, antagonism of mAChR M₄ activity can be determined by measurement of calcium flux in response to agonist, e.g. acetylcholine, in cells loaded with a Ca²⁺-sensitive fluorescent dye (e.g., Fluo-4) and co-expression of a chimeric or promiscuous G protein. In some embodiments, the calcium flux can be measured as an increase in fluorescent static ratio. In some embodiments, antagonist activity can be analyzed as a concentration-dependent increase in the EC₈₀ acetylcholine response (i.e. the response of mAChR M₄ at a concentration of acetylcholine that yields 80% of the maximal response).

In some embodiments, the disclosed compounds antagonize mAChR M₄ as a decrease in calcium fluorescence in mAChR M₄-transfected CHO-K1 cells in the presence of the compound, compared to the response of equivalent CHO-K1 cells in the absence of the compound. In some embodiments, a disclosed compound antagonizes the mAChR M₄ response with an IC₅₀ of less than about 10 μM, less than about 5 μM, less than about 1 μM, less than about 500 nM, of less than about 100 nM, or less than about 50 nM. In some embodiments, the mAChR M₄-transfected CHO-K1 cells are transfected with human mAChR M₄. In some embodiments, the mAChR M₄-transfected CHO-K1 cells are transfected with rat mAChR M₄. In some embodiments, the mAChR M₄-transfected CHO-K1 cells are transfected with mAChR M₄ from dog or cynomolgus monkey.

The disclosed compounds may antagonize mAChR M₄ response in mAChR M₄-transfected CHO-K1 cells with an IC₅₀ less than the IC₅₀ for one or more of mAChR M₁, M₂, M₃ or M₅-transfected CHO-K1 cells. That is, a disclosed compound can have selectivity for the mAChR M₄ receptor vis-à-vis one or more of the mAChR M₁, M₂, M₃ or M₅ receptors. For example, in some embodiments, a disclosed compound can antagonize mAChR M₄ response with an IC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₁. In some embodiments, a disclosed compound can antagonize mAChR M₄ response with an IC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₂. In some embodiments, a disclosed compound can antagonize mAChR M₄ response with an IC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₃. In some embodiments, a disclosed compound can antagonize mAChR M₄ response with an IC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₅. In some embodiments, a disclosed compound can antagonize mAChR M₄ response with an IC₅₀ of 5-fold less, about 10-fold less, about 20-fold less, about 30-fold less than that for the M₂-M₅ receptors, of about 50-fold less, about 100-fold less, about 200-fold less, about 300-fold less, about 400-fold less, or greater than about 500-fold less than that for the mAChR M₁, M₂, M₃, or M₅ receptors.

The disclosed compounds may antagonize mAChR M₄ response in M₄-transfected CHO-K1 cells with an IC₅₀ of less than about 10 μM and exhibit a selectivity for the M₄ receptor vis-à-vis one or more of the mAChR M₁, M₂, M₃, or M₅ receptors. For example, in some embodiments, the compound can have an IC₅₀ of less than about 10 μM, of less than about 5 μM, of less than about 1 μM, of less than about 500 nM, of less than about 100 nM, or of less than about 50 nM; and the compound can also antagonize mAChR M₄ response with an IC₅₀ of about 5-fold less, 10-fold less, 20-fold less, 30-fold less, 50-fold less, 100-fold less, 200-fold less, 300-fold less, 400-fold less, or greater than about 500-fold less than that for mAChR M₁. In some embodiments, the compound can have an IC₅₀ of less than about 10 μM, of less than about 5 μM, of less than about 1 μM, of less than about 500 nM, of less than about 100 nM, or of less than about 50 nM; and the compound can also antagonize mAChR M₄ response with an IC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₂. In some embodiments, the compound can have an IC₅₀ of less than about 10 μM, of less than about 5 μM, of less than about 1 μM, of less than about 500 nM, of less than about 100 nM, or of less than about 50 nM; and the compound can also antagonize mAChR M₄ response with an IC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₃. In some embodiments, the compound can have an IC₅₀ of less than about 10 μM, of less than about 5 μM, of less than about 1 μM, of less than about 500 nM, of less than about 100 nM, or of less than about 50 nM; and the compound can also antagonize mAChR M₄ response with an IC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₅. In some embodiments, the compound can have an IC₅₀ of less than about 10 μM, of less than about 5 μM, of less than about 1 μM, of less than about 500 nM, of less than about 100 nM, or of less than about 50 nM; and the compound can also antagonize mAChR M₄ response with IC₅₀ of 5-fold less, about 10-fold less, about 20-fold less, about 30-fold less than that for the M₂-M₅ receptors, of about 50-fold less, about 100-fold less, about 200-fold less, about 300-fold less, about 400-fold less, M₂, M₃, or M₅ receptors, or greater than about 500-fold less than that for the mAChR M₁, M₂, M₃, or M₅ receptors.

In vivo efficacy for disclosed compounds in models that predict antiparkinsonian activity can be measured in a number of preclinical rat models. For example, disclosed compounds may reverse deficits in motor function induced by the dopamine receptor antagonist in mice or rats. Also, these compounds may reverse deficits in motor function that are observed with other manipulations that reduce dopaminergic signaling, such as selective lesions of dopamine neurons. In addition, it is possible that these compounds will have efficacy in animal models of dystonia and may increase attention, cognitive function, and measures of motivation in animal models.

### 3. PHARMACEUTICAL COMPOSITIONS AND FORMULATIONS

The disclosed compounds may be incorporated into pharmaceutical compositions suitable for administration to a subject (such as a patient, which may be a human or non-human). The disclosed compounds may also be provided as formulations, such as spray-dried dispersion formulations.

The pharmaceutical compositions and formulations may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of a compound of formula (I), may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions and formulations may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington's Pharmaceutical Sciences,” (Meade Publishing Co., Easton, Pa.). Therapeutic compositions must typically be sterile and stable under the conditions of manufacture and storage.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, Pa.) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Del. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of an active compound (e.g., a compound of formula (I)) and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound of formula (I)), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I)), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

The pharmaceutical composition or formulation may antagonize mAChR M₄ with an IC₅₀ of less than about 10 μM, less than about 5 μM, less than about 1 μM, less than about 500 nM, or less than about 100 nM. The pharmaceutical composition or formulation may antagonize mAChR M₄ with an IC₅₀ of between about 10 μM and about 1 nM, about 1 μM and about 1 nM, about 100 nM and about 1 nM, or between about 10 nM and about 1 nM.

a. Spray-Dried Dispersion Formulations

The disclosed compounds may be formulated as a spray-dried dispersion (SDD). An SDD is a single-phase, amorphous molecular dispersion of a drug in a polymer matrix. It is a solid solution with the compound molecularly “dissolved” in a solid matrix. SDDs are obtained by dissolving drug and a polymer in an organic solvent and then spray-drying the solution. The use of spray drying for pharmaceutical applications can result in amorphous dispersions with increased solubility of Biopharmaceutics Classification System (BCS) class II (high permeability, low solubility) and class IV (low permeability, low solubility) drugs. Formulation and process conditions are selected so that the solvent quickly evaporates from the droplets, thus allowing insufficient time for phase separation or crystallization. SDDs have demonstrated long-term stability and manufacturability. For example, shelf lives of more than 2 years have been demonstrated with SDDs. Advantages of SDDs include, but are not limited to, enhanced oral bioavailability of poorly water-soluble compounds, delivery using traditional solid dosage forms (e.g., tablets and capsules), a reproducible, controllable and scalable manufacturing process and broad applicability to structurally diverse insoluble compounds with a wide range of physical properties.

Thus, in one embodiment, the disclosure may provide a spray-dried dispersion formulation comprising a compound of formula (I).

### 4. METHODS OF USE

The disclosed compounds, pharmaceutical compositions and formulations may be used in methods for treatment of disorders, such as neurological and/or psychiatric disorders, associated with muscarinic acetylcholine receptor dysfunction. The disclosed compounds and pharmaceutical compositions may also be used in methods for decreasing muscarinic acetylcholine receptor activity in a mammal. The methods further include cotherapeutic methods for improving treatment outcomes. In the methods of use described herein, additional therapeutic agent(s) may be administered simultaneously or sequentially with the disclosed compounds and compositions.

a. Treating Disorders

The disclosed compounds, pharmaceutical compositions and formulations may be used in methods for treating, preventing, ameliorating, controlling, reducing, or reducing the risk of a variety of disorders, or symptoms of the disorders, in which a patient would benefit from antagonism of mAChR M₄. In some embodiments, the disorder may be a neurodegenerative disorder, a movement disorder, or a brain disorder. The methods may comprise administering to a subject in need of such treatment a therapeutically effective amount of the compound of formula (I) or a pharmaceutically acceptable salt thereof, or a pharmaceutical composition comprising a therapeutically effective amount of a compound of formula (I) or a pharmaceutically acceptable salt thereof.

Disorders in which a patient would benefit from antagonism of mAChR M₄ may include neurodegenerative disorders and movement disorders. For example, exemplary disorders may include Parkinson's disease, drug-induced Parkinsonism, dystonia, Tourette's syndrome, dyskinesias (e.g., tardive dyskinesia or levodopa-induced dyskinesia), schizophrenia, cognitive deficits associated with schizophrenia, excessive daytime sleepiness (e.g., narcolepsy), attention deficit hyperactivity disorder (ADHD), Huntington's disease, chorea (e.g., chorea associated with Huntington's disease), cerebral palsy, and progressive supranuclear palsy.

In some embodiments, the disclosure provides a method for treating motor symptoms in a subject having Parkinson's disease, comprising administering to a subject in need thereof a therapeutically effective amount of the compound of formula (I) or a pharmaceutically acceptable salt thereof, or a pharmaceutical composition comprising a therapeutically effective amount of a compound of formula (I) or a pharmaceutically acceptable salt thereof. In some embodiments, the motor symptoms are selected from bradykinesia, tremor, rigidity, gait dysfunction, and postural instability. The method may treat the motor symptoms, control the motor symptoms, and/or reduce the motor symptoms in the subject.

In some embodiments, the disclosure provides a method for treating motor symptoms in a subject having dystonia, comprising administering to the subject a therapeutically effective amount of the compound of formula (I) or a pharmaceutically acceptable salt thereof, or a pharmaceutical composition comprising a therapeutically effective amount of a compound of formula (I) or a pharmaceutically acceptable salt thereof. The method may treat the motor symptoms, control the motor symptoms, and/or reduce the motor symptoms in the subject. For example, treatment may reduce muscle contractions or spasms in a subject having dystonia.

In some embodiments, the disclosure provides a method for treating motor symptoms in a subject having tardive dyskinesia, comprising administering to the subject a therapeutically effective amount of the compound of formula (I) or a pharmaceutically acceptable salt thereof, or a pharmaceutical composition comprising a therapeutically effective amount of a compound of formula (I) or a pharmaceutically acceptable salt thereof. The method may treat the motor symptoms, control the motor symptoms, and/or reduce the motor symptoms in the subject. For example, treatment may reduce involuntary movements in a subject having tardive dyskinesia.

In some embodiments, the disclosure provides a method of preventing or delaying tardive dyskinesia in a subject at risk of developing tardive dyskinesia, comprising administering to the subject a therapeutically effective amount of the compound of formula (I) or a pharmaceutically acceptable salt thereof, or a pharmaceutical composition comprising a therapeutically effective amount of a compound of formula (I) or a pharmaceutically acceptable salt thereof. For example, the subject may be a subject being treated with a neuroleptic medication (e.g., a typical antipsychotic or an atypical antipsychotic), a dopamine antagonist, or an antiemetic.

In some embodiments, the disclosure provides a method of treating catalepsy in a subject suffering from schizophrenia, comprising administering to the subject a therapeutically effective amount of the compound of formula (I) or a pharmaceutically acceptable salt thereof, or a pharmaceutical composition comprising a therapeutically effective amount of a compound of formula (I) or a pharmaceutically acceptable salt thereof. For example, the subject suffering from schizophrenia may have catalepsy induced by a neuroleptic agent (e.g., a typical antipsychotic or an atypical antipsychotic).

In some embodiments, the disclosure provides a method of treating a brain disorder characterized by altered dopamine and cholinergic signaling that could benefit from antagonism of mAChR M₄, comprising administering to the subject a therapeutically effective amount of the compound of formula (I) or a pharmaceutically acceptable salt thereof, or a pharmaceutical composition comprising a therapeutically effective amount of a compound of formula (I) or a pharmaceutically acceptable salt thereof. For example, the treatment may increase motivation or goal-directed behavior in patients suffering from disorders characterized by reduced motivation for goal-directed behavior, such as schizophrenia and other brain disorders.

In some embodiments, the disclosure provides a method for increasing wakefulness and/or reducing excessive daytime sleepiness in a subject in need thereof, comprising administering to the subject a therapeutically effective amount of the compound of formula (I) or a pharmaceutically acceptable salt thereof, or a pharmaceutical composition comprising a therapeutically effective amount of a compound of formula (I) or a pharmaceutically acceptable salt thereof. In some embodiments, the subject is a subject suffering from narcolepsy.

In some embodiments, the disclosure provides a method of increasing attention in a subject (e.g., a subject suffering from an attention deficit disorder such as ADHD) in a subject in need thereof, comprising administering to the subject a therapeutically effective amount of the compound of formula (I) or a pharmaceutically acceptable salt thereof, or a pharmaceutical composition comprising a therapeutically effective amount of a compound of formula (I) or a pharmaceutically acceptable salt thereof.

In some embodiments, the disclosure provides a method for treating motor symptoms in a subject having a drug-induced movement disorder, comprising administering the subject a therapeutically effective amount of the compound of formula (I) or a pharmaceutically acceptable salt thereof, or a pharmaceutical composition comprising a therapeutically effective amount of a compound of formula (I) or a pharmaceutically acceptable salt thereof. In some embodiments, the drug-induced movement disorder is selected from drug-induced parkinsonism, tardive dyskinesia, tardive dystonia, akathisia, myoclonus, and tremor. The method may treat the motor symptoms, control the motor symptoms, and/or reduce the motor symptoms in the subject.

The compounds and compositions may be further useful in a method for the prevention, treatment, control, amelioration, or reduction of risk of the diseases, disorders and conditions noted herein. The compounds and compositions may be further useful in a method for the prevention, treatment, control, amelioration, or reduction of risk of the aforementioned diseases, disorders and conditions, in combination with other agents.

In the treatment of conditions such as those that would benefit from antagonism of mAChR M₄, an appropriate dosage level may be about 0.01 to 500 mg per kg patient body weight per day, which can be administered in single or multiple doses. The dosage level may be about 0.1 to about 250 mg/kg per day, or about 0.5 to about 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5 or 5 to 50 mg/kg per day. For oral administration, the compositions may be provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, or 1000 milligrams of the active ingredient for the symptomatic adjustment of the dosage to the patient to be treated. The compounds can be administered on a regimen of 1 to 4 times per day, preferably once or twice per day. This dosage regimen can be adjusted to provide the optimal therapeutic response. It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Thus, in some embodiments, the disclosure relates to a method for antagonizing the mAChR M₄ receptor in at least one cell, comprising the step of contacting the at least one cell with at least one disclosed compound or at least one product of a disclosed method in an amount effective to antagonize mAChR M₄ in the at least one cell. In some embodiments, the cell is mammalian, for example, human. In some embodiments, the cell has been isolated from a subject prior to the contacting step. In some embodiments, contacting is via administration to a subject.

In some embodiments, the invention relates to a method for antagonizing the mAChR M₄ receptor in a subject, comprising the step of administering to the subject at least one disclosed compound or at least one product of a disclosed method in a dosage and amount effective to antagonize the mAChR M₄ receptor in the subject. In some embodiments, the subject is mammalian, for example, human. In some embodiments, the mammal has been diagnosed with a need for mAChR M₄ antagonism prior to the administering step. In some embodiments, the mammal has been diagnosed with a need for mAChR M₄ antagonism prior to the administering step. In some embodiments, the method further comprises the step of identifying a subject in need of mAChR M₄ antagonism.

b. Antagonism of the Muscarinic Acetylcholine Receptor

In some embodiments, the disclosure relates to a method for antagonizing mAChR M₄ in a mammal, comprising the step of administering to the mammal an effective amount of at least one disclosed compound or a pharmaceutically acceptable salt thereof, or a pharmaceutical composition comprising at least one disclosed compound or pharmaceutically acceptable salt thereof.

In some embodiments, antagonism of the muscarinic acetylcholine receptor decreases muscarinic acetylcholine receptor activity.

In some embodiments, the compound administered antagonizes mAChR M₄ with an IC₅₀ of less than about 10 μM, less than about 5 μM, less than about 1 μM, less than about 500 nM, or less than about 100 nM. In some embodiments, the compound administered antagonizes mAChR M₄ with an IC₅₀ of between about 10 μM and about 1 nM, about 1 μM and about 1 nM, about 100 nM and about 1 nM, or about 10 nM and about 1 nM.

In some embodiments, the mammal is a human. In some embodiments, the mammal has been diagnosed with a need for reduction of muscarinic acetylcholine receptor activity prior to the administering step. In some embodiments, the method further comprises the step of identifying a mammal in need of reducing muscarinic acetylcholine receptor activity. In some embodiments, the antagonism of the muscarinic acetylcholine receptor treats a disorder associated with muscarinic acetylcholine receptor activity in the mammal. In some embodiments, the muscarinic acetylcholine receptor is mAChR M₄.

In some embodiments, antagonism of the muscarinic acetylcholine receptor in a mammal is associated with the treatment of a disorder associated with a muscarinic receptor dysfunction, such as a disorder disclosed herein. In some embodiments, the muscarinic receptor is mAChR M₄.

In some embodiments, the disclosure provides a method for antagonizing the muscarinic acetylcholine receptor in a cell, comprising the step of contacting the cell with an effective amount of at least one disclosed compound or a pharmaceutically acceptable salt thereof. In some embodiments, the cell is mammalian (e.g., human). In some embodiments, the cell has been isolated from a mammal prior to the contacting step. In some embodiments, contacting is via administration to a mammal.

c. Cotherapeutic Methods

The present disclosure is further directed to administration of a mAChR M₄ antagonist, such as a selective mAChR M₄ antagonist, for improving treatment outcomes. That is, in some embodiments, the disclosure relates to a cotherapeutic method comprising a step of administering to a mammal an effective amount and dosage of at least one disclosed compound, or a pharmaceutically acceptable salt thereof.

In some embodiments, administration improves treatment outcomes in the context of cognitive or behavioral therapy. Administration in connection with cognitive or behavioral therapy can be continuous or intermittent. Administration need not be simultaneous with therapy and can be before, during, and/or after therapy. For example, cognitive or behavioral therapy can be provided within 1, 2, 3, 4, 5, 6, 7 days before or after administration of the compound. As a further example, cognitive or behavioral therapy can be provided within 1, 2, 3, or 4 weeks before or after administration of the compound. As a still further example, cognitive or behavioral therapy can be provided before or after administration within a period of time of 1, 2, 3, 4, 5, 6, 7, 8, 9, or 10 half-lives of the administered compound.

In some embodiments, administration may improve treatment outcomes in the context of physical or occupational therapy. Administration in connection with physical or occupational therapy can be continuous or intermittent. Administration need not be simultaneous with therapy and can be before, during, and/or after therapy. For example, physical or occupational therapy can be provided within 1, 2, 3, 4, 5, 6, 7 days before or after administration of the compound. As a further example, physical or occupational therapy can be provided within 1, 2, 3, or 4 weeks before or after administration of the compound. As a still further example, physical or occupational therapy can be provided before or after administration within a period of time of 1, 2, 3, 4, 5, 6, 7, 8, 9, or 10 half-lives of the administered compound.

It is understood that the disclosed cotherapeutic methods can be used in connection with the disclosed compounds, compositions, kits, and uses.

d. Combination Therapies

In the methods of use described herein, additional therapeutic agent(s) may be administered simultaneously or sequentially with the disclosed compounds and compositions. Sequential administration includes administration before or after the disclosed compounds and compositions. In some embodiments, the additional therapeutic agent or agents may be administered in the same composition as the disclosed compounds. In other embodiments, there may be an interval of time between administration of the additional therapeutic agent and the disclosed compounds. In some embodiments, administration of an additional therapeutic agent with a disclosed compound may allow lower doses of the other therapeutic agents and/or administration at less frequent intervals. When used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula (I). The above combinations include combinations of a compound of the present invention not only with one other active compound, but also with two or more other active compounds.

The disclosed compounds can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefor, contemporaneously or sequentially with a disclosed compound. When a disclosed compound is used contemporaneously with one or more other drugs, a pharmaceutical composition in unit dosage form containing such drugs and the disclosed compound may be used. However, the combination therapy can also be administered on overlapping schedules. It is also envisioned that the combination of one or more active ingredients and a disclosed compound can be more efficacious than either as a single agent. Thus, when used in combination with one or more other active ingredients, the disclosed compounds and the other active ingredients can be used in lower doses than when each is used singly.

The pharmaceutical compositions and methods of the present invention can further comprise other therapeutically active compounds as noted herein which are usually applied in the treatment of the above mentioned pathological conditions.

The above combinations include combinations of a disclosed compound not only with one other active compound, but also with two or more other active compounds. Likewise, disclosed compounds can be used in combination with other drugs that are used in the prevention, treatment, control, amelioration, or reduction of risk of the diseases or conditions for which disclosed compounds are useful. Such other drugs can be administered, by a route and in an amount commonly used therefor, contemporaneously or sequentially with a compound of the present invention. When a compound of the present invention is used contemporaneously with one or more other drugs, a pharmaceutical composition containing such other drugs in addition to a disclosed compound is preferred. Accordingly, the pharmaceutical compositions include those that also contain one or more other active ingredients, in addition to a compound of the present invention.

The weight ratio of a disclosed compound to the second active ingredient can be varied and will depend upon the effective dose of each ingredient. Generally, an effective dose of each will be used. Thus, for example, when a compound of the present invention is combined with another agent, the weight ratio of a disclosed compound to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations a disclosed compound and other active agents can be administered separately or in conjunction. In addition, the administration of one element can be prior to, concurrent to, or subsequent to the administration of other agent(s).

Accordingly, the disclosed compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent can be coadministered, either in concomitant therapy or in a fixed combination.

In some embodiments, the compound can be employed in combination with any other agent that is used to treat a disorder described herein, such as a standard of care therapy for a disorder that would benefit from mAChR M₄ antagonism, such as a disorder described herein. For example, in some embodiments, the compound can be employed in combination with a Parkinsonian drug (e.g., L-DOPA, or carbidopa/levodopa) an mGlu₄ positive allosteric modulator, an mGlu₅ negative allosteric modulator, an A₂A inhibitor, a T-type calcium channel antagonist, a VMAT2 inhibitor, a muscle relaxant (e.g., baclofen), an anticholinergic agent, an antiemetic, a typical or atypical neuroleptic agent (e.g., risperidone, ziprasidone, haloperidol, pimozide, fluphenazine), an antihypertensive agent (e.g., clonidine or guanfacine), a tricyclic antidepressant (e.g., amitriptyline, butriptyline, clomipramine, desipramine, dosulepin, doxepin, imipramine, iprindole, lofepramine, nortriptyline, protriptyline, or trimipramine) an agent that increases extracellular dopamine levels (e.g., amphetamine, methylphenidate, or lisdexamfetamine), an agent for treating excessive daytime sleepiness (e.g., sodium oxybate or a wakefulness-promoting agent such as armodafinil or modafinil), and a norepinephrine reuptake inhibitor (including selective NRIs, e.g., atomoxetine, and non-selective NRIs, e.g., bupropion).

e. Modes of Administration

Methods of treatment may include any number of modes of administering a disclosed composition. Modes of administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the agent may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire™). In the pharmaceutical composition, the agent may also be dispersed in a microparticle, e.g. a nanoparticulate composition.

For parenteral administration, the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration, the agent can be in the form of an aqueous, lipid, oily or other kind of solution or suspension or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

### 5. Kits

In one aspect, the disclosure provides a kit comprising at least one disclosed compound or a pharmaceutically acceptable salt thereof, or a pharmaceutical composition comprising at least one disclosed compound or a pharmaceutically acceptable salt thereof and one or more of:
(a) at least one agent known to increase mAChR M₄ activity;
(b) at least one agent known to decrease mAChR M₄ activity;
(c) at least one agent known to treat a disorder associated with mAChR M₄, such as a disorder described herein; and
(d) instructions for administering the compound.

In some embodiments, the at least one disclosed compound and the at least one agent are co-formulated. In some embodiments, the at least one disclosed compound and the at least one agent are co-packaged. The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

That the disclosed kits can be employed in connection with disclosed methods of use.

The kits may further comprise information, instructions, or both that use of the kit will provide treatment for medical conditions in mammals (particularly humans). The information and instructions may be in the form of words, pictures, or both, and the like. In addition or in the alternative, the kit may include the compound, a composition, or both; and information, instructions, or both, regarding methods of application of compound, or of composition, preferably with the benefit of treating or preventing medical conditions in mammals (e.g., humans).

The compounds and processes of the invention will be better understood by reference to the following examples, which are intended as an illustration of and not a limitation upon the scope of the invention.

### 6. EXAMPLES

All NMR spectra were recorded on a 400 MHz AMX Bruker NMR spectrometer. ¹H chemical shifts are reported in 6 values in ppm downfield with the deuterated solvent as the internal standard. Data are reported as follows: chemical shift, multiplicity (s=singlet, bs=broad singlet, d=doublet, t=triplet, q=quartet, dd=doublet of doublets, m=multiplet, ABq=AB quartet), coupling constant, integration. Reversed-phase LCMS analysis was performed using an Agilent 1200 system comprised of a binary pump with degasser, high-performance autosampler, thermostatted column compartment, C18 column, diode-array detector (DAD) and an Agilent 6150 MSD with the following parameters. The gradient conditions were 5% to 95% acetonitrile with the aqueous phase 0.1% TFA in water over 1.4 minutes. Samples were separated on a Waters Acquity UPLC BEH C18 column (1.7 μm, 1.0×50 mm) at 0.5 mL/min, with column and solvent temperatures maintained at 55° C. The DAD was set to scan from 190 to 300 nm, and the signals used were 220 nm and 254 nm (both with a band width of 4 nm). The MS detector was configured with an electrospray ionization source, and the low-resolution mass spectra were acquired by scanning from 140 to 700 AMU with a step size of 0.2 AMU at 0.13 cycles/second, and peak width of 0.008 minutes. The drying gas flow was set to 13 liters per minute at 300° C. and the nebulizer pressure was set to 30 psi. The capillary needle voltage was set at 3000 V, and the fragmentor voltage was set at 100V. Data acquisition was performed with Agilent Chemstation and Analytical Studio Reviewer software.

Abbreviations used in the examples that follow are:

AcOH is acetic acid;

Boc is tert-butyloxycarbonyl;

BrettPhos-Pd-G3 is [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate (CAS Number 1470372-59-8);

DCE is 1,2-dichloroethane;

DCM is dichloromethane;

DIPEA is N,N-diisopropylethylamine;

DMF is N,N-dimethylformamide;

DMSO is dimethylsulfoxide;

eq or equiv is equivalent(s);

EtOAc is ethyl acetate;

EtOH is ethanol;

Et₃N is triethylamine;

HATU is 2-(7-aza-1H-benzotriazole-1-yl)-1,1,3,3-tetramethyluronium hexafluorophosphate;

h or h. is hour(s);

hex is hexane;

IPA is isopropyl alcohol;

m-CPBA is meta-chloroperoxybenzoic acid;

LCMS is liquid chromatography mass spectrometry;

MeCN is acetonitrile;

MeOH is methanol;

min or min. is minute(s);

NMP is N-methyl-2-pyrrolidone;

Pd(dppf)Cl₂ is [1,1′-Bis(diphenylphosphino)ferrocene]dichloropalladium(II);

RP-HPLC is reverse phase high-performance liquid chromatography;

RuPhos-Pd-G3 is (2-dicyclohexylphosphino-2′,6′-diisopropoxy-1,1′-biphenyl)[2-(2′-amino-1,1′-biphenyl)]palladium(II) methanesulfonate (CAS Number 1445085-77-7);

rt, RT, or r.t. is room temperature;

TFA is trifluoroacetic acid;

THF is tetrahydrofuran.

### Example 1. (3aR,5s,6aS)—N-(6-(2-Chloro-5-fluorophenyl)pyridazin-3-yl)-2-(tetrahydro-2H-thiopyran-4-yl)octahydrocyclopenta[c]pyrrol-5-amine

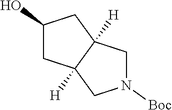 [SMILES: CC(C)(C)OC(=O)N1C[C@H]2C[C@@H](O)C[C@H]2C1]

tert-Butyl (3aR,5r,6aS)-5-hydroxy-3,3a,4,5,6,6a-hexahydro-1H-cyclopenta[c]pyrrole-2-carboxylate. To a solution of tert-butyl (3aR,6aS)-5-oxohexahydrocyclopenta[c]pyrrole-2(1H)-carboxylate (10.0 g, 44.4 mmol) in THF (300 mL) at −78° C. was added a solution of 1.0 M lithium tri-tert-butoxyaluminum hydride solution (53.3 mL, 53.3 mmol) dropwise. The resulting solution was stirred at −78° C. for 2 h, after which time the reaction mixture was warmed to 0° C. and quenched with the slow addition of H₂O (17.0 mL), 1 M NaOH solution (17.0 mL) and H₂O (51.0 mL) sequentially. The mixture was stirred at 0° C. for 1 h, after which time solids were removed by filtration with diethyl ether (3×200 mL). The filtrate was diluted with EtOAc (500 mL) and sat. NH₄Cl solution (300 mL), and the aqueous layer was extracted with EtOAc (3×500 mL). The combined organic extracts were dried with MgSO₄, filtered and concentrated under reduced pressure to give a crude mixture of the title compound as a yellow oil which was carried to the next step without further purification. ¹H-NMR (400 MHz, CDCl₃) δ 4.30 (pent, J=6.4 Hz, 1H), 3.54-3.46 (m, 2H), 3.34 (dd, J=11.2, 3.7 Hz, 2H), 2.65-2.56 (m, 2H), 2.20-2.13 (m, 2H), 1.53-1.47 (m, 2H), 1.45 (s, 9H); d.r.=97:3; ESI-MS=[M+H]⁺−tButyl=172.0.

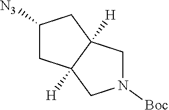 [SMILES: CC(C)(C)OC(=O)N1C[C@H]2C[C@H](N=[N+]=[N-])C[C@H]2C1]

tert-Butyl (3aR,5s,6aS)-5-azido-3,3a,4,5,6,6a-hexahydro-1H-cyclopenta[c]pyrrole-2-carboxylate. To a solution of tert-butyl (3aR,5r,6aS)-5-hydroxyhexahydrocyclopenta[c]pyrrole-2(1H)-carboxylate (10.1 g, 44.4 mmol) in DCM (250 mL), mesyl chloride (4.12 mL, 53.3 mmol), 4-dimethylaminopyridine (0.06 mL, 0.44 mmol), and N,N-diisopropylethylamine (11.6 mL, 66.6 mmol) were added. The reaction mixture was stirred at r.t. overnight. Upon completion, the reaction mixture was quenched with sat. NaHCO₃ (100 mL), and extracted with DCM (3×200 mL). The combined organic extracts were dried with Na₂SO₄, filtered, and concentrated under reduced pressure to give a crude mixture of the mesylate intermediate as an oil which was carried to the next step without further purification. ES-MS=[M+H]⁺−tButyl=250.0.

A mixture of tert-butyl (3aR,5r,6aS)-5-((methylsulfonyl)oxy)hexahydrocyclopenta[c]pyrrole-2(1H)-carboxylate (13.6 g, 44.4 mmol), sodium azide (7.2 g, 111.0 mmol), and tetrabutylammonium iodide (16.4 mg, 0.04 mmol) in DMF (200 mL) was stirred at 60° C. After stirring overnight, the reaction was cooled to r.t. and diluted with EtOAc (200 mL) and H₂O (100 mL). The organic layer was washed with H₂O, and the aqueous layer was back extracted 1× with EtOAc (200 mL). The combined organic extracts were dried with Na₂SO₄, and the solvents were filtered and concentrated under reduced pressure. The crude residue was purified by column chromatography on silica gel (0-100% EtOAc in hexanes) to provide the title compound as a clear oil (6.9 g, 62% over 3 steps). ¹H-NMR (400 MHz, CDCl₃) δ 4.14-4.10 (m, 1H), 3.50-3.48 (m, 2H), 3.22-3.16 (m, 2H), 2.84-2.78 (m, 2H), 2.03-1.97 (m, 2H), 1.76-1.68 (m, 2H), 1.45 (s, 9H); ES-MS=[M+H]⁺−tButyl=197.0.

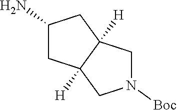 [SMILES: CC(C)(C)OC(=O)N1C[C@H]2C[C@H](N)C[C@H]2C1]

tert-Butyl (3aR,5s,6aS)-5-amino-3,3a,4,5,6,6a-hexahydro-1H-cyclopenta[c]pyrrole-2-carboxylate. tert-Butyl (3aR,5s,6aS)-5-azido-3,3a,4,5,6,6a-hexahydro-1H-cyclopenta[c]pyrrole-2-carboxylate (6.4 g, 25.3 mmol) was dissolved in THF (400 mL), and 20% wt Pd(OH)₂/C (1.8 g, 2.5 mmol) was added. The resulting mixture was stirred under H2 (balloon) at 0° C. for 8 h, then slowly warmed to r.t. and stirred overnight, after which time the reaction mixture was filtered through a pad of Celite with EtOAc, and concentrated under reduced pressure. The crude residue was purified by column chromatography on silica gel (0-100% DCM, MeOH, NH₄OH (89:10:1) in DCM) to provide the title compound as a solid (5.3 g, 93%). ¹H-NMR (400 MHz, MeOD) δ 3.54-3.43 (m, 3H), 3.33-3.32 (m, 2H), 3.17-3.12 (m, 2H), 2.86-2.80 (m, 2H), 1.81-1.75 (m, 2H), 1.70-1.62 (m, 2H), 1.47 (s, 9H); ES-MS [M+H]⁺=227.0.

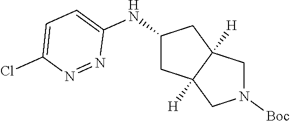 [SMILES: CC(C)(C)OC(=O)N1C[C@H]2C[C@H](Nc3ccc(Cl)nn3)C[C@H]2C1]

tert-Butyl (3aR,5s,6aS)-5-[(6-chloropyridazin-3-yl)amino]-3,3a,4,5,6,6a-hexahydro-1H-cyclopenta[c]pyrrole-2-carboxylate. tert-Butyl (3aR,5s,6aS)-5-amino-3,3a,4,5,6,6a-hexahydro-1H-cyclope-nta[c]pyrrole-2-carboxylate (1.0 g, 4.42 mmol) and 3,6-dichloropyridazine (1.97 g, 13.3 mmol) were dissolved in t-butanol (10 mL), and DIPEA (2.31 mL, 13.3 mmol) was added. The resulting solution was heated to 150° C. under microwave irradiation for 2 h, after which time the solvents were concentrated under reduced pressure and the crude residue was purified by column chromatography on silica gel (3-100% EtOAc in hexanes) to give the title compound as a tan solid. ¹H-NMR (400 MHz, DMSO-d6) δ 7.34 (d, J=9.4 Hz, 1H), 7.15 (d, J=6.6 Hz, 1H), 6.86 (d, J=9.4 Hz, 1H), 4.37-4.24 (m, 1H), 3.49-3.45 (m, 2H), 3.07 (dd, J=11.2, 4.0 Hz, 2H), 2.76 (br, 2H), 1.89-1.83 (m, 2H), 1.77-1.70 (m, 2H), 1.40 (s, 9H); ES-MS=[M+H]⁺−tButyl=283.0.

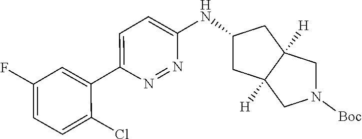 [SMILES: CC(C)(C)OC(=O)N1C[C@H]2C[C@H](Nc3ccc(-c4cc(F)ccc4Cl)nn3)C[C@H]2C1]

tert-Butyl (3aR,5s,6aS)-5-((6-(2-chloro-5-fluorophenyl)pyridazin-3-yl)amino)hexahydrocyclopenta[c]pyrrole-2(1H)-carboxylate. tert-Butyl (3aR,5s,6aS)-5-[(6-chloropyridazin-3-yl)amino]-3,3a,4,5,6,6a-hexahydro-1H-cyclopenta[c]pyrrole-2-carboxylate (187 mg, 0.55 mmol), 2-chloro-5-fluorophenylboronic acid (144 mg, 0.83 mmol), potassium carbonate (232 mg, 1.65 mmol) and BrettPhos-Pd-G3 (50 mg, 0.055 mmol) were combined in a vial which was sealed and placed under an inert atmosphere. 5:1 (v/v) 1,4-Dioxane/H₂O solution (3.5 mL, degassed) was then added via syringe. The resulting mixture was stirred at 100° C. for 2.5 h, after which time the reaction mixture was cooled to r.t. and diluted with DCM and sat. NaHCO₃. The aqueous layer was extracted with DCM, and the combined organic extracts were filtered through a phase separator and concentrated. The crude residue was purified by column chromatography on silica gel (3-50% EtOAc in hexanes) to give the title compound as a white solid (125 mg, 52%). ¹H-NMR (400 MHz, DMSO-d6) δ 7.62 (dd, J=8.8, 5.2 Hz, 1H), 7.55 (d, J=9.3 Hz, 1H), 7.45 (dd, J=9.3, 3.1 Hz, 1H), 7.36-7.31 (m, 1H), 7.18 (d, J=6.7 Hz, 1H), 6.87 (d, J=9.3 Hz, 1H), 4.50-4.42 (m, 1H), 3.52-3.47 (m, 2H), 3.10 (dd, J=11.2, 4.0 Hz, 2H), 2.80 (br, 2H), 1.93-1.88 (m, 2H), 1.82-1.75 (m, 2H), 1.41 (s, 9H); ES-MS [M+H]⁺=433.0.

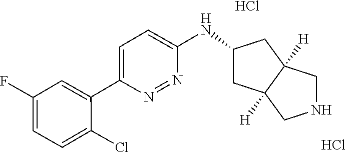 [SMILES: Cl ; Fc1ccc(Cl)c(-c2ccc(N[C@@H]3C[C@H]4CNC[C@H]4C3)nn2)c1 ; Cl]

(3aR,5s,6aS)—N-(6-(2-Chloro-5-fluorophenyl)pyridazin-3-yl)octahydrocyclopenta[c]pyrrol-5-amine dihydrochloride. tert-Butyl (3aR,5s,6aS)-5-((6-(2-chloro-5-fluorophenyl)pyridazin-3-yl)amino)hexahydrocyclopenta[c]pyrrole-2(1H)-carboxylate (868 mg, 2.00 mmol) was dissolved in 1,4-dioxane (10 mL) and MeOH (1 mL), and a 4 M solution of HCl in dioxanes (5 mL) was added dropwise. The resulting mixture was stirred at r.t. for 30 min, after which time solvents were concentrated under reduced pressure to give the title compound as a white solid which was used directly without further purification (813 mg, 100%). ES-MS [M+H]⁺=333.4.

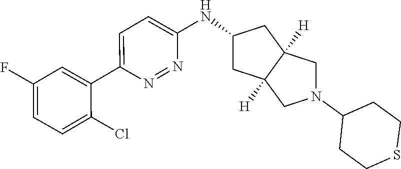 [SMILES: Fc1ccc(Cl)c(-c2ccc(N[C@H]3C[C@@H]4CN(C5CCSCC5)C[C@@H]4C3)nn2)c1]

(3aR,5s,6aS)—N-(6-(2-Chloro-5-fluorophenyl)pyridazin-3-yl)-2-(tetrahydro-2H-thiopyran-4-yl)octahydrocyclopenta[c]pyrrol-5-amine. (3aR,5s,6aS)—N-(6-(2-Chloro-5-fluorophenyl)pyridazin-3-yl)octahydrocyclopenta[c]pyrrol-5-amine dihydrochloride was dissolved in DCM (0.5 mL), THF (0.5 mL) and AcOH (0.1 mL) and tetrahydro-4H-thiopyran-4-one (36 mg, 0.31 mmol) was added. The resulting mixture was stirred at r.t. for 10 min, after which time sodium triacetoxyborohydride (39 mg, 0.18 mmol) was added. The resulting mixture was heated to 40° C. and stirred for 1 h, after which time the reaction mixture was quenched with sat. NaHCO₃, and extracted with 3:1 chloroform/IPA (v/v). The combined organic extracts were filtered through a phase separator and concentrated, and crude residue was purified by RP-HPLC (5-35% MeCN in 0.1% TFA aqueous solution over 5 min). The fractions containing product were basified with sat. NaHCO₃, and extracted with 3:1 chloroform/IPA (v/v). The combined organic extracts were filtered through a phase separator and concentrated to give the title compound as a white solid (11.4 mg, 43%). ¹H-NMR (400 MHz, CDCl₃) δ 7.62 (d, J=9.3 Hz, 1H), 7.48 (dd, J=9.2, 3.1 Hz, 1H), 7.41 (dd, J=8.8, 5.1 Hz, 1H), 7.07-7.02 (m, 1H), 6.70 (d, J=9.3 Hz, 1H), 5.02 (d, J=7.0 Hz, 1H), 4.42-4.34 (m, 1H), 2.83-2.72 (m, 6H), 2.58-2.51 (m, 2H), 2.40 (d, J=5.0 Hz, 2H), 2.22-2.10 (m, 3H), 1.98 (dd, J=11.9, 5.3 Hz, 2H), 1.82-1.72 (m, 4H); ES-MS [M+H]⁺=433.2.

### Example 2. (3aR,5s,6aS)—N-(6-(2,5-difluorophenyl)pyridazin-3-yl)-2-(2,2-dimethyltetrahydro-2H-pyran-4-yl)octahydrocyclopenta[c]pyrrol-5-amine

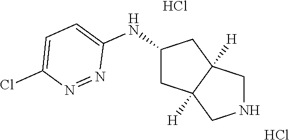 [SMILES: Cl ; Clc1ccc(N[C@@H]2C[C@H]3CNC[C@H]3C2)nn1 ; Cl]

(3aR,5s,6aS)—N-(6-Chloropyridazin-3-yl)octahydrocyclopenta[c]pyrrol-5-amine dihydrochloride. tert-Butyl (3aR,5s,6aS)-5-[(6-chloropyridazin-3-yl)amino]-3,3a,4,5,6,6a-hexahydro-1H-cyclopenta[c]pyrrole-2-carboxylate (326 mg, 0.96 mmol) was dissolved in 1,4-dioxane (4 mL) and MeOH (0.5 mL), and 4 M HCl in dioxanes solution (3.6 mL) was added dropwise. The resulting mixture was stirred at r.t. for 1 h, after which time the solvents were concentrated under reduced pressure, and the resulting off white solid was used directly without further purification (300 mg, 100%). ES-MS [M+H]⁺=239.3.

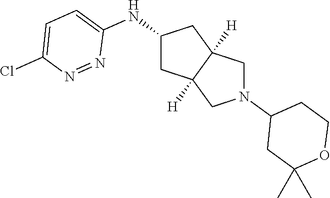 [SMILES: CC1(C)CC(N2C[C@H]3C[C@H](Nc4ccc(Cl)nn4)C[C@H]3C2)CCO1]

(3aR,5s,6aS)—N-(6-Chloropyridazin-3-yl)-2-(2,2-dimethyltetrahydro-2H-pyran-4-yl)octahydrocyclopenta[c]pyrrol-5-amine. (3aR,5s,6aS)—N-(6-Chloropyridazin-3-yl)octahydrocyclopenta[c]pyrrol-5-amine dihydrochloride (300 mg, 0.96 mmol) was dissolved in DCM (3 mL), THF (3 mL) and AcOH (0.5 mL), and 2,2-dimethyltetrahydro-4H-pyran-4-one (370 mg, 2.89 mmol) was added, followed by sodium triacetoxyborohydride (612 mg, 2.89 mmol). The resulting solution was stirred at 40° C. for 1 h, after which time the reaction was quenched with sat. NaHCO₃, and extracted with 3:1 chloroform/IPA (v/v). The combined organic extracts were filtered through a phase separator and concentrated. The crude residue was taken up in DMSO and purified directly by RP-HPLC (5-35% MeCN in 0.1% TFA aqueous solution over 20 min). The fractions containing product were basified with sat. NaHCO₃, and extracted with 3:1 chloroform/IPA (v/v). The combined organic extracts were dried with MgSO₄, and the solvents were filtered and concentrated under reduced pressure to give the title compound as a white solid (138 mg, 41%). ES-MS [M+H]⁺=351.3.

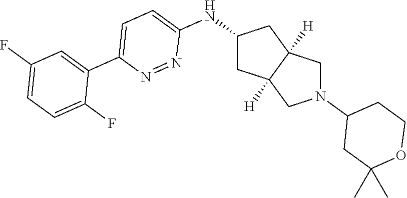 [SMILES: CC1(C)CC(N2C[C@H]3C[C@H](Nc4ccc(-c5cc(F)ccc5F)nn4)C[C@H]3C2)CCO1]

(3aR,5s,6aS)—N-(6-(2,5-Difluorophenyl)pyridazin-3-yl)-2-(2,2-dimethyltetrahydro-2H-pyran-4-yl)octahydrocyclopenta[c]pyrrol-5-amine. (3aR,5s,6aS)—N-(6-Chloropyridazin-3-yl)-2-(2,2-dimethyltetrahydro-2H-pyran-4-yl)octahydrocyclopenta[c]pyrrol-5-amine (15.3 mg, 0.044 mmol), 2,5-difluorophenylboronic acid (10.3 mg, 0.065 mmol), potassium carbonate (18.3 mg, 0.13 mmol), and BrettPhos-Pd-G3 (4.0 mg, 0.004 mmol) were combined in a vial which was sealed and placed under an inert atmosphere. 5:1 1,4-Dioxane/H₂O solution (v/v, 1 mL, degassed) was then added via syringe. The resulting mixture was stirred at 100° C. for 2 h, after which time the reaction mixture was cooled to r.t. and the solvents were concentrated. The crude residue was taken up in DMSO, and the solids were removed by syringe filtration. The crude residue was purified directly by RP-HPLC (5-35% MeCN in 0.1% TFA aqueous solution over 5 min). Fractions containing product were basified with sat. NaHCO₃, and extracted with 3:1 chloroform/IPA (v/v). The combined organic extracts were filtered through a phase separator and concentrated to give the title compound as a yellow oil (3.4 mg, 18%). ¹H-NMR (400 MHz, CDCl₃) δ 7.90-7.85 (m, 1H), 7.70 (dd, J=9.4, 2.1 Hz, 1H), 7.12-7.00 (m, 2H), 6.68 (d, J=9.4 Hz, 1H), 4.79 (d, J=7.2 Hz, 1H), 4.48-4.39 (m, 1H). 3.81-3.76 (m, 1H), 3.68-3.62 (m, 1H), 2.90-2.85 (m, 2H), 2.80-2.71 (m, 2H), 2.36-2.28 (m, 1H), 2.27-2.22 (m, 2H), 2.04-1.97 (m, 2H), 1.79-1.65 (m, 4H), 1.50-1.41 (m, 1H), 1.37-1.31 (m, 1H), 1.24 (s, 3H), 1.22 (s, 3H). ES-MS [M+H]⁺=429.4.

### Example 3. (3aR,5s,6aS)-2-(2,2-Dimethyltetrahydro-2H-pyran-4-yl)-N-(6-morpholinopyridazin-3-yl)octahydrocyclopenta[c]pyrrol-5-amine

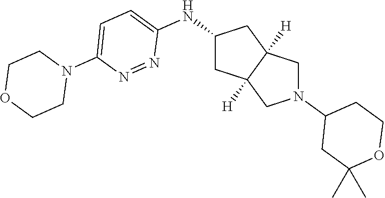 [SMILES: CC1(C)CC(N2C[C@H]3C[C@H](Nc4ccc(N5CCOCC5)nn4)C[C@H]3C2)CCO1]

(3aR,5s,6aS)—N-(6-Chloropyridazin-3-yl)-2-(2,2-dimethyltetrahydro-2H-pyran-4-yl)octahydrocyclopenta[c]pyrrol-5-amine (15.3 mg, 0.044 mmol) and morpholine (0.019 mL, 0.22 mmol) were dissolved in NMP (1 mL), and conc. HCl (0.018 mL, 0.22 mmol) was added, followed by DIPEA (0.038 mL, 0.22 mmol). The resulting solution was heated to 200° C. under microwave irradiation for 1 h, after which time crude residue was purified directly by RP-HPLC (10-50% MeCN in 0.05% NH₄OH aqueous solution over 10 min). The fractions containing product were concentrated to give the title compound as a colorless oil (5.6 mg, 32%). ¹H-NMR (400 MHz, CDCl₃) δ 6.88 (d, J=9.6 Hz, 1H), 6.63 (d, J=9.6 Hz, 1H), 4.48-4.39 (m, 1H), 4.23 (br, 1H), 3.84-3.76 (m, 5H), 3.66-3.60 (m, 1H), 3.41-3.39 (m, 4H), 3.09 (br, 2H), 2.80 (br, 2H), 2.46 (br, 1H), 2.22 (br, 2H), 2.03-1.97 (m, 2H), 1.79-1.74 (m, 2H), 1.65-1.51 (m, 3H), 1.44-1.36 (m, 1H), 1.24 (s, 3H), 1.20 (s, 3H). ES-MS [M+H]⁺=402.2.

### Example 4. (3aR,5s,6aS)—N-(6-(2-Chloro-5-fluorophenyl)pyridazin-3-yl)-2-(1-(tetrahydro-2H-pyran-4-yl)cyclopropyl)octahydrocyclopenta[c]pyrrol-5-amine

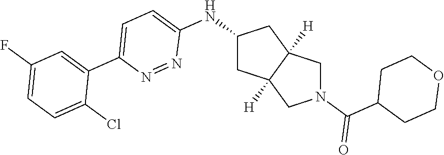 [SMILES: O=C(C1CCOCC1)N1C[C@H]2C[C@H](Nc3ccc(-c4cc(F)ccc4Cl)nn3)C[C@H]2C1]

((3aR,5s,6aS)-5-((6-(2-Chloro-5-fluorophenyl)pyridazin-3-yl)amino)hexahydrocyclopenta[c]pyrrol-2(1H)-yl)(tetrahydro-2H-pyran-4-yl)methanone. (3aR,5s,6aS)—N-(6-(2-Chloro-5-fluorophenyl)pyridazin-3-yl)octahydrocyclopenta[c]pyrrol-5-amine hydrochloride (53.4 mg, 0.14 mmol) and 4-oxanoic acid (22.6 mg, 0.17 mmol) were dissolved in DMF (1 mL), and DIPEA (0.076 mL, 0.43 mmol) was added, followed by HATU (82.5 mg, 0.22 mmol). The resulting solution was stirred at r.t. for 1 h, after which time the reaction mixture was purified directly by RP-HPLC (25-65% MeCN in 0.05% NH₄OH aqueous solution over 10 min). Fractions containing product were concentrated to give the title compound as a colorless oil (39 mg, 61%). ES-MS [M+H]⁺=445.0.

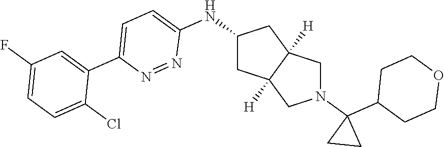 [SMILES: Fc1ccc(Cl)c(-c2ccc(N[C@H]3C[C@@H]4CN(C5(C6CCOCC6)CC5)C[C@@H]4C3)nn2)c1]

(3aR,5s,6aS)—N-(6-(2-Chloro-5-fluorophenyl)pyridazin-3-yl)-2-(1-(tetrahydro-2H-pyran-4-yl)cyclopropyl)octahydrocyclopenta[c]pyrrol-5-amine. To a solution of ethylmagnesium bromide (0.062 mL, 0.062 mmol, 1.0 M solution) in THF (0.2 mL) was added titanium(IV) isopropoxide (0.008 mL, 0.026 mmol) in 0.1 mL THF at −78° C. The resulting solution was stirred at −78° C. for 30 min under an inert atmosphere, after which time ((3aR,5s,6aS)-5-((6-(2-chloro-5-fluorophenyl)pyridazin-3 yl)amino)hexahydrocyclopenta[c]pyrrol-2(1H)-yl)(tetrahydro-2H-pyran-4-yl)methanone (11 mg, 0.025 mmol (in 0.3 mL THF)) was added dropwise. The resulting solution was warmed to r.t. and then stirred at reflux for 1 h, after which time the reaction mixture was cooled to 0° C. and another 2.5 eq ethylmagnesium bromide (1.0 M solution, 5 eq total) and 1.05 eq titanium(IV) isopropoxide (in 0.1 mL THF, 2.1 eq total) were added dropwise. The resulting brown solution was warmed to r.t. and stirred for 1 h, after which time the reaction was quenched with H₂O and diluted with 3:1 chloroform/IPA (v/v). The aqueous layer was extracted with 3:1 chloroform/IPA (v/v), and combined organic extracts were filtered through a phase separator and concentrated. The crude residue was purified by RP-HPLC (65-95% MeCN in 0.05% NH₄OH aqueous solution over 5 min), and the fractions containing product were concentrated to give the title compound as a tan solid (1.1 mg, 10%). ¹H-NMR (400 MHz, MeOD) δ 7.58 (d, J=9.4 Hz, 1H), 7.56 (dd, J=8.8, 5.0 Hz, 1H), 7.37 (dd, J=9.0, 3.1 Hz, 1H), 7.24-7.19 (m, 1H), 6.94 (d, J=9.4 Hz, 1H), 4.54-4.48 (m, 1H), 3.99-3.95 (m, 2H), 3.45-3.39 (m, 2H), 2.67-2.64 (m, 4H), 2.46-2.42 (1, 2H), 1.94-1.83 (m, 4H), 1.63-1.49 (m, 5H), 0.70 (dd, J=6.5, 5.0 Hz, 2H), 0.44 (dd, J=6.2, 4.8 Hz, 2H). ES-MS [M+H]⁺=457.4.

### Example 5. N-(2-Fluorophenyl)-6-(((3aR,5s,6aS)-2-((tetrahydro-2H-pyran-4-yl)methyl)octahydrocyclopenta[c]pyrrol-5-yl)amino)pyridazine-3-carboxamide

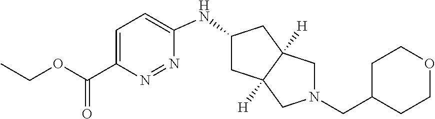 [SMILES: CCOC(=O)c1ccc(N[C@H]2C[C@@H]3CN(CC4CCOCC4)C[C@@H]3C2)nn1]

Ethyl 6-(((3aR,5s,6aS)-2-((tetrahydro-2H-pyran-4-yl)methyl)octahydrocyclopenta[c]pyrrol-5-yl)amino)pyridazine-3-carboxylate. (3aR,5s,6aS)—N-(6-Chloropyridazin-3-yl)-2-((tetrahydro-2H-pyran-4-yl)methyl)octahydrocyclopenta[c]pyrrol-5-amine (200 mg, 0.59 mmol), sodium acetate (99 mg, 1.19 mmol), and Pd(dppf)Cl₂ DCM (49 mg, 0.059 mmol) were dissolved in EtOH (5 mL) and DMF (1 mL). The resulting solution was purged under vacuum and then stirred under an atmosphere of CO (balloon) at 70° C. for 3 h, after which time the reaction mixture was cooled to r.t. and diluted with EtOAc and H₂O. The organic layer was removed, and the aqueous layer was basified with sat. NaHCO₃, and extracted with 3:1 chloroform/IPA (v/v) solution. The combined organic extracts were dried with MgSO₄, and the solvents were filtered and concentrated under reduced pressure to give the title compound as a tan solid, which was used without further purification (194 mg, 87%). ES-MS [M+H]⁺=375.5.

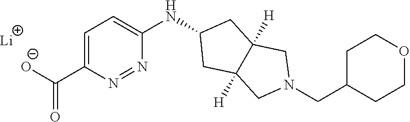 [SMILES: [Li+] ; O=C([O-])c1ccc(N[C@H]2C[C@@H]3CN(CC4CCOCC4)C[C@@H]3C2)nn1]

Lithium 6-(((3aR,5s,6aS)-2-((tetrahydro-2H-pyran-4-yl)methyl)octahydrocyclopenta[c]pyrrol-5-yl)amino)pyridazine-3-carboxylate. Ethyl 6-(((3aR,5s,6aS)-2-((tetrahydro-2H-pyran-4-yl)methyl)octahydrocyclopenta[c]pyrrol-5-yl)amino)pyridazine-3-carboxylate (194 mg, 0.52 mmol) was dissolved in THF (2 mL) and a solution of LiOH (39 mg, 1.55 mmol) in H₂O (2 mL) was added dropwise. The resulting solution was stirred at r.t. for 1 h, after which time the solvents were concentrated under reduced pressure and the resulting brown solid was dried and used without further purification (182 mg, 100%). ES-MS [M+H]⁺=347.2.

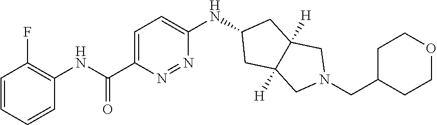 [SMILES: O=C(Nc1ccccc1F)c1ccc(N[C@H]2C[C@@H]3CN(CC4CCOCC4)C[C@@H]3C2)nn1]

N-(2-Fluorophenyl)-6-(((3aR,5s,6aS)-2-((tetrahydro-2H-pyran-4-yl)methyl)octahydrocyclopenta[c]pyrrol-5-yl)amino)pyridazine-3-carboxamide. Lithium 6-(((3aR,5s,6aS)-2-((tetrahydro-2H-pyran-4-yl)methyl)octahydrocyclopenta[c]pyrrol-5-yl)amino)pyridazine-3-carboxylate (18.2 mg, 0.052 mmol) was dissolved in DMF (1 mL) and DIPEA (45 μL, 0.26 mmol) was added, followed by 2-fluoroaniline (11.5 mg, 0.10 mmol) and HATU (29 mg, 0.078 mmol). The resulting solution was stirred at r.t. for 2 h, after which time the reaction mixture was purified directly by RP-HPLC (5-35% MeCN in 0.1% TFA aqueous solution over 5 min). The fractions containing product were basified with sat. NaHCO₃, and extracted with 3:1 chloroform/IPA (v/v). The combined organic extracts were filtered through a phase separator and concentrated to give the title compound as a white solid (9.5 mg, 42%). ¹H-NMR (400 MHz, CDCl₃) δ 10.04 (d, J=1.7 Hz, 1H), 8.50 (td, J=8.2, 1.4 Hz, 1H), 8.04 (d, J=9.3 Hz, 1H), 7.19-7.04 (m, 3H), 6.75 (d, J=9.3 Hz, 1H), 5.39 (br, 1H), 4.45 (br, 1H), 3.97 (dd, J=11.2, 3.6 Hz, 2H), 3.39 (td, J=11.8, 1.8 Hz, 2H), 2.86-2.59 (m, 4H), 2.44-2.27 (m, 4H), 2.00 (dd, J=12.0, 4.9 Hz, 2H), 1.82-1.66 (m, 5H), 1.35-1.24 (m, 2H); ES-MS [M+H]⁺=440.2.

### Example 6. (3aR,5s,6aS)—N-(6-(2-Chloro-5-fluorophenyl)pyridazin-3-yl)-2-(3-methoxypropyl)octahydrocyclopenta[c]pyrrol-5-amine

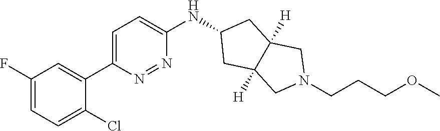 [SMILES: COCCCN1C[C@H]2C[C@H](Nc3ccc(-c4cc(F)ccc4Cl)nn3)C[C@H]2C1]

(3aR,5s,6aS)—N-(6-(2-Chloro-5-fluorophenyl)pyridazin-3-yl)octahydrocyclopenta[c]pyrrol-5-amine dihydrochloride (20.3 mg, 0.050 mmol) was dissolved in DMF (1 mL) and cesium carbonate (49 mg, 0.15 mmol) was added, followed by 1-bromo-3-methoxypropane (38 mg, 0.25 mmol). The resulting solution was stirred at 70° C. overnight, after which time the solids were removed via syringe filtration, and the crude residue was purified by RP-HPLC (5-35% MeCN in 0.1% TFA aqueous solution over 5 min). Fractions containing product were basified with sat. NaHCO₃, and extracted with 3:1 chloroform/IPA (v/v). The combined organic extracts were filtered through a phase separator and concentrated to give the title compound as a white solid (3.2 mg, 16%). ¹H-NMR (400 MHz, MeOD) δ 7.46 (d, J=9.3 Hz, 1H), 7.44 (dd, J=8.8, 5.0 Hz, 1H), 7.25 (dd, J=9.0, 3.1 Hz, 1H), 7.12-7.07 (m, 1H), 6.82 (d, J=9.4 Hz, 1H), 4.45-4.38 (m, 1H), 3.35 (t, J=6.2 Hz, 2H), 3.23 (s, 3H), 2.92-2.87 (m, 2H), 2.74-2.69 (m, 2H), 2.49-2.45 (m, 2H), 2.17-2.14 (m, 2H), 1.91-1.85 (m, 2H), 1.74-1.61 (m, 4H). ES-MS [M+H]⁺=405.4.

### Example 7. 1-((3aR,5s,6aS)-5-((6-(2-Chloro-5-fluorophenyl)pyridazin-3-yl)amino)hexahydrocyclopenta[c]pyrrol-2(1H)-yl)-2-methylpropan-2-ol

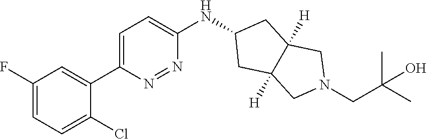 [SMILES: CC(C)(O)CN1C[C@H]2C[C@H](Nc3ccc(-c4cc(F)ccc4Cl)nn3)C[C@H]2C1]

(3aR,5s,6aS)—N-(6-(2-Chloro-5-fluorophenyl)pyridazin-3-yl)octahydrocyclopenta[c]pyrrol-5-amine hydrochloride (19.7 mg, 0.053 mmol) was dissolved in EtOH (1 mL), and DIPEA (0.028 mL, 0.16 mmol) was added, followed by isobutylene oxide (0.014 mL, 0.16 mmol). The resulting solution was heated to 70° C. for 4 h, after which time the reaction mixture was cooled to r.t., and the solvents were concentrated. The crude residue was purified by RP-HPLC (5-35% MeCN in 0.1% TFA aqueous solution over 5 min). The fractions containing product were basified with sat. NaHCO₃, and extracted with 3:1 chloroform/IPA (v/v). The combined organic extracts were filtered through a phase separator and concentrated to give the title compound as a white solid (11 mg, 51%). ¹H-NMR (400 MHz, CDCl₃) δ 7.62 (d, J=9.3 Hz, 1H), 7.47 (dd, J=9.2, 3.1 Hz, 1H), 7.41 (dd, J=8.8, 5.0 Hz, 1H), 7.07-7.02 (m, 1H), 6.71 (d, J=9.3 Hz, 1H), 5.03 (d, J=4.8 Hz, 1H), 4.41 (br, 1H), 2.96 (br, 2H), 2.85 (br, 2H), 2.67 (br, 2H), 2.55 (br, 2H), 2.04-1.96 (m, 2H), 1.86-1.79 (m, 2H), 1.24 (m, 6H); ES-MS [M+H]⁺=405.4.

### Example 8. (3aR,5s,6aS)—N-(6-(Cyclohexylsulfonyl)pyridazin-3-yl)-2-((tetrahydro-2H-pyran-4-yl)methyl)octahydrocyclopenta[c]pyrrol-5-amine

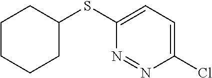 [SMILES: Clc1ccc(SC2CCCCC2)nn1]

3-Chloro-6-(cyclohexylthio)pyridazine. A solution of 3,6-dichloropyridazine (500.0 mg, 3.36 mmol) in DMF (25.0 mL) was treated with cyclohexyl mercaptan (0.41 mL, 3.36 mmol) and K₂CO₃ (1391.6 mg, 10.1 mmol). The resulting mixture was heated to 90° C. for 3 h. At this time, the reaction mixture was cooled to r.t., poured into a solution of water (10 mL) and the mixture was extracted with DCM (3×20.0 mL). The combined organic layers were dried over Na₂SO₄, filtered, and concentrated under reduced pressure. The crude residue was purified by column chromatography on silica gel (0-100% EtOAc in hexanes) to provide the title compound as a white solid (763.2 mg, 99%). ¹H NMR (400 MHz, CDCl₃) δ 7.25-7.19 (m, 2H), 4.09 (m, 1H), 2.20-2.11 (m, 2H), 1.81-1.71 (m, 2H), 1.69-1.61 (m, 1H), 1.57-1.41 (m, 4H), 1.37-1.25 (m, 1H); ES-MS [M+H]⁺=229.0.

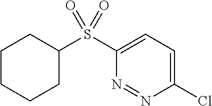 [SMILES: O=S(=O)(c1ccc(Cl)nn1)C1CCCCC1]

3-Chloro-6-(cyclohexylsulfonyl)pyridazine. 3-Chloro-6-(cyclohexylthio)pyridazine (354.9 mg, 1.55 mmol) was treated with m-CPBA (669.4 mg, 3.88 mmol) in DCM (12.0 mL). The mixture was stirred at r.t. for 12 h. Upon completion, the reaction mixture was quenched with sat. aq. NaHCO₃ (5.0 mL) and extracted with DCM (3×20.0 mL). The combined organic layers were dried over Na₂SO₄, filtered, and concentrated. The crude residue was purified by column chromatography on silica gel (0-100% EtOAc in hexanes) to provide the title compound as a white solid (384.2 mg, 94%). ES-MS [M+H]⁺=261.0.

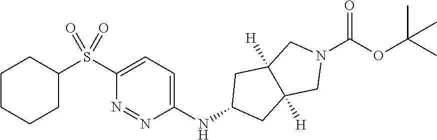 [SMILES: CC(C)(C)OC(=O)N1C[C@H]2C[C@H](Nc3ccc(S(=O)(=O)C4CCCCC4)nn3)C[C@H]2C1]

tert-Butyl (3aR,5s,6aS)-5-((6-(cyclohexylsulfonyl)pyridazin-3-yl)amino)hexahydrocyclopenta[c]pyrrole-2(1H)-carboxylate. 3-Chloro-6-cyclohexylsulfonyl-pyridazine (384.2 mg, 1.47 mmol), tert-butyl (3aR,5s,6aS)-5-aminohexahydrocyclopenta[c]pyrrole-2(1H)-carboxylate (400.2 mg, 1.77 mmol), and Et₃N (0.62 mL, 4.42 mmol) were heated at 100° C. in DMF (8.5 mL) for 3 h. Upon completion, the reaction mixture was cooled to r.t., diluted with DCM (10.0 mL), filtered, and then concentrated under reduced pressure. The crude residue was purified by column chromatography on silica gel (0-100% EtOAc in hexanes) to provide the title compound as a solid (424.1 mg, 63%). ES-MS=[M+H]⁺−tButyl=395.0.

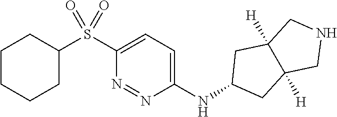 [SMILES: O=S(=O)(c1ccc(N[C@@H]2C[C@H]3CNC[C@H]3C2)nn1)C1CCCCC1]

(3aR,5s,6aS)—N-(6-(Cyclohexylsulfonyl)pyridazin-3-yl)octahydrocyclopenta[c]pyrrol-5-amine. tert-Butyl (3aR,5s,6aS)-5-((6-(cyclohexylsulfonyl)pyridazin-3-yl)amino)hexahydrocyclopenta[c]pyrrole-2(1H)-carboxylate (424.1 mg, 0.94 mmol) was dissolved in 1,4-dioxane (6.0 mL) and MeOH (1.0 mL), and 4 M HCl in dioxane solution (3.5 mL, 14.1 mmol) was added dropwise. The resulting mixture was stirred at r.t. for 1 h, after which time the solvents were concentrated under reduced pressure. The crude residue was purified by column chromatography on silica gel (0-20% MeOH in DCM) to provide the title compound as a white solid (327.3 mg, 99%). ¹H NMR (400 MHz, CD₃OD) δ 7.70 (d, J=9.4 Hz, 1H), 6.95 (d, J=9.5 Hz, 1H), 4.48 (m, 1H), 3.37 (tt, J=12.0, 3.4 Hz, 1H), 3.09 (dd, J=11.5, 7.4 Hz, 2H), 2.85-2.76 (m, 2H), 2.68 (dd, J=11.5, 4.2 Hz, 2H), 2.06-1.97 (m, 2H), 1.93-1.80 (m, 6H), 1.74-1.65 (m, 1H), 1.47 (qd, J=12.4, 3.1 Hz, 2H), 1.32 (qt, J=12.1, 2.7 Hz, 2H), 1.21 (tt, J=12.5, 2.8 Hz, 1H); ES-MS [M+H]⁺=351.0.

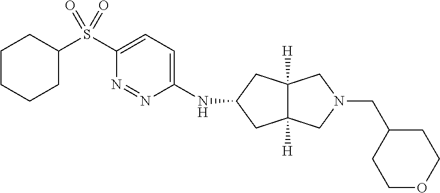 [SMILES: O=S(=O)(c1ccc(N[C@H]2C[C@@H]3CN(CC4CCOCC4)C[C@@H]3C2)nn1)C1CCCCC1]

(3aR,5s,6aS)—N-(6-(Cyclohexylsulfonyl)pyridazin-3-yl)-2-((tetrahydro-2H-pyran-4-yl)methyl)octahydrocyclopenta[c]pyrrol-5-amine. (3aR,5s,6aS)—N-(6-(Cyclohexylsulfonyl)pyridazin-3-yl)octahydrocyclopenta[c]pyrrol-5-amine (15.0 mg, 0.05 mmol) was dissolved in DCM (0.7 mL). To this reaction mixture, 4-oxanaldehyde (14.0 μL, 0.14 mmol) was added, followed by sodium triacetoxyborohydride (28.7 mg, 0.14 mmol). The resulting solution was stirred at r.t. for 1 h, after which time LCMS indicated product formation. The reaction was quenched with sat. NaHCO₃ (0.2 mL), extracted with 3:1 chloroform/IPA (v/v, 3×3.0 mL), and the organic extracts were filtered through a phase separator and concentrated. The crude residue was taken up in DMSO and the solids were removed by syringe filtration. The crude residue was purified by RP-HPLC (5-95% MeCN in 0.1% TFA aqueous solution over 5 min). The fractions containing product were concentrated and further purified by RP-HPLC (5%-95% MeCN in 0.05% NH₄OH aqueous solution over 5 min). The fractions containing product were concentrated to give the title compound as a white solid (11.2 mg, 55%). ¹H NMR (400 MHz, CDCl₃) δ 7.78 (d, J=9.3 Hz, 1H), 6.67 (d, J=9.4 Hz, 1H), 5.28 (br s, 1H), 4.36 (br s, 1H), 3.97 (dd, J=11.1, 3.6 Hz, 2H), 3.55 (tt, J=12.2, 3.4 Hz, 1H), 3.39 (td, J=12.0, 1.7 Hz, 2H), 2.79-2.66 (m, 2H), 2.55-2.45 (m, 2H), 2.42-2.33 (m, 2H), 2.24 (d, J=6.7 Hz, 2H), 2.07 (m, 2H), 2.02-1.92 (m, 2H), 1.92-1.83 (m, 2H), 1.72 (m, 6H), 1.61-1.49 (m, 2H), 1.36-1.16 (m, 5H); ES-MS [M+H]⁺=449.0.

### Example 9. 7-Cyclopropyl-5,5-dimethyl-3-(((3aR,5s,6aS)-2-((tetrahydro-2H-pyran-4-yl)methyl)octahydrocyclopenta[c]pyrrol-5-yl)amino)-5,7-dihydro-6H-pyrrolo[2,3-c]pyridazin-6-one

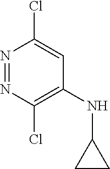 [SMILES: Clc1cc(NC2CC2)c(Cl)nn1]

3,6-Dichloro-N-cyclopropylpyridazin-4-amine. 3,6-Dichloro-4-fluoropyridazine (200.0 mg, 1.20 mmol) was dissolved in THF (3.0 mL). Cyclopropylamine (0.66 mL, 9.58 mmol) was then added dropwise. The vial was sealed and the mixture was stirred at r.t. for 1 h. The mixture was diluted with EtOAc (3.0 mL) and H₂O (1.0 mL) and then extracted with EtOAc (4×10.0 mL). The organic layers were combined, dried with Na₂SO₄, filtered and concentrated under reduced pressure. The crude residue was purified by column chromatography on silica gel (0-100% EtOAc in hexanes) to provide the title compound as a white solid (243.6 mg, 99%). ES-MS [M+H]⁺=204.0.

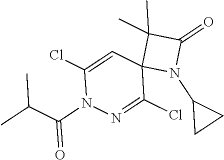 [SMILES: CC(C)C(=O)N1N=C(Cl)C2(C=C1Cl)N(C1CC1)C(=O)C2(C)C]

5,8-Dichloro-1-cyclopropyl-7-isobutyryl-3,3-dimethyl-1,6,7-triazaspiro[3.5]nona-5,8-dien-2-one. 3,6-Dichloro-N-cyclopropyl-pyridazin-4-amine (244.4 mg, 1.20 mmol) was dissolved in dry DCM (15.0 mL) in a N2 flushed flask, then Et₃N (0.45 mL, 3.23 mmol) and isobutyryl chloride (0.28 m1, 2.64 mmol) were added. The mixture was stirred at r.t. for 18 h. H₂O (3.0 mL) and DCM (20.0 mL) were then added and the mixture was extracted with DCM (3×20.0 mL). The organic layers were combined and dried with Na₂SO₄, filtered and concentrated under reduced pressure. The crude residue was purified by column chromatography on silica gel (0-100% EtOAc in hexanes) to provide the title compound as a white solid (285.7 mg, 69%). ¹H NMR (400 MHz, CDCl₃) δ 5.24 (s, 1H), 3.48 (m, 1H), 2.60-2.50 (m, 1H), 1.25 (d, J=6.9 Hz, 3H), 1.23 (s, 3H), 1.16 (d, J=6.8 Hz, 3H), 1.15 (s, 3H), 1.08-1.00 (m, 1H), 0.89-0.71 (m, 3H).

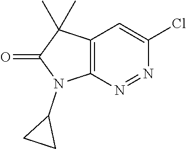 [SMILES: CC1(C)C(=O)N(C2CC2)c2nnc(Cl)cc21]

3-Chloro-7-cyclopropyl-5,5-dimethyl-5,7-dihydro-6H-pyrrolo[2,3-c]pyridazin-6-one. 5,8-Dichloro-1-cyclopropyl-7-isobutyryl-3,3-dimethyl-1,6,7-triazaspiro[3.5]nona-5,8-dien-2-one (285.7 mg, 0.83 mmol) was dissolved in DMF (15.0 mL) in a vial. Then Cs₂CO₃ (544.2 mg, 1.66 mmol) was added. The vial was sealed and heated to 80° C. for 4 h. The crude mixture was then filtered and concentrated under reduced pressure. The crude residue was purified by column chromatography on silica gel (0-100% EtOAc in hexanes) to provide the title compound as a white solid (185.3 mg, 94%). ¹H NMR (400 MHz, CDCl₃) δ 7.20 (s, 1H), 2.95-2.87 (m, 1H), 1.38 (s, 6H), 1.10-1.06 (m, 4H); ES-MS [M+H]⁺=238.0.

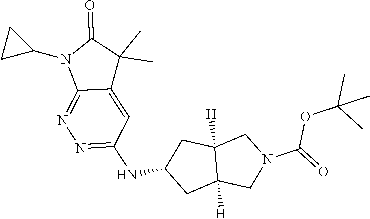 [SMILES: CC(C)(C)OC(=O)N1C[C@H]2C[C@H](Nc3cc4c(nn3)N(C3CC3)C(=O)C4(C)C)C[C@H]2C1]

tert-Butyl (3aR,5s,6aS)-5-((7-cyclopropyl-5,5-dimethyl-6-oxo-6,7-dihydro-5H-pyrrolo[2,3-c]pyridazin-3-yl)amino)hexahydrocyclopenta[c]pyrrole-2(1H)-carboxylate. 3-Chloro-7-cyclopropyl-5,5-dimethyl-pyrrolo[2,3-c]pyridazin-6-one (31.5 mg, 0.13 mmol), sodium tert-butoxide (25.5 mg, 0.27 mmol), t-BuXPhos (2.8 mg, 0.01 mmol), and Pd₂(dba)₃ (6.1 mg, 0.01 mmol) were combined and dissolved in toluene (0.5 mL). Then, tert-butyl (3aR,5s,6aS)-5-aminohexahydrocyclopenta[c]pyrrole-2(1H)-carboxylate (30.0 mg, 0.13 mmol) was added. The reaction was stirred at 100° C. for 7 h, at which time the reaction was filtered over Celite and washed with 5% MeOH:DCM and then concentrated under reduced pressure. The crude residue was purified by RP-HPLC (5-95% MeCN in 0.1% TFA aqueous solution over 5 min). The fractions containing product were basified with NaHCO₃, and extracted with 3:1 chloroform/IPA (v/v). The organic extracts were combined and passed through a phase separator and concentrated to yield the title compound as a solid (19.5 mg, 34%). ES-MS [M+H]⁺=428.0.

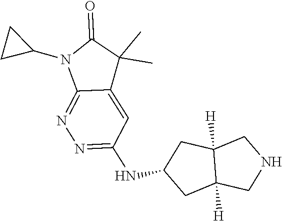 [SMILES: CC1(C)C(=O)N(C2CC2)c2nnc(N[C@@H]3C[C@H]4CNC[C@H]4C3)cc21]

7-Cyclopropyl-5,5-dimethyl-3-(((3aR,5s,6aS)-octahydrocyclopenta[c]pyrrol-5-yl)amino)-5,7-dihydro-6H-pyrrolo[2,3-c]pyridazin-6-one. tert-Butyl (3aR,5s,6aS)-5-((7-cyclopropyl-5,5-dimethyl-6-oxo-6,7-dihydro-5H-pyrrolo[2,3-c]pyridazin-3-yl)amino)hexahydrocyclopenta[c]pyrrole-2(1H)-carboxylate (19.5 mg, 0.05 mmol) was dissolved in 1,4-dioxane (1.0 mL) and MeOH (0.2 mL), and 4 M HCl in dioxanes solution (0.17 mL, 0.68 mmol) was added dropwise. The resulting mixture was stirred at r.t. for 1 h, after which time the solvents were concentrated under reduced pressure, and the crude reaction mixture was used for the next step without further purification. ES-MS [M+H]⁺=328.0.

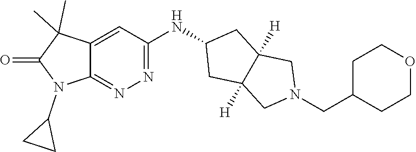 [SMILES: CC1(C)C(=O)N(C2CC2)c2nnc(N[C@H]3C[C@@H]4CN(CC5CCOCC5)C[C@@H]4C3)cc21]

7-Cyclopropyl-5,5-dimethyl-3-(((3aR,5s,6aS)-2-((tetrahydro-2H-pyran-4-yl)methyl)octahydrocyclopenta[c]pyrrol-5-yl)amino)-5,7-dihydro-6H-pyrrolo[2,3-c]pyridazin-6-one. 7-Cyclopropyl-5,5-dimethyl-3-(((3aR,5s,6aS)-octahydrocyclopenta[c]pyrrol-5-yl)amino)-5,7-dihydro-6H-pyrrolo[2,3-c]pyridazin-6-one (14.9 mg, 0.05 mmol) was dissolved in DCM (1.0 mL) and 4-oxanaldehyde (14.0 μL, 0.14 mmol) was added, followed by sodium triacetoxyborohydride (29.0 mg, 0.14 mmol). The resulting solution was stirred at r.t. for 1 h, after which time the reaction was quenched with sat. NaHCO₃ (0.2 mL) and extracted with 3:1 chloroform/IPA (v/v, 3×2.0 mL). The organic extracts were filtered through a phase separator and concentrated under reduced pressure. The residue was taken up in DMSO and solids were removed by syringe filtration. The crude residue was purified by RP-HPLC (5-95% MeCN in 0.1% TFA aqueous solution over 5 min). The fractions containing product were concentrated and further purified by RP-HPLC (5%-95% MeCN in 0.05% NH₄OH aqueous solution over 5 min). The fractions containing product were concentrated to give the title compound as a white solid (2.8 mg, 14% over 2 steps). ¹H NMR (400 MHz, CDCl₃) δ 6.44 (s, 1H), 4.42 (m, 1H), 4.28 (d, J=7.0 Hz, 1H), 3.96 (dd, J=11.1, 3.7 Hz, 2H), 3.39 (td, J=12.0, 1.7 Hz, 2H), 2.92-2.82 (m, 1H), 2.70 (m, 2H), 2.61 (m, 2H), 2.26 (m, 4H), 2.01-1.92 (m, 2H), 1.72-1.62 (m, 5H), 1.34 (s, 6H), 1.31-1.22 (m, 2H), 1.12-1.04 (m, 4H); ES-MS [M+H]⁺=426.0.

### Example 10. Representative Synthetic Procedures

### Representative Synthesis 1. N-(4-(5-(((3aR,5s,6aS)-2-(3,3-Dimethylbutyl)octahydrocyclopenta[c]pyrrol-5-yl)amino)pyrazin-2-yl)phenyl)acetamide

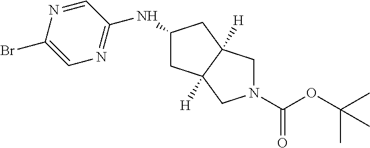 [SMILES: CC(C)(C)OC(=O)N1C[C@H]2C[C@H](Nc3cnc(Br)cn3)C[C@H]2C1]

tert-Butyl (3aR,5s,6aS)-5-((5-bromopyrazin-2-yl)amino)hexahydrocyclopenta[c]pyrrole-2(1H)-carboxylate. To a solution of tert-butyl (3aR,5s,6aS)-5-aminohexahydrocyclopenta[c]pyrrole-2(1H)-carboxylate (274 mg, 1.21 mmol) in NMP (4.5 mL) was added 2-bromo-5-chloropyrazine (585 mg, 3.02 mmol) and N,N-diisopropylethylamine (0.63 mL, 3.63 mmol). The mixture was stirred at 180° C. under microwave irradiation for 1 h. Solids were removed by syringe filtration, and crude residue was purified by RP-HPLC (20-70% MeCN in 0.05% NH₄OH aqueous solution over 20 min). Fractions containing product were combined and concentrated to give the title compound as a brown oil (148 mg, 32%). ES-MS [M+H]⁺=383.2.

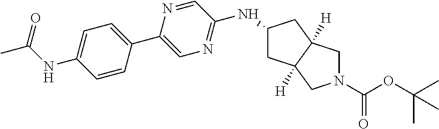 [SMILES: CC(=O)Nc1ccc(-c2cnc(N[C@H]3C[C@@H]4CN(C(=O)OC(C)(C)C)C[C@@H]4C3)cn2)cc1]

tert-Butyl (3aR,5s,6aS)-5-((5-(4-acetamidophenyl)pyrazin-2-yl)amino)hexahydrocyclopenta[c]pyrrole-2(1H)-carboxylate. tert-Butyl (3aR,5s,6aS)-5-((5-bromopyrazin-2-yl)amino)hexahydrocyclopenta[c]pyrrole-2(1H)-carboxylate (98.2 mg, 0.26 mmol), potassium carbonate (108 mg, 0.77 mmol), RuPhos Pd G3 (21.5 mg, 0.03 mmol), and 4-acetylaminophenyl boronic acid (55 mg, 0.31 mmol) were combined in a 2 mL vial, and 5:1 1,4-dioxane/H₂O solution (1.5 mL, degassed) was added. The solution was stirred at 100° C. for 3 h, after which the reaction was cooled to r.t. and diluted with sat. NaHCO₃ and DCM. The aqueous layer was extracted with DCM, and the combined organic extracts were filtered through a phase separator and concentrated. The crude residue was purified by column chromatography (0-10% MeOH in EtOAc). Fractions containing product were concentrated to give the title compound as a brown oil (70 mg, 62%). ES-MS [M+H]⁺=438.5.

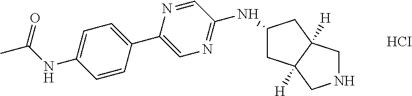

N-(4-(5-(((3aR,5s,6aS)-Octahydrocyclopenta[c]pyrrol-5-yl)amino)pyrazin-2-yl)phenyl)acetamide hydrochloride. tert-Butyl (3aR,5s,6aS)-5-((5-(4-acetamidophenyl)pyrazin-2-yl)amino)hexahydrocyclopenta[c]pyrrole-2(1H)-carboxylate (98.2 mg, 0.26 mmol) was dissolved in 1,4-dioxane (1 mL) and a 4M solution of HCl in dioxanes (4 mL) was added dropwise. The resulting solution was stirred for 2 h at r.t., after which time solvents were concentrated under reduced pressure to give the title compound as a tan solid which was used directly without further purification (59 mg, 100%). ES-MS [M+H]⁺=338.3.

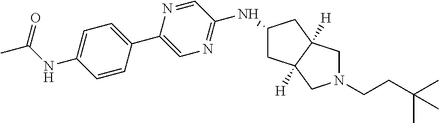 [SMILES: CC(=O)Nc1ccc(-c2cnc(N[C@H]3C[C@@H]4CN(CCC(C)(C)C)C[C@@H]4C3)cn2)cc1]

N-(4-(5-(((3aR,5s,6aS)-2-(3,3-Dimethylbutyl)octahydrocyclopenta[c]pyrrol-5-yl)amino)pyrazin-2-yl)phenyl)acetamide. To a solution of N-(4-(5-(((3aR,5s,6aS)-Octahydrocyclopenta[c]pyrrol-5-yl)amino)pyrazin-2-yl)phenyl)acetamide hydrochloride (13.3 mg, 0.04 mmol) in THF (0.25 mL) and DCE (0.25 mL) was added 3,3-dimethylbutyraldehyde (10.7 mg, 0.11 mmol), and the resulting solution was allowed to stir for 6 h. Sodium triacetoxyborohydride (22.6 mg, 0.11 mmol) was then added. The resulting solution was stirred at r.t. overnight, after which time solvents were concentrated, and crude residue was taken up in MeOH. Solids were removed by syringe filtration, and the solution was purified via RP-HPLC (10-50% MeCN in 0.1% aq TFA solution over 5 min). Fractions containing product were basified with NaHCO₃, and extracted with 3:1 chloroform/IPA. The organic extracts were combined and passed through a phase separator and concentrated to give the title compound as a white solid (7 mg, 45%). ES-MS [M+H]⁺=422.4.

### Representative Synthesis 2. (3aR,5s,6aS)—N-(5-(1,3-Dimethyl-1H-pyrazol-4-yl)pyridin-2-yl)-2-((tetrahydro-2H-pyran-4-yl)methyl)octahydrocyclopenta[c]pyrrol-5-amine

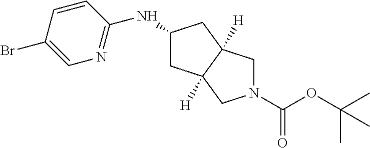 [SMILES: CC(C)(C)OC(=O)N1C[C@H]2C[C@H](Nc3ccc(Br)cn3)C[C@H]2C1]

tert-Butyl (3aR,5s,6aS)-5-((5-bromopyridin-2-yl)amino)hexahydrocyclopenta[c]pyrrole-2(1H)-carboxylate. To a solution of (3aR,5s,6aS)-5-aminohexahydrocyclopenta[c]pyrrole-2(1H)-carboxylate (365 mg, 1.61 mmol) in NMP (10 mL) was added 5-bromo-2-fluoropyridine (851 mg, 4.84 mmol) and N,N-diisopropylethylamine (0.84 mL, 4.83 mmol). The mixture was stirred at 180° C. under microwave irradiation for 1 h. Solids were removed by syringe filtration, and the crude residue was purified via RP-HPLC (20-70% MeCN in 0.05% NH₄OH aqueous solution over 20 min). Fractions containing product were combined and concentrated to give the title compound as a colorless oil (119 mg, 19%). ES-MS [M+H]⁺=382.2

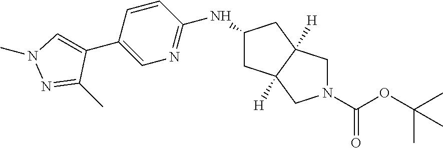 [SMILES: Cc1nn(C)cc1-c1ccc(N[C@H]2C[C@@H]3CN(C(=O)OC(C)(C)C)C[C@@H]3C2)nc1]

tert-Butyl (3aR,5s,6aS)-5-((5-(1,3-dimethyl-1H-pyrazol-4-yl)pyridin-2-yl)amino)hexahydrocyclopenta[c]pyrrole-2(1H)-carboxylate. tert-Butyl (3aR,5s,6aS)-5-((5-bromopyridin-2-yl)amino)hexahydrocyclopenta[c]pyrrole-2(1H)-carboxylate (141 mg, 0.37 mmol), potassium carbonate (156 mg, 1.11 mmol), RuPhos Pd G3 (30.9 mg, 0.04 mmol), and 1,3-dimethyl-1H-pyrazole-4-boronic acid pinacol ester (98.4 mg, 0.44 mmol) were combined in a vial, and 5:1 1,4-dioxane/H₂O solution (2 mL, degassed) was added. The resulting mixture was stirred at 100° C. for 3 h, after which time the reaction was cooled to r.t. and diluted with sat. NaHCO₃ and DCM. The aqueous layer was extracted with DCM, and the combined organic extracts were filtered through a phase separator and concentrated. The crude residue was purified by column chromatography (0-10% MeOH in EtOAc) and concentrated to give the title compound as a colorless oil (70 mg, 47%). ES-MS [M+H]⁺=398.5.

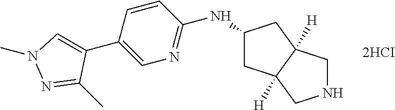 [SMILES: Cc1nn(C)cc1-c1ccc(N[C@@H]2C[C@H]3CNC[C@H]3C2)nc1]

(3aR,5s,6aS)—N-(5-(1,3-Dimethyl-1H-pyrazol-4-yl)pyridin-2-yl)octahydrocyclopenta[c]pyrrol-5-amine dihydrochloride. tert-Butyl (3aR,5s,6aS)-5-((5-(1,3-dimethyl-1H-pyrazol-4-yl)pyridin-2-yl)amino)hexahydrocyclopenta[c]pyrrole-2(1H)-carboxylate (69.5 mg, 0.17 mmol) was dissolved in 1,4-dioxane (1 mL) and a 4M solution of HCl in dioxanes (5 mL) was added dropwise. The resulting solution was stirred at r.t. for 2 h, after which time solvents were concentrated under reduced pressure to give the title compound as a tan solid which was used directly without further purification (65 mg, 100%). ES-MS [M+H]⁺=298.4.

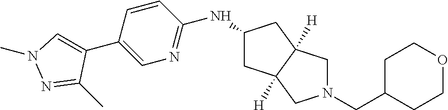 [SMILES: Cc1nn(C)cc1-c1ccc(N[C@H]2C[C@@H]3CN(CC4CCOCC4)C[C@@H]3C2)nc1]

(3aR,5s,6aS)—N-(5-(1,3-Dimethyl-1H-pyrazol-4-yl)pyridin-2-yl)-2-((tetrahydro-2H-pyran-4-yl)methyl)octahydrocyclopenta[c]pyrrol-5-amine. To a solution of (3aR,5s,6aS)—N-(5-(1,3-dimethyl-1H-pyrazol-4-yl)pyridin-2-yl)octahydrocyclopenta[c]pyrrol-5-amine dihydrochloride (16.2 mg, 0.04 mmol) in THF (0.25 mL) and DCE (0.25 mL) was added tetrahydro-2H-pyran-4-carbaldehyde (14.9 mg, 0.13 mmol), and the resulting solution was stirred at r.t. for 6 h. Sodium triacetoxyborohydride (27.8 mg, 0.13 mmol) was then added. The resulting solution was stirred overnight at r.t., at which time the reaction mixture was concentrated, and the crude residue was taken up in MeOH. Solids were removed by syringe filtration, and the solution was purified by RP-HPLC (5-35% MeCN in 0.1% aq TFA solution over 5 min). Fractions containing product were basified with NaHCO₃, and extracted with 3:1 chloroform/IPA. The organic extracts were combined and passed through a phase separator and concentrated to yield the title compound as a white solid (3.5 mg, 20%). ES-MS [M+H]⁺=396.0.

### Representative Synthesis 3. 5-Phenyl-N-((3aR,5s,6aS)-2-((tetrahydro-2H-pyran-4-yl)methyl)octahydrocyclopenta[c]pyrrol-5-yl)thiazol-2-amine

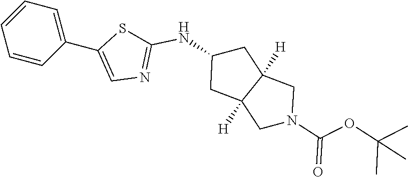 [SMILES: CC(C)(C)OC(=O)N1C[C@H]2C[C@H](Nc3ncc(-c4ccccc4)s3)C[C@H]2C1]

tert-Butyl (3aR,5s,6aS)-5-((5-phenylthiazol-2-yl)amino)hexahydrocyclopenta[c]pyrrole-2(1H)-carboxylate. tert-Butyl (3aR,5s,6aS)-5-aminohexahydrocyclopenta[c]pyrrole-2(1H)-carboxylate (160 mg, 0.71 mmol) and 2-chloro-5-phenylthiazole (277 mg, 1.42 mmol) were dissolved in NMP (2 mL) and DIPEA (0.37 mL, 2.12 mmol) was added. The resulting solution was stirred under microwave irradiation at 180° C. for 1 h, after which time the reaction mixture was purified directly by column chromatography (5-100% EtOAc in hexanes) to give the title compound as an orange oil (56 mg, 20%). ES-MS [M+H]⁺=386.2.

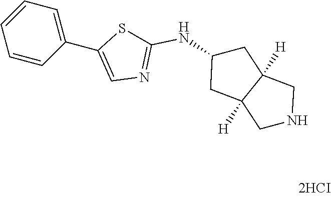 [SMILES: c1ccc(-c2cnc(N[C@@H]3C[C@H]4CNC[C@H]4C3)s2)cc1]

N-((3aR,5s,6aS)-Octahydrocyclopenta[c]pyrrol-5-yl)-5-phenylthiazol-2-amine dihydrochloride. tert-Butyl (3aR,5s,6aS)-5-((5-phenylthiazol-2-yl)amino)hexahydrocyclopenta[c]pyrrole-2(1H)-carboxylate (56 mg, 0.14 mmol) was dissolved in 1,4-dioxane (1 mL) and 4M HCl in dioxanes solution (0.72 mL, 2.88 mmol) was added dropwise. The resulting solution was stirred at r.t. for 1 h, after which time solvents were concentrated under reduced pressure to give the title compound as brown solid which was dried under vacuum and used without further purification (52 mg, 100%). ES-MS [M+H]⁺=286.2.

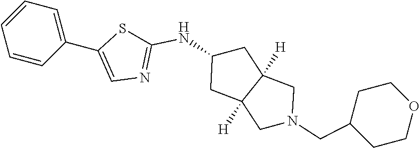 [SMILES: c1ccc(-c2cnc(N[C@H]3C[C@@H]4CN(CC5CCOCC5)C[C@@H]4C3)s2)cc1]

5-Phenyl-N-((3aR,5s,6aS)-2-((tetrahydro-2H-pyran-4-yl)methyl)octahydrocyclopenta[c]pyrrol-5-yl)thiazol-2-amine. N-((3aR,5s,6aS)-Octahydrocyclopenta[c]pyrrol-5-yl)-5-phenylthiazol-2-amine dihydrochloride (17.2 mg, 0.048 mmol) was dissolved in DCM (0.5 mL) and THF (0.5 mL) and tetrahydro-2H-pyran-4-carbaldehyde (27 mg, 0.24 mmol) was added, followed by sodium triacetoxyborohydride (31 mg, 0.14 mmol). The resulting solution was stirred at r.t. for 1.5 h, after which time the reaction mixture was quenched with sat. NaHCO₃, and the aqueous layer was extracted with 3:1 chloroform/IPA. The combined organic extracts were filtered through a phase separator and concentrated, and the crude residue was purified by RP-HPLC (5-35% MeCN in 0.1% aq TFA solution over 5 min). Fractions containing product were basified with NaHCO₃, and extracted with 3:1 chloroform/IPA. The organic extracts were combined and passed through a phase separator and concentrated to yield the title compound as a white solid (5.4 mg, 29%). ¹H-NMR (400 MHz, CDCl₃) δ 7.42-7.37 (m, 2H), 7.35-7.31 (m, 3H), 7.23-7.18 (m, 1H), 5.33 (d, J=4.8 Hz, 1H), 4.13-4.04 (m, 1H), 3.98 (dd, J=11.0, 3.9 Hz, 2H), 3.40 (td, J=12.0, 1.6 Hz, 2H), 2.76 (br, 2H), 2.60 (br, 2H), 2.44 (br, 2H), 2.32 (br, 2H), 1.97 (dd, J=12.0, 3.7 Hz, 2H), 1.81-1.73 (m, 5H), 1.36-1.25 (m, 2H). ES-MS [M+H]⁺=384.4.

### Representative Synthesis 4. N-[4-[6-[[(3aR,5r,6aS)-2-(3,3-Dimethylbutyl)-3,3a,4,5,6,6a-hexahydro-1H-cyclopenta[c]pyrrol-5-yl]amino]pyridazin-3-yl]phenyl]acetamide

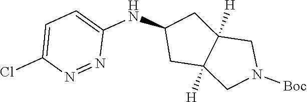 [SMILES: CC(C)(C)OC(=O)N1C[C@H]2C[C@@H](Nc3ccc(Cl)nn3)C[C@H]2C1]

tert-Butyl (3aR,5r,6aS)-5-((6-chloropyridazin-3-yl)amino)hexahydrocyclopenta[c]pyrrole-2(1H)-carboxylate. cis-N-Boc-5-oxo-octahydrocyclopenta[c]pyrrole (100 mg, 0.44 mmol) was dissolved in THF (1 mL) and DCE (1 mL), and 3-amino-6-chloropyridazine (288 mg, 2.22 mmol) was added, and the resulting solution was stirred for 10 min. Sodium triacetoxyborohydride (376 mg, 1.78 mmol) was then added, and the resulting solution was heated to 60° C. and stirred overnight, after which time the reaction was diluted with DCM and 3:1 chloroform/IPA solution, and the aqueous layer was extracted with 3:1 chloroform/IPA. The combined organic extracts were filtered through a phase separator and concentrated, and crude residue was purified by RP-HPLC. Fractions containing product were basified with sat. NaHCO₃, and extracted with 3:1 chloroform/iPA, and the combined organic extracts were filtered through a phase separator and concentrated to give the title compound as a brown oil (15.1 mg, 10%). ES-MS [M+H]⁺=339.3.

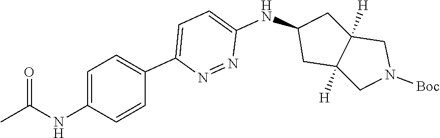 [SMILES: CC(=O)Nc1ccc(-c2ccc(N[C@@H]3C[C@@H]4CN(C(=O)OC(C)(C)C)C[C@@H]4C3)nn2)cc1]

tert-Butyl (3aR,5r,6aS)-5-((6-(4-acetamidophenyl)pyridazin-3-yl)amino)hexahydrocyclopenta[c]pyrrole-2(1H)-carboxylate. tert-Butyl (3aR,5r,6aS)-5-((6-chloropyridazin-3-yl)amino)hexahydrocyclopenta[c]pyrrole-2(1H)-carboxylate (15.1 mg, 0.045 mmol), K₂CO₃ (18.7 mg, 0.13 mmol), 4-acetylaminophenylboronic acid (9.6 mg, 0.053 mmol) and RuPhos-Pd-G3 (3.7 mg, 0.004 mmol) were combined in a sealed vial and placed under an inert atmosphere. 5:1 1,4-Dioxane/H₂O solution (0.6 mL, degassed) was then added via syringe. The resulting mixture was heated to 120° C. under microwave irradiation for 30 min, after which time the reaction was cooled to r.t. and diluted with sat. NaHCO₃, and DCM. The aqueous layer was extracted with DCM, and the combined organic extracts were filtered through a phase separator and concentrated. The crude residue was purified by column chromatography (hex/EtOAc) to give the title compound as a brown oil (3.9 mg, 20%). ES-MS [M+H]⁺=438.4.

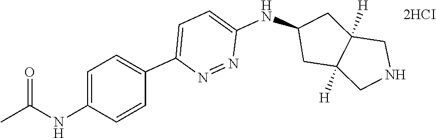 [SMILES: CC(=O)Nc1ccc(-c2ccc(N[C@H]3C[C@H]4CNC[C@H]4C3)nn2)cc1]

N-(4-(6-(((3aR,5r,6aS)-Octahydrocyclopenta[c]pyrrol-5-yl)amino)pyridazin-3-yl)phenyl)acetamide dihydrochloride. tert-Butyl (3aR,5r,6aS)-5-((6-(4-acetamidophenyl)pyridazin-3-yl)amino)hexahydrocyclopenta[c]pyrrole-2(1H)-carboxylate (3.9 mg, 0.009 mmol) was dissolved in 1,4-dioxanes (0.5 mL) and 4M HCl in dioxanes solution (0.5 mL) was added dropwise. The resulting solution was stirred at r.t. for 30 min, after which time the solvents were concentrated under reduced pressure and the resulting white solid was used directly without further purification (3.9 mg, 100%). ES-MS [M+H]⁺=338.4.

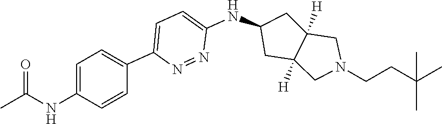 [SMILES: CC(=O)Nc1ccc(-c2ccc(N[C@@H]3C[C@@H]4CN(CCC(C)(C)C)C[C@@H]4C3)nn2)cc1]

N-[4-[6-[[(3aR,5r,6aS)-2-(3,3-Dimethylbutyl)-3,3a,4,5,6,6a-hexahydro-1H-cyclopenta[c]pyrrol-5-yl]amino]pyridazin-3-yl]phenyl]acetamide. N-(4-(6-(((3aR,5r,6aS)-Octahydrocyclopenta[c]pyrrol-5-yl)amino)pyridazin-3-yl)phenyl)acetamide dihydrochloride (3.3 mg, 0.009 mmol) was dissolved in THF (0.25 mL) and DCE (0.25 mL), and 3,3-dimethylbutyraldehyde (4.3 mg, 0.004 mmol) was added. The resulting mixture was stirred at r.t. for 6 h, after which time sodium triacetoxyborohydride (9.2 mg, 0.044 mmol) was then added, and the resulting solution was stirred at r.t. overnight, after which time the solvents were concentrated, and the crude residue was purified directly by RP-HPLC. Fractions containing product were basified with sat. NaHCO₃, and the aqueous layer was extracted with 3:1 chloroform/IPA. The combined organic extracts were filtered through a phase separator and concentrated to give the title compound as a white solid (1.8 mg, 49%). ¹H-NMR (400 MHz, CDCl₃) δ 7.95 (d, J=8.6 Hz, 2H), 7.58 (d, J=8.6 Hz, 2H), 7.51 (d, J=9.3 Hz, 1H), 6.54 (d, J=9.3 Hz, 1H), 4.67-4.62 (m, 1H), 2.81 (d, J=9.6 Hz, 2H), 2.75-2.67 (m, 2H), 2.47-2.43 (m, 2H), 2.22-2.15 (m, 7H), 1.74-1.44 (m, 4H), 0.94 (s, 9H). ES-MS [M+H]⁺=422.4.

### Representative Synthesis 5. (3aR,5r,6aS)-2-(3,3-Dimethylbutyl)-N-[4-(1,3-dimethylpyrazol-4-yl)-2,3-difluoro-phenyl]-3,3a,4,5,6,6a-hexahydro-1H-cyclopenta[c]pyrrol-5-amine

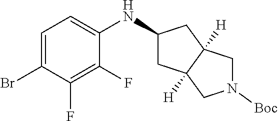 [SMILES: CC(C)(C)OC(=O)N1C[C@H]2C[C@@H](Nc3ccc(Br)c(F)c3F)C[C@H]2C1]

tert-Butyl (3aR,5r,6aS)-5-((4-bromo-2,3-difluorophenyl)amino)hexahydrocyclopenta[c]pyrrole-2(1H)-carboxylate. To a stirring solution of 4-bromo-2,3-difluoroaniline (415 mg, 2.00 mmol) in DCM (8 mL) and acetic acid (2 mL) was added cis-N-boc-5-oxo-octahydrocyclopenta[c]pyrrole (300 mg, 1.33 mmol), followed by sodium triacetoxyborohydride (423 mg, 2.00 mmol). The resulting suspension was stirred at r.t. overnight, after which time the reaction was quenched with sat. NaHCO₃, and the aqueous layer was extracted with DCM. The combined organic extracts were dried with MgSO₄, and were filtered and concentrated. The crude residue was purified by RP-HPLC (65-95% MeCN in 0.05% NH₄OH aqueous solution over 20 min), and fractions containing the product were diluted with H₂O, and extracted with DCM. The combined organic extracts were dried with MgSO₄, filtered and concentrated to give the title compound as an off white solid (142 mg, 26%). ES-MS [M+H−tbutyl]⁺=361.3.

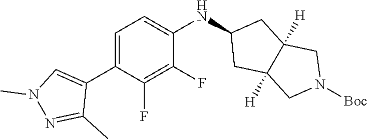 [SMILES: Cc1nn(C)cc1-c1ccc(N[C@@H]2C[C@@H]3CN(C(=O)OC(C)(C)C)C[C@@H]3C2)c(F)c1F]

tert-Butyl (3aR,5r,6aS)-5-((4-(1,3-dimethyl-1H-pyrazol-4-yl)-2,3-difluorophenyl)amino)hexahydrocyclopenta[c]pyrrole-2(1H)-carboxylate. tert-Butyl (3aR,5r,6aS)-5-((4-bromo-2,3-difluorophenyl)amino)hexahydrocyclopenta[c]pyrrole-2(1H)-carboxylate (114 mg, 0.27 mmol), 1,3-dimethyl-1H-pyrazole-4-boronic acid pinacol ester (73 mg, 0.33 mmol), K₂CO₃ (115 mg, 0.82 mmol) and RuPhos-Pd-G3 (23 mg, 0.03 mmol) were combined in a sealed vial, which was placed under an inert atmosphere. 5:1 1,4-Dioxane/H₂O solution (2.4 mL, degassed) was then added via syringe, and the resulting solution was stirred at 100° C. for 3 h, after which time the reaction was cooled to r.t. and diluted with DCM and sat. NaHCO₃. The aqueous layer was extracted with DCM, and the combined organic extracts were filtered through a phase separator and concentrated. The crude residue was purified by column chromatography (5-100% EtOAc in hexanes) to give the title compound as a brown oil (116 mg, 98%). ES-MS [M+H]+=433.5.

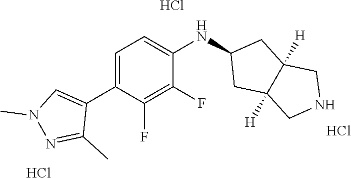 [SMILES: Cl ; Cc1nn(C)cc1-c1ccc(N[C@H]2C[C@H]3CNC[C@H]3C2)c(F)c1F ; Cl ; Cl]

(3aR,5r,6aS)—N-(4-(1,3-Dimethyl-1H-pyrazol-4-yl)-2,3-difluorophenyl)octahydrocyclopenta[c]pyrrol-5-amine trihydrochloride. tert-Butyl (3aR,5r,6aS)-5-((4-(1,3-dimethyl-1H-pyrazol-4-yl)-2,3-difluorophenyl)amino)hexahydrocyclopenta[c]pyrrole-2(1H)-carboxylate (116 mg, 0.27 mmol) was dissolved in 1,4-dioxane (2 mL) and 4M HCl in dioxanes solution (2 mL) was added dropwise. The resulting cloudy solution was stirred at r.t. for 30 min, after which time solvents were concentrated under reduced pressure to give the title compound as an off white solid which was used directly without further purification (118 mg, 100%). ES-MS [M+H]+=333.5.

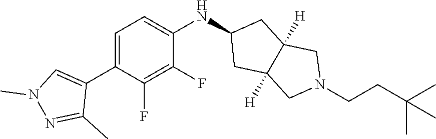 [SMILES: Cc1nn(C)cc1-c1ccc(N[C@@H]2C[C@@H]3CN(CCC(C)(C)C)C[C@@H]3C2)c(F)c1F]

(3aR,5r,6aS)-2-(3,3-Dimethylbutyl)-N-[4-(1,3-dimethylpyrazol-4-yl)-2,3-difluoro-phenyl]-3,3a,4,5,6,6a-hexahydro-1H-cyclopenta[c]pyrrol-5-amine. (3aR,5r,6aS)—N-(4-(1,3-Dimethyl-1H-pyrazol-4-yl)-2,3-difluorophenyl)octahydrocyclopenta[c]pyrrol-5-amine trihydrochloride (24 mg, 0.053 mmol) was dissolved in NMP (1 mL), and 3,3-dimethylbutyraldehyde (27 mg, 0.27 mmol) was added, followed by sodium triacetoxyborohydride (57 mg, 0.27 mmol). The resulting mixture was stirred at r.t. for 2 h, after which time the reaction mixture was quenched with sat. NaHCO₃ and diluted with 3:1 chloroform/IPA. The aqueous layer was extracted with 3:1 chloroform/IPA, and the organic extracts were filtered through a phase separator and concentrated. The crude residue was purified by RP-HPLC (20-50% MeCN in 0.1% TFA aqueous solution over 5 min), and fractions containing the product were basified with sat. NaHCO₃, and extracted with 3:1 chloroform/IPA. The organic extracts were filtered through a phase separator and concentrated to give the title compound as a colorless oil (7.1 mg, 32%). ES-MS [M+H]+=417.5.

### Representative Synthesis 6. (3aR,5r,6aS)-5-[6-(1,3-Dimethylpyrazol-4-yl)pyridazin-3-yl]oxy-2-(2,3,3-trimethylbutyl)-3,3a,4,5,6,6a-hexahydro-1H-cyclopenta[c]pyrrole

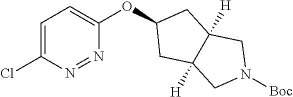 [SMILES: CC(C)(C)OC(=O)N1C[C@H]2C[C@@H](Oc3ccc(Cl)nn3)C[C@H]2C1]

tert-Butyl (3aR,5r,6aS)-5-(6-chloropyridazin-3-yl)oxy-3,3a,4,5,6,6a-hexahydro-1H-cyclopenta[c]pyrrole-2-carboxylate. To a solution of tert-butyl (3aR,5r,6aS)-5-hydroxy-3,3a,4,5,6,6a-hexahydro-1H-cyclopenta[c]pyrrole-2-carboxylate (prepared as described in Example 1) (500 mg, 2.20 mmol, 1.0 eq.) in THF (11.0 mL, 0.2 M) at 0° C. was added NaH (60% dispersion in mineral oil, 176 mg, 4.40 mmol, 2.0 eq.). After stirring for 5 min, 3,6-dichloropyridazine (491 mg, 3.30 mmol, 1.5 eq.) in THF (1.5 mL) was added. After stirring at r.t. for 16 h, the mixture was diluted with water and extracted with DCM (3×). The combined extracts were dried over Na₂SO₄, filtered and concentrated. The crude material was purified using flash chromatography on silica gel (0-40% EtOAc/hexanes) to provide the title compound as a white solid (660 mg, 89%). ¹H-NMR (400 MHz, CDCl₃) δ 7.37 (d, J=9.2 Hz, 1H), 6.93 (d, J=9.2 Hz, 1H), 5.65-5.59 (m, 1H), 3.56 (br, 2H), 3.37 (br, 2H), 2.76-2.68 (m, 2H), 2.46 (br, 2H), 1.08 (br, 2H), 1.47 (s, 9H); ES-MS [M+H]⁺=[M+H]⁺−Boc=240.4.

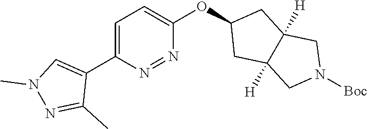 [SMILES: Cc1nn(C)cc1-c1ccc(O[C@@H]2C[C@@H]3CN(C(=O)OC(C)(C)C)C[C@@H]3C2)nn1]

tert-Butyl (3aR,5r,6aS)-5-[6-(1,3-dimethylpyrazol-4-yl)pyridazin-3-yl]oxy-3,3a,4,5,6,6a-hexahydro-1H-cyclopenta[c]pyrrole-2-carboxylate. tert-Butyl (3aR,5r,6aS)-5-(6-chloropyridazin-3-yl)oxy-3,3a,4,5,6,6a-hexahydro-1H-cyclopenta[c]pyrrole-2-carboxylate (339 mg, 1.0 mmol, 1.0 eq.), 1,3-dimethyl-1H-pyrazole-4-boronic acid pinacol ester (444.2 mg, 2.0 mmol, 2.0 eq.), K₂CO₃ (420.6 mg, 3.0 mmol, 3.0 eq.) and BrettPhos-Pd-G3 (45.4 mg, 0.05 mmol, 0.05 eq.) were charged into a reaction vial. A degassed mixture of 5:1 (v/v) 1,4-dioxane/H₂O (6.6 mL) was added. The resulting suspension was stirred at 100° C. for 1 h. After cooling to r.t., the reaction mixture was filtered through a pad of Celite which was washed thoroughly with EtOAc. The filtrate was concentrated under reduced pressure. The crude residue was purified by flash column chromatography (0-60% EtOAc/hexanes then 60-100% EtOAc/DCM) to give the title compound as a viscous oil (350 mg, 87%). ES-MS [M+H]⁺=400.4.

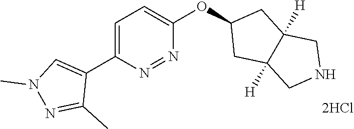 [SMILES: Cc1nn(C)cc1-c1ccc(O[C@H]2C[C@H]3CNC[C@H]3C2)nn1]

(3aR,5r,6aS)-5-[6-(1,3-Dimethylpyrazol-4-yl)pyridazin-3-yl]oxy-1,2,3,3a,4,5,6,6a-octahydrocyclopenta[c]pyrrole dihydrochloride. tert-Butyl (3aR,5r,6aS)-5-[6-(1,3-dimethylpyrazol-4-yl)pyridazin-3-yl]oxy-3,3a,4,5,6,6a-hexahydro-1H-cyclopenta[c]pyrrole-2-carboxylate (350 mg, 0.88 mmol, 1.0 eq.) was dissolved in 1,4-dioxane (4.0 mL). A 4M HCl in 1,4-dioxane solution (2.0 mL, 8.76 mmol, 10.0 eq.) was added dropwise. After stirring 1 h at r.t., solvents were removed under reduced pressure. The crude material was azeotroped with toluene (3×) to provide the title compound as a white solid which was used without further purification as the HCl salt (326 mg, 99%). ES-MS [M+H]⁺=300.4.

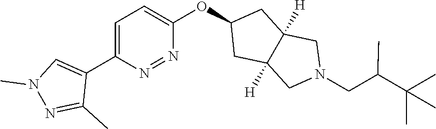 [SMILES: Cc1nn(C)cc1-c1ccc(O[C@@H]2C[C@@H]3CN(CC(C)C(C)(C)C)C[C@@H]3C2)nn1]

(3aR,5r,6aS)-5-[6-(1,3-Dimethylpyrazol-4-yl)pyridazin-3-yl]oxy-2-(2,3,3-trimethylbutyl)-3,3a,4,5,6,6a-hexahydro-1H-cyclopenta[c]pyrrole. (3aR,5r,6aS)-5-[6-(1,3-Dimethylpyrazol-4-yl)pyridazin-3-yl]oxy-1,2,3,3a,4,5,6,6a-octahydrocyclopenta[c]pyrrole dihydrochloride (25 mg, 0.067 mmol, 1.0 eq.) was suspended in DCM (1.0 mL) and acetic acid (0.1 mL). 2,3,3-Trimethylbutanal (42.6 μL, 0.336 mmol, 5.0 eq.) was added. The mixture was stirred at r.t. for 30 min and sodium triacetoxyborohydride (71.2 mg, 0.336 mmol. 5.0 eq.) was added. The resulting solution was stirred at r.t. for 16 h, after which time the reaction mixture was quenched with sat. soln. NaHCO₃ and extracted with chloroform/IPA (3:1, v/v). The combined extracts were filtered through a phase separator and concentrated. The crude residue was purified by RP-HPLC, fractions containing the product were basified with sat. soln. NaHCO₃, and extracted with chloroform/IPA (3:1, v/v). The combined extracts were filtered through a phase separator and concentrated to give the title compound as a colorless oil (10.2 mg, 38%). ES-MS [M+H]⁺=398.5.

### Representative Synthesis 7. N-[[(3aR,5s,6aS)-2-(3,3-Dimethylbutyl)-3,3a,4,5,6,6a-hexahydro-1H-cyclopenta[c]pyrrol-5-yl]methyl]-6-(2,4-dimethylpyrazol-3-yl)pyridazin-3-amine

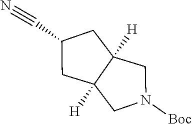 [SMILES: CC(C)(C)OC(=O)N1C[C@H]2C[C@H](C#N)C[C@H]2C1]

tert-Butyl (3aR,5s,6aS)-5-cyano-3,3a,4,5,6,6a-hexahydro-1H-cyclopenta[c]pyrrole-2-carboxylate. Solid potassium tert-butoxide (996.2 mg, 8.88 mmol, 2.0 eq.) was added portion wise to a solution of cis-tert-butyl 5-oxohexahydrocyclopenta[c]pyrrole-2(1H)-carboxylate (1.0 g, 4.44 mmol, 1.0 eq.), tosylmethyl isocyanide (870 mg, 4.44 mmol, 1.0 eq.) in monoglyme (15.52 mL, 0.285 M) and ethanol (0.44 mL, 7.55 mmol, 1.7 eq.) at 0° C. The reaction mixture was stirred for 15 min at 0° C., then warmed to r.t. and allowed to stir for additional 1.5 h. Upon completion, the precipitate (TosK) was removed via filtration and the solid was washed with EtOAc. The combined organic layers were concentrated under reduced pressure. The crude product was purified using flash column chromatography on silica gel (0-80% EtOAc/hexanes) to provide the title compound as a viscous oil (532 mg, 51%). ES-MS [M+H]⁺=[M+H]⁺−tButyl=181.2.

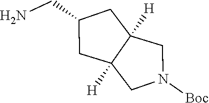 [SMILES: CC(C)(C)OC(=O)N1C[C@H]2C[C@H](CN)C[C@H]2C1]

tert-Butyl (3aR,5s,6aS)-5-(aminomethyl)-3,3a,4,5,6,6a-hexahydro-1H-cyclopenta[c]pyrrole-2-carboxylate. To a solution of tert-butyl (3aR,5s,6aS)-5-cyano-3,3a,4,5,6,6a-hexahydro-1H-cyclopenta[c]pyrrole-2-carboxylate (532 mg, 2.25 mmol, 1.0 eq.) in THF (11.24 mL, 0.2 M) at 0° C. was added dropwise a solution of lithium aluminum hydride (1M in THF, 2.25 mL, 2.25 mmol, 1.0 eq.). After 2 h at 0° C., the reaction mixture was slowly added to an aqueous saturated Rochelle's salt solution (10 mL). Ethyl acetate (20 mL) was added. The mixture was allowed to stir overnight. The organic layer was separated. The aqueous layer was extracted with EtOAc (3×). The combined extracts were dried over Na₂SO₄, filtered and concentrated to provide the title compound (350 mg, 65%) which was used in the next reaction without further purification. ES-MS [M+H]⁺=[M+H]⁺−tButyl=185.2.

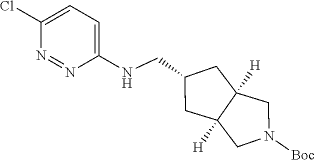 [SMILES: CC(C)(C)OC(=O)N1C[C@H]2C[C@H](CNc3ccc(Cl)nn3)C[C@H]2C1]

tert-Butyl (3aR,5s,6aS)-5-[[(6-chloropyridazin-3-yl)amino]methyl]-3,3a,4,5,6,6a-hexahydro-1H-cyclopenta[c]pyrrole-2-carboxylate. tert-Butyl (3aR,5s,6aS)-5-(aminomethyl)-3,3a,4,5,6,6a-hexahydro-1H-cyclopenta[c]pyrrole-2-carboxylate (350 mg, 1.45 mmol, 1.0 eq.) was dissolved in n-BuOH (7.3 mL, 0.2 M), and DIPEA (0.760 mL, 4.37 mmol, 3.0 eq.) was added followed by 3,6-dichloropyridazine (651 mg, 4.37 mmol, 3.0 eq.). The resulting suspension was heated to 100° C. overnight, after which time the reaction was cooled to r.t., and diluted with DCM and sat. soln. NaHCO₃. The aqueous layer was extracted with DCM (3×). The combined extracts were dried over Na₂SO₄, filtered and concentrated. The crude residue was purified by flash column chromatography on silica gel (0-30% EtOAc/hexanes then 30-50% EtOAc/DCM) to provide the title compound as a viscous oil (210 mg, 41%). ES-MS [M+H]⁺=353.4.

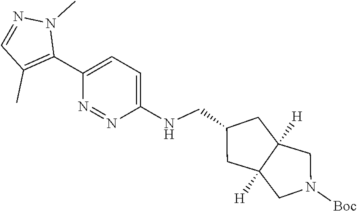 [SMILES: Cc1cnn(C)c1-c1ccc(NC[C@H]2C[C@@H]3CN(C(=O)OC(C)(C)C)C[C@@H]3C2)nn1]

tert-Butyl (3aR,5s,6aS)-5-[[[6-(2,4-dimethylpyrazol-3-yl)pyridazin-3-yl]amino]methyl]-3,3a,4,5,6,6a-hexahydro-1H-cyclopenta[c]pyrrole-2-carboxylate. tert-Butyl (3aR,5s,6aS)-5-[[(6-chloropyridazin-3-yl)amino]methyl]-3,3a,4,5,6,6a-hexahydro-1H-cyclopenta[c]pyrrole-2-carboxylate (210 mg, 0.595 mmol, 1.0 eq.), 1,4-dimethylpyrazole-5-boronic acid pinacol ester (224.7 mg, 1.01 mmol, 1.7 eq.), K₂CO₃ (250.5 mg, 1.79 mmol, 3.0 eq.) and BrettPhos-Pd-G3 (24 mg, 0.03 mmol) were charged into a reaction vial. A degassed mixture of 5:1 (v/v) 1,4-dioxane/H₂O (3.0 mL) was added. The resulting suspension was stirred at 100° C. for 1 h. After cooling to r.t., the reaction mixture was filtered through a pad of Celite which was washed thoroughly with EtOAc. The filtrate was concentrated under reduced pressure. The crude residue was purified by flash column chromatography (0-100% EtOAc/DCM) to give the title compound as a viscous oil (200 mg, 81%). ES-MS [M+H]⁺=413.0.

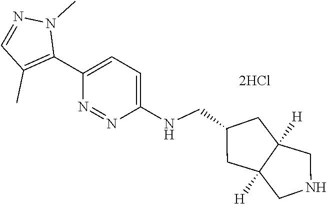 [SMILES: Cc1cnn(C)c1-c1ccc(NC[C@@H]2C[C@H]3CNC[C@H]3C2)nn1]

N-[[(3aR,5s,6aS)-1,2,3,3a,4,5,6,6a-Octahydrocyclopenta[c]pyrrol-5-yl]methyl]-6-(2,4-dimethylpyrazol-3-yl)pyridazin-3-amine. tert-Butyl (3aR,5s,6aS)-5-[[[6-(2,4-dimethylpyrazol-3-yl)pyridazin-3-yl]amino]methyl]-3,3a,4,5,6,6a-hexahydro-1H-cyclopenta[c]pyrrole-2-carboxylate (200 mg, 0.485 mmol, 1.0 eq.) was dissolved in 1,4-dioxane (2.0 mL). 4M HCl in 1,4-dioxane solution (1.82 mL, 7.27 mmol, 15.0 eq.) was added dropwise. After stirring 30 min at r.t., solvents were removed under reduced pressure. The crude material was azeotroped with toluene (3×) to provide the title compound as a pale yellow solid which was used without further purification as the HCl salt. ES-MS [M+H]⁺=313.2.

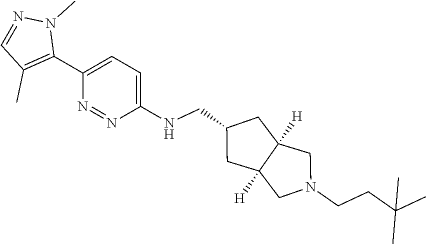 [SMILES: Cc1cnn(C)c1-c1ccc(NC[C@H]2C[C@@H]3CN(CCC(C)(C)C)C[C@@H]3C2)nn1]

N-[[(3aR,5s,6aS)-2-(3,3-Dimethylbutyl)-3,3a,4,5,6,6a-hexahydro-1H-cyclopenta[c]pyrrol-5-yl]methyl]-6-(2,4-dimethylpyrazol-3-yl)pyridazin-3-amine. N-[[(3aR,5s,6aS)-1,2,3,3a,4,5,6,6a-Octahydrocyclopenta[c]pyrrol-5-yl]methyl]-6-(2,4-dimethylpyrazol-3-yl)pyridazin-3-amine dihydrochloride (15 mg, 0.045 mmol, 1.0 eq.) was suspended in DCM (1 mL) and acetic acid (0.1 mL). 3,3-Dimethylbutyraldehyde (28.1 μL, 0.224 mmol, 5.0 eq.) was added. The mixture was stirred at 50° C. for 30 min and sodium triacetoxyborohydride (47.5 mg, 0.224 mmol. 5.0 eq.) was added. The resulting solution was stirred at r.t. overnight, after which time the reaction mixture was quenched with sat. soln. NaHCO₃, and extracted with chloroform/IPA (3:1, v/v). The combined extracts were filtered through a phase separator and concentrated. The crude residue was purified by RP-HPLC, and fractions containing the product were basified with sat. soln. NaHCO₃, and extracted with chloroform/IPA (3:1, v/v). The combined extracts were filtered through a phase separator and concentrated to give the title compound as a colorless oil (5.2 mg, 30%). ES-MS [M+H]⁺=397.0.

### Representative Synthesis 8. (3aR,6aS)-2-(3,3-Dimethylbutyl)-N-[6-(2,4-dimethylpyrazol-3-yl)pyridazin-3-yl]-3,3a,4,5,6,6a-hexahydro-1H-cyclopenta[c]pyrrole-5-carboxamide

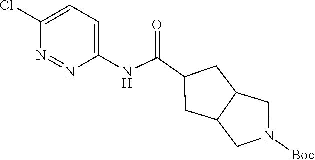 [SMILES: CC(C)(C)OC(=O)N1CC2CC(C(=O)Nc3ccc(Cl)nn3)CC2C1]

tert-Butyl 5-[[6-chloropyridazin-3-yl]carbamoyl]-3,3a,4,5,6,6a-hexahydro-1H-cyclopenta[c]pyrrole-2-carboxylate. 2-[(tert-Butoxy)carbonyl]-octahydrocyclopenta[c]pyrrole-5-carboxylic acid (255.3 mg, 1.0 mmol, 1.0 eq.), DIPEA (0.35 mL, 2.0 mmol, 2.0 eq.), and HATU (456.3 mg, 1.2 mol, 1.2 eq.) were dissolved in THF (5.0 mL, 0.2 M). The mixture was stirred for 15 min. 3-Amino-6-chloropyridazine (194.3 mg, 1.5 mmol, 1.5 eq.) was added. After 3 h at 80° C., the mixture was diluted with DCM and water. The organic layer was separated. The aqueous layer was extracted with CHCl₃/IPA mixture (3:1, 3×). The combined organic layers were dried over Na₂SO₄, filtered and concentrated. The crude residue was purified using flash column chromatography on silica gel (0-86% EtOAc/hexanes) to provide the title compound as a yellow powder (250 mg, 68% yield). ES-MS [M+H]⁺=[M+H]⁺−tButyl=311.0.

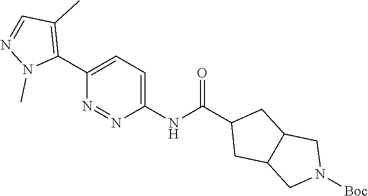 [SMILES: Cc1cnn(C)c1-c1ccc(NC(=O)C2CC3CN(C(=O)OC(C)(C)C)CC3C2)nn1]

tert-Butyl 5-[[6-(2,4-dimethylpyrazol-3-yl)pyridazin-3-yl]carbamoyl]-3,3a,4,5,6,6a-hexahydro-1H-cyclopenta[c]pyrrole-2-carboxylate. tert-Butyl 5-[[6-chloropyridazin-3-yl]carbamoyl]-3,3a,4,5,6,6a-hexahydro-1H-cyclopenta[c]pyrrole-2-carboxylate (250 mg, 0.682 mmol, 1.0 eq.), 1,4-dimethylpyrazole-5-boronic acid pinacol ester (257.3 mg, 1.16 mmol, 1.7 eq.), K₂CO₃ (286.8 mg, 2.05 mmol, 3.0 eq.) and BrettPhos-Pd-G3 (28.5 mg, 0.03 mmol, 0.05 eq.) were charged into a reaction vial. A degassed mixture of 5:1 (v/v) 1,4-dioxane/H₂O (3.0 mL) was added. The resulting suspension was stirred at 100° C. for 1 h. After cooling to r.t., the reaction mixture was filtered through a pad of Celite which was washed thoroughly with EtOAc. The filtrate was concentrated under reduced pressure. The crude residue was purified by flash column chromatography (0-100% EtOAc/DCM) to give the title compound as a viscous oil (180 mg, 63% yield). ES-MS [M+H]⁺=427.4.

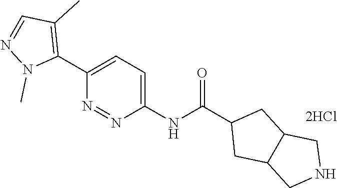 [SMILES: Cc1cnn(C)c1-c1ccc(NC(=O)C2CC3CNCC3C2)nn1]

N-[6-(2,4-Dimethylpyrazol-3-yl)pyridazin-3-yl]-1,2,3,3a,4,5,6,6a-octahydrocyclopenta[c]pyrrole-5-carboxamide dihydrochloride. tert-Butyl 5-[[6-(2,4-dimethylpyrazol-3-yl)pyridazin-3-yl]carbamoyl]-3,3a,4,5,6,6a-hexahydro-1H-cyclopenta[c]pyrrole-2-carboxylate (180 mg, 0.422 mmol, 1.0 eq.) was dissolved in 1,4-dioxane (2.0 mL). 4M HCl in 1,4-dioxane solution (1.0 mL, 4.0 mmol, 9.5 eq.) was added dropwise. After stirring 30 min at r.t., solvents were removed under reduced pressure. The crude material was azeotroped with toluene (3×) to provide the title compound as an off white solid which was used without further purification as the HCl salt. ES-MS [M+H]⁺=327.4.

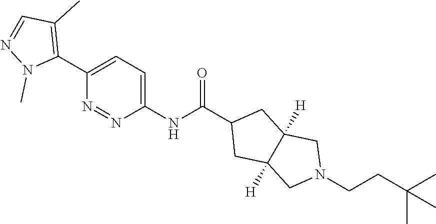 [SMILES: Cc1cnn(C)c1-c1ccc(NC(=O)C2C[C@@H]3CN(CCC(C)(C)C)C[C@@H]3C2)nn1]

(3aR,6aS)-2-(3,3-Dimethylbutyl)-N-[6-(2,4-dimethylpyrazol-3-yl)pyridazin-3-yl]-3,3a,4,5,6,6a-hexahydro-1H-cyclopenta[c]pyrrole-5-carboxamide. N-[6-(2,4-Dimethylpyrazol-3-yl)pyridazin-3-yl]-1,2,3,3a,4,5,6,6a-octahydrocyclopenta[c]pyrrole-5-carboxamide dihydrochloride (25 mg, 0.063 mmol, 1.0 eq.) was suspended in DCM (1.5 mL) and THF (1.5 mL). 3,3-Dimethylbutyraldehyde (39.3 μL, 0.313 mmol, 5.0 eq.) was added. The mixture was stirred at r.t. for 30 min and sodium triacetoxyborohydride (66.3 mg, 0.313 mmol. 5.0 eq.) was added. The resulting solution was stirred at r.t. overnight, after which time the reaction mixture was quenched with sat. soln. NaHCO₃, and extracted with chloroform/IPA (3:1, v/v). The combined extracts were filtered through a phase separator and concentrated. The crude residue was purified by RP-HPLC to provide the desired product as two separable (endo and exo) isomers. Major isomer (3.5 mg, 14% yield): ¹H-NMR (400 MHz, CDCl₃) δ 10.62 (s, 1H), 8.55 (d, J=9.3 Hz, 1H), 7.54 (d, J=9.2 Hz, 1H), 7.39 (s, 1H), 4.01 (s, 3H), 2.96-2.88 (m, 1H), 2.81-2.78 (m, 2H), 2.77-2.66 (m, 2H), 2.51-2.46 (m, 2H), 2.38-2.55 (m, 4H), 2.16 (s, 3H), 1.92-1.84 (m, 2H), 1.49-1.45 (m, 2H), 0.91 (s, 9H); ES-MS [M+H]⁺=411.4. Minor isomer (1.0 mg, 4% yield): ¹H-NMR (400 MHz, CDCl₃) δ 8.51 (d, J=9.2 Hz, 1H), 8.50 (s, 1H), 7.51 (d, J=9.2 Hz, 1H), 7.40 (s, 1H), 4.02 (s, 3H), 3.13-3.05 (m, 1H), 2.87-2.77 (m, 2H), 2.74-2.69 (m, 2H), 2.36-2.32 (m, 2H), 2.23-2.19 (m, 2H), 2.13 (s, 3H), 2.09-2.02 (m, 2H), 1.89-1.83 (m, 2H), 1.43-1.39 (m, 2H), 0.92 (s, 9H); ES-MS [M+H]⁺=411.4.

### Representative Synthesis 9. (3aR,6aS)-2-(3,3-Dimethylbutyl)-5-[[6-(2,4-dimethylpyrazol-3-yl)pyridazin-3-yl]oxymethyl]-3,3a,4,5,6,6a-hexahydro-1H-cyclopenta[c]pyrrole

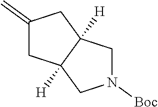 [SMILES: C=C1C[C@@H]2CN(C(=O)OC(C)(C)C)C[C@@H]2C1]

tert-Butyl (3aR,6aS)-5-methylene-1,3,3a,4,6,6a-hexahydrocyclopenta[c]pyrrole-2-carboxylate. To a suspension of methyl(triphenyl)phosphonium iodide (6.67 g, 16.5 mmol, 2.2 eq.) in THF (37.5 mL, 0.2 M) at 0° C. was added potassium tert-butoxide (1.68 g, 15 mmol, 2.0 eq.). After stirring at 0° C. for 30 min, the reaction mixture was allowed to warm to r.t. After 30 min, the reaction mixture was cooled back down to 0° C. and a solution of cis-tert-butyl 5-oxohexahydrocyclopenta[c]pyrrole-2(1H)-carboxylate (1.69 g, 7.5 mmol, 1.0 eq.) in THF (37.5 mL) was added. The resulting mixture was stirred at r.t. for 16 h. Diethyl ether (100 mL) was added and the mixture was filtered. The solid was washed with diethyl ether (3×). The combined filtrates were concentrated. The crude material was purified using flash column chromatography on silica gel (0-40% EtOAc/hexanes) to provide the title compound as a colorless oil (1.50 g, 89%). ¹H-NMR (400 MHz, CDCl₃) δ 4.91 (dd, J=4.0, 2.1 Hz, 2H), 3.54 (br, 2H), 3.18 (d, J=9.5 Hz, 1H), 3.09 (d, J=7.8 Hz, 1H), 2.69 (br, 2H), 2.58 (dd, J=6.6, 16.5 Hz, 2H), 2.22 (d, J=1.9 Hz, 1H), 2.19 (d, J=1.9 Hz, 1H), 1.48 (s, 9H); ES-MS [M+H]⁺=[M+H]⁺−tButyl=168.4.

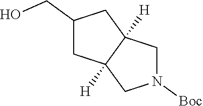 [SMILES: CC(C)(C)OC(=O)N1C[C@H]2CC(CO)C[C@H]2C1]

tert-Butyl (3aR,6aS)-5-(hydroxymethyl)-3,3a,4,5,6,6a-hexahydro-1H-cyclopenta[c]pyrrole-2-carboxylate. Under nitrogen atmosphere, borane dimethyl sulfide complex (2.0 M in THF, 15.78 mL, 31.57 mmol, 4.7 eq.) was diluted in THF (35.0 mL) and cooled to 0° C. Neat 2,3-dimethylbut-2-ene (3.77 mL, 31.57 mmol, 4.7 eq.) was added dropwise. After 3 h at 0° C., a solution of tert-butyl (3aR,6aS)-5-methylene-1,3,3a,4,6,6a-hexahydrocyclopenta[c]pyrrole-2-carboxylate (1.5 g, 6.72 mmol, 1.0 eq.) in THF (15 mL) was added slowly. The resulting mixture was warmed to r.t. and stirred for 16 h. After cooling to 0° C., a solution of 10% NaOH (15.0 mL) was added slowly followed by hydrogen peroxide solution (33% in water, 11.9 mL). The ice bath was removed. After 2 h at r.t., the solvents were removed under reduced pressure. The residue was re-dissolved in water and diethyl ether. The layers were separated. The aqueous layer was extracted with diethyl ether (3×). The combined extracts were dried over Na₂SO₄, filtered and concentrated. The crude material was purified using flash chromatography on silica gel (0-50% EtOAc/hexanes) to provide the title compound as a viscous oil (1.30 g, 78%) (mixture of endo/exo isomers, 3:1). ¹H-NMR (400 MHz, CDCl₃) (major isomer) δ 3.60 (d, J=6.1 Hz, 2H), 3.54 (d, J=6.0 Hz, 1H), 3.48-3.46 (m, 2H), 3.02 (br, 2H), 2.62 (m, 2H), 2.26-2.18 (m, 1H), 2.05 (m, 2H), 1.48 (s, 9H), 1.20-1.12 (m, 2H); ES-MS [M+H]⁺=[M+H]⁺−tButyl=186.0.

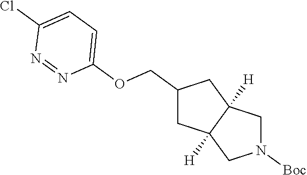 [SMILES: CC(C)(C)OC(=O)N1C[C@H]2CC(COc3ccc(Cl)nn3)C[C@H]2C1]

tert-Butyl (3aR,6aS)-5-[(6-chloropyridazin-3-yl)oxymethyl]-3,3a,4,5,6,6a-hexahydro-1H-cyclopenta[c]pyrrole-2-carboxylate. To a solution of tert-butyl (3aR,6aS)-5-(hydroxymethyl)-3,3a,4,5,6,6a-hexahydro-1H-cyclopenta[c]pyrrole-2-carboxylate (543 mg, 2.25 mmol, 1.0 eq.) in THF (11.25 mL, 0.2 M) at 0° C. was added NaH (60% dispersion in mineral oil, 180 mg, 4.5 mmol, 2.0 eq.). After stirring for 5 min, 3,6-dichloropyridazine (502.8 mg, 3.375 mmol, 1.5 eq.) in THF (1.5 mL) was added. After 16 h, the mixture was diluted with water and extracted with DCM (3×). The combined extracts were dried over Na₂SO₄, filtered and concentrated. The crude material was purified using flash chromatography on silica gel (0-40% EtOAc/hexanes) to provide the title compound as a white solid (580 mg, 73%) (a mixture of endo/exo isomers, 3:1). ¹H-NMR (400 MHz, CDCl₃) (major isomer) δ 7.38 (d, J=9.2 Hz, 1H), 6.97 (d, J=9.2 Hz, 1H), 4.47 (br, 2H), 3.49 (br, 2H), 3.24 (br, 2H), 2.70-2.63 (m, 2H), 2.58-2.49 (m, 1H), 2.17-2.14 (m, 2H), 1.47 (s, 9H), 1.34-1.26 (m, 2H); ES-MS [M+H]⁺=[M+H]⁺−Boc=254.0.

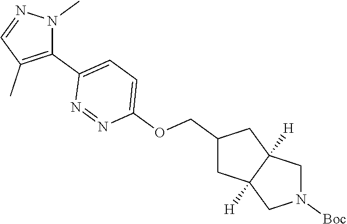 [SMILES: Cc1cnn(C)c1-c1ccc(OCC2C[C@@H]3CN(C(=O)OC(C)(C)C)C[C@@H]3C2)nn1]

tert-Butyl (3aR,6aS)-5-[[6-(2,4-dimethylpyrazol-3-yl)pyridazin-3-yl]oxymethyl]-3,3a,4,5,6,6a-hexahydro-1H-cyclopenta[c]pyrrole-2-carboxylate. tert-Butyl (3aR,6aS)-5-[(6-chloropyridazin-3-yl)oxymethyl]-3,3a,4,5,6,6a-hexahydro-1H-cyclopenta[c]pyrrole-2-carboxylate (350 mg, 0.989 mmol, 1.0 eq.), 1,4-dimethylpyrazole-5-boronic acid pinacol ester (439.4 mg, 1.97 mmol, 2.0 eq.), K₂CO₃ (416.3 mg, 2.97 mmol, 3.0 eq.) and BrettPhos-Pd-G3 (44.9 mg, 0.05 mmol, 0.05 eq.) were charged into a reaction vial. A degassed mixture of 5:1 (v/v) 1,4-dioxane/H₂O (6.5 mL) was added. The resulting suspension was stirred at 100° C. for 1 h. After cooling to r.t., the reaction mixture was filtered through a pad of Celite which was washed thoroughly with EtOAc. The filtrate was concentrated under reduced pressure. The crude residue was purified by flash column chromatography (0-100% EtOAc/DCM) to give the title compound as a light tan solid (380 mg, 93%) (mixture of endo/exo isomers, 3:1). ¹H-NMR (400 MHz, CDCl₃) (major isomer) δ 7.46 (d, J=9.2 Hz, 1H), 7.41 (s, 1H), 7.09 (d, J=9.1 Hz, 1H), 4.57 (br, 2H), 4.04 (s, 3H), 3.49 (br, 2H), 3.29 (br, 2H), 2.73-2.64 (m, 2H), 2.63-2.54 (m, 1H), 2.20-2.16 (m, 2H), 2.15 (s, 3H), 1.48 (s, 9H), 1.38-1.27 (m, 2H); ES-MS [M+H]⁺=414.5.

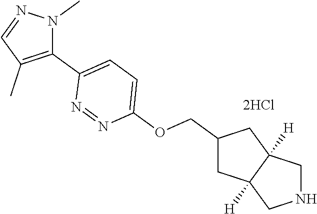 [SMILES: Cc1cnn(C)c1-c1ccc(OCC2C[C@H]3CNC[C@H]3C2)nn1 ; "2HCl"]

(3aR,6aS)-5-[[6-(2,4-Dimethylpyrazol-3-yl)pyridazin-3-yl]oxymethyl]-1,2,3,3a,4,5,6,6a-octahydrocyclopenta[c]pyrrole dihydrochloride. tert-Butyl (3aR,6aS)-5-[[6-(2,4-dimethylpyrazol-3-yl)pyridazin-3-yl]oxymethyl]-3,3a,4,5,6,6a-hexahydro-1H-cyclopenta[c]pyrrole-2-carboxylate (380 mg, 0.919 mmol, 1.0 eq.) was dissolved in 1,4-dioxane (2.0 mL). 4M HCl in 1,4-dioxane solution (2.5 mL, 10.0 mmol, 10.9 eq.) was added dropwise. After stirring 30 min at r.t., solvents were removed under reduced pressure. The crude material was azeotroped with toluene (3×) to provide the title compound as a pale yellow solid which was used without further purification as the HCl salt. ES-MS [M+H]⁺=314.2.

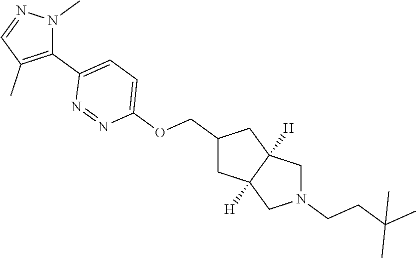 [SMILES: Cc1cnn(C)c1-c1ccc(OCC2C[C@@H]3CN(CCC(C)(C)C)C[C@@H]3C2)nn1]

(3aR,6aS)-2-(3,3-Dimethylbutyl)-5-[[6-(2,4-dimethylpyrazol-3-yl)pyridazin-3-yl]oxymethyl]-3,3a,4,5,6,6a-hexahydro-1H-cyclopenta[c]pyrrole. (3aR,6aS)-5-[[6-(2,4-Dimethylpyrazol-3-yl)pyridazin-3-yl]oxymethyl]-1,2,3,3a,4,5,6,6a-octahydrocyclopenta[c]pyrrole dihydrochloride (15 mg, 0.039 mmol, 1.0 eq.) was suspended in DCM (0.5 mL) and THF (0.5 mL). 3,3-Dimethylbutyraldehyde (24.4 μL, 0.194 mmol, 5.0 eq.) was added. The mixture was stirred at 50° C. for 30 min and sodium triacetoxyborohydride (41.1 mg, 0.194 mmol. 5.0 eq.) was added. The resulting solution was stirred at 50° C. for 2 h, after which time the reaction mixture was quenched with sat. soln. NaHCO₃, and extracted with chloroform/IPA (3:1, v/v). The combined extracts were filtered through a phase separator and concentrated. The crude residue was purified by RP-HPLC, and fractions containing the product were basified with sat. soln. NaHCO₃, and extracted with chloroform/IPA (3:1, v/v). The combined extracts were filtered through a phase separator and concentrated to give the title compound as a colorless oil (8.2 mg, 53%) (a mixture of endo/exo isomers, 3:1). ¹H-NMR (400 MHz, CDCl₃) (major isomer) δ 7.41 (d, J=9.2 Hz, 1H), 7.32 (s, 1H), 6.99 (d, J=9.1 Hz, 1H), 4.49 (d, J=6.8 Hz, 2H), 3.96 (s, 3H), 2.57-2.48 (m, 4H), 2.37-2.26 (m, 5H), 2.12-2.05 (m, 2H), 2.07 (s, 3H), 1.37-1.33 (m, 2H), 1.22-1.14 (m, 2H), 0.83 (s, 9H); ES-MS [M+H]⁺=398.4; (minor isomer) δ 7.43 (d, J=9.2 Hz, 1H), 7.32 (s, 1H), 6.99 (d, J=9.1 Hz, 1H), 4.44 (d, J=6.7 Hz, 2H), 3.96 (s, 3H), 2.92-2.85 (m, 2H), 2.72-2.66 (m, 2H), 2.59-2.51 (m, 1H), 2.36-2.31 (m, 2H), 2.07 (s, 3H), 2.01-1.94 (m, 2H), 1.68-1.63 (m, 2H), 1.54-1.46 (m, 2H), 1.38-1.34 (m, 2H), 0.83 (s, 9H); ES-MS [M+H]⁺=398.4.

### Representative Synthesis 10. 6-(1,4-Dimethyl-1H-pyrazol-5-yl)-N-(2-((3aR,6aS)-2-(3,3-dimethylbutyl)octahydrocyclopenta[c]pyrrol-5-yl)ethyl)pyridazin-3-amine

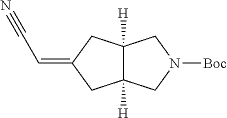 [SMILES: CC(C)(C)OC(=O)N1C[C@H]2C/C(=C/C#N)C[C@H]2C1]

tert-Butyl (3aR,6aS,E)-5-(cyanomethylene)hexahydrocyclopenta[c]pyrrole-2(1H)-carboxylate. To a round bottom flask, under nitrogen, was added diethyl cyanomethylphosphonate (1.6 mL, 8.9 mmol) dissolved in THF (20 mL) and cooled to −78° C. Sodium tert-butoxide (640 mg, 6.7 mmol) was added to the reaction and the mixture was stirred for 30 minutes at −78° C. At this time, a solution of cis-tert-butyl 5-oxohexahydrocyclopenta[c]pyrrole-2(1H)-carboxylate (1.0 g, 4.4 mmol) in THF (10 mL) was added and the reaction was allowed to slowly warm to room temperature over the course of 18 hours. Upon completion as determined by LCMS, the reaction was quenched by the addition of a saturated aqueous NH₄Cl solution and the mixture was extracted with ethyl acetate (3×35 mL). The organic layers were pooled, dried over sodium sulfate, filtered, and concentrated. The crude product was purified using Teledyne ISCO Combi-Flash system (liquid loading with DCM, 24G column, 0-60% EtOAc/Hex, 25 min run) to give the product (935 mg, 3.77 mmol, 85% yield) as a clear oil. LCMS (90 sec method): R_{T}=0.751, >95% @ 215 and 254 nM, m/z=193.2 [M+H−tBu]⁺. ¹H NMR (400 MHz, chloroform-d): δ 5.29-5.26 (m, 1H), 3.56 (bs, 2H), 3.17-3.06 (m, 2H), 2.92-2.72 (m, 4H), 2.59-2.55 (m, 1H); 2.46-2.40 (m. 1H), 1.45 (s, 9H).

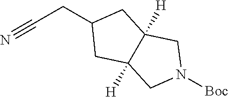 [SMILES: CC(C)(C)OC(=O)N1C[C@H]2CC(CC#N)C[C@H]2C1]

tert-Butyl (3aR,6aS)-5-(cyanomethyl)hexahydrocyclopenta[c]pyrrole-2(1H)-carboxylate. A round bottom flask containing a magnetic stir bar was equipped with a 3-way Schlenk adapter and evacuated then purged with nitrogen (×3). Palladium on activated carbon (10% by weight) (200 mg, 0.19 mmol) was added to the flask, followed by methanol (10 mL) then a solution of tert-butyl (3aR,6aS,E)-5-(cyanomethylene)hexahydrocyclopenta[c]pyrrole-2(1H)-carboxylate (935 mg, 3.8 mmol) in methanol (2 mL) The flask was equipped with a 3-way Schlenk adapter and evacuated then purged with nitrogen (×3). To the 3-way adapter was added a balloon containing H2 gas and the system was evacuated then purged with H2 (×3). The reaction was then allowed to stir 18 hours under H2 atmosphere and then analyzed by LC-MS (observe desired product [M+H−tBu]=195). Upon completion, the Pd/C catalyst was filtered off through a celite pad, the pad was washed twice with methanol, and the solvent was removed under a constant stream of air to afford tert-butyl (3aR,6aS)-5-(cyanomethyl)-3,3a,4,5,6,6a-hexahydro-1H-cyclopenta[c]pyrrole-2-carboxylate (928.9 mg, 3.71 mmol, 98% yield). The material was taken forward without further purification. LCMS (90 sec method): R_{T}=0.767, m/z=195.4 [M+H−tBu]⁺.

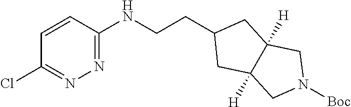 [SMILES: CC(C)(C)OC(=O)N1C[C@H]2CC(CCNc3ccc(Cl)nn3)C[C@H]2C1]

tert-Butyl (3aR,6aS)-5-(2-((6-chloropyridazin-3-yl)amino)ethyl)hexahydrocyclopenta[c]pyrrole-2(1H)-carboxylate. To a solution of tert-butyl (3aR,6aS)-5-(cyanomethyl)hexahydrocyclopenta[c]pyrrole-2(1H)-carboxylate (464 mg, 1.9 mmol) in THF (12.4 mL) at 0° C. was added borane dimethyl sulfide complex (2M in THF, 3.7 mL, 7.4 mmol) dropwise. The mixture was stirred for 1 h at 0° C. then added slowly to a vial containing ethanol at 0° C. to quench excess borane. The mixture was stirred for 20 minutes then allowed to warm to ambient temperature and evaporated to dryness. The crude product was used without further purification. LCMS (90 sec method): R_{T}=0.582, >95% @ 215 and 254 nM, m/z=199.4 [M+H−tBu]⁺.

Into two 20-mL microwave vials was equally divided a solution of tert-butyl (3aR,6aS)-5-(2-aminoethyl)hexahydrocyclopenta[c]pyrrole-2(1H)-carboxylate (0.47 g, 1.9 mmol) and N,N-diisopropylethylamine (1.9 mL, 11. mmol) dissolved in 1-butanol (4.6 mL). Next, 3,6-dichloropyridazine (1.3 mL, 9.3 mmol) was added, the vials sealed, and the mixtures microwave irradiated for 45 minutes at 130° C. After LCMS analysis, the reaction was concentrated and crude product was purified using Teledyne ISCO Combi-Flash system (liquid loading with DCM, 24G column, 20% ethyl acetate/hexanes, 6 min; then 0-80% EtOAc/DCM, 25 min run) to afford tert-butyl (3aR,6aS)-5-[2-[(6-chloropyridazin-3-yl)amino]ethyl]-3,3a,4,5,6,6a-hexahydro-1H-cyclopenta[c]pyrrole-2-carboxylate (519 mg, 1.41 mmol, 77% yield over 2 steps). LCMS (90 sec method): R_{T}=0.768, >95% @ 215 and 254 nM, m/z=367.2 [M+H]⁺. ¹H NMR (400 MHz, chloroform-d): δ 7.15 (d, J=9.3 Hz, 1H), 6.60 (d, J=9.3 Hz, 1H), 4.76 (bs, 1H), 3.55-3.35 (m, 4H), 3.21-3.07, (m, 2H), 2.74-2.53 (m, 2H), 2.15-2.07 (m, 2H), 2.04-1.94 (m, 1H), 1.72 (q, J=7.2 Hz, 2H), 1.67-1.61 (m, 2H), 1.45 (s, 9H).

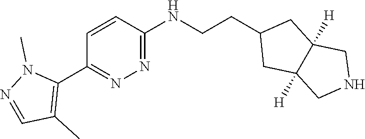 [SMILES: Cc1cnn(C)c1-c1ccc(NCCC2C[C@H]3CNC[C@H]3C2)nn1]

tert-Butyl (3aR,6aS)-5-(2-((6-(1,4-dimethyl-1H-pyrazol-5-yl)pyridazin-3-yl)amino)ethyl)hexahydrocyclopenta[c]pyrrole-2(1H)-carboxylate. To a microwave vial was added 1,4-dimethylpyrazole-5-boronic acid pinacol ester (393 mg, 1.8 mmol), tert-butyl (3aR,6aS)-5-(2-((6-chloropyridazin-3-yl)amino)ethyl)hexahydrocyclopenta[c]pyrrole-2(1H)-carboxylate (259 mg, 0.71 mmol), potassium carbonate (0.22 mL, 3.5 mmol), and RuPhos-Pd-G3 (59 mg, 0.07 mmol) dissolved in 1,4-dioxane/water (4:1) (7.0 mL, degassed). The vial was purged with N₂, sealed, and subjected to microwave irradiation for 30 minutes at 120° C. Upon completion, as determined by LCMS, the reaction mixture was filtered over celite, the celite plug was washed with DCM, and saturated aqueous NaHCO₃ was added to the filtrate. The DCM layer was then isolated and the aqueous layer was extracted with chloroform/IPA (4:1) (3×10 mL). The organic layers were passed through a phase separator and concentrated. The crude product was purified using Teledyne ISCO Combi-Flash system (liquid loading with DCM, 12G column, 0-50% EtOAc/DCM, 10 min run; then 0-7% MeOH/DCM/0.1% NH₄OH) to afford tert-butyl (3aR,6aS)-5-[2-[[6-(2,4-dimethylpyrazol-3-yl)pyridazin-3-yl]amino]ethyl]-3,3a,4,5,6,6a-hexahydro-1H-cyclopenta[c]pyrrole-2-carboxylate (229 mg, 0.54 mmol, 76% yield). LCMS (90 sec method): R_{T}=0.802, >95% @ 215 and 254 nM, m/z=427.5 [M+H]⁺.

To a solution of tert-butyl (3aR,6aS)-5-(2-((6-(1,4-dimethyl-1H-pyrazol-5-yl)pyridazin-3-yl)amino)ethyl)hexahydrocyclopenta[c]pyrrole-2(1H)-carboxylate (229 mg, 0.54 mmol) in DCM (2 mL) was added 4M hydrogen chloride in dioxane (0.86 mL, 3.43 mmol) and the mixture was stirred for 5 hours. Upon completion as determined by LCMS, the reaction was concentrated to afford the product (194 mg, 0.54 mmol, 98% yield). The material was carried forward without further purification. LCMS (90 sec method): R_{T}=0.372, >95% @ 215 and 254 nM, m/z=327.5 [M+H]⁺. ¹H NMR (400 MHz, Methanol-d₄): δ 7.96 (d, J=9.7 Hz, 1H), 7.73-7.66 (m, 1H), 7.49 (s, 1H), 3.98 (s, 3H), 3.54-3.47 (m, 3H), 3.22-3.18 (m, 2H), 2.95-2.91 (m, 3H), 2.23-2.24 (m, 2H). 2.19 (s, 3H), 2.16-2.03 (m, 1H), 1.93-1.84 (m, 3H), 1.22-1.14 (m, 2H).

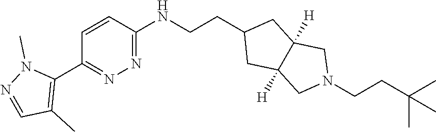 [SMILES: Cc1cnn(C)c1-c1ccc(NCCC2C[C@@H]3CN(CCC(C)(C)C)C[C@@H]3C2)nn1]

6-(1,4-Dimethyl-1H-pyrazol-5-yl)-N-(2-((3aR,6aS)-2-(3,3-dimethylbutyl)octahydrocyclopenta[c]pyrrol-5-yl)ethyl)pyridazin-3-amine. To a vial was added tert-butyl (3aR,6aS)-5-(2-((6-(1,4-dimethyl-1H-pyrazol-5-yl)pyridazin-3-yl)amino)ethyl)hexahydrocyclopenta[c]pyrrole-2(1H)-carboxylate (10 mg, 0.03 mmol) in DCE (0.5 mL)/THF (0.5 mL). Next, 3,3-dimethylbutyraldehyde (22 μL, 0.18 mmol) was added followed by sodium triacetoxyborohydride (29 mg, 0.14 mmol). The resulting suspension was stirred at ambient temperature for 18 hours then analyzed by LCMS. The reaction was quenched with a saturated aqueous NaHCO₃ solution, and extracted with 3:1 chloroform/IPA. The solvents were concentrated. The crude product was dissolved in DMSO (1 mL) and purified using the Gilson (Acidic, 30×50 mm column, 15-60% ACN/0.1% aqueous TFA, 4 min run). Fractions containing the product were basified with a saturated aqueous NaHCO₃ solution and extracted with 3:1 chloroform/IPA. The solvents were concentrated to give title compound as a white solid (74% yield). LCMS (90 sec method): R_{T}=0.693, >95% @ 215 and 254 nM, m/z=411.4 [M+H]⁺. ¹H NMR (400 MHz, chloroform-d): δ 7.36 (s, 1H), 7.27 (d, J=9.2 Hz, 1H), 6.69 (d, J=9.2 Hz, 1H), 4.88 (bs, 1H), 3.99 (s, 3H), 3.49-3.42 (m, 2H), 2.56-2.47 (m, 3H), 2.41-2.35 (m, 2H), 2.29-2.25 (m, 2H), 2.10 (s, 3H), 2.18-2.04 (m, 2H), 1.94-1.80 (m, 2H), 1.78-1.72 (m, 2H), 1.71-1.63 (m, 1H), 1.43-1.38 (m, 2H), 1.11-1.03 (m, 2H), 0.89 (s, 9H).

### Representative Synthesis 11. N-(4′-(((3aR,5s,6aS)-2-(3,3-Dimethylbutyl)octahydrocyclopenta[c]pyrrol-5-yl)oxy)-[1,1′-biphenyl]-4-yl)acetamide

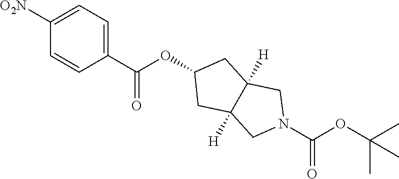 [SMILES: CC(C)(C)OC(=O)N1C[C@H]2C[C@H](OC(=O)c3ccc([N+](=O)[O-])cc3)C[C@H]2C1]

tert-Butyl (3aR,5s,6aS)-5-((4-nitrobenzoyl)oxy)hexahydrocyclopenta[c]pyrrole-2(1H)-carboxylate. To a solution of tert-butyl (3aR,5r,6aS)-5-hydroxyhexahydrocyclopenta[c]pyrrole-2(1H)-carboxylate (1.01 g, 4.44 mmol), triphenylphosphine (1.40 g, 5.33 mmol), and 4-nitrobenzoic acid (890 mg, 5.33 mmol) in diethyl ether (15 mL) was added diisopropyl azodicarboxylate (1.05 mL, 5.33 mmol) at −78° C. The reaction mixture was warmed to r.t. and stirred for 18 h, after which time the reaction mixture was quenched with the addition of MeOH (2 mL), and stirred for 15 min. Solvents were concentrated under reduced pressure, and the crude residue was purified by column chromatography (3-30% EtOAc in hexanes) to give the title compound as a colorless oil that solidified upon standing (1.67 g, 100%, 80% purity after chromatography). ES-MS [M+H−tbutyl]⁺=321.3.

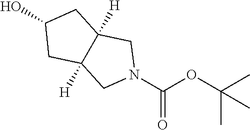 [SMILES: CC(C)(C)OC(=O)N1C[C@H]2C[C@H](O)C[C@H]2C1]

tert-Butyl (3aR,5s,6aS)-5-hydroxyhexahydrocyclopenta[c]pyrrole-2(1H)-carboxylate. tert-Butyl (3aR,5s,6aS)-5-((4-nitrobenzoyl)oxy)hexahydrocyclopenta[c]pyrrole-2(1H)-carboxylate (1.67 g, 4.44 mmol) was dissolved in THF (30 mL) and potassium trimethylsilanolate (2.85 g, 22.2 mmol) was added. The resulting cloudy brown mixture was stirred at r.t. for 2 h, after which time solvents were concentrated under reduced pressure, and the crude residue was diluted in DCM and H₂O. The aqueous layer was extracted with DCM, and the combined organic extracts were dried with MgSO₄. Solvents were filtered and concentrated under reduced pressure, and the crude residue was purified by column chromatography (0-1% MeOH in DCM) to give the title compound as a white solid (435 mg, 43%). ¹H-NMR (400 MHz, CDCl₃) δ 4.50-4.45 (m, 1H), 3.54-3.46 (m, 2H), 3.16 (br, 2H), 2.89-2.79 (m, 2H), 1.92-1.86 (m, 2H), 1.73-1.66 (m, 2H), 1.45 (s, 9H). ES-MS [M+H−tbutyl]⁺=172.4.

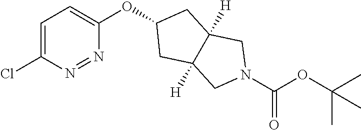 [SMILES: CC(C)(C)OC(=O)N1C[C@H]2C[C@H](Oc3ccc(Cl)nn3)C[C@H]2C1]

tert-Butyl (3aR,5s,6aS)-5-((6-chloropyridazin-3-yl)oxy)hexahydrocyclopenta[c]pyrrole-2(1H)-carboxylate. tert-Butyl (3aR,5s,6aS)-5-hydroxyhexahydrocyclopenta[c]pyrrole-2(1H)-carboxylate (430 mg, 1.89 mmol) was dissolved in THF (10 mL) and NaH (91 mg, 3.78 mmol, 60% dispersion in mineral oil) was added at 0° C. After stirring for 5 mins, 3,6-dichloropyridazine (423 mg, 2.84 mmol) was added and the resulting solution was warmed to r.t. and stirred for 70 h, after which time the reaction mixture was diluted with DCM and H₂O. The aqueous layer was extracted with DCM, and the combined organic extracts were dried with MgSO₄. Solvents were filtered and concentrated under reduced pressure, and the crude residue was purified by column chromatography (3-30% EtOAc in hexanes) to give the title compound as a white solid (477 mg, 74%). ¹H-NMR (400 MHz, CDCl₃) δ 7.35 (d, J=9.2 Hz, 1H), 6.88 (d, J=9.2 Hz, 1H), 5.75-5.71 (m, 1H), 3.54 (br, 2H), 3.22 (br, 2H), 2.91-2.81 (m, 2H), 2.21-2.13 (m, 2H), 1.96 (dt, J=14.5, 5.6 Hz, 2H), 1.46 (s, 9H). ES-MS [M+H−tbutyl]⁺=284.4.

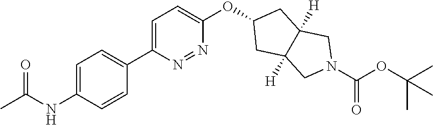 [SMILES: CC(=O)Nc1ccc(-c2ccc(O[C@H]3C[C@@H]4CN(C(=O)OC(C)(C)C)C[C@@H]4C3)nn2)cc1]

tert-Butyl (3aR,5s,6aS)-5-((6-(4-acetamidophenyl)pyridazin-3-yl)oxy)hexahydrocyclopenta[c]pyrrole-2(1H)-carboxylate. tert-Butyl (3aR,5s,6aS)-5-((6-chloropyridazin-3-yl)oxy)hexahydrocyclopenta[c]pyrrole-2(1H)-carboxylate (228 mg, 0.67 mmol), 4-acetylaminophenyl boronic acid (144 mg, 0.81 mmol), potassium carbonate (282 mg, 2.01 mmol) and RuPhos Pd G3 (56 mg, 0.067 mmol) were combined in a sealed vial, which was placed under an inert atmosphere. 5:1 Dioxanes/H₂O solution (4 mL, degassed) was then added via syringe, and the resulting solution was stirred at 100° C. for 1 h, after which time the reaction was cooled to r.t. and diluted with DCM and sat. NaHCO₃. The aqueous layer was extracted with DCM, and the combined organic extracts were filtered through a phase separator and concentrated. The crude residue was purified by column chromatography (12-100% EtOAc in hexanes) to give the title compound as a white solid (94 mg, 32%). ES-MS [M+H−tbutyl]⁺=383.3.

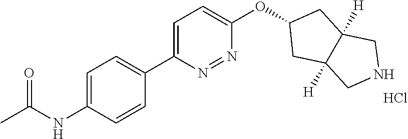 [SMILES: CC(=O)Nc1ccc(-c2ccc(O[C@@H]3C[C@H]4CNC[C@H]4C3)nn2)cc1 ; Cl]

N-(4-(6-(((3aR,5s,6aS)-Octahydrocyclopenta[c]pyrrol-5-yl)oxy)pyridazin-3-yl)phenyl)acetamide hydrochloride. tert-Butyl (3aR,5s,6aS)-5-((6-(4-acetamidophenyl)pyridazin-3-yl)oxy)hexahydrocyclopenta[c]pyrrole-2(1H)-carboxylate (94 mg, 0.21 mmol) was dissolved in 1,4-dioxane (2 mL) and 4M HCl in dioxanes solution (2 mL) was added dropwise. The resulting solution was stirred at r.t. for 30 min, after which time solvents were concentrated under reduced pressure to give the title compound as a yellow solid which was used directly without further purification (80 mg, 100%). ES-MS [M+H]⁺=339.4.

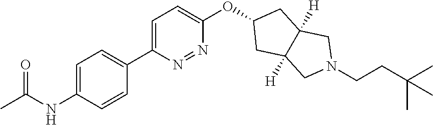 [SMILES: CC(=O)Nc1ccc(-c2ccc(O[C@H]3C[C@@H]4CN(CCC(C)(C)C)C[C@@H]4C3)nn2)cc1]

N-(4′-(((3aR,5s,6aS)-2-(3,3-Dimethylbutyl)octahydrocyclopenta[c]pyrrol-5-yl)oxy)-[1,1′-biphenyl]-4-yl)acetamide. N-(4-(6-(((3aR,5s,6aS)-Octahydrocyclopenta[c]pyrrol-5-yl)oxy)pyridazin-3-yl)phenyl)acetamide hydrochloride (16 mg, 0.043 mmol) was dissolved in THF (0.5 mL) and DCM (0.5 mL) and 3,3-dimethylbutyraldehyde (21 mg, 0.21 mmol) was added, followed by sodium triacetoxyborohydride (45 mg, 0.21 mmol). The resulting mixture was stirred at r.t. for 1 h, after which time the reaction mixture was quenched with sat. NaHCO₃ and diluted with 3:1 chloroform/IPA. The aqueous layer was extracted with 3:1 chloroform/IPA, and the organic extracts were filtered through a phase separator and concentrated. The crude residue was purified by RP-HPLC (12-42% MeCN in 0.1% TFA aqueous solution over 5 min), and fractions containing product were basified with sat. NaHCO₃, and extracted with 3:1 chloroform/IPA. The organic extracts were filtered through a phase separator and concentrated to give the title compound as a white solid (5.4 mg, 30%). ¹H-NMR (400 MHz, CDCl₃) δ 7.97 (d, J=8.6 Hz, 2H), 7.73 (d, J=9.2 Hz, 1H), 7.63 (d, J=8.6 Hz, 2H), 7.43 (s, 1H), 6.95 (d, J=9.2 Hz, 1H), 5.80 (p, J=4.7 Hz, 1H), 2.83-2.74 (m, 2H), 2.55-2.51 (m, 2H), 2.44-2.37 (m, 3H), 2.21 (s, 3H), 2.16-2.09 (m, 2H), 1.94 (dt, J=13.6, 5.0 Hz, 2H), 1.79-1.65 (m, 1H), 1.43-1.39 (m, 2H), 0.90 (s, 9H). ES-MS [M+H]⁺=423.0.

### Representative Synthesis 12. 3aR,5s,6aS)—N-(6-Morpholinopyridazin-3-yl)-2-((tetrahydro-2H-pyran-4-yl)methyl)octahydrocyclopenta[c]pyrrol-5-amine

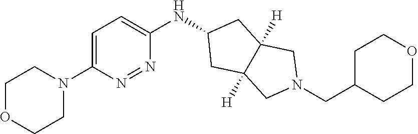 [SMILES: c1cc(N2CCOCC2)nnc1N[C@H]1C[C@@H]2CN(CC3CCOCC3)C[C@@H]2C1]

(3aR,5s,6aS)—N-(6-Chloropyridazin-3-yl)-2-((tetrahydro-2H-pyran-4-yl)methyl)octahydrocyclopenta[c]pyrrol-5-amine (20 mg, 0.059 mmol) and morpholine (26 μL, 030 mmol) were combined in a microwave vial, and NMP (1 mL) was added, followed by conc. HCl (25 μL, 30 mmol) and N,N-diisopropylethylamine (52 μL, 0.30 mmol). The resulting solution was heated under microwave irradiation at 200° C. for 1 h, after which time the reaction mixture was purified directly by RP-HPLC (20-60% MeCN in 0.05% NH₄OH aqueous solution over 5 min). Fractions containing product were concentrated to give the title compound as a slightly brown solid (13 mg, 55%). ¹H-NMR (400 MHz, CDCl₃) δ 6.86 (d, J=9.6 Hz, 1H), 6.61 (d, J=9.6 Hz, 1H), 4.38-4.30 (m, 1H), 4.20 (d, J=6.8 Hz, 1H), 3.95 (dd, J=11.4, 3.6 Hz, 2H), 3.83-3.81 (m, 4H), 3.40-3.34 (m, 6H), 2.73-2.65 (m, 4H), 2.26-2.24 (m, 4H), 1.93 (dd, J=12.6, 5.6 Hz, 2H), 1.72-1.58 (m, 5H), 1.32-1.21 (m, 2H). ES-MS [M+H]⁺=388.4.

### Representative Synthesis 13. (3aR,5s,6aS)—N-(6-(4-Fluorophenoxy)pyridazin-3-yl)-2-((tetrahydro-2H-pyran-4-yl)methyl)octahydrocyclopenta[c]pyrrol-5-amine

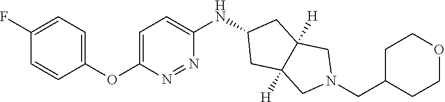 [SMILES: Fc1ccc(Oc2ccc(N[C@H]3C[C@@H]4CN(CC5CCOCC5)C[C@@H]4C3)nn2)cc1]

(3aR,5s,6aS)—N-(6-Chloropyridazin-3-yl)-2-((tetrahydro-2H-pyran-4-yl)methyl)octahydrocyclopenta[c]pyrrol-5-amine (25 mg, 0.074 mmol), potassium phosphate tribasic (32 mg, 0.15 mmol), 4-fluorophenol (33 mg, 0.30 mmol), palladium(II) acetate (1.7 mg, 0.007 mmol) and t-butylXPhos (4.7 mg, 0.011 mmol) were combined in a vial, which was sealed and placed under an inert atmosphere. Toluene (1 mL) was then added via syringe, and the resulting mixture was heated to 100° C. overnight, after which time solvents were concentrated, and the crude residue was taken up in DMSO. Solids were removed by syringe filtration, and the crude residue was purified by RP-HPLC (5-35% MeCN in 0.1% aq TFA solution over 5 min). Fractions containing product were basified with NaHCO₃, and extracted with 3:1 chloroform/IPA. The organic extracts were combined and passed through a phase separator and concentrated to yield the title compound as a slightly yellow oil (3.3 mg, 11%). ¹H-NMR (400 MHz, CDCl₃) δ 7.16-7.10 (m, 3H), 7.08-7.02 (m, 2H), 6.98 (d, J=9.4 Hz, 1H), 4.44 (br, 1H), 3.97 (dd, J=11.0, 2.9 Hz, 2H), 3.38 (td, J=12.0, 1.8 Hz, 2H), 3.06 (br, 2H), 2.57 (br, 2H), 2.37 (br, 2H), 2.09-1.97 (m, 2H), 1.94-1.48 (m, 8H), 1.42-1.26 (m, 2H). ES-MS [M+H]⁺=413.2.

The compounds shown in Table 1 may be prepared similarly to the compounds described above, with appropriate starting materials. Additional starting materials that may be used to prepare compounds of the invention include tetrahydro-2H-pyran-4-carbaldehyde, (S)-(1,4-dioxan-2-yl)methanol), (R)-(1,4-dioxan-2-yl)methanol), (S)-1,4-dioxane-2-carboxylic acid, (R)-1,4-dioxane-2-carboxylic acid, (S)-tetrahydro-2H-pyran-2-carboxylic acid, (R)-tetrahydro-2H-pyran-2-carboxylic acid, 4-methoxytetrahydro-2H-pyran-4-carboxylic acid, 3-methyltetrahydro-2H-pyran-3-carboxylic acid, 2-methyltetrahydro-2H-pyran-2-carboxylic acid, 4-ethyltetrahydro-2H-pyran-4-carboxylic acid, 3,3-difluorotetrahydro-2H-pyran-4-carboxylic acid, (S)-tetrahydrofuran-3-carboxylic acid, (R)-tetrahydrofuran-3-carboxylic acid, (R)-(tetrahydrofuran-3-yl)methanol, 2-(tetrahydro-2H-pyran-4-yl)acetaldehyde, 4-methyltetrahydro-2H-pyran-4-carbaldehyde, 4-methyltetrahydro-2H-pyran-4-carboxylic acid, rac-(1R,2S,4S)-2-(bromomethyl)-7-oxabicyclo[2.2.1]heptane, rac-(1R,2R,4S)-2-(bromomethyl)-7-oxabicyclo[2.2.1]heptane, rac-(3aR,6aS)-hexahydro-2H-cyclopenta[b]furan-3a-carboxylic acid, tetrahydro-4H-pyran-4-one, 2,2-dimethyltetrahydro-4H-pyran-4-one, 2,2,6,6-tetramethyltetrahydro-4H-pyran-4-one, tetrahydro-4H-thiopyran-4-one, tetrahydro-4H-thiopyran-4-one 1,1-dioxide, dihydro-2H-pyran-3(4H)-one, oxetan-3-one, 2-oxaspiro[3.3]heptan-6-one, 1,6-dioxaspiro[2.5]octane, 3,3-dimethylbutyraldehyde, cyclohexanecarbaldehyde, cycloheptanecarbaldehyde, cyclopentanone, cyclohexanone, 4,4-dimethylcyclohexan-1-one, 4,4-difluorocyclohexan-1-one, cycloheptanone, 1,3-dihydro-2H-inden-2-one, picolinaldehyde, 6-methylpicolinaldehyde, 6-methoxypicolinaldehyde, 4-chloropicolinaldehyde, 6-chloropicolinaldehyde, 5-fluoropicolinaldehyde, 6-fluoropicolinaldehyde, 3-methylpicolinaldehyde, 1-(pyridin-2-yl)ethan-1-one, 6,7-dihydro-4H-pyrazolo[5,1-c][1,4]oxazine-2-carbaldehyde, 2,2-difluorobenzo[d][1,3]dioxole-5-carbaldehyde, and pyridazine-4-carbaldehyde, 1-fluorocyclohexane-1-carboxylic acid, 2-fluorobenzaldehyde, 2,3-difluorobenzaldehyde, 2,4-difluorobenzaldehyde, 2,6-difluorobenzoic acid and 3,6-dichloro-N-cyclopropylpyridazin-4-amine.

TABLE 1

| Cpd. | | | ES-MS |
|---|---|---|---|
| No. | Name | Structure | [M + 1]⁺ |
| 1 | (3aR,5s,6aS)-N-(6-(2-chloro-5- fluorophenyl)pyridazin-3-yl)-2-(3- methoxypropyl)octahydrocyclopenta[c]pyrrol- 5-amine | 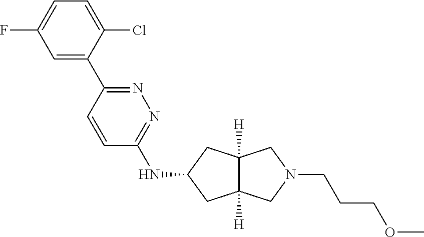 [SMILES: COCCCN1C[C@H]2C[C@H](Nc3ccc(-c4cc(F)ccc4Cl)nn3)C[C@H]2C1] | 405.4 |
| 2 | (3aR,5s,6aS)-N-(6-(2-chloro-5- fluorophenyl)pyridazin-3-yl)-2-(tetrahydro- 2H-pyran-4-yl)octahydrocyclopenta[c]pyrrol- 5-amine | 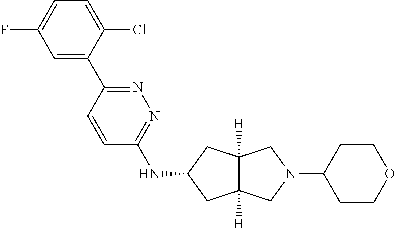 [SMILES: Fc1ccc(Cl)c(-c2ccc(N[C@H]3C[C@@H]4CN(C5CCOCC5)C[C@@H]4C3)nn2)c1] | 417.2 |
| 3 | N-cyclopropyl-6-(((3aR,5s,6aS)-2- ((tetrahydro-2H-pyran-4- yl)methyl)octahydrocyclopenta[c]pyrrol-5- yl)amino)pyridazine-3-carboxamide | 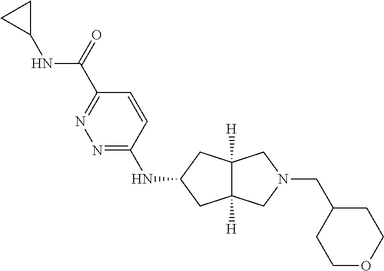 [SMILES: O=C(NC1CC1)c1ccc(N[C@H]2C[C@@H]3CN(CC4CCOCC4)C[C@@H]3C2)nn1] | 386.2 |
| 4 | N-cyclohexyl-6-(((3aR,5s,6aS)-2- ((tetrahydro-2H-pyran-4- yl)methyl)octahydrocyclopenta[c]pyrrol-5- yl)amino)pyridazine-3-carboxamide | 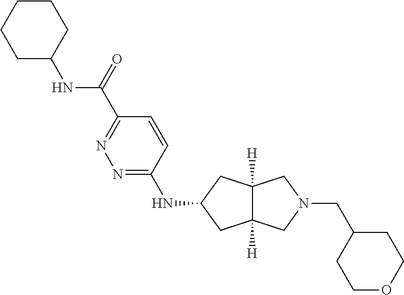 [SMILES: O=C(NC1CCCCC1)c1ccc(N[C@H]2C[C@@H]3CN(CC4CCOCC4)C[C@@H]3C2)nn1] | 428.2 |
| 5 | piperidin-1-yl(6-(((3aR,5s,6aS)-2- ((tetrahydro-2H-pyran-4- yl)methyl)octahydrocyclopenta[c]pyrrol-5- yl)amino)pyridazin-3-yl)methanone | 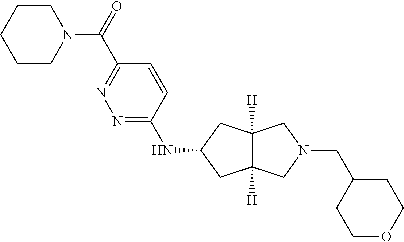 [SMILES: O=C(c1ccc(N[C@H]2C[C@@H]3CN(CC4CCOCC4)C[C@@H]3C2)nn1)N1CCCCC1] | 414.2 |
| 6 | morpholino(6-(((3aR,5s,6aS)-2-((tetrahydro- 2H-pyran-4- yl)methyl)octahydrocyclopenta[c]pyrrol-5- yl)amino)pyridazin-3-yl)methanone | 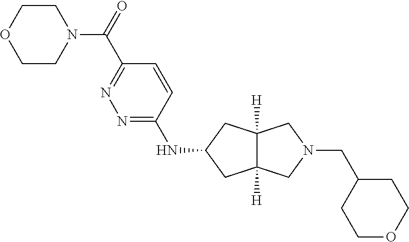 [SMILES: O=C(c1ccc(N[C@H]2C[C@@H]3CN(CC4CCOCC4)C[C@@H]3C2)nn1)N1CCOCC1] | 416.2 |
| 7 | N-phenyl-6-(((3aR,5s,6aS)-2-((tetrahydro-2H- pyran-4- yl)methyl)octahydrocyclopenta[c]pyrrol-5- yl)amino)pyridazine-3-carboxamide | 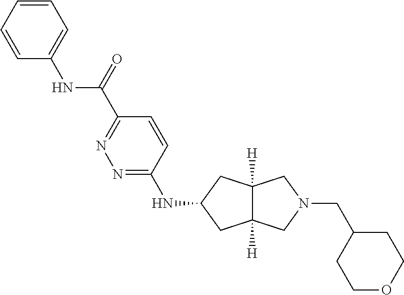 [SMILES: O=C(Nc1ccccc1)c1ccc(N[C@H]2C[C@@H]3CN(CC4CCOCC4)C[C@@H]3C2)nn1] | 422.2 |
| 8 | N-(2-fluorophenyl)-6-(((3aR,5s,6aS)-2- ((tetrahydro-2H-pyran-4- yl)methyl)octahydrocyclopenta[c]pyrrol-5- yl)amino)pyridazine-3-carboxamide | 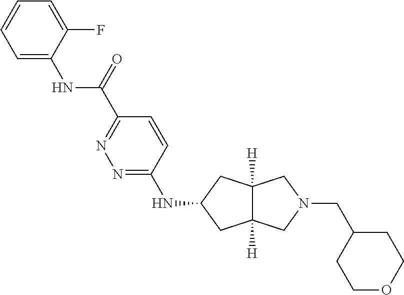 [SMILES: O=C(Nc1ccccc1F)c1ccc(N[C@H]2C[C@@H]3CN(CC4CCOCC4)C[C@@H]3C2)nn1] | 440.2 |
| 9 | N-(3-fluorophenyl)-6-(((3aR,5s,6aS)-2- ((tetrahydro-2H-pyran-4- yl)methyl)octahydrocyclopenta[c]pyrrol-5- yl)amino)pyridazine-3-carboxamide | 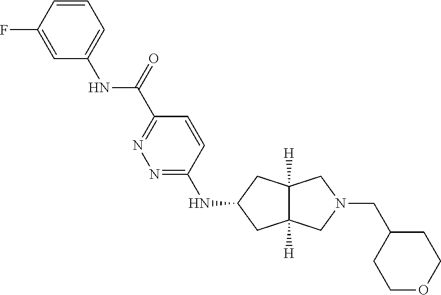 [SMILES: O=C(Nc1cccc(F)c1)c1ccc(N[C@H]2C[C@@H]3CN(CC4CCOCC4)C[C@@H]3C2)nn1] | 440.2 |
| 10 | N-(4-fluorophenyl)-6-(((3aR,5s,6aS)-2- ((tetrahydro-2H-pyran-4- yl)methyl)octahydrocyclopenta[c]pyrrol-5- yl)amino)pyridazine-3-carboxamide | 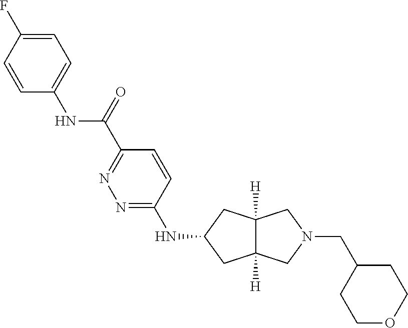 [SMILES: O=C(Nc1ccc(F)cc1)c1ccc(N[C@H]2C[C@@H]3CN(CC4CCOCC4)C[C@@H]3C2)nn1] | 440.2 |
| 11 | N-(2-chloro-5-fluorophenyl)-6- (((3aR,5s,6aS)-2-((tetrahydro-2H-pyran-4- yl)methyl)octahydrocyclopenta[c]pyrrol-5- yl)amino)pyridazine-3-carboxamide | 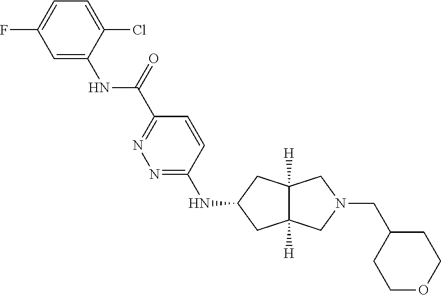 [SMILES: O=C(Nc1cc(F)ccc1Cl)c1ccc(N[C@H]2C[C@@H]3CN(CC4CCOCC4)C[C@@H]3C2)nn1] | 474.2 |
| 12 | (3aR,5s,6aS)-N-(6-(2-chloro-5- fluorophenyl)pyridazin-3-yl)-2- cyclohexyloctahydrocyclopenta[c]pyrrol-5- amine | 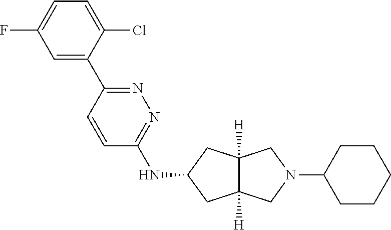 [SMILES: Fc1ccc(Cl)c(-c2ccc(N[C@H]3C[C@@H]4CN(C5CCCCC5)C[C@@H]4C3)nn2)c1] | 415.2 |
| 13 | (3aR,5s,6aS)-N-(6-(2-chloro-5- fluorophenyl)pyridazin-3-yl)-2-(tetrahydro- 2H-pyran-3-yl)octahydrocyclopenta[c]pyrrol- 5-amine | 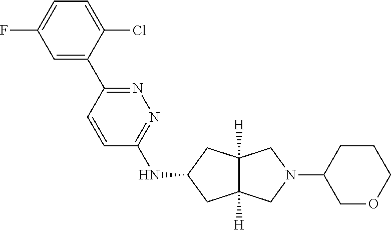 [SMILES: Fc1ccc(Cl)c(-c2ccc(N[C@H]3C[C@@H]4CN(C5CCCOC5)C[C@@H]4C3)nn2)c1] | 417.2 |
| 14 | (3aR,5s,6aS)-N-(6-(2-chloro-5- fluorophenyl)pyridazin-3-yl)-2-(oxetan-3- yl)octahydrocyclopenta[c]pyrrol-5-amine | 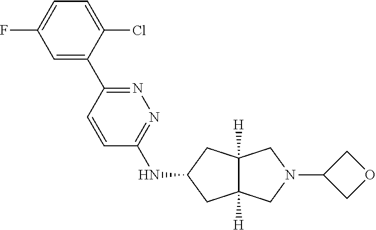 [SMILES: Fc1ccc(Cl)c(-c2ccc(N[C@H]3C[C@@H]4CN(C5COC5)C[C@@H]4C3)nn2)c1] | 389.2 |
| 15 | (3aR,5s,6aS)-N-(6-(2-chloro-5- fluorophenyl)pyridazin-3-yl)-2-(tetrahydro- 2H-thiopyran-4- yl)octahydrocyclopenta[c]pyrrol-5-amine | 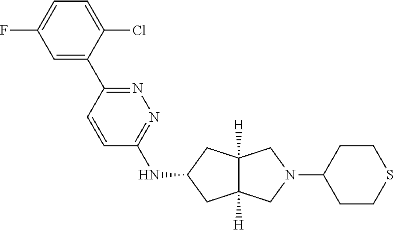 [SMILES: Fc1ccc(Cl)c(-c2ccc(N[C@H]3C[C@@H]4CN(C5CCSCC5)C[C@@H]4C3)nn2)c1] | 433.2 |
| 16 | (3aR,5s,6aS)-N-(6-(2-chloro-5- fluorophenyl)pyridazin-3-yl)-2-(2,2- dimethyltetrahydro-2H-pyran-4- yl)octahydrocyclopenta[c]pyrrol-5-amine | 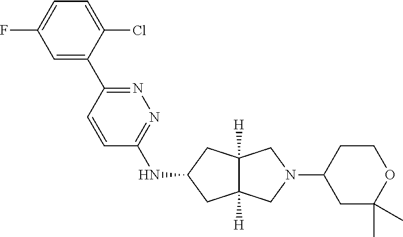 [SMILES: CC1(C)CC(N2C[C@H]3C[C@H](Nc4ccc(-c5cc(F)ccc5Cl)nn4)C[C@H]3C2)CCO1] | 445.2 |
| 17 | 2-((3aR,5s,6aS)-5-((6-(2-chloro-5- fluorophenyl)pyridazin-3- yl)amino)hexahydrocyclopenta[c]pyrrol- 2(1H)-yl)benzonitrile | 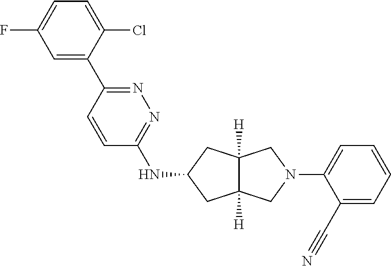 [SMILES: N#Cc1ccccc1N1C[C@H]2C[C@H](Nc3ccc(-c4cc(F)ccc4Cl)nn3)C[C@H]2C1] | 434.3 |
| 18 | (3aR,5s,6aS)-N-(6-(2-chloro-5- fluorophenyl)pyridazin-3-yl)-2-(2,2,6,6- tetramethyltetrahydro-2H-pyran-4- yl)octahydrocyclopenta[c]pyrrol-5-amine | 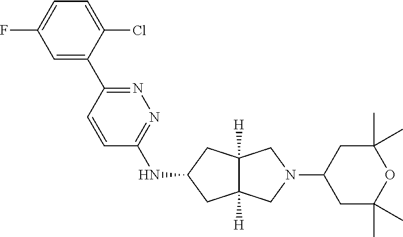 [SMILES: CC1(C)CC(N2C[C@H]3C[C@H](Nc4ccc(-c5cc(F)ccc5Cl)nn4)C[C@H]3C2)CC(C)(C)O1] | 473.2 |
| 19 | N-((3aR,5s,6aS)-2-(2,2-dimethyltetrahydro- 2H-pyran-4-yl)octahydrocyclopenta[c]pyrrol- 5-yl)-5-phenylthiazol-2-amine | 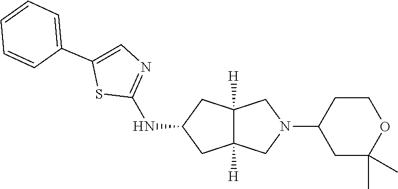 [SMILES: CC1(C)CC(N2C[C@H]3C[C@H](Nc4ncc(-c5ccccc5)s4)C[C@H]3C2)CCO1] | 398.4 |
| 20 | N-(4-(6-(((3aR,5s,6aS)-2-(2,2- dimethyltetrahydro-2H-pyran-4- yl)octahydrocyclopenta[c]pyrrol-5- yl)amino)pyridazin-3-yl)phenyl)acetamide | 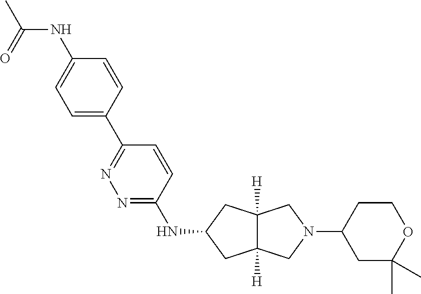 [SMILES: CC(=O)Nc1ccc(-c2ccc(N[C@H]3C[C@@H]4CN(C5CCOC(C)(C)C5)C[C@@H]4C3)nn2)cc1] | 450.2 |
| 21 | (3aR,5s,6aS)-2-(2,2-dimethyltetrahydro-2H- pyran-4-yl)-N-(6-(imidazo[1,2-a]pyridin-6- yl)pyridazin-3- yl)octahydrocyclopenta[c]pyrrol-5-amine | 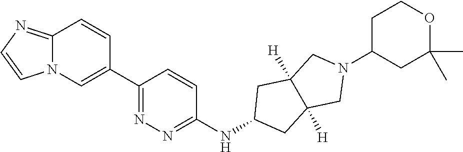 [SMILES: CC1(C)CC(N2C[C@H]3C[C@H](Nc4ccc(-c5ccc6nccn6c5)nn4)C[C@H]3C2)CCO1] | 433.3 |
| 22 | (3aR,5s,6aS)-N-(6-(1,3-dimethyl-1H-pyrazol- 4-yl)pyridazin-3-yl)-2-(2,2- dimethyltetrahydro-2H-pyran-4- yl)octahydrocyclopenta[c]pyrrol-5-amine | 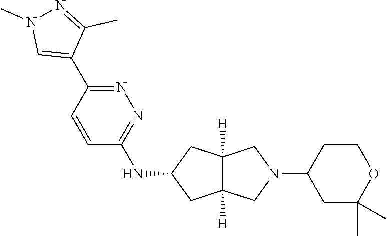 | 411.2 |
| 23 | (3aR,5s,6aS)-N-(6-(phenylsulfonyl)pyridazin- 3-yl)-2-((tetrahydro-2H-pyran-4- yl)methyl)octahydrocyclopenta[c]pyrrol-5- amine | 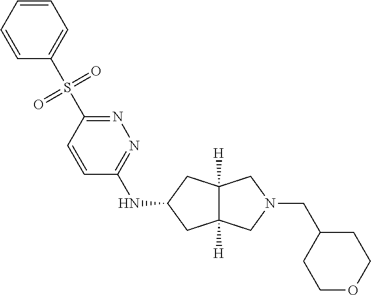 [SMILES: O=S(=O)(c1ccccc1)c1ccc(N[C@H]2C[C@@H]3CN(CC4CCOCC4)C[C@@H]3C2)nn1] | 443 |
| 24 | (3aR,5s,6aS)-2-((1,5-dimethyl-1H-pyrazol-3- yl)methyl)-N-(6-(phenylsulfonyl)pyridazin-3- yl)octahydrocyclopenta[c]pyrrol-5-amine | 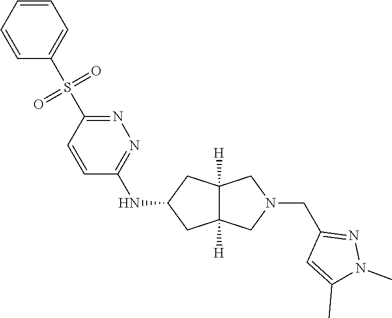 [SMILES: Cc1cc(CN2C[C@H]3C[C@H](Nc4ccc(S(=O)(=O)c5ccccc5)nn4)C[C@H]3C2)nn1C] | 453 |
| 25 | (3aR,5s,6aS)-N-(6-(phenylsulfonyl)pyridazin- 3-yl)-2-(pyridin-2- ylmethyl)octahydrocyclopenta[c]pyrrol-5- amine | 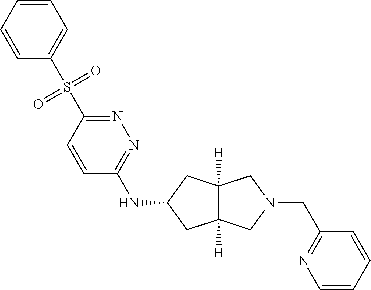 [SMILES: O=S(=O)(c1ccccc1)c1ccc(N[C@H]2C[C@@H]3CN(Cc4ccccn4)C[C@@H]3C2)nn1] | 436 |
| 26 | (3aR,5s,6aS)-2-((2,2- difluorobenzo[d][1,3]dioxol-5-yl)methyl)-N- (6-(phenylsulfonyl)pyridazin-3- yl)octahydrocyclopenta[c]pyrrol-5-amine | 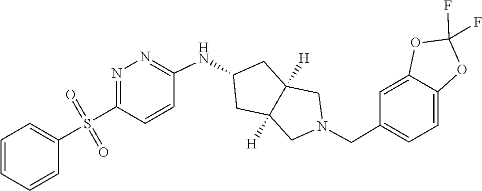 [SMILES: O=S(=O)(c1ccccc1)c1ccc(N[C@H]2C[C@@H]3CN(Cc4ccc5c(c4)OC(F)(F)O5)C[C@@H]3C2)nn1] | 515 |
| 27 | (3aR,5s,6aS)-2-(benzo[d][1,3]dioxol-5- ylmethyl)-N-(6-(phenylsulfonyl)pyridazin-3- yl)octahydrocyclopenta[c]pyrrol-5-amine | 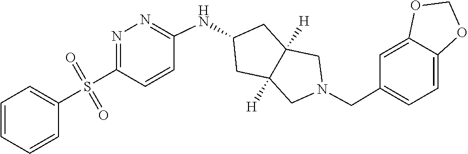 [SMILES: O=S(=O)(c1ccccc1)c1ccc(N[C@H]2C[C@@H]3CN(Cc4ccc5c(c4)OCO5)C[C@@H]3C2)nn1] | 479 |
| 28 | (3aR,5s,6aS)-N-(6-(phenylsulfinyl)pyridazin- 3-yl)-2-((tetrahydro-2H-pyran-4- yl)methyl)octahydrocyclopenta[c]pyrrol-5- amine | 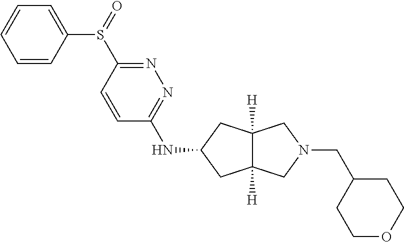 [SMILES: O=S(c1ccccc1)c1ccc(N[C@H]2C[C@@H]3CN(CC4CCOCC4)C[C@@H]3C2)nn1] | 427 |
| 29 | (3aR,5s,6aS)-2-(2,2-dimethyltetrahydro-2H- pyran-4-yl)-N-(6-(phenylsulfinyl)pyridazin-3- yl)octahydrocyclopenta[c]pyrrol-5-amine | 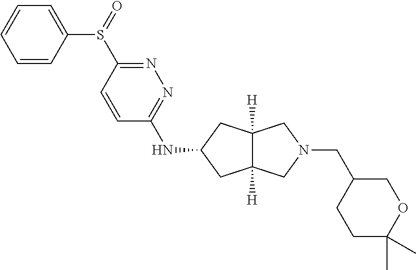 [SMILES: CC1(C)CCC(CN2C[C@H]3C[C@H](Nc4ccc(S(=O)c5ccccc5)nn4)C[C@H]3C2)CO1] | 441 |
| 30 | (3aR,5s,6aS)-2-(benzo[d][1,3]dioxol-5- ylmethyl)-N-(6-(phenylsulfinyl)pyridazin-3- yl)octahydrocyclopenta[c]pyrrol-5-amine | 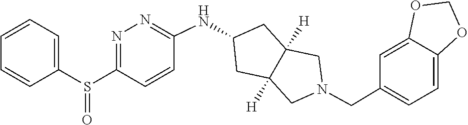 [SMILES: O=S(c1ccccc1)c1ccc(N[C@H]2C[C@@H]3CN(Cc4ccc5c(c4)OCO5)C[C@@H]3C2)nn1] | 463 |
| 31 | (3aR,5s,6aS)-2-(4-fluoro-3-methylbenzyl)-N- (6-(phenylsulfonyl)pyridazin-3- yl)octahydrocyclopenta[c]pyrrol-5-amine | 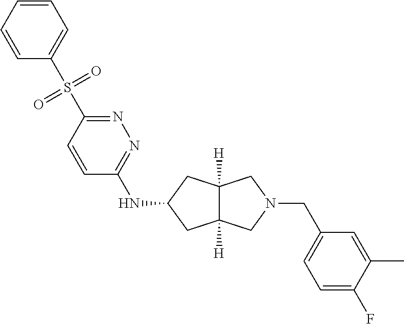 [SMILES: Cc1cc(CN2C[C@H]3C[C@H](Nc4ccc(S(=O)(=O)c5ccccc5)nn4)C[C@H]3C2)ccc1F] | 467 |
| 32 | (3aR,5s,6aS)-2-(4-fluoro-3-methylbenzyl)-N- (6-(phenylsulfinyl)pyridazin-3- yl)octahydrocyclopenta[c]pyrrol-5-amine | 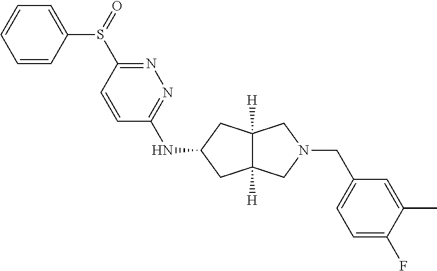 [SMILES: Cc1cc(CN2C[C@H]3C[C@H](Nc4ccc(S(=O)c5ccccc5)nn4)C[C@H]3C2)ccc1F] | 451 |
| 33 | (3aR,5s,6aS)-N-(6- (cyclohexylsulfonyl)pyridazin-3-yl)-2- (pyridin-2- ylmethyl)octahydrocyclopenta[c]pyrrol-5- amine | 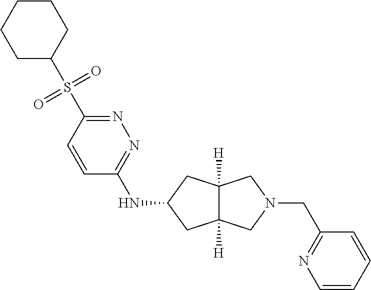 [SMILES: O=S(=O)(c1ccc(N[C@H]2C[C@@H]3CN(Cc4ccccn4)C[C@@H]3C2)nn1)C1CCCCC1] | 442 |
| 34 | (3aR,5s,6aS)-N-(6- (cyclohexylsulfonyl)pyridazin-3-yl)-2- ((tetrahydro-2H-pyran-4- yl)methyl)octahydrocyclopenta[c]pyrrol-5- amine | 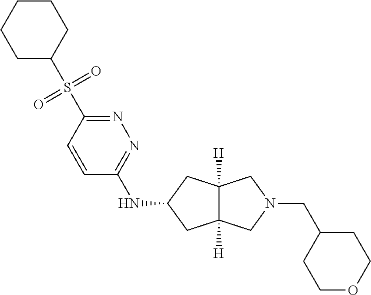 [SMILES: O=S(=O)(c1ccc(N[C@H]2C[C@@H]3CN(CC4CCOCC4)C[C@@H]3C2)nn1)C1CCCCC1] | 449 |
| 35 | (3aR,5s,6aS)-N-(6- (cyclohexylsulfonyl)pyridazin-3-yl)-2-((1,5- dimethyl-1H-pyrazol-3- yl)methyl)octahydrocyclopenta[c]pyrrol-5- amine | 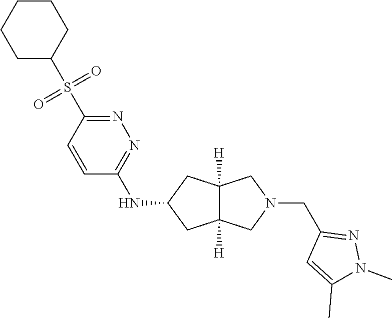 [SMILES: Cc1cc(CN2C[C@H]3C[C@H](Nc4ccc(S(=O)(=O)C5CCCCC5)nn4)C[C@H]3C2)nn1C] | 459 |
| 36 | (3aR,5s,6aS)-N-(6- (cyclohexylsulfonyl)pyridazin-3-yl)-2-(2,2- dimethyltetrahydro-2H-pyran-4- yl)octahydrocyclopenta[c]pyrrol-5-amine | 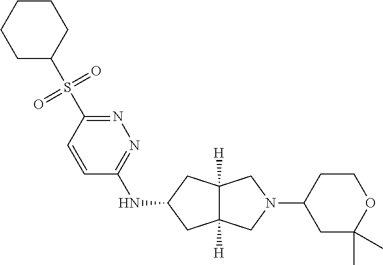 [SMILES: CC1(C)CC(N2C[C@H]3C[C@H](Nc4ccc(S(=O)(=O)C5CCCCC5)nn4)C[C@H]3C2)CCO1] | 463 |
| 37 | (3aR,5s,6aS)-2-(benzo[d][1,3]dioxol-5- ylmethyl)-N-(6- (cyclohexylsulfonyl)pyridazin-3- yl)octahydrocyclopenta[c]pyrrol-5-amine | 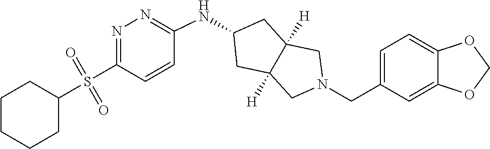 [SMILES: O=S(=O)(c1ccc(N[C@H]2C[C@@H]3CN(Cc4ccc5c(c4)OCO5)C[C@@H]3C2)nn1)C1CCCCC1] | 485 |
| 38 | (3aR,5s,6aS)-N-(6- (cyclohexylsulfonyl)pyridazin-3-yl)-2-(4- fluoro-3- methylbenzyl)octahydrocyclopenta[c]pyrrol- 5-amine | 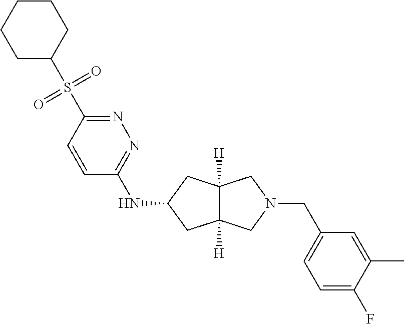 [SMILES: Cc1cc(CN2C[C@H]3C[C@H](Nc4ccc(S(=O)(=O)C5CCCCC5)nn4)C[C@H]3C2)ccc1F] | 473 |
| 39 | N⁵-cyclopropyl-6-(phenylsulfonyl)-N³- ((3aR,5s,6aS)-2-((tetrahydro-2H-pyran-4- yl)methyl)octahydrocyclopenta[c]pyrrol-5- yl)pyridazine-3,5-diamine | 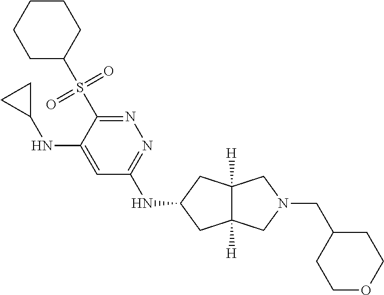 [SMILES: O=S(=O)(c1nnc(N[C@H]2C[C@@H]3CN(CC4CCOCC4)C[C@@H]3C2)cc1NC1CC1)C1CCCCC1] | 498 |
| 40 | (3aR,5s,6aS)-2-((3-methylpyridin-2- yl)methyl)-N-(6-(phenylsulfonyl)pyridazin-3- yl)octahydrocyclopenta[c]pyrrol-5-amine | 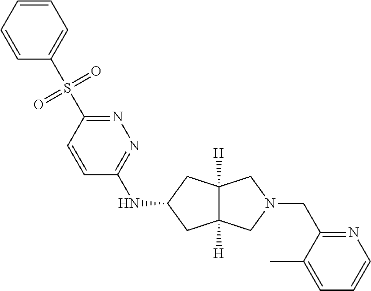 [SMILES: Cc1cccnc1CN1C[C@H]2C[C@H](Nc3ccc(S(=O)(=O)c4ccccc4)nn3)C[C@H]2C1] | 450 |
| 41 | (3aR,5s,6aS)-N-(6-(phenylsulfonyl)pyridazin- 3-yl)-2-((3-(trifluoromethyl)pyridin-2- yl)methyl)octahydrocyclopenta[c]pyrrol-5- amine | 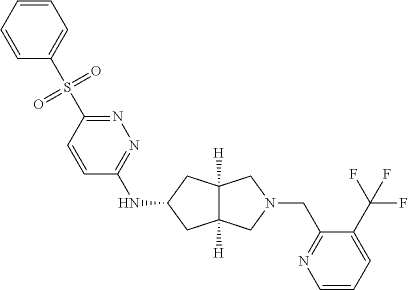 [SMILES: O=S(=O)(c1ccccc1)c1ccc(N[C@H]2C[C@@H]3CN(Cc4ncccc4C(F)(F)F)C[C@@H]3C2)nn1] | 504 |
| 42 | (3aR,5s,6aS)-2-((6- methylbenzo[d][1,3]dioxol-5-yl)methyl)-N- (6-(phenylsulfonyl)pyridazin-3- yl)octahydrocyclopenta[c]pyrrol-5-amine | 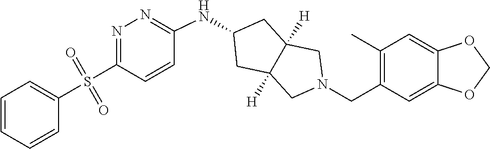 [SMILES: Cc1cc2c(cc1CN1C[C@H]3C[C@H](Nc4ccc(S(=O)(=O)c5ccccc5)nn4)C[C@H]3C1)OCO2] | 493 |
| 43 | (3aR,5s,6aS)-N-(6- (cyclohexylsulfonyl)pyridazin-3-yl)-2-((3- methylpyridin-2- yl)methyl)octahydrocyclopenta[c]pyrrol-5- amine | 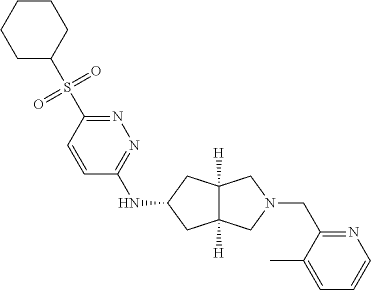 [SMILES: Cc1cccnc1CN1C[C@H]2C[C@H](Nc3ccc(S(=O)(=O)C4CCCCC4)nn3)C[C@H]2C1] | 456 |
| 44 | (3aR,5s,6aS)-N-(6- (cyclohexylsulfonyl)pyridazin-3-yl)-2-((6- methylbenzo[d][1,3]dioxol-5- yl)methyl)octahydrocyclopenta[c]pyrrol-5- amine | 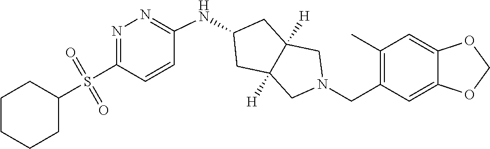 [SMILES: Cc1cc2c(cc1CN1C[C@H]3C[C@H](Nc4ccc(S(=O)(=O)C5CCCCC5)nn4)C[C@H]3C1)OCO2] | 499 |
| 45 | (3aR,5s,6aS)-2-((5-bromo-3-methylpyridin-2- yl)methyl)-N-(6- (cyclohexylsulfonyl)pyridazin-3- yl)octahydrocyclopenta[c]pyrrol-5-amine | 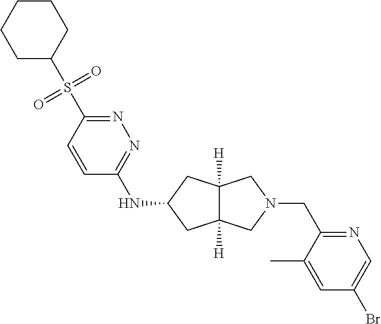 [SMILES: Cc1cc(Br)cnc1CN1C[C@H]2C[C@H](Nc3ccc(S(=O)(=O)C4CCCCC4)nn3)C[C@H]2C1] | 534 |
| 46 | 4-(((3aR,5s,6aS)-5-((6- (cyclohexylsulfonyl)pyridazin-3- yl)amino)hexahydrocyclopenta[c]pyrrol- 2(1H)-yl)methyl)tetrahydro-2H-pyran-4-ol | 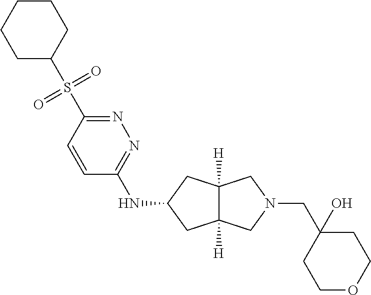 [SMILES: O=S(=O)(c1ccc(N[C@H]2C[C@@H]3CN(CC4(O)CCOCC4)C[C@@H]3C2)nn1)C1CCCCC1] | 465 |
| 47 | (3aR,5s,6aS)-2-((1H-indol-5-yl)methyl)-N-(6- (cyclohexylsulfonyl)pyridazin-3- yl)octahydrocyclopenta[c]pyrrol-5-amine | 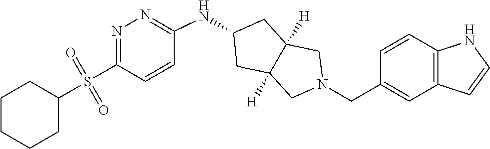 [SMILES: O=S(=O)(c1ccc(N[C@H]2C[C@@H]3CN(Cc4ccc5[nH]ccc5c4)C[C@@H]3C2)nn1)C1CCCCC1] | 480 |
| 48 | (3aR,5s,6aS)-2-((1H-indol-6-yl)methyl)-N-(6- (cyclohexylsulfonyl)pyridazin-3- yl)octahydrocyclopenta[c]pyrrol-5-amine | 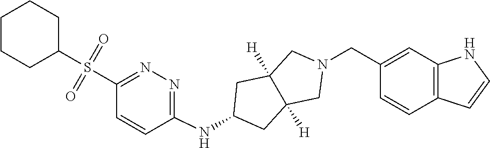 [SMILES: O=S(=O)(c1ccc(N[C@H]2C[C@@H]3CN(Cc4ccc5cc[nH]c5c4)C[C@@H]3C2)nn1)C1CCCCC1] | 480 |
| 49 | (3aR,5s,6aS)-N-(6- (cyclohexylsulfonyl)pyridazin-3-yl)-2-(3,3- dimethylbutyl)octahydrocyclopenta[c]pyrrol- 5-amine | 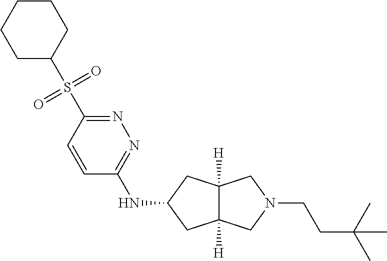 [SMILES: CC(C)(C)CCN1C[C@H]2C[C@H](Nc3ccc(S(=O)(=O)C4CCCCC4)nn3)C[C@H]2C1] | 435 |
| 50 | (3aR, 5s,6aS)-2-(3,3-dimethylbutyl)-N-(6- (phenylsulfonyl)pyridazin-3- yl)octahydrocyclopenta[c]pyrrol-5-amine | 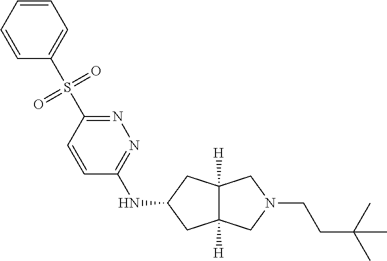 [SMILES: CC(C)(C)CCN1C[C@H]2C[C@H](Nc3ccc(S(=O)(=O)c4ccccc4)nn3)C[C@H]2C1] | 429 |
| 51 | (3aR,5s,6aS)-N-(6-chloropyridazin-3-yl)-2- (2,2-dimethyltetrahydro-2H-pyran-4- yl)octahydrocyclopenta[c]pyrrol-5-amine | 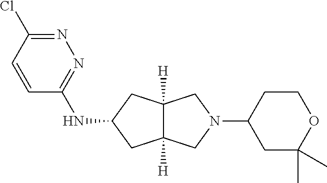 [SMILES: CC1(C)CC(N2C[C@H]3C[C@H](Nc4ccc(Cl)nn4)C[C@H]3C2)CCO1] | 351.3 |
| 52 | (3aR,5s,6aS)-2-(2,2-dimethyltetrahydro-2H- pyran-4-yl)-N-(6-(2-fluorophenyl)pyridazin- 3-yl)octahydrocyclopenta[c]pyrrol-5-amine | 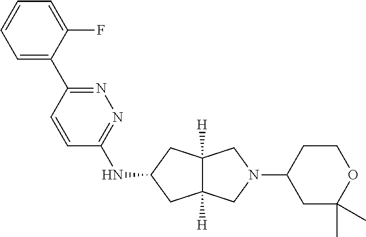 [SMILES: CC1(C)CC(N2C[C@H]3C[C@H](Nc4ccc(-c5ccccc5F)nn4)C[C@H]3C2)CCO1] | 411.5 |
| 53 | (3aR,5s,6aS)-2-(2,2-dimethyltetrahydro-2H- pyran-4-yl)-N-(6-(3-fluorophenyl)pyridazin- 3-yl)octahydrocyclopenta[c]pyrrol-5-amine | 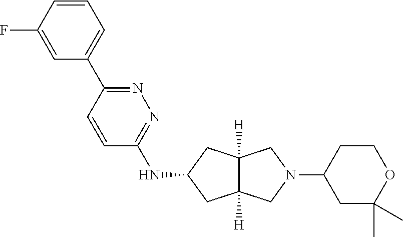 [SMILES: CC1(C)CC(N2C[C@H]3C[C@H](Nc4ccc(-c5cccc(F)c5)nn4)C[C@H]3C2)CCO1] | 411.4 |
| 54 | (3aR,5s,6aS)-2-(2,2-dimethyltetrahydro-2H- pyran-4-yl)-N-(6-(4-fluorophenyl)pyridazin- 3-yl)octahydrocyclopenta[c]pyrrol-5-amine | 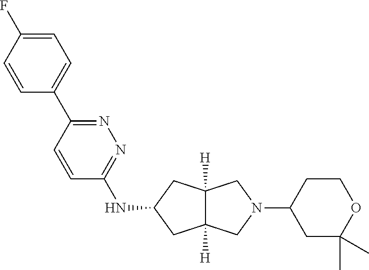 [SMILES: CC1(C)CC(N2C[C@H]3C[C@H](Nc4ccc(-c5ccc(F)cc5)nn4)C[C@H]3C2)CCO1] | 411.5 |
| 55 | (3aR,5s,6aS)-N-(6-(2,5- difluorophenyl)pyridazin-3-yl)-2-(2,2- dimethyltetrahydro-2H-pyran-4- yl)octahydrocyclopenta[c]pyrrol-5-amine | 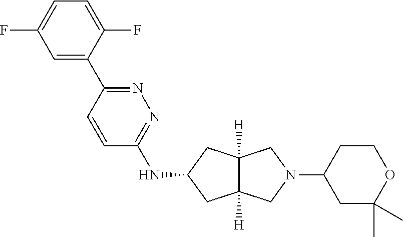 [SMILES: CC1(C)CC(N2C[C@H]3C[C@H](Nc4ccc(-c5cc(F)ccc5F)nn4)C[C@H]3C2)CCO1] | 429.4 |
| 56 | (3aR,5s,6aS)-2-(2,2-dimethyltetrahydro-2H- pyran-4-yl)-N-(6-(pyridin-3-yl)pyridazin-3- yl)octahydrocyclopenta[c]pyrrol-5-amine | 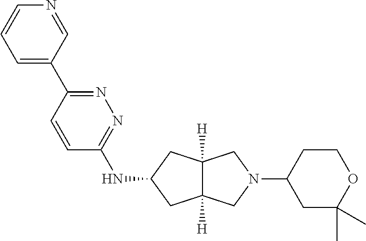 [SMILES: CC1(C)CC(N2C[C@H]3C[C@H](Nc4ccc(-c5cccnc5)nn4)C[C@H]3C2)CCO1] | 394.4 |
| 57 | (3aR,5s,6aS)-N-(6-(1,4-dimethyl-1H-pyrazol- 5-yl)pyridazin-3-yl)-2-(2,2- dimethyltetrahydro-2H-pyran-4- yl)octahydrocyclopenta[c]pyrrol-5-amine | 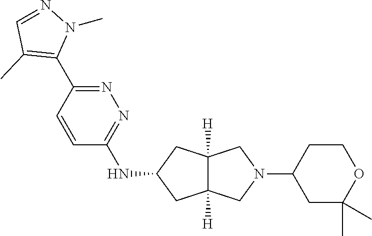 | 411.5 |
| 58 | (3aR,5s,6aS)-N-(6-(4,4-difluoropiperidin-1- yl)pyridazin-3-yl)-2-(2,2-dimethyltetrahydro- 2H-pyran-4-yl)octahydrocyclopenta[c]pyrrol- 5-amine | 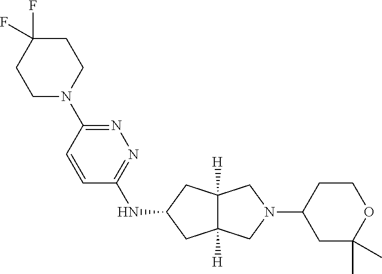 [SMILES: CC1(C)CC(N2C[C@H]3C[C@H](Nc4ccc(N5CCC(F)(F)CC5)nn4)C[C@H]3C2)CCO1] | 436.5 |
| 59 | N-(5-(6-(((3aR,5s,6aS)-2-(2,2- dimethyltetrahydro-2H-pyran-4- yl)octahydrocyclopenta[c]pyrrol-5- yl)amino)pyridazin-3-yl)pyridin-2- yl)acetamide | 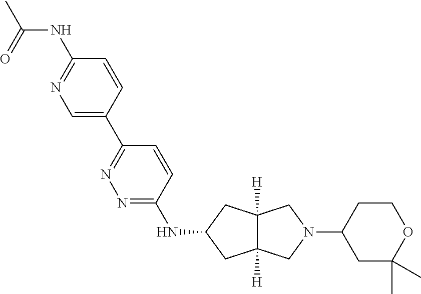 [SMILES: CC(=O)Nc1ccc(-c2ccc(N[C@H]3C[C@@H]4CN(C5CCOC(C)(C)C5)C[C@@H]4C3)nn2)cn1] | 451.3 |
| 60 | (3aR,5s,6aS)-2-(2,2-dimethyltetrahydro-2H- pyran-4-yl)-N-(6-morpholinopyridazin-3- yl)octahydrocyclopenta[c]pyrrol-5-amine | 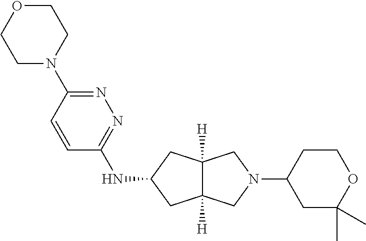 [SMILES: CC1(C)CC(N2C[C@H]3C[C@H](Nc4ccc(N5CCOCC5)nn4)C[C@H]3C2)CCO1] | 402.2 |
| 61 | N-((3aR,5s,6aS)-2-(2,2-dimethyltetrahydro- 2H-pyran-4-yl)octahydrocyclopenta[c]pyrrol- 5-yl)-[1,2,4]triazolo[4,3-b]pyridazin-6-amine | 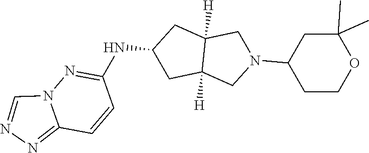 [SMILES: CC1(C)CC(N2C[C@H]3C[C@H](Nc4ccc5nncn5n4)C[C@H]3C2)CCO1] | 357.2 |
| 62 | (3aR,5s,6aS)-N-(6-(2-methyl-2H-indazol-5- yl)pyridazin-3-yl)-2-(tetrahydro-2H-pyran-4- yl)octahydrocyclopenta[c]pyrrol-5-amine | 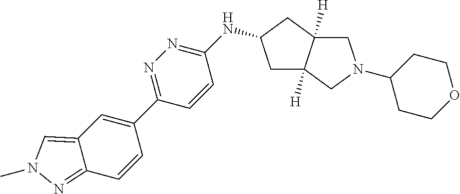 [SMILES: Cn1cc2cc(-c3ccc(N[C@H]4C[C@@H]5CN(C6CCOCC6)C[C@@H]5C4)nn3)ccc2n1] | 419 |
| 63 | (3aR,5s,6aS)-2-(2,2-dimethyltetrahydro-2H- pyran-4-yl)-N-(6-(2-methyl-2H-indazol-5- yl)pyridazin-3- yl)octahydrocyclopenta[c]pyrrol-5-amine | 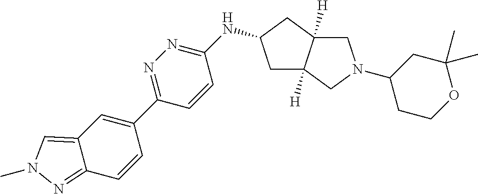 | 447 |
| 64 | (3aR,5s,6aS)-N-(6-(2-chloro-5- fluorophenyl)pyridazin-3-yl)-2-(1- (tetrahydro-2H-pyran-4- yl)cyclopropyl)octahydrocyclopenta[c]pyrrol- 5-amine | 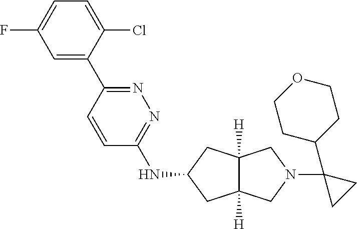 [SMILES: Fc1ccc(Cl)c(-c2ccc(N[C@H]3C[C@@H]4CN(C5(C6CCOCC6)CC5)C[C@@H]4C3)nn2)c1] | 457.4 |
| 65 | (3aR,5s,6aS)-2-(2,2-dimethyltetrahydro-2H- pyran-4-yl)-N-(6-(phenylsulfinyl)pyridazin-3- yl)octahydrocyclopenta[c]pyrrol-5-amine | 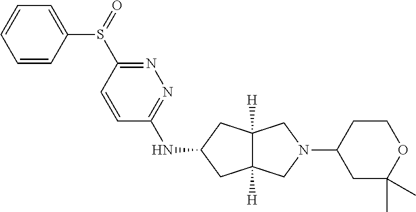 [SMILES: CC1(C)CC(N2C[C@H]3C[C@H](Nc4ccc(S(=O)c5ccccc5)nn4)C[C@H]3C2)CCO1] | 441.3 |
| 66 | (3aR,5s,6aS)-N-(6- (cyclohexylsulfonyl)pyridazin-3-yl)-2-(2,2- dimethyltetrahydro-2H-pyran-4- yl)octahydrocyclopenta[c]pyrrol-5-amine | 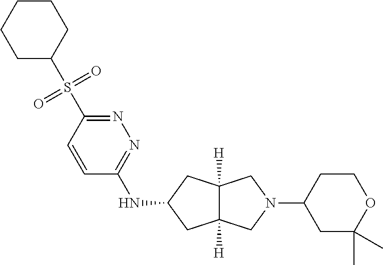 [SMILES: CC1(C)CC(N2C[C@H]3C[C@H](Nc4ccc(S(=O)(=O)C5CCCCC5)nn4)C[C@H]3C2)CCO1] | 463.4 |
| 67 | (3aR,5s,6aS)-N-(6-(2-chloro-5- fluorophenyl)pyridazin-3-yl)-2-(4,4- difluorocyclohexyl)octahydrocyclopenta[c] pyrrol-5-amine | 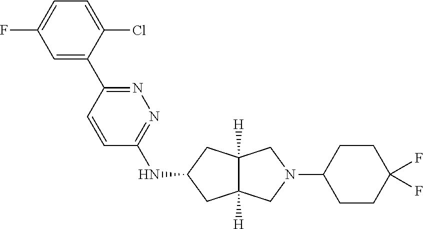 [SMILES: Fc1ccc(Cl)c(-c2ccc(N[C@H]3C[C@@H]4CN(C5CCC(F)(F)CC5)C[C@@H]4C3)nn2)c1] | 451 |
| 68 | (3aR,5s,6aS)-N-(6-(2-chloro-5- fluorophenyl)pyridazin-3-yl)-2-(4,4- dimethylcyclohexyl)octahydrocyclopenta[c] pyrrol-5-amine | 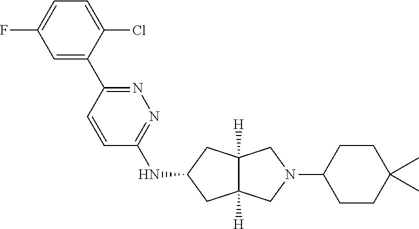 [SMILES: CC1(C)CCC(N2C[C@H]3C[C@H](Nc4ccc(-c5cc(F)ccc5Cl)nn4)C[C@H]3C2)CC1] | 443 |
| 69 | (3aR,5s,6aS)-N-(6-(2-chloro-5- fluorophenyl)pyridazin-3-yl)-2-(2- oxaspiro[3.3]heptan-6- yl)octahydrocyclopenta[c]pyrrol-5-amine | 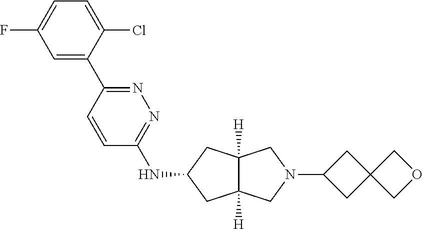 [SMILES: Fc1ccc(Cl)c(-c2ccc(N[C@H]3C[C@@H]4CN(C5CC6(COC6)C5)C[C@@H]4C3)nn2)c1] | 429.3 |
| 70 | 7-cyclopropyl-5,5-dimethyl-3-(((3aR,5s,6aS)- 2-((tetrahydro-2H-pyran-4- yl)methyl)octahydrocyclopenta[c]pyrrol-5- yl)amino)-5,7-dihydro-6H-pyrrolo [2,3- c]pyridazin-6-one | 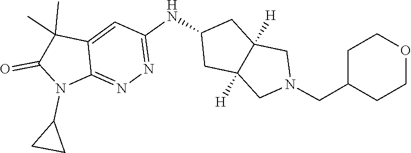 [SMILES: CC1(C)C(=O)N(C2CC2)c2nnc(N[C@H]3C[C@@H]4CN(CC5CCOCC5)C[C@@H]4C3)cc21] | 426.3 |
| 71 | 7-allyl-3-(((3aR,5s,6aS)-2-(3,3- dimethylbutyl)octahydrocyclopenta[c]pyrrol- 5-yl)amino)-5,5-dimethyl-5,7-dihydro-6H- pyrrolo[2,3-c]pyridazin-6-one | 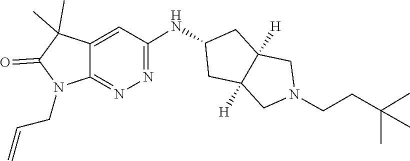 [SMILES: C=CCN1C(=O)C(C)(C)c2cc(N[C@H]3C[C@@H]4CN(CCC(C)(C)C)C[C@@H]4C3)nnc21] | 412.4 |
| 72 | 3-(((3aR,5s,6aS)-2-(3,3- dimethylbutyl)octahydrocyclopenta[c]pyrrol- 5-yl)amino)-5,5-dimethyl-7-propyl-5,7- dihydro-6H-pyrrolo[2,3-c]pyridazin-6-one | 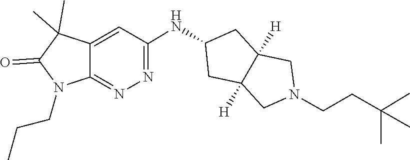 [SMILES: CCCN1C(=O)C(C)(C)c2cc(N[C@H]3C[C@@H]4CN(CCC(C)(C)C)C[C@@H]4C3)nnc21] | 414.4 |
| 73 | 1-((3aR,5s,6aS)-5-((6-(2-Chloro-5- fluorophenyl)pyridazin-3- yl)amino)hexahydrocyclopenta[c]pyrrol- 2(1H)-yl)-2-methylpropan-2-ol | 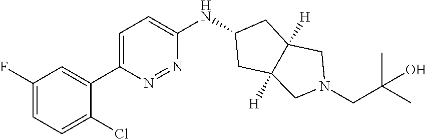 [SMILES: CC(C)(O)CN1C[C@H]2C[C@H](Nc3ccc(-c4cc(F)ccc4Cl)nn3)C[C@H]2C1] | 405.4 |
| 74 | (3aR,5s,6aS)-N-(6-(2-chloro-5- fluorophenyl)pyridazin-3-yl)-2- cyclopentyloctahydrocyclopenta[c]pyrrol-5- amine | 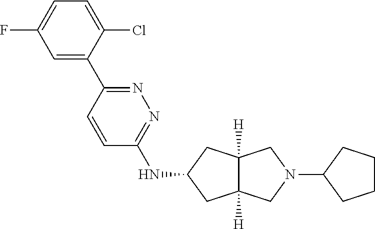 [SMILES: Fc1ccc(Cl)c(-c2ccc(N[C@H]3C[C@@H]4CN(C5CCCC5)C[C@@H]4C3)nn2)c1] | 401.5 |
| 75 | (3aR,5s,6aS)-N-(6-(2-chloro-5- fluorophenyl)pyridazin-3-yl)-2- cycloheptyloctahydrocyclopenta[c]pyrrol-5- amine | 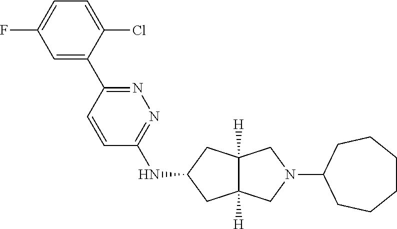 [SMILES: Fc1ccc(Cl)c(-c2ccc(N[C@H]3C[C@@H]4CN(C5CCCCCC5)C[C@@H]4C3)nn2)c1] | 429.4 |
| 76 | (3aR,5s,6aS)-N-(6-(2-chloro-5- fluorophenyl)pyridazin-3-yl)-2-(2,3-dihydro- 1H-inden-2-yl)octahydrocyclopenta[c]pyrrol- 5-amine | 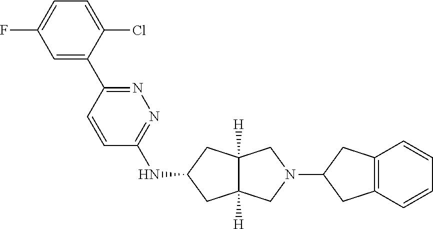 [SMILES: Fc1ccc(Cl)c(-c2ccc(N[C@H]3C[C@@H]4CN(C5Cc6ccccc6C5)C[C@@H]4C3)nn2)c1] | 449.2 |
| 77 | (3aR,5s,6aS)-N-(6-(2-chloro-5- fluorophenyl)pyridazin-3-yl)-2- (spiro[5.5]undecan-3- yl)octahydrocyclopenta[c]pyrrol-5-amine | 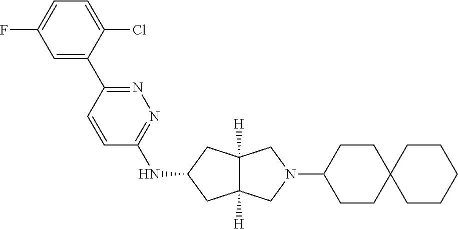 [SMILES: Fc1ccc(Cl)c(-c2ccc(N[C@H]3C[C@@H]4CN(C5CCC6(CCCCC6)CC5)C[C@@H]4C3)nn2)c1] | 483.4 |
| 78 | (3aR,5s,6aS)-N-(6-(2-chloro-5- fluorophenyl)pyridazin-3-yl)-2-(3- oxaspiro[5.5]undecan-9- yl)octahydrocyclopenta[c]pyrrol-5-amine | 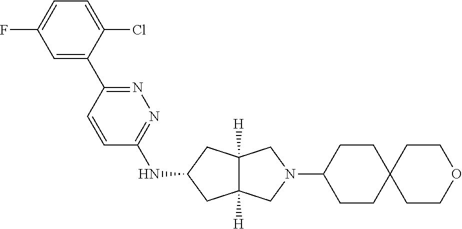 [SMILES: Fc1ccc(Cl)c(-c2ccc(N[C@H]3C[C@@H]4CN(C5CCC6(CCOCC6)CC5)C[C@@H]4C3)nn2)c1] | 485.4 |
| 79 | 4-((3aR,5s,6aS)-5-((6-(2-chloro-5- fluorophenyl)pyridazin-3- yl)amino)hexahydrocyclopenta[c]pyrrol- 2(1H)-yl)tetrahydro-2H-thiopyran 1,1-dioxide | 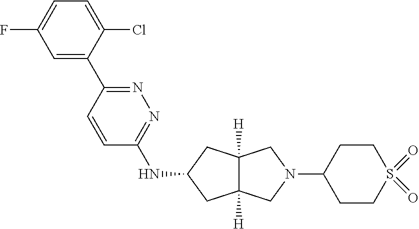 [SMILES: O=S1(=O)CCC(N2C[C@H]3C[C@H](Nc4ccc(-c5cc(F)ccc5Cl)nn4)C[C@H]3C2)CC1] | 465.3 |

### Biological Activity

A. Cell Lines Expressing Muscarinic Acetylcholine Receptors

Human or rat M₄ cDNA, along with the chimeric G protein G_{qi5}, were transfected into Chinese hamster ovary (CHO-K1) cells purchased from the American Type Culture Collection using Lipofectamine2000. M₄/G_{qi5}/CHO cells were grown in Ham's F-12 medium containing 10% heat-inactivated fetal bovine serum (FBS), 20 mM HEPES, 500 μg/mL G418 sulfate, and 200 μg/mL Hygromycin B.

B. Cell-Based Functional Assay of Muscarinic Acetylcholine Receptor Activity

For high throughput measurement of agonist-evoked increases in intracellular calcium, CHO-K1 cells stably expressing muscarinic receptors were plated in growth medium lacking G418 and hygromycin at 15,000 cells/20 μL/well in Greiner 384-well black-walled, tissue culture (TC)-treated, clear-bottom plates (VWR). Cells were incubated overnight at 37° C. and 5% CO₂. The next day, cells were washed using an ELX 405 (BioTek) with assay buffer; the final volume was then aspirated to 20 μL. Next, 20 μL of a 2.3 μM stock of Fluo-4/acetoxymethyl ester (Invitrogen, Carlsbad, Calif.), prepared as a 2.3 mM stock in DMSO and mixed in a 1:1 ratio with 10% (w/v) Pluronic F-127 and diluted in assay buffer, was added to the wells and the cell plates were incubated for 50 min at 37° C. and 5% CO₂. Dye was removed by washing with the ELX 405 and the final volume was aspirated to 20 μL. Compound master plates were formatted in a 10 point concentration-response curve (CRC) format (1:3 dilutions) in 100% DMSO with a starting concentration of 10 or 1 mM using a BRAVO liquid handler (Agilent). Test compound CRCs were then transferred to daughter plates (240 nL) using the Echo acoustic plate reformatter (Labcyte, Sunnyvale, Calif.) and then diluted into assay buffer (40 μL) to a 2× stock using a Thermo Fisher Combi (Thermo Fisher Scientific, Waltham, Mass.).

Calcium flux was measured using the Functional Drug Screening System (FDSS) 6000 or 7000 (Hamamatsu Corporation, Tokyo, Japan) as an increase in the fluorescent static ratio. Compounds were applied to cells (20 μL, 2×) using the automated system of the FDSS at 2 seconds into the protocol and the data were collected at 1 Hz. At 143 s, 10 μL of an EC20 concentration of the muscarinic receptor agonist acetylcholine was added (5×), followed by the addition of 12 μL of an EC₈₀ concentration of acetylcholine at the 268 s time point (5×). Agonist activity was analyzed as a concentration-dependent increase in calcium mobilization upon compound addition. Positive allosteric modulator activity was analyzed as a concentration-dependent increase in the EC₂₀ acetylcholine response. Antagonist activity was analyzed as a concentration-dependent decrease in the EC₈₀ acetylcholine response; for the purposes of the tables herein, an IC₅₀ (inhibitory concentration 50) was calculated as a concentration-dependent decrease of the response elicited by an EC₈₀ concentration of acetylcholine. Concentration-response curves were generated using a four-parameter logistical equation in XLFit curve fitting software (IDBS, Bridgewater, N.J.) for Excel (Microsoft, Redmond, Wash.) or Prism (GraphPad Software, Inc., San Diego, Calif.) or the Dotmatics software platform (Dotmatics, Bishop's Stortford, UK).

The above described assay was also operated in a second mode where an appropriate fixed concentration of the present compounds were added to the cells after establishment of a fluorescence baseline for about 3 seconds, and the response in cells was measured. 140 s later, a full concentration-response range consisting of increasing concentrations of agonist was added and the calcium response (maximum-local minima response) was measured. The EC₅₀ values for the agonist in the presence or absence of test compound were determined by nonlinear curve fitting. A decrease in the EC₅₀ value of the agonist with increasing concentrations of the present compounds (a leftward shift of the agonist concentration-response curve) is an indication of the degree of muscarinic positive allosteric modulation at a given concentration of the present compound. An increase in the EC₅₀ value of the agonist with increasing concentrations of the present compounds (a rightward shift of the agonist concentration response curve) is an indication of the degree of muscarinic antagonism at a given concentration of the present compound. The second mode also indicates whether the present compounds also affect the maximum response of the muscarinic receptor to agonists.

C. Activity of Compounds in a mAChR M₄ Cell-Based Assay

Compounds were synthesized as described above. Activity (IC₅₀ and Eₘᵢₙ) was determined in the mAChR M₄ cell-based functional assay as described above and the data are shown in Table 2.

TABLE 2

| | Human M₄ | | Rat M₄ | |
|---|---|---|---|---|
| Cpd. | | Eₘᵢₙ | IC₅₀ | Eₘᵢₙ |
| No. | IC₅₀ (nM) | (%)* | (nM) | (%)* |
| 1 | 42.2 | 4 | 740 | 5 |
| 2 | 64.7 | 5 | 1270 | 6 |
| 3 | 417 | 9 | | |
| 4 | 535 | 6 | | |
| 5 | 5.45 | 14 | >10,000 | 14 |
| 6 | 11.7 | 21 | 7430 | 13 |
| 7 | 572 | 4 | >10,000 | 22 |
| 8 | 355 | 4 | >10,000 | 25 |
| 9 | 713 | 3 | | |
| 10 | 543 | 4 | 4190 | 9 |
| 11 | 2060 | 4 | >10,000 | 29 |
| 12 | 2.85 | 4 | 445 | 5 |
| 13 | 80.2 | 3 | 1400 | 17 |
| 14 | 5030 | 21 | >10,000 | 51 |
| 15 | 10.2 | 7 | 884 | 5 |
| 16 | 1.90 | 4 | 97.3 | 4 |
| 17 | 10,000 | 57 | | |
| 18 | 15.0 | 4 | 513 | 4 |
| 19 | | | >10,000 | 70 |
| 20 | 18.7 | 3 | 129 | 4 |
| 21 | 36.2 | 7 | 808 | 8 |
| 22 | 4.52 | 6 | 6390 | 11 |
| 23 | 65.6 | 4 | 1520 | 11 |
| 24 | 906 | 8 | >10,000 | 35 |
| 25 | 436 | 6 | >10,000 | 26 |
| 26 | 1000 | 3 | 3000 | 14 |
| 27 | 12.8 | 2 | 791 | 4 |
| 28 | 57.2 | 5 | 113 | 8 |
| 29 | 69.1 | 5 | 102 | 10 |
| 30 | 7.8 | 4 | 196 | 4 |
| 31 | 518 | 4 | 1160 | 3 |
| 32 | 242 | 3 | 970 | 3 |
| 33 | 105 | 5 | 4460 | 14 |
| 34 | 7.75 | 4 | 119 | 5 |
| 35 | 181 | 6 | 4810 | 19 |
| 36 | 51.5 | 5 | 460 | 7 |
| 37 | 3.84 | 4 | 160 | 4 |
| 38 | 188 | 4 | 591 | 3 |
| 39 | 70.7 | 4 | 288 | 4 |
| 40 | 221 | 5 | 2890 | 15 |
| 41 | 742 | 6 | 5780 | 20 |
| 42 | 1000 | 3 | 3110 | 14 |
| 43 | 76.3 | 5 | 1260 | 11 |
| 44 | 720 | 3 | 2050 | 6 |
| 45 | 20.3 | 4 | 292 | 3 |
| 46 | 1230 | 9 | >10,000 | 27 |
| 47 | 116 | 4 | 1570 | 9 |
| 48 | 183 | 3 | 4540 | 9 |
| 49 | 5.75 | 3 | 63.8 | 4 |
| 50 | 18.0 | 3 | 130 | 4 |
| 51 | 8560 | 9 | >10,000 | 38 |
| 52 | 12.1 | 3 | 776 | 6 |
| 53 | 9.28 | 3 | 295 | 5 |
| 54 | 32.9 | 3 | 534 | 5 |
| 55 | 13.5 | 3 | 425 | 5 |
| 56 | 37.7 | 4 | 7250 | 14 |
| 57 | 28.9 | 2 | 2030 | 9 |
| 58 | 29.8 | 3 | 506 | 6 |
| 59 | 76.1 | 4 | 319 | 6 |
| 60 | 49.4 | 5 | 5170 | 14 |
| 61 | >10,000 | 52 | | |
| 62 | 1730 | 9 | 2130 | 11 |
| 63 | 9.53 | 3 | 374 | 5 |
| 64 | 161 | 4 | >10,000 | 32 |
| 65 | 69.1 | 5 | 102 | 10 |
| 66 | 51.5 | 5 | 460 | 7 |
| 67 | 2.42 | 4 | 436 | 13 |
| 68 | 2.79 | 3 | 102 | 2 |
| 69 | 11.8 | 4 | 905 | 17 |
| 70 | 12.8 | 4 | 3970 | 13 |
| 71 | 12.4 | 3 | 369 | 5 |
| 72 | 10.5 | 4 | 217 | 4 |
| 73 | 13.5 | 3 | 1360 | 8 |
| 74 | 27.3 | 2 | 933 | 4 |
| 75 | 3.2 | 2 | 41.8 | 3 |
| 76 | 189 | 3 | 350 | 4 |
| 77 | 25.7 | 3 | 387 | 7 |
| 78 | 2.3 | 3 | 102 | 3 |
| 79 | 1750 | 8 | >10,000 | 42 |

*% ACh maximum at 30 μM.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered embodiments:

E1. A compound of formula (I):

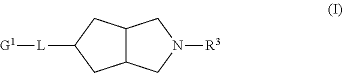

or a pharmaceutically acceptable salt thereof, wherein:
G¹ is
   a) a 5- or 6-membered monocyclic heteroaryl having 1, 2, or 3 heteroatoms independently selected from N, O, and S, the monocyclic heteroaryl being substituted with R^{1a} and 0-2 R^{1b};
   b) a phenyl substituted with R^{1a} and 0-2 R^{1b}; or
   c) an 8- to 12-membered fused bicyclic heteroaryl optionally substituted with 1-5 R²;
R^{1a} is G^{1a}, —O-G^{1a}, —SO₂-G^{1a}, —S(O)-G^{1a}, —C(O)NR^{1c}R^{1d}, or halogen;
G^{1a} is a 6- to 12-membered aryl, a 5- to 12-membered heteroaryl, a 4- to 12-membered heterocyclyl, or a C₃₋₁₂carbocyclyl, wherein G^{1a} is optionally substituted with 1-5 substituents independently selected from the group consisting of halogen, cyano, C₁₋₄alkyl, C₁₋₄haloalkyl, —OR¹⁰, —N(R¹⁰)₂, and —NR¹⁰C(O)R¹⁰;
R^{1b}, at each occurrence, is independently halogen, cyano, C₁₋₄alkyl, C₁₋₄haloalkyl, —OR¹¹, or —N(R¹¹)₂;
R¹⁰ is hydrogen, C₁₋₄alkyl, C₁₋₄haloalkyl, G^{1a}, or —C₁₋₃alkylene-G^{1a};
R^{1d} is hydrogen, C₁₋₄alkyl, C₁₋₄haloalkyl, C₃₋₄cycloalkyl, or —C₁₋₃alkylene-C₃₋₄cycloalkyl, or R^{1c} and R^{1d}, together with a nitrogen atom to which they attach form a 4- to 8-membered heterocyclic ring optionally substituted with 1-4 substituents independently selected from the group consisting of halogen and C₁₋₄alkyl;
R¹⁰ and R¹¹, at each occurrence, are independently hydrogen, C₁₋₄alkyl, C₁₋₄haloalkyl, C₃₋₄cycloalkyl, or C₁₋₃alkylene-C₃₋₄cycloalkyl, wherein alternatively two R¹⁰ and/or two R¹¹, together with a nitrogen to which the two R¹⁰ or two R¹¹ attach form a 4- to 6-membered heterocyclic ring optionally substituted with 1-4 substituents independently selected from the group consisting of halogen and C₁₋₄alkyl;
R², at each occurrence, is independently halogen, cyano, oxo, C₁₋₄alkyl, C₁₋₄haloalkyl, C₂₋₄alkenyl, C₃₋₆cycloalkyl, or C₁₋₃alkylene-C₃₋₄cycloalkyl;
L is NR, O, —NR—C(O)—; —NR—C₁₋₃alkylene-, or —O—C₁₋₃alkylene-;
R is hydrogen, C₁₋₄alkyl, C₃₋₄cycloalkyl, or —C₁₋₃alkylene-C₃₋₄cycloalkyl;
R³ is G², -L¹-G², -L²-G², -L²-L¹-G², —C₂₋₆alkylene-R^{3a}, or C₃₋₇alkyl;
L¹ is C₁₋₃alkylene;
L² is 1,1-cyclopropylene;
G² is a 6- to 12-membered aryl, a 5- to 12-membered heteroaryl, a 4- to 12-membered heterocyclyl, or a C₃₋₁₂carbocyclyl optionally fused to a phenyl, wherein G² is optionally substituted with 1-5 substituents independently selected from the group consisting of halogen, cyano, C₁₋₄alkyl, C₁₋₄haloalkyl, —OR¹³, —N(R¹³)₂, —C₁₋₃alkylene-OR¹³, and —C₁₋₃alkylene-N(R¹³)₂;
R^{3a} is —OR¹⁴ or —N(R¹⁴)₂; and
R¹³ and R¹⁴, at each occurrence, are independently hydrogen, C₁₋₄alkyl, C₁₋₄haloalkyl, C₃₋₄cycloalkyl, or C₁₋₃alkylene-C₃₋₄cycloalkyl, wherein alternatively two R¹³ or two R¹⁴ together with a nitrogen to which the two R¹³ or two R¹⁴ attach form a 4- to 6-membered heterocyclic ring optionally substituted with 1-4 substituents independently selected from the group consisting of halogen and C₁₋₄alkyl;
provided that R³ is G², -L²-G², -L²-L¹-G², or —C₂₋₆alkylene-R^{3a}, when R^{1a} is Ga, —O-G¹, or halogen.

E1.1. The compound of E1, or a pharmaceutially acceptable salt thereof, wherein G¹-L- is not

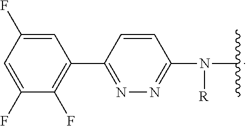

E1.2. The compound of E1 or E1.1, or a pharmaceutially acceptable salt thereof, wherein R^{1b} is —N(R¹¹)₂.

E1.3. The compound of E1 or E1.1, or a pharmaceutially acceptable salt thereof, wherein R^{1b} is CF₃,

E1.4. The compound of E1 or E1.1, or a pharmaceutially acceptable salt thereof, wherein R^{1b} is CN.

E2. The compound of any of E1-E1.4, or a pharmaceutically acceptable salt thereof, wherein G¹ is the 5- or 6-membered monocyclic heteroaryl.

E3. The compound of E2, or a pharmaceutically acceptable salt thereof, wherein the ring system of the 5- or 6-membered monocyclic heteroaryl has 2 or 1 heteroatoms independently selected from the group consisting of N and S.

E4. The compound of E3, wherein the ring system of the 5- to 6-membered monocyclic heteroaryl is a pyridazinyl or a thiazolyl.

E4.1. The compound of any of E1-E4, or a pharmaceutically acceptable salt thereof, wherein G¹ has zero R^{b} substituents.

E4.2. The compound of any of E1-E4, or a pharmaceutically acceptable salt thereof, wherein G¹ has one R^{b} substituent.

E5. The compound of E4, or a pharmaceutically acceptable salt thereof, wherein G¹ is

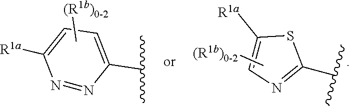

E5.1. The compound of E5, or a pharmaceutically acceptable salt thereof, wherein G¹ is

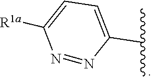

E5.2. The compound of E5, or a pharmaceutically acceptable salt thereof, wherein G¹ is

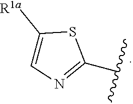

E5.3. The compound of E5, or a pharmaceutically acceptable salt thereof, wherein G¹ is

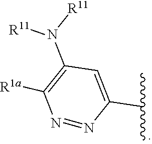

E5.4. The compound of E5.3, or a pharmaceutically acceptable salt thereof, wherein G¹ is

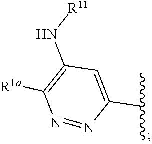

and R¹¹ is C₃₋₄cycloalkyl.

E5.5. The compound of E5.4, or a pharmaceutically acceptable salt thereof, wherein G¹ is

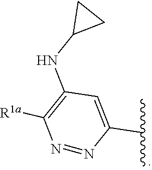

E6. The compound of any of E1-E5.5, or a pharmaceutically acceptable salt thereof, wherein R^{1a} is G^{1a}.

E6.1. The compound of any of E1-E5.5, or a pharmaceutically acceptable salt thereof, wherein R^{1a} is —O-G^{1a}.

E6.2. The compound of any of E1-E5.5, or a pharmaceutically acceptable salt thereof, wherein R^{1a} is halogen.

E7. The compound of any of E1-E5.5, or a pharmaceutically acceptable salt thereof, wherein R^{1a} is —SO₂-G^{1a}, —S(O)-G^{1a}, or —C(O)NG^{1a}R^{1d}.

E7.1. The compound of E7, or a pharmaceutically acceptable salt thereof, wherein R^{1a} is —SO₂-G^{1a}.

E7.2. The compound of E7, or a pharmaceutically acceptable salt thereof, wherein R^{1a} is —S(O)-G^{1a}.

E7.3. The compound of E7, or a pharmaceutically acceptable salt thereof, wherein R^{1a} is —C(O)NG^{1a}R^{1d}.

E7.4. The compound of E7.3, or a pharmaceutically acceptable salt thereof, wherein R^{1d} is hydrogen.

E8. The compound of any of E1-E7.4, or a pharmaceutically acceptable salt thereof, wherein G^{1a} is the 6- to 12-membered aryl.

E9. The compound of E8, or a pharmaceutically acceptable salt thereof, wherein the ring system of the 6- to 12-membered aryl is a phenyl.

E9.1. The compound of E9, or a pharmaceutically acceptable salt thereof, wherein G^{1a} is phenyl,

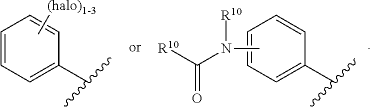

E9.2. The compound of E9 or E9.1, or a pharmaceutically acceptable salt thereof, wherein G^{1a} is phenyl

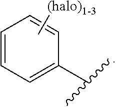

E9.3. The compound of any of E8-E9.2, or a pharmaceutically acceptable salt thereof, wherein G^{1a} is not

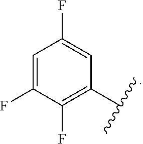 [SMILES: *c1cc(F)cc(F)c1F]

E9.4. The compound of any of E8-E9.3, or a pharmaceutically acceptable salt thereof, wherein G^{1a} is phenyl,

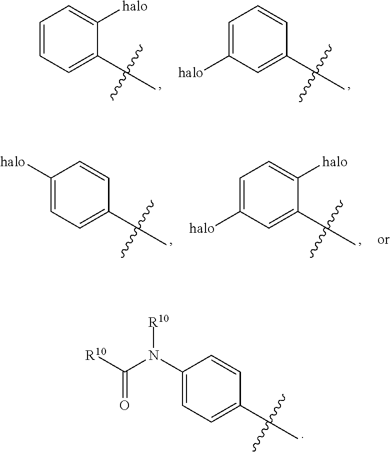

E9.5. The compound of E9.4, or a pharmaceutically acceptable salt thereof, wherein G^{1a} is phenyl,

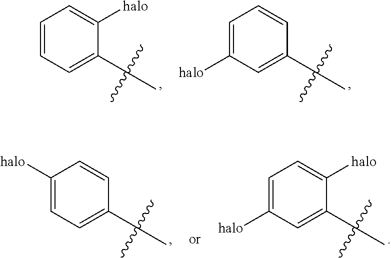

E9.6. The compound of E9.5, or a pharmaceutically acceptable salt thereof, wherein G^{1a} is phenyl,

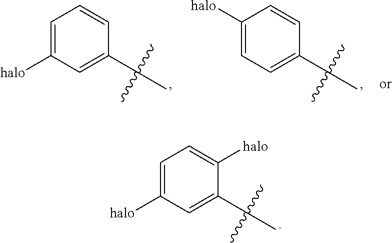

E9.7. The compound of any of E9.4-E9.6, or a pharmaceutically acceptable salt thereof, wherein the halo substituent in G^{1a} is fluoro or chloro.

E9.8. The compound of E9.4, or a pharmaceutically acceptable salt thereof, wherein G^{1a} is

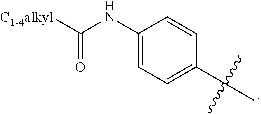

E9.9. The compound of E9.4 or a pharmaceutically acceptable salt thereof, wherein G^{1a} is phenyl,

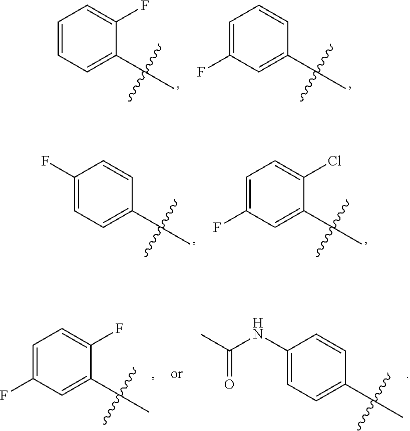 [SMILES: *c1ccccc1F ; *c1cccc(F)c1 ; *c1ccc(F)cc1 ; *c1cc(F)ccc1Cl ; *c1cc(F)ccc1F ; *c1ccc(NC(C)=O)cc1]

E9.10. The compound of E9.9, or a pharmaceutically acceptable salt thereof, wherein G^{1a} is phenyl

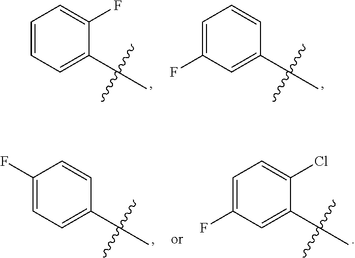 [SMILES: *c1ccccc1F ; *c1cccc(F)c1 ; *c1ccc(F)cc1 ; *c1cc(F)ccc1Cl]

E10. The compound of any of E1-E7.4, or a pharmaceutically acceptable salt thereof, wherein G^{1a} is the 5- to 12-membered heteroaryl.

E11. The compound of E10, or a pharmaceutically acceptable salt thereof, wherein the ring system of the 5- to 12-membered heteroaryl is a pyridinyl, pyrazolyl, indazolyl, or imidazopyridinyl.

E11.1. The compound of E11, or a pharmaceutically acceptable salt thereof, wherein G^{1a} is

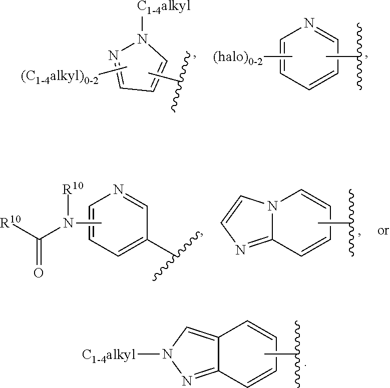

E11.2. The compound of E11.1, or a pharmaceutically acceptable salt thereof, wherein the halo substituent in G^{1a} is fluoro or chloro.

E11.3. The compound of E11.1, or a pharmaceutically acceptable salt thereof, wherein G^{1a} is

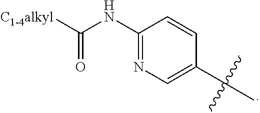

E11.4. The compound of E11.1, or a pharmaceutically acceptable salt thereof, wherein G^{1a} is

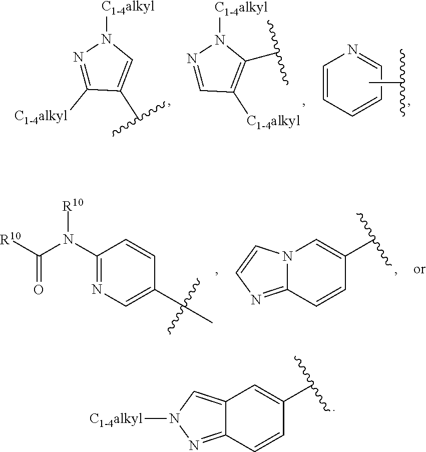

E11.5. The compound of E11.4, or a pharmaceutically acceptable salt thereof, wherein G^{1a} is

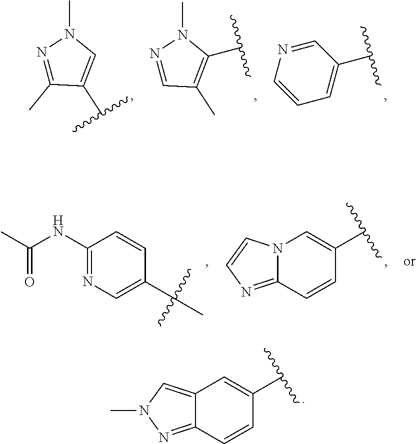

E12. The compound of any of E1-E7.4, or a pharmaceutically acceptable salt thereof, wherein G^{1a} is the 4- to 12-membered heterocyclyl.

E13. The compound of E12, or a pharmaceutically acceptable salt thereof, wherein the ring system of the 4- to 12-membered heterocyclyl is a 4- to 8-membered monocyclic heterocyclyl ring system containing 1-2 heteroatoms independently selected from the group consisting of N and O.

E13.1. The compound of E13, or a pharmaceutically acceptable salt thereof, wherein the 4- to 8-membered monocyclic heterocyclyl ring system containing 1-2 heteroatoms independently selected from the group consisting of N and O is morphline or piperidine.

E13.2. The compound of E13, or a pharmaceutically acceptable salt thereof, wherein G^{1a} is

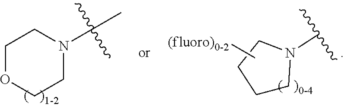

E13.3. The compound of E13.2, or a pharmaceutically acceptable salt thereof, wherein G^{1a} is or

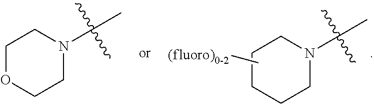

E13.4. The compound of E13.3, or a pharmaceutically acceptable salt thereof, wherein G^{1a} is

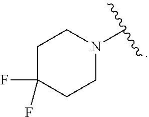 [SMILES: *N1CCC(F)(F)CC1]

E14. The compound of any of E1-E7.4, or a pharmaceutically acceptable salt thereof, wherein G^{1a} is the C₃₋₁₂carbocyclyl.

E15. The compound of E14, or a pharmaceutically acceptable salt thereof, wherein the ring system of the C₃₋₁₂carbocyclyl is a C₃₋₈cycloalkyl ring system.

E15.1. The compound of E15, or a pharmaceutically acceptable salt thereof, wherein G^{1a} is C₃₋₈cycloalkyl.

E15.2. The compound of E15.1, or a pharmaceutically acceptable salt thereof, wherein G^{1a} is cyclopropyl.

E15.3. The compound of E15.1, or a pharmaceutically acceptable salt thereof, wherein G^{1a} is cyclohexyl.

E16. The compound of any of E1-E6.1, or a pharmaceutically acceptable salt thereof, wherein G^{1a} is phenyl, C₃₋₆cycloalkyl,

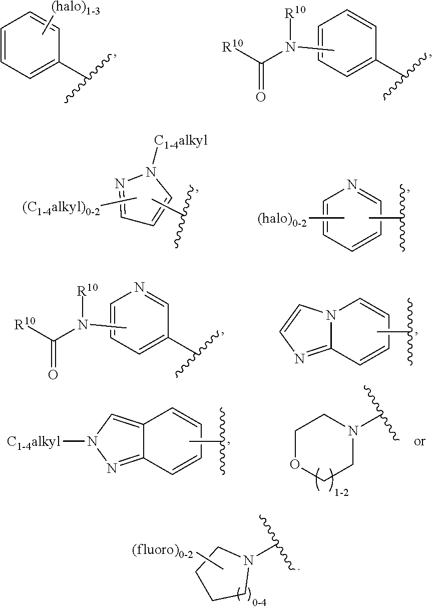

E17. The compound of E16, or a pharmaceutically acceptable salt thereof, wherein G^{1a} is phenyl,

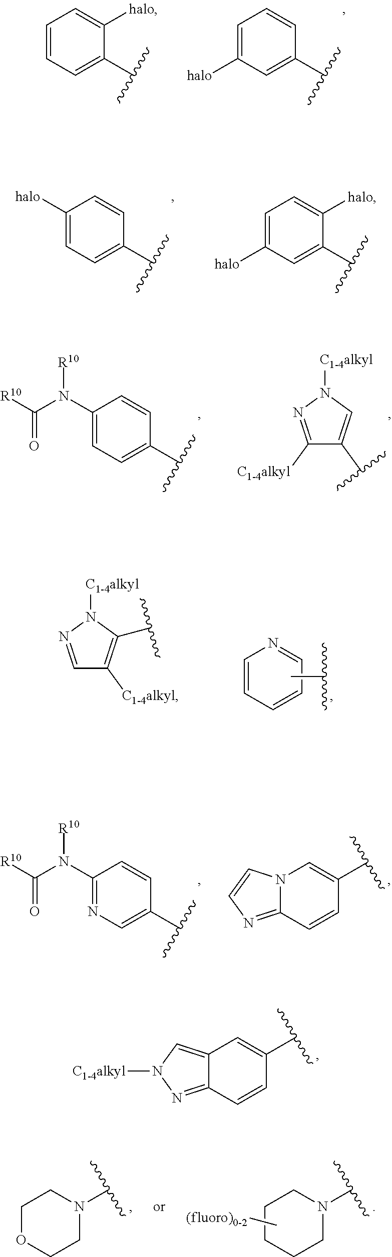

E18. The compound of any of E1-E5.5 or E7, or a pharmaceutically acceptable salt thereof, wherein G^{1a} is phenyl, C₃₋₆cycloalkyl, or

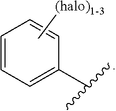

E19. The compound of E18, or a pharmaceutically acceptable salt thereof, wherein G^{1a} is phenyl, cyclopropyl, cyclohexyl,

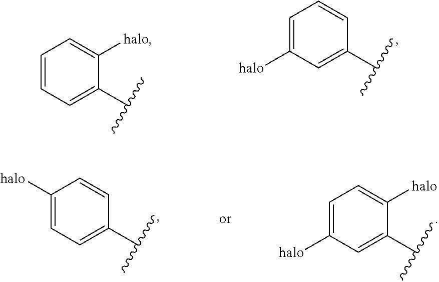

E20. The compound of any of E1-E5.5, or a pharmaceutically acceptable salt thereof, wherein R^{1a} is —C(O)NR^{1c}R^{1d}; and R^{1c} and R^{1d}, together with the nitrogen atom to which they attach form a 4- to 8-membered heterocyclic ring optionally substituted with 1-4 substituents independently selected from the group consisting of halogen and C₁₋₄alkyl.

E20.1. The compound of E20, or a pharmaceutically acceptable salt thereof, wherein the optionally substituted heterocycle formed by R^{1c} and R^{1d} is optionally substituted morpholine or a piperidine.

E20.2. The compound of E20.1, or a pharmaceutically acceptable salt thereof, wherein R^{1a} is

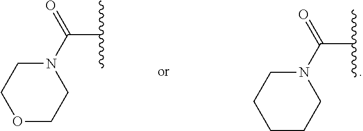 [SMILES: *C(=O)N1CCOCC1 ; *C(=O)N1CCCCC1]

E21. The compound of E1, or a pharmaceutically acceptable salt thereof, wherein G¹ is the 8- to 12-membered fused bicyclic heteroaryl.

E22. The compound of E21, or a pharmaceutically acceptable salt thereof, wherein the ring system of the 8- to 12-membered fused bicyclic heteroaryl is a 9-membered fused bicyclic aromatic ring system having four double bonds and a nitrogen atom at the ring junction.

E23. The compound of E22, or a pharmaceutically acceptable salt thereof, wherein the 9-membered fused bicyclic aromatic ring system having four double bonds and a nitrogen atom at the ring junction is a [1,2,4]triazolo[4,3-b]pyridazinyl.

E24. The compound of E21, or a pharmaceutically acceptable salt thereof, wherein the ring system of the 8- to 12-membered fused bicyclic heteroaryl is a pyridazin-3-yl fused to a pyrrolidine.

E25. The compound of E24, or a pharmaceutically acceptable salt thereof, wherein the pyridazin-3-yl fused to a pyrrolidine is a pyrrolo[2,3-c]pyridazin-3-yl.

E26. The compound of E21, or a pharmaceutically acceptable salt thereof, wherein G¹ is

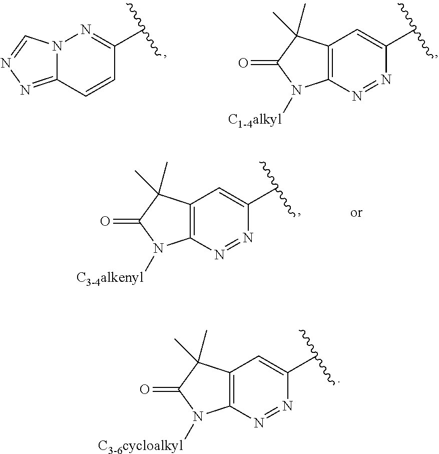

E26.1. The compound of E26, or a pharmaceutically acceptable salt thereof, wherein G¹ is

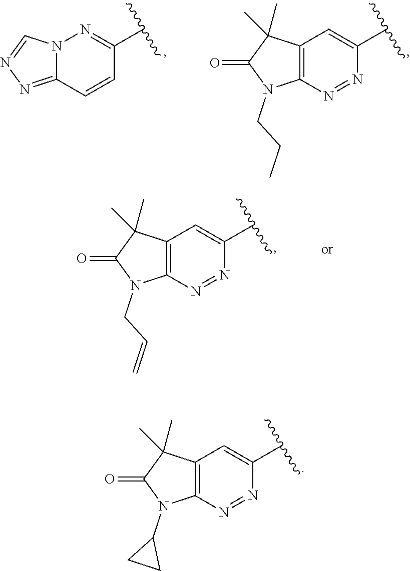 [SMILES: *c1ccc2nncn2n1 ; *c1cc2c(nn1)N(CCC)C(=O)C2(C)C ; *c1cc2c(nn1)N(CC=C)C(=O)C2(C)C ; *c1cc2c(nn1)N(C1CC1)C(=O)C2(C)C]

E27. The compound of any of E1-E26.1, or a pharmaceutically acceptable salt thereof, wherein R³ is G².

E28. The compound of any of E1-E5.5, E7-E15.3, or E18-E26.1, or a pharmaceutically acceptable salt thereof, wherein R³ is -L¹-G².

E29. The compound of any of E1-26.1, or a pharmaceutically acceptable salt thereof, wherein R³ is -L²-G².

E30. The compound of any of E1-E29, or a pharmaceutically acceptable salt thereof, wherein G² is the 4- to 12-membered heterocyclyl.

E31. The compound of E30, or a pharmaceutically acceptable salt thereof, wherein the ring system of the 4- to 12-membered heterocyclyl is a 4- to 8-membered monocyclic heterocyclyl ring system or a 7- to 12-membered spiro heterocyclyl ring system, wherein the heterocyclyls contain one heteroatom selected from O and S.

E32. The compound of E31, or a pharmaceutically acceptable salt thereof, wherein the ring system of the 4- to 12-membered heterocyclyl is an oxetanyl, a tetrahydropyranyl, a tetrahydrothiopyranyl, a 2-oxaspiro[3.3]heptanyl, or a 3-oxaspiro[5.5]undecanyl.

E32.1. The compound of E32, or a pharmaceutically acceptable salt thereof, wherein G² is

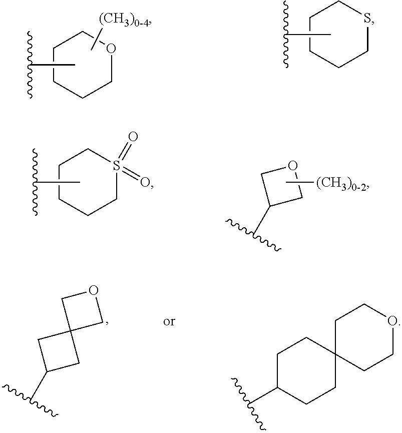

E32.2. The compound of E32.1, or a pharmaceutically acceptable salt thereof, wherein G² is

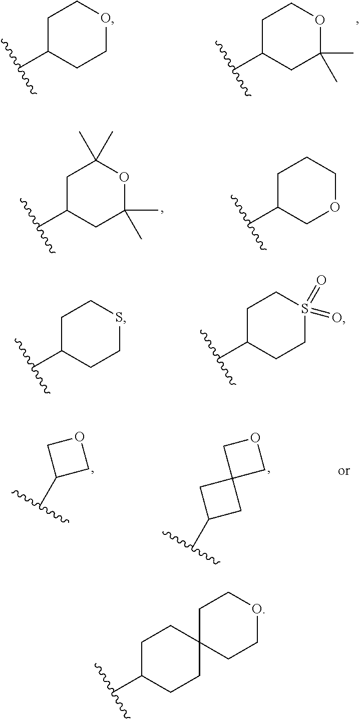

E32.3. The compound of E32, or a pharmaceutically acceptable salt thereof, wherein G² is

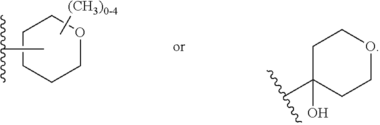

E32.4. The compound of any of E32-E32.3, or a pharmaceutically acceptable salt thereof, wherein G² is

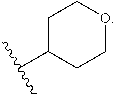 [SMILES: *C1CCOCC1]

E33. The compound of any of E1-E29, or a pharmaceutically acceptable salt thereof, wherein G² is the 6- to 12-membered aryl.

E34. The compound of E33, or a pharmaceutically acceptable salt thereof, wherein the ring system of the 6- to 12-membered aryl is a phenyl or a phenyl bonded to the parent molecule and fused to a 5- to 7-membered heterocycle containing 1-2 oxygen atoms.

E34.1. The compound of E34, or a pharmaceutically acceptable salt thereof, wherein the ring system of the 6- to 12-membered aryl is a phenyl.

E34.2. The compound of E34.1, or a pharmaceutically acceptable salt thereof, wherein G² is

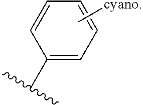

E34.3. The compound of E34.2, or a pharmaceutically acceptable salt thereof, wherein G² is

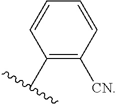 [SMILES: *c1ccccc1C#N]

E34.4. The compound of E34.1, or a pharmaceutically acceptable salt thereof, wherein G² is

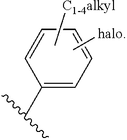

E34.5. The compound of E34.4, or a pharmaceutically acceptable salt thereof, wherein G² is

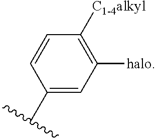

E34.6. The compound of E34.4, or a pharmaceutically acceptable salt thereof, wherein G² is

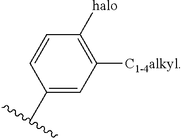

E34.7. The compound of E34.6, or a pharmaceutically acceptable salt thereof, wherein G² is

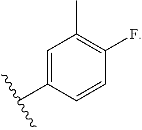 [SMILES: *c1ccc(F)c(C)c1]

E34.8. The compound of E34, or a pharmaceutically acceptable salt thereof, wherein the ring system of the 6- to 12-membered aryl is phenyl bonded to the parent molecule and fused to a 5- to 7-membered heterocycle containing 1-2 oxygen atoms.

E34.9. The compound of E34.8, or a pharmaceutically acceptable salt thereof, wherein G² is

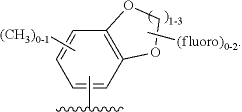

E34.10. The compound of E34.9, or a pharmaceutically acceptable salt thereof, wherein G² is

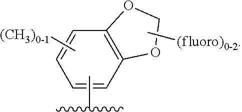

E34.11. The compound of E34.10, or a pharmaceutically acceptable salt thereof, wherein G² is

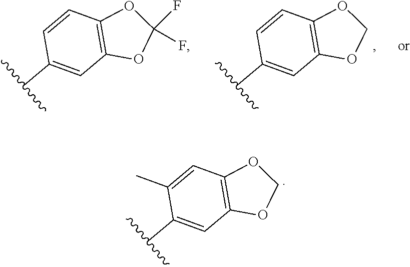 [SMILES: *c1ccc2c(c1)OC(F)(F)O2 ; *c1ccc2c(c1)OCO2 ; *c1cc2c(cc1C)OCO2]

E35. The compound of any of E1-E29, or a pharmaceutically acceptable salt thereof, wherein G² is the 5- to 12-membered heteroaryl.

E36. The compound of E35, or a pharmaceutically acceptable salt thereof, wherein the ring system of the 5- to 12-membered heteroaryl is a pyrazolyl, pyridinyl, or indolyl.

E36.1. The compound of E36, or a pharmaceutically acceptable salt thereof, wherein G² is

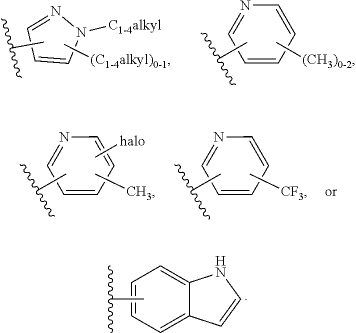

E36.2. The compound of E36 or E36.1, or a pharmaceutically acceptable salt thereof, wherein G² is

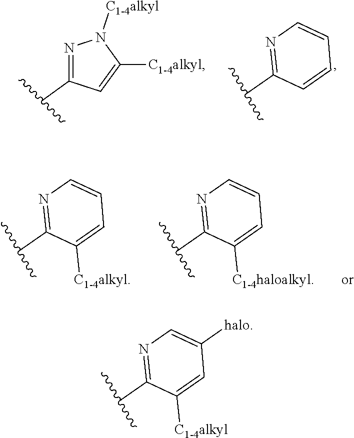

E36.3. The compound of E36 or E36.1, or a pharmaceutically acceptable salt thereof, wherein G² is

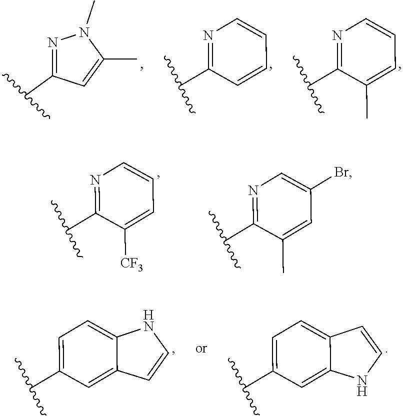

E37. The compound of any of E1-E29, or a pharmaceutically acceptable salt thereof, wherein G² is the C₃₋₁₂carbocyclyl optionally fused to a phenyl.

E38. The compound of E37, or a pharmaceutically acceptable salt thereof, wherein the ring system of the C₃₋₁₂carbocyclyl optionally fused to a phenyl is a C₃₋₈cycloalkyl optionally fused to a phenyl or the ring system of the C₃₋₁₂carbocyclyl optionally fused to a phenyl is a spiro[5.5]undecanyl.

E38.1. The compound of E38, or a pharmaceutically acceptable salt thereof, wherein G² is

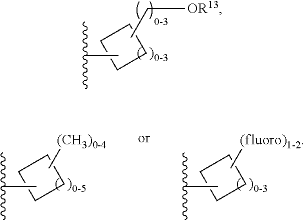

E38.2. The compound of E38, or a pharmaceutically acceptable salt thereof, wherein G² is

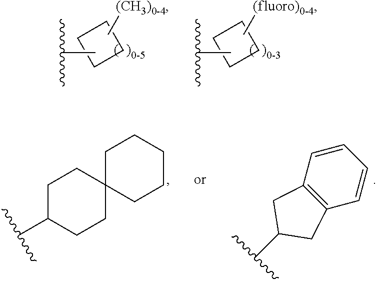

E38.3. The compound of E38 or E38.2, or a pharmaceutically acceptable salt thereof, wherein G² is

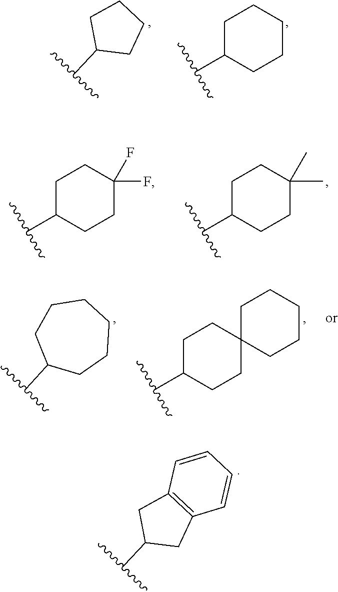

E39. The compound of any of E1-E29, or a pharmaceutically acceptable salt thereof, wherein G² is

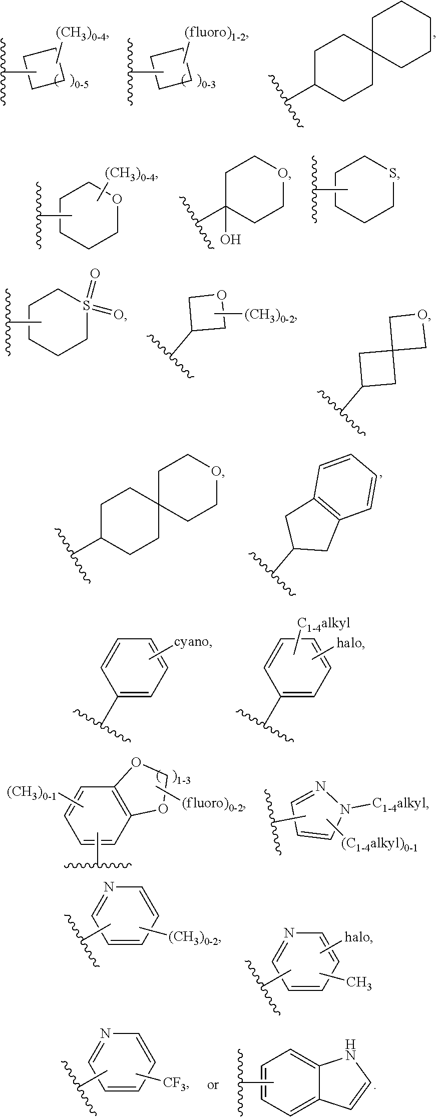

E40. The compound of E27, or a pharmaceutically acceptable salt thereof, wherein G² is

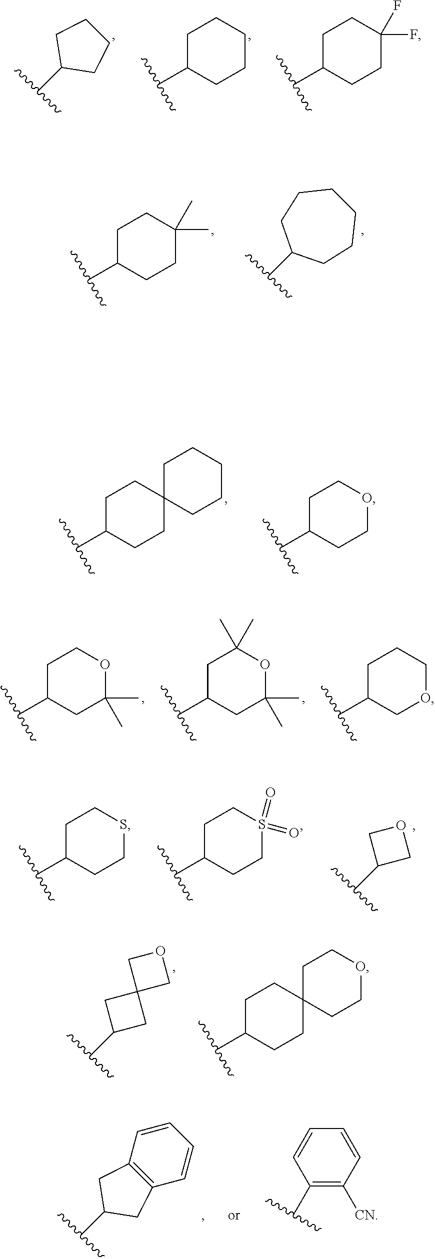

E41. The compound of E28, or a pharmaceutically acceptable salt thereof, wherein G² is

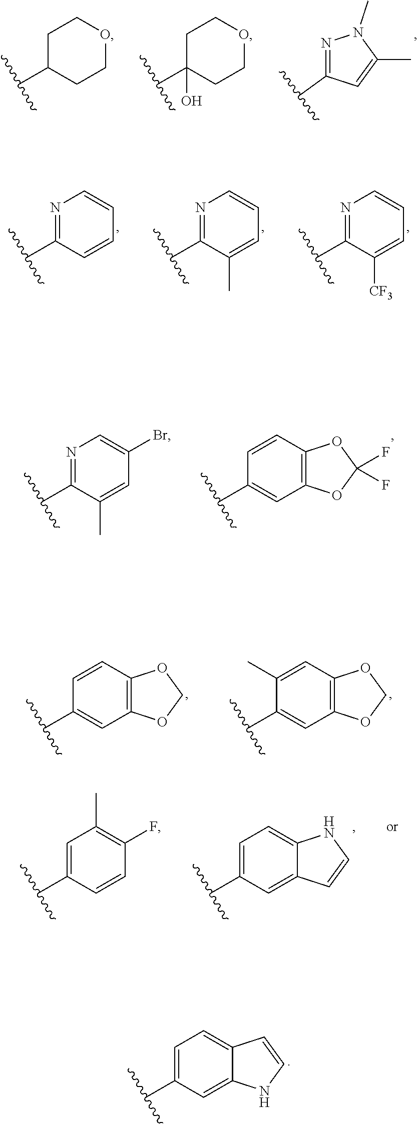

E42. The compound of E29, or a pharmaceutically acceptable salt thereof, wherein G² is

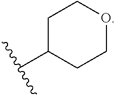 [SMILES: *C1CCOCC1]

E43. The compound of any of E1-E5.5, E7-E15.3, E18-E26.1, E28, E30-E39, or E41, or a pharmaceutically acceptable salt thereof, wherein L¹ is CH₂.

E44. The compound of any of E1-E5.5, E7-E15.3, or E18-E26.1, or a pharmaceutically acceptable salt thereof, wherein R³ is C₃₋₇alkyl.

E44.1. The compound of E44, or a pharmaceutically acceptable salt thereof, wherein R³ is 3,3-dimethylbutyl.

E45. The compound of any of E1-E26.1, or a pharmaceutically acceptable salt thereof, wherein R³ is —C₂₋₄alkylene-OR¹⁴.

E45.1. The compound of E45, or a pharmaceutically acceptable salt thereof, wherein R¹⁴ is C₁₋₄alkyl.

E45.2. The compound of E45.1, or a pharmaceutically acceptable salt thereof, wherein R¹⁴ is methyl.

E45.3. The compound of E45, or a pharmaceutically acceptable salt thereof, wherein R¹⁴ is hydrogen.

E45.4. The compound of E45, or a pharmaceutically acceptable salt thereof, wherein R³ is —(CH₂)₃—OCH₃.

E45.5. The compound of E45, or a pharmaceutically acceptable salt thereof, wherein R³ is —(CH₂)C(CH₃)₂OH.

E46. The compound of any of E1-E45.5, or a pharmaceutically acceptable salt thereof, wherein L is NR.

E47. The compound of any of E1-E45.5, or a pharmaceutically acceptable salt thereof, wherein L is —NR—C₁₋₃alkylene-.

E48. The compound of any of E1-E45.5, or a pharmaceutically acceptable salt thereof, wherein L is —NR—C(O)—.

E49. The compound of any of E1-E48, or a pharmaceutically acceptable salt thereof, wherein R is hydrogen.

E50. The compound of any of E1-E45.5, or a pharmaceutically acceptable salt thereof, wherein L is O.

E51. The compound of any of E1-E45.5, or a pharmaceutically acceptable salt thereof, wherein L is —O—C₁₋₃alkylene-.

E52. The compound of any of E1-E46 of formula (I-A)

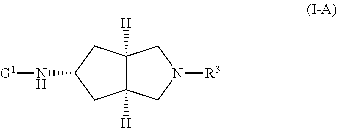

or a pharmaceutically acceptable salt thereof.

E52.1. The compound of any of E1-E46 of formula (I-B)

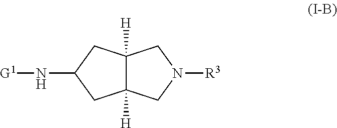

or a pharmaceutically acceptable salt thereof.

E52.2. The compound of any of E1-E46 of formula (I-C)

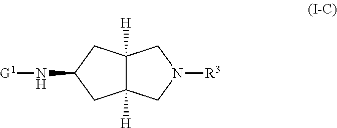

or a pharmaceutically acceptable salt thereof.

E53. The compound of E1, wherein the compound is selected from the group consisting of:
(3aR,5s,6aS)—N-(6-(2-chloro-5-fluorophenyl)pyridazin-3-yl)-2-(3-methoxypropyl)octahydrocyclopenta[c]pyrrol-5-amine;
((3aR,5s,6aS)—N-(6-(2-chloro-5-fluorophenyl)pyridazin-3-yl)-2-(tetrahydro-2H-pyran-4-yl)octahydrocyclopenta[c]pyrrol-5-amine;
N-cyclopropyl-6-(((3aR,5s,6aS)-2-((tetrahydro-2H-pyran-4-yl)methyl)octahydrocyclopenta[c]pyrrol-5-yl)amino)pyridazine-3-carboxamide;
N-cyclohexyl-6-(((3aR,5s,6aS)-2-((tetrahydro-2H-pyran-4-yl)methyl)octahydrocyclopenta[c]pyrrol-5-yl)amino)pyridazine-3-carboxamide;
piperidin-1-yl(6-(((3aR,5s,6aS)-2-((tetrahydro-2H-pyran-4-yl)methyl)octahydrocyclopenta[c]pyrrol-5-yl)amino)pyridazin-3-yl)methanone;
morpholino(6-(((3aR,5s,6aS)-2-((tetrahydro-2H-pyran-4-yl)methyl)octahydrocyclopenta[c]pyrrol-5-yl)amino)pyridazin-3-yl)methanone;
N-phenyl-6-(((3aR,5s,6aS)-2-((tetrahydro-2H-pyran-4-yl)methyl)octahydrocyclopenta[c]pyrrol-5-yl)amino)pyridazine-3-carboxamide;
N-(2-fluorophenyl)-6-(((3aR,5s,6aS)-2-((tetrahydro-2H-pyran-4-yl)methyl)octahydrocyclopenta[c]pyrrol-5-yl)amino)pyridazine-3-carboxamide;
N-(3-fluorophenyl)-6-(((3aR,5s,6aS)-2-((tetrahydro-2H-pyran-4-yl)methyl)octahydrocyclopenta[c]pyrrol-5-yl)amino)pyridazine-3-carboxamide;
N-(4-fluorophenyl)-6-(((3aR,5s,6aS)-2-((tetrahydro-2H-pyran-4-yl)methyl)octahydrocyclopenta[c]pyrrol-5-yl)amino)pyridazine-3-carboxamide;
N-(2-chloro-5-fluorophenyl)-6-(((3aR,5s,6aS)-2-((tetrahydro-2H-pyran-4-yl)methyl)octahydrocyclopenta[c]pyrrol-5-yl)amino)pyridazine-3-carboxamide;
(3aR,5s,6aS)—N-(6-(2-chloro-5-fluorophenyl)pyridazin-3-yl)-2-cyclohexyloctahydrocyclopenta[c]pyrrol-5-amine;
(3aR,5s,6aS)—N-(6-(2-chloro-5-fluorophenyl)pyridazin-3-yl)-2-(tetrahydro-2H-pyran-3-yl)octahydrocyclopenta[c]pyrrol-5-amine;
(3aR,5s,6aS)—N-(6-(2-chloro-5-fluorophenyl)pyridazin-3-yl)-2-(oxetan-3-yl)octahydrocyclopenta[c]pyrrol-5-amine;
(3aR,5s,6aS)—N-(6-(2-chloro-5-fluorophenyl)pyridazin-3-yl)-2-(tetrahydro-2H-thiopyran-4-yl)octahydrocyclopenta[c]pyrrol-5-amine;
(3aR,5s,6aS)—N-(6-(2-chloro-5-fluorophenyl)pyridazin-3-yl)-2-(2,2-dimethyltetrahydro-2H-pyran-4-yl)octahydrocyclopenta[c]pyrrol-5-amine;
2-((3aR,5s,6aS)-5-((6-(2-chloro-5-fluorophenyl)pyridazin-3-yl)amino)hexahydrocyclopenta[c]pyrrol-2(1H)-yl)benzonitrile;
(3aR,5s,6aS)—N-(6-(2-chloro-5-fluorophenyl)pyridazin-3-yl)-2-(2,2,6,6-tetramethyltetrahydro-2H-pyran-4-yl)octahydrocyclopenta[c]pyrrol-5-amine;
N-((3aR,5s,6aS)-2-(2,2-dimethyltetrahydro-2H-pyran-4-yl)octahydrocyclopenta[c]pyrrol-5-yl)-5-phenylthiazol-2-amine;
N-(4-(6-(((3aR,5s,6aS)-2-(2,2-dimethyltetrahydro-2H-pyran-4-yl)octahydrocyclopenta[c]pyrrol-5-yl)amino)pyridazin-3-yl)phenyl)acetamide;
(3aR,5s,6aS)-2-(2,2-dimethyltetrahydro-2H-pyran-4-yl)-N-(6-(imidazo[1,2-a]pyridin-6-yl)pyridazin-3-yl)octahydrocyclopenta[c]pyrrol-5-amine;
(3aR,5s,6aS)—N-(6-(1,3-dimethyl-1H-pyrazol-4-yl)pyridazin-3-yl)-2-(2,2-dimethyltetrahydro-2H-pyran-4-yl)octahydrocyclopenta[c]pyrrol-5-amine;
(3aR,5s,6aS)—N-(6-(phenylsulfonyl)pyridazin-3-yl)-2-((tetrahydro-2H-pyran-4-yl)methyl)octahydrocyclopenta[c]pyrrol-5-amine;
(3aR,5s,6aS)-2-((1,5-dimethyl-1H-pyrazol-3-yl)methyl)-N-(6-(phenylsulfonyl)pyridazin-3-yl)octahydrocyclopenta[c]pyrrol-5-amine;
(3aR,5s,6aS)—N-(6-(phenylsulfonyl)pyridazin-3-yl)-2-(pyridin-2-ylmethyl)octahydrocyclopenta[c]pyrrol-5-amine;
(3aR,5s,6aS)-2-((2,2-difluorobenzo[d][1,3]dioxol-5-yl)methyl)-N-(6-(phenylsulfonyl)pyridazin-3-yl)octahydrocyclopenta[c]pyrrol-5-amine;
(3aR,5s,6aS)-2-(benzo[d][1,3]dioxol-5-ylmethyl)-N-(6-(phenylsulfonyl)pyridazin-3-yl)octahydrocyclopenta[c]pyrrol-5-amine;
(3aR,5s,6aS)—N-(6-(phenylsulfinyl)pyridazin-3-yl)-2-((tetrahydro-2H-pyran-4-yl)methyl)octahydrocyclopenta[c]pyrrol-5-amine;
(3aR,5s,6aS)-2-(2,2-dimethyltetrahydro-2H-pyran-4-yl)-N-(6-(phenylsulfinyl)pyridazin-3-yl)octahydrocyclopenta[c]pyrrol-5-amine;
(3aR,5s,6aS)-2-(benzo[d][1,3]dioxol-5-ylmethyl)-N-(6-(phenylsulfinyl)pyridazin-3-yl)octahydrocyclopenta[c]pyrrol-5-amine;
(3aR,5s,6aS)-2-(4-fluoro-3-methylbenzyl)-N-(6-(phenylsulfonyl)pyridazin-3-yl)octahydrocyclopenta[c]pyrrol-5-amine;
(3aR,5s,6aS)-2-(4-fluoro-3-methylbenzyl)-N-(6-(phenylsulfinyl)pyridazin-3-yl)octahydrocyclopenta[c]pyrrol-5-amine;
(3aR,5s,6aS)—N-(6-(cyclohexylsulfonyl)pyridazin-3-yl)-2-(pyridin-2-ylmethyl)octahydrocyclopenta[c]pyrrol-5-amine;
(3aR,5s,6aS)—N-(6-(cyclohexylsulfonyl)pyridazin-3-yl)-2-((tetrahydro-2H-pyran-4-yl)methyl)octahydrocyclopenta[c]pyrrol-5-amine;
(3aR,5s,6aS)—N-(6-(cyclohexylsulfonyl)pyridazin-3-yl)-2-((1, 5-dimethyl-1H-pyrazol-3-yl)methyl)octahydrocyclopenta[c]pyrrol-5-amine;
(3aR,5s,6aS)—N-(6-(cyclohexylsulfonyl)pyridazin-3-yl)-2-(2,2-dimethyltetrahydro-2H-pyran-4-yl)octahydrocyclopenta[c]pyrrol-5-amine;
(3aR,5s,6aS)-2-(benzo[d][1,3]dioxol-5-ylmethyl)-N-(6-(cyclohexylsulfonyl)pyridazin-3-yl)octahydrocyclopenta[c]pyrrol-5-amine;
(3aR,5s,6aS)—N-(6-(cyclohexylsulfonyl)pyridazin-3-yl)-2-(4-fluoro-3-methylbenzyl)octahydrocyclopenta[c]pyrrol-5-amine;
N⁵-cyclopropyl-6-(phenylsulfonyl)-N³-((3aR,5s,6aS)-2-((tetrahydro-2H-pyran-4-yl)methyl)octahydrocyclopenta[c]pyrrol-5-yl)pyridazine-3,5-diamine;
(3aR,5s,6aS)-2-((3-methylpyridin-2-yl)methyl)-N-(6-(phenylsulfonyl)pyridazin-3-yl)octahydrocyclopenta[c]pyrrol-5-amine;
(3aR,5s,6aS)—N-(6-(phenylsulfonyl)pyridazin-3-yl)-2-((3-(trifluoromethyl)pyridin-2-yl)methyl)octahydrocyclopenta[c]pyrrol-5-amine;
(3aR,5s,6aS)-2-((6-methylbenzo[d][1,3]dioxol-5-yl)methyl)-N-(6-(phenylsulfonyl)pyridazin-3-yl)octahydrocyclopenta[c]pyrrol-5-amine;
(3aR,5s,6aS)—N-(6-(cyclohexylsulfonyl)pyridazin-3-yl)-2-((3-methylpyridin-2-yl)methyl)octahydrocyclopenta[c]pyrrol-5-amine;
(3aR,5s,6aS)—N-(6-(cyclohexylsulfonyl)pyridazin-3-yl)-2-((6-methylbenzo[d][1,3]dioxol-5-yl)methyl)octahydrocyclopenta[c]pyrrol-5-amine;
(3aR,5s,6aS)-2-((5-bromo-3-methylpyridin-2-yl)methyl)-N-(6-(cyclohexylsulfonyl)pyridazin-3-yl)octahydrocyclopenta[c]pyrrol-5-amine;
4-(((3aR,5s,6aS)-5-((6-(cyclohexylsulfonyl)pyridazin-3-yl)amino)hexahydrocyclopenta[c]pyrrol-2(1H)-yl)methyl)tetrahydro-2H-pyran-4-ol;
(3aR,5s,6aS)-2-((1H-indol-5-yl)methyl)-N-(6-(cyclohexylsulfonyl)pyridazin-3-yl)octahydrocyclopenta[c]pyrrol-5-amine;
(3aR,5s,6aS)-2-((1H-indol-6-yl)methyl)-N-(6-(cyclohexylsulfonyl)pyridazin-3-yl)octahydrocyclopenta[c]pyrrol-5-amine;
(3aR,5s,6aS)—N-(6-(cyclohexylsulfonyl)pyridazin-3-yl)-2-(3, 3-dimethylbutyl)octahydrocyclopenta[c]pyrrol-5-amine;
(3aR,5s,6aS)-2-(3,3-dimethylbutyl)-N-(6-(phenylsulfonyl)pyridazin-3-yl)octahydrocyclopenta[c]pyrrol-5-amine;
(3aR,5s,6aS)—N-(6-chloropyridazin-3-yl)-2-(2,2-dimethyltetrahydro-2H-pyran-4-yl)octahydrocyclopenta[c]pyrrol-5-amine;
(3aR,5s,6aS)-2-(2,2-dimethyltetrahydro-2H-pyran-4-yl)-N-(6-(2-fluorophenyl)pyridazin-3-yl)octahydrocyclopenta[c]pyrrol-5-amine;
(3aR,5s,6aS)-2-(2,2-dimethyltetrahydro-2H-pyran-4-yl)-N-(6-(3-fluorophenyl)pyridazin-3-yl)octahydrocyclopenta[c]pyrrol-5-amine;
(3aR,5s,6aS)-2-(2,2-dimethyltetrahydro-2H-pyran-4-yl)-N-(6-(4-fluorophenyl)pyridazin-3-yl)octahydrocyclopenta[c]pyrrol-5-amine;
(3aR,5s,6aS)—N-(6-(2,5-difluorophenyl)pyridazin-3-yl)-2-(2,2-dimethyltetrahydro-2H-pyran-4-yl)octahydrocyclopenta[c]pyrrol-5-amine;
(3aR,5s,6aS)-2-(2,2-dimethyltetrahydro-2H-pyran-4-yl)-N-(6-(pyridin-3-yl)pyridazin-3-yl)octahydrocyclopenta[c]pyrrol-5-amine;
(3aR,5s,6aS)—N-(6-(1,4-dimethyl-1H-pyrazol-5-yl)pyridazin-3-yl)-2-(2,2-dimethyltetrahydro-2H-pyran-4-yl)octahydrocyclopenta[c]pyrrol-5-amine;
(3aR,5s,6aS)—N-(6-(4,4-difluoropiperidin-1-yl)pyridazin-3-yl)-2-(2,2-dimethyltetrahydro-2H-pyran-4-yl)octahydrocyclopenta[c]pyrrol-5-amine;
N-(5-(6-(((3aR,5s,6aS)-2-(2,2-dimethyltetrahydro-2H-pyran-4-yl)octahydrocyclopenta[c]pyrrol-5-yl)amino)pyridazin-3-yl)pyridin-2-yl)acetamide;
(3aR,5s,6aS)-2-(2,2-dimethyltetrahydro-2H-pyran-4-yl)-N-(6-morpholinopyridazin-3-yl)octahydrocyclopenta[c]pyrrol-5-amine;
N-((3aR,5s,6aS)-2-(2,2-dimethyltetrahydro-2H-pyran-4-yl)octahydrocyclopenta[c]pyrrol-5-yl)-[1,2,4]triazolo[4,3-b]pyridazin-6-amine;
(3aR,5s,6aS)—N-(6-(2-methyl-2H-indazol-5-yl)pyridazin-3-yl)-2-(tetrahydro-2H-pyran-4-yl)octahydrocyclopenta[c]pyrrol-5-amine;
(3aR,5s,6aS)-2-(2,2-dimethyltetrahydro-2H-pyran-4-yl)-N-(6-(2-methyl-2H-indazol-5-yl)pyridazin-3-yl)octahydrocyclopenta[c]pyrrol-5-amine;
(3aR,5s,6aS)—N-(6-(2-chloro-5-fluorophenyl)pyridazin-3-yl)-2-(1-(tetrahydro-2H-pyran-4-yl)cyclopropyl)octahydrocyclopenta[c]pyrrol-5-amine;
(3aR,5s,6aS)-2-(2,2-dimethyltetrahydro-2H-pyran-4-yl)-N-(6-(phenylsulfinyl)pyridazin-3-yl)octahydrocyclopenta[c]pyrrol-5-amine;
(3aR,5s,6aS)—N-(6-(cyclohexylsulfonyl)pyridazin-3-yl)-2-(2,2-dimethyltetrahydro-2H-pyran-4-yl)octahydrocyclopenta[c]pyrrol-5-amine;
(3aR,5s,6aS)—N-(6-(2-chloro-5-fluorophenyl)pyridazin-3-yl)-2-(4,4-difluorocyclohexyl)octahydrocyclopenta[c]pyrrol-5-amine;
(3aR,5s,6aS)—N-(6-(2-chloro-5-fluorophenyl)pyridazin-3-yl)-2-(4,4-dimethylcyclohexyl)octahydrocyclopenta[c]pyrrol-5-amine;
(3aR,5s,6aS)—N-(6-(2-chloro-5-fluorophenyl)pyridazin-3-yl)-2-(2-oxaspiro[3.3]heptan-6-yl)octahydrocyclopenta[c]pyrrol-5-amine;
7-cyclopropyl-5,5-dimethyl-3-(((3aR,5s,6aS)-2-((tetrahydro-2H-pyran-4-yl)methyl)octahydrocyclopenta[c]pyrrol-5-yl)amino)-5,7-dihydro-6H-pyrrolo[2,3-c]pyridazin-6-one;
7-allyl-3-(((3aR,5s,6aS)-2-(3,3-dimethylbutyl)octahydrocyclopenta[c]pyrrol-5-yl)amino)-5,5-dimethyl-5,7-dihydro-6H-pyrrolo[2,3-c]pyridazin-6-one;
3-(((3aR,5s,6aS)-2-(3,3-dimethylbutyl)octahydrocyclopenta[c]pyrrol-5-yl)amino)-5,5-dimethyl-7-propyl-5,7-dihydro-6H-pyrrolo[2,3-c]pyridazin-6-one;
1-((3aR,5s,6aS)-5-((6-(2-Chloro-5-fluorophenyl)pyridazin-3-yl)amino)hexahydrocyclopenta[c]pyrrol-2(1H)-yl)-2-methylpropan-2-ol;
(3aR,5s,6aS)—N-(6-(2-chloro-5-fluorophenyl)pyridazin-3-yl)-2-cyclopentyloctahydrocyclopenta[c]pyrrol-5-amine;
(3aR,5s,6aS)—N-(6-(2-chloro-5-fluorophenyl)pyridazin-3-yl)-2-cycloheptyloctahydrocyclopenta[c]pyrrol-5-amine;
(3aR,5s,6aS)—N-(6-(2-chloro-5-fluorophenyl)pyridazin-3-yl)-2-(2,3-dihydro-1H-inden-2-yl)octahydrocyclopenta[c]pyrrol-5-amine;
(3aR,5s,6aS)—N-(6-(2-chloro-5-fluorophenyl)pyridazin-3-yl)-2-(spiro[5.5]undecan-3-yl)octahydrocyclopenta[c]pyrrol-5-amine;
(3aR,5s,6aS)—N-(6-(2-chloro-5-fluorophenyl)pyridazin-3-yl)-2-(3-oxaspiro[5.5]undecan-9-yl)octahydrocyclopenta[c]pyrrol-5-amine; and
4-((3aR,5s,6aS)-5-((6-(2-chloro-5-fluorophenyl)pyridazin-3-yl)amino)hexahydrocyclopenta[c]pyrrol-2(1H)-yl)tetrahydro-2H-thiopyran 1,1-dioxide;

or a pharmaceutically acceptable salt thereof.

E54. The compound of any of E1-E53, or a pharmaceutically acceptable salt thereof, wherein the compound is isotopically labeled.

E55. A pharmaceutical composition comprising the compound of any of E1-E54, or a pharmaceutically acceptable salt thereof, and a pharmaceutically acceptable carrier.

E56. A method for antagonizing mAChR M₄ in a subject, comprising administering to the subject a therapeutically effective amount of the compound of any of E1-E54, or a pharmaceutically acceptable salt thereof, or the pharmaceutical composition of E55.

E57. A method for treating a disorder in a subject, wherein the subject would benefit from antagonism of mAChR M₄, comprising administering to the mammal a therapeutically effective amount of the compound of any of E1-E54, or a pharmaceutically acceptable salt thereof, or the pharmaceutical composition of E55.

E58. The method of E57, wherein the disorder is a neurodegenerative disorder, a movement disorder, or a brain disorder.

E59. The method of E58, wherein the disorder is a movement disorder.

E60. The method of E58, wherein the disorder is selected from Parkinson's disease, drug-induced Parkinsonism, dystonia, Tourette's syndrome, dyskinesias, schizophrenia, cognitive deficits associated with schizophrenia, excessive daytime sleepiness, attention deficit hyperactivity disorder (ADHD), Huntington's disease, chorea, cerebral palsy, and progressive supranuclear palsy.

E61. A method for treating motor symptoms in a subject, comprising administering to a subject in need thereof a therapeutically effective amount of the compound of any of E1-E54, or a pharmaceutically acceptable salt thereof, or the pharmaceutical composition of E55.

E62. The method of E61, wherein the subject has a disorder selected from Parkinson's disease, drug-induced Parkinsonism, dystonia, Tourette's syndrome, dyskinesias, schizophrenia, cognitive deficits associated with schizophrenia, excessive daytime sleepiness, attention deficit hyperactivity disorder (ADHD), Huntington's disease, chorea, cerebral palsy, and progressive supranuclear palsy.

E63. A compound of any of E1-E54, or a pharmaceutically acceptable salt thereof, or the pharmaceutical composition of E55, for use in the treatment of a neurodegenerative disorder, a movement disorder, or a brain disorder.

E64. The use of a compound of any of E1-E54, or a pharmaceutically acceptable salt thereof, or the pharmaceutical composition of E55, for the preparation of a medicament for the treatment of a neurodegenerative disorder, a movement disorder, or a brain disorder.

What is claimed is:

1. A compound of formula (I): 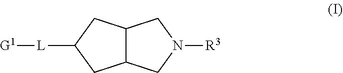
or a pharmaceutically acceptable salt thereof, wherein:
G¹ is
a) a 6- or 5-membered monocyclic heteroaryl having 2, 1, or 3 heteroatoms independently selected from N, O, and S, the monocyclic heteroaryl being substituted with R^{1a} and 0-1 R^{1b};
b) a phenyl substituted with R^{1a} and 0-1 R^{1b}; or
c) an 8- to 12-membered fused bicyclic heteroaryl optionally substituted with 1-5 R²;
R^{1a} is G^{1a}, —O-G^{1a}, —SO₂-G^{1a}, —S(O)-G^{1a}, —C(O)NR^{1c}R^{1d}, or halogen;
G^{1a} is a 6- to 12-membered aryl, a 5- to 12-membered heteroaryl, a 4- to 12-membered heterocyclyl, or a C₃₋₁₂carbocyclyl, wherein G^{1a} is optionally substituted with 1-5 substituents independently selected from the group consisting of halogen, cyano, C₁₋₄alkyl, C₁₋₄haloalkyl, —OR¹⁰, —N(R¹⁰)₂, and —NR¹⁰C(O)R¹⁰;
R^{1b} is —N(R¹¹)₂;
R^{1c} is hydrogen, C₁₋₄alkyl, C₁₋₄haloalkyl, G^{1a}, or —C₁₋₃alkylene-G^{1a};
R^{1d} is hydrogen, C₁₋₄alkyl, C₁₋₄haloalkyl, C₃₋₄cycloalkyl, or —C₁₋₃alkylene-C₃₋₄cycloalkyl, or R^{1c} and R^{1d}, together with a nitrogen atom to which they attach form a 4- to 8-membered heterocyclic ring optionally substituted with 1-4 substituents independently selected from the group consisting of halogen and C₁₋₄alkyl;
R¹⁰ and R¹¹, at each occurrence, are independently hydrogen, C₁₋₄alkyl, C₁₋₄haloalkyl, C₃₋₄cycloalkyl, or C₁₋₃alkylene-C₃₋₄cycloalkyl, wherein alternatively two R¹⁰ and/or two R¹¹, together with a nitrogen to which the two R¹⁰ or two R¹¹ attach form a 4- to 6-membered heterocyclic ring optionally substituted with 1-4 substituents independently selected from the group consisting of halogen and C₁₋₄alkyl;
R², at each occurrence, is independently halogen, cyano, oxo, C₁₋₄alkyl, C₁₋₄haloalkyl, C₂₋₄alkenyl, C₃₋₆cycloalkyl, or C₁₋₃alkylene-C₃₋₄cycloalkyl;
L is NR, O, —NR—C(O)—; —NR—C₁₋₃alkylene-, or —O—C₁₋₃alkylene-;
R is hydrogen, C₁₋₄alkyl, C₃₋₄cycloalkyl, or —C₁₋₃alkylene-C₃₋₄cycloalkyl;
R³ is G², -L¹-G², -L²-G², -L²-L¹-G², —C₂₋₆alkylene-R^{3a}, or C₃₋₇alkyl;
L¹ is C₁₋₃alkylene;
L² is 1,1-cyclopropylene;
G² is a 6- to 12-membered aryl, a 5- to 12-membered heteroaryl, a 4- to 12-membered heterocyclyl, or a C₃₋₁₂carbocyclyl optionally fused to a phenyl, wherein G² is optionally substituted with 1-5 substituents independently selected from the group consisting of halogen, cyano, C₁₋₄alkyl, C₁₋₄haloalkyl, —OR¹³, —N(R¹³)₂, —C₁₋₃alkylene-OR¹³, and —C₁₋₃alkylene-N(R¹³)₂;
R^{3a} is —OR¹⁴ or —N(R¹⁴)₂; and
R¹³ and R¹⁴, at each occurrence, are independently hydrogen, C₁₋₄alkyl, C₁₋₄haloalkyl, C₃₋₄cycloalkyl, or C₁₋₃alkylene-C₃₋₄cycloalkyl, wherein alternatively two R¹³ or two R¹⁴ together with a nitrogen to which the two R¹³ or two R¹⁴ attach form a 4- to 6-membered heterocyclic ring optionally substituted with 1-4 substituents independently selected from the group consisting of halogen and C₁₋₄alkyl;
provided that R³ is G², -L²-G², -L²-L¹-G², or —C₂₋₆alkylene-R^{3a}, when R^{1a} is G^{1a}, —O-G^{1a}, or halogen; and G¹-L- is not

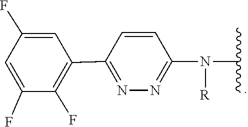

2. The compound of claim 1, or a pharmaceutically acceptable salt thereof, wherein G¹ is

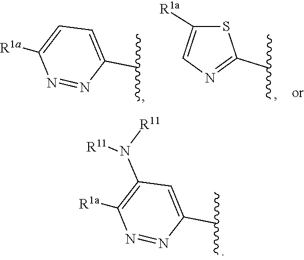

3. The compound of claim 1 or 2, or a pharmaceutically acceptable salt thereof, wherein R^{1a} is G^{1a}.

4. The compound of claim 1 or 2, or a pharmaceutically acceptable salt thereof, wherein R^{1a} is —SO₂-G^{1a}, —S(O)-G^{1a}, or —C(O)NG^{1a}R^{1d}.

5. The compound of any of claims 1-4, or a pharmaceutically acceptable salt thereof, wherein G^{1a} is the 6- to 12-membered aryl.

6. The compound of claim 5, or a pharmaceutically acceptable salt thereof, wherein the ring system of the 6- to 12-membered aryl is a phenyl.

7. The compound of any of claims 1-4, or a pharmaceutically acceptable salt thereof, wherein G^{1a} is the 5- to 12-membered heteroaryl.

8. The compound of claim 7, or a pharmaceutically acceptable salt thereof, wherein the ring system of the 5- to 12-membered heteroaryl is a pyridinyl, pyrazolyl, indazolyl, or imidazopyridinyl.

9. The compound of any of claims 1-4, or a pharmaceutically acceptable salt thereof, wherein G^{1a} is the 4- to 12-membered heterocyclyl.

10. The compound of claim 9, or a pharmaceutically acceptable salt thereof, wherein the ring system of the 4- to 12-membered heterocyclyl is a 4- to 8-membered monocyclic heterocyclyl ring system containing 1-2 heteroatoms independently selected from the group consisting of N and O.

11. The compound of any of claims 1-4, or a pharmaceutically acceptable salt thereof, wherein G^{1a} is the C₃₋₁₂carbocyclyl.

12. The compound of claim 11, or a pharmaceutically acceptable salt thereof, wherein the ring system of the C₃₋₁₂carbocyclyl is a C₃₋₈cycloalkyl ring system.

13. The compound of any of claims 1-3, or a pharmaceutically acceptable salt thereof, wherein G^{1a} is phenyl, C₃₋₆cycloalkyl,

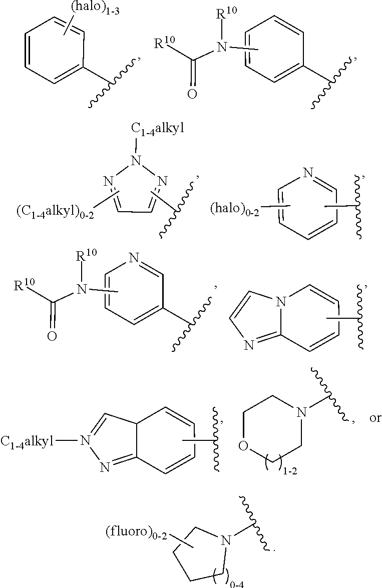

14. The compound of claim 13, or a pharmaceutically acceptable salt thereof, wherein G^{1a} is phenyl,

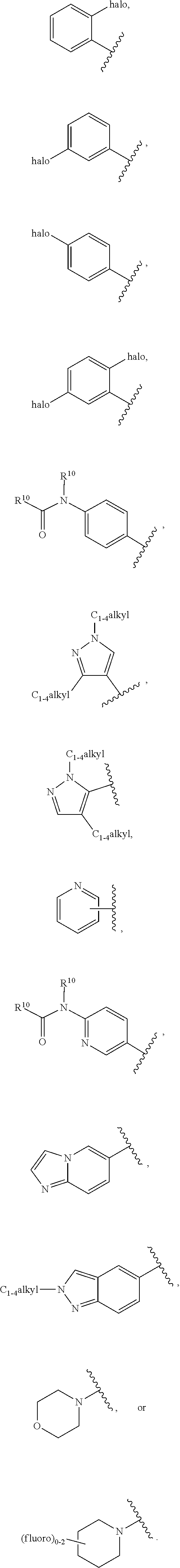

15. The compound of any of claims 1-2 or 4, or a pharmaceutically acceptable salt thereof, wherein G^{1a} is phenyl, C₃₋₆cycloalkyl, or

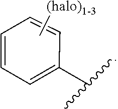

16. The compound of claim 15, or a pharmaceutically acceptable salt thereof, wherein G^{1a} is phenyl, cyclopropyl, cyclohexyl,

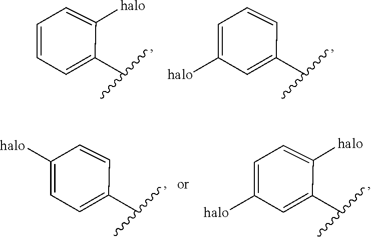

17. The compound of claim 1 or 2, or a pharmaceutically acceptable salt thereof, wherein R^{1a} is —C(O)NR^{1c}R^{1d}; and R^{1c} and R^{1d}, together with the nitrogen atom to which they attach form a 4- to 8-membered heterocyclic ring optionally substituted with 1-4 substituents independently selected from the group consisting of halogen and C₁₋₄alkyl.

18. The compound of claim 1, or a pharmaceutically acceptable salt thereof, wherein G¹ is the 8- to 12-membered fused bicyclic heteroaryl.

19. The compound of claim 18, or a pharmaceutically acceptable salt thereof, wherein G¹ is

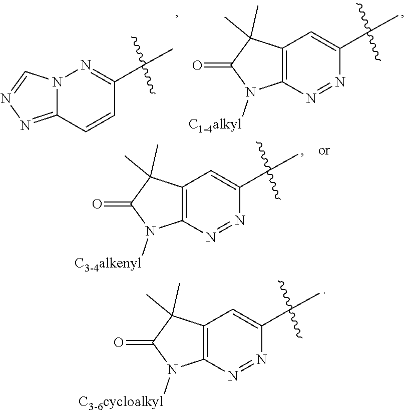

20. The compound of any of claims 1-19, or a pharmaceutically acceptable salt thereof, wherein R³ is G².

21. The compound of any of claims 1-2, 4-12 or 15-19, or a pharmaceutically acceptable salt thereof, wherein R³ is -L¹-G².

22. The compound of any of claims 1-19, or a pharmaceutically acceptable salt thereof, wherein R³ is -L²-G².

23. The compound of any of claims 1-22, or a pharmaceutically acceptable salt thereof, wherein G² is the 4- to 12-membered heterocyclyl.

24. The compound of claim 23, or a pharmaceutically acceptable salt thereof, wherein the ring system of the 4- to 12-membered heterocyclyl is a 4- to 8-membered monocyclic heterocyclyl ring system or a 7- to 12-membered spiro heterocyclyl ring system, wherein the heterocyclyls contain one heteroatom selected from O and S.

25. The compound of claim 24, or a pharmaceutically acceptable salt thereof, wherein the ring system of the 4- to 12-membered heterocyclyl is an oxetanyl, a tetrahydropyranyl, a tetrahydrothiopyranyl, a 2-oxaspiro[3.3]heptanyl, or a 3-oxaspiro[5.5]undecanyl.

26. The compound of any of claims 1-22, or a pharmaceutically acceptable salt thereof, wherein G² is the 6- to 12-membered aryl.

27. The compound of claim 26, or a pharmaceutically acceptable salt thereof, wherein the ring system of the 6- to 12-membered aryl is a phenyl or a phenyl bonded to the parent molecule and fused to a 5- to 7-membered heterocycle containing 1-2 oxygen atoms.

28. The compound of any of claims 1-22, or a pharmaceutically acceptable salt thereof, wherein G² is the 5- to 12-membered heteroaryl.

29. The compound of claim 28, or a pharmaceutically acceptable salt thereof, wherein the ring system of the 5- to 12-membered heteroaryl is a pyrazolyl, pyridinyl, or indolyl.

30. The compound of any of claims 1-22, or a pharmaceutically acceptable salt thereof, wherein G² is the C₃₋₁₂carbocyclyl optionally fused to a phenyl.

31. The compound of claim 30, or a pharmaceutically acceptable salt thereof, wherein the ring system of the C₃₋₁₂carbocyclyl optionally fused to a phenyl is a C₃₋₈cycloalkyl optionally fused to a phenyl or the ring system of the C₃₋₁₂carbocyclyl optionally fused to a phenyl is a spiro[5.5]undecanyl.

32. The compound of claim 20, or a pharmaceutically acceptable salt thereof, wherein G² is

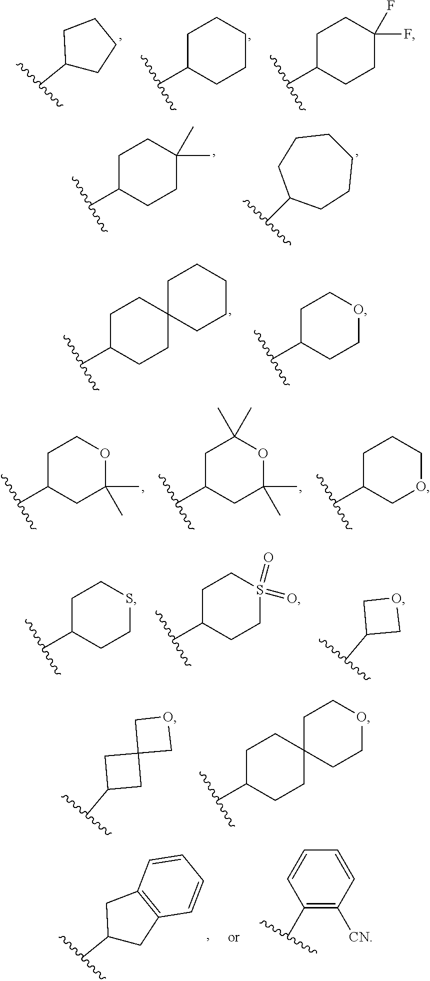

33. The compound of claim 21, or a pharmaceutically acceptable salt thereof, wherein G² is

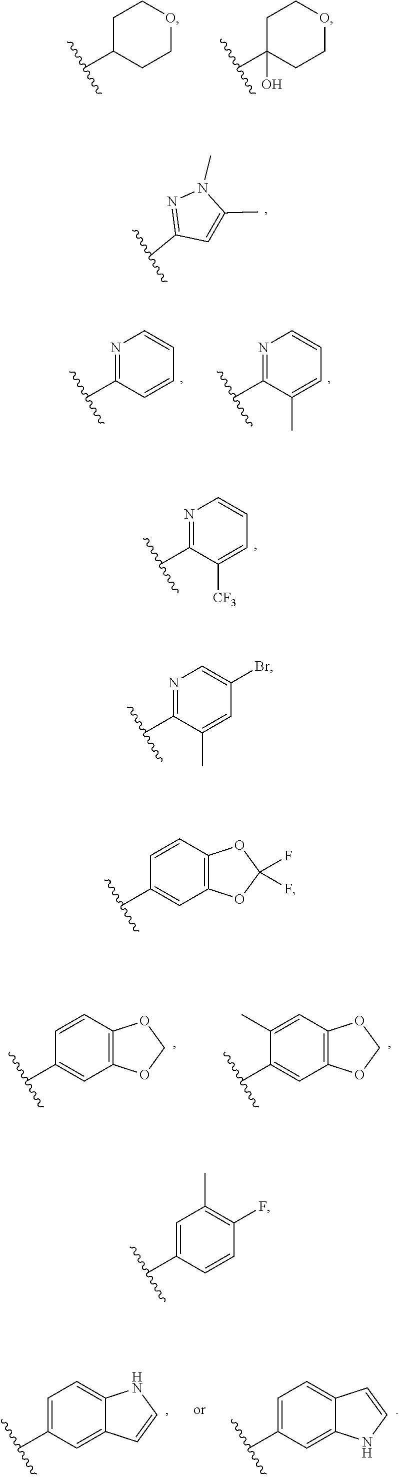

34. The compound of claim 22, or a pharmaceutically acceptable salt thereof, wherein G² is

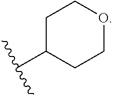 [SMILES: *C1CCOCC1]

35. The compound of any of claims 1-2, 4-12, or 15-19, or a pharmaceutically acceptable salt thereof, wherein R³ is C₃₋₇alkyl.

36. The compound of any of claims 1-19, or a pharmaceutically acceptable salt thereof, wherein R³ is —C₂₋₄alkylene-OR¹⁴.

37. The compound of any of claims 1-36, or a pharmaceutically acceptable salt thereof, wherein L is NR.

38. The compound of claim 37, or a pharmaceutically acceptable salt thereof, wherein R is hydrogen.

39. The compound of claim 1, wherein the compound is selected from the group consisting of:
(3aR,5s,6aS)—N-(6-(2-chloro-5-fluorophenyl)pyridazin-3-yl)-2-(3-methoxypropyl)octahydrocyclopenta[c]pyrrol-5-amine;
((3aR,5s,6aS)—N-(6-(2-chloro-5-fluorophenyl)pyridazin-3-yl)-2-(tetrahydro-2H-pyran-4-yl)octahydrocyclopenta[c]pyrrol-5-amine;
N-cyclopropyl-6-(((3aR,5s,6aS)-2-((tetrahydro-2H-pyran-4-yl)methyl)octahydrocyclopenta[c]pyrrol-5-yl)amino)pyridazine-3-carboxamide;
N-cyclohexyl-6-(((3aR,5s,6aS)-2-((tetrahydro-2H-pyran-4-yl)methyl)octahydrocyclopenta[c]pyrrol-5-yl)amino)pyridazine-3-carboxamide;
piperidin-1-yl(6-(((3aR,5s,6aS)-2-((tetrahydro-2H-pyran-4-yl)methyl)octahydrocyclopenta[c]pyrrol-5-yl)amino)pyridazin-3-yl)methanone;
morpholino(6-(((3aR,5s,6aS)-2-((tetrahydro-2H-pyran-4-yl)methyl)octahydrocyclopenta[c]pyrrol-5-yl)amino)pyridazin-3-yl)methanone;
N-phenyl-6-(((3aR,5s,6aS)-2-((tetrahydro-2H-pyran-4-yl)methyl)octahydrocyclopenta[c]pyrrol-5-yl)amino)pyridazine-3-carboxamide;
N-(2-fluorophenyl)-6-(((3aR,5s,6aS)-2-((tetrahydro-2H-pyran-4-yl)methyl)octahydrocyclopenta[c]pyrrol-5-yl)amino)pyridazine-3-carboxamide;
N-(3-fluorophenyl)-6-(((3aR,5s,6aS)-2-((tetrahydro-2H-pyran-4-yl)methyl)octahydrocyclopenta[c]pyrrol-5-yl)amino)pyridazine-3-carboxamide;
N-(4-fluorophenyl)-6-(((3aR,5s,6aS)-2-((tetrahydro-2H-pyran-4-yl)methyl)octahydrocyclopenta[c]pyrrol-5-yl)amino)pyridazine-3-carboxamide;
N-(2-chloro-5-fluorophenyl)-6-(((3aR,5s,6aS)-2-((tetrahydro-2H-pyran-4-yl)methyl)octahydrocyclopenta[c]pyrrol-5-yl)amino)pyridazine-3-carboxamide;
(3aR,5s,6aS)—N-(6-(2-chloro-5-fluorophenyl)pyridazin-3-yl)-2-cyclohexyloctahydrocyclopenta[c]pyrrol-5-amine;
(3aR,5s,6aS)—N-(6-(2-chloro-5-fluorophenyl)pyridazin-3-yl)-2-(tetrahydro-2H-pyran-3-yl)octahydrocyclopenta[c]pyrrol-5-amine;
(3aR,5s,6aS)—N-(6-(2-chloro-5-fluorophenyl)pyridazin-3-yl)-2-(oxetan-3-yl)octahydrocyclopenta[c]pyrrol-5-amine;
(3aR,5s,6aS)—N-(6-(2-chloro-5-fluorophenyl)pyridazin-3-yl)-2-(tetrahydro-2H-thiopyran-4-yl)octahydrocyclopenta[c]pyrrol-5-amine;
(3aR,5s,6aS)—N-(6-(2-chloro-5-fluorophenyl)pyridazin-3-yl)-2-(2,2-dimethyltetrahydro-2H-pyran-4-yl)octahydrocyclopenta[c]pyrrol-5-amine;
2-((3aR,5s,6aS)-5-((6-(2-chloro-5-fluorophenyl)pyridazin-3-yl)amino)hexahydrocyclopenta[c]pyrrol-2(1H)-yl)benzonitrile;
(3aR,5s,6aS)—N-(6-(2-chloro-5-fluorophenyl)pyridazin-3-yl)-2-(2,2,6,6-tetramethyltetrahydro-2H-pyran-4-yl)octahydrocyclopenta[c]pyrrol-5-amine;
N-((3aR,5s,6aS)-2-(2,2-dimethyltetrahydro-2H-pyran-4-yl)octahydrocyclopenta[c]pyrrol-5-yl)-5-phenylthiazol-2-amine;
N-(4-(6-(((3aR,5s,6aS)-2-(2,2-dimethyltetrahydro-2H-pyran-4-yl)octahydrocyclopenta[c]pyrrol-5-yl)amino)pyridazin-3-yl)phenyl)acetamide;
(3aR,5s,6aS)-2-(2,2-dimethyltetrahydro-2H-pyran-4-yl)-N-(6-(imidazo[1,2-a]pyridin-6-yl)pyridazin-3-yl)octahydrocyclopenta[c]pyrrol-5-amine;
(3aR,5s,6aS)—N-(6-(1,3-dimethyl-1H-pyrazol-4-yl)pyridazin-3-yl)-2-(2,2-dimethyltetrahydro-2H-pyran-4-yl)octahydrocyclopenta[c]pyrrol-5-amine;
(3aR,5s,6aS)—N-(6-(phenylsulfonyl)pyridazin-3-yl)-2-((tetrahydro-2H-pyran-4-yl)methyl)octahydrocyclopenta[c]pyrrol-5-amine;
(3aR,5s,6aS)-2-((1,5-dimethyl-1H-pyrazol-3-yl)methyl)-N-(6-(phenylsulfonyl)pyridazin-3-yl)octahydrocyclopenta[c]pyrrol-5-amine;
(3aR,5s,6aS)—N-(6-(phenylsulfonyl)pyridazin-3-yl)-2-(pyridin-2-ylmethyl)octahydrocyclopenta[c]pyrrol-5-amine;
(3aR,5s,6aS)-2-((2,2-difluorobenzo[d][1,3]dioxol-5-yl)methyl)-N-(6-(phenylsulfonyl)pyridazin-3-yl)octahydrocyclopenta[c]pyrrol-5-amine;
(3aR,5s,6aS)-2-(benzo[d][1,3]dioxol-5-ylmethyl)-N-(6-(phenylsulfonyl)pyridazin-3-yl)octahydrocyclopenta[c]pyrrol-5-amine;
(3aR,5s,6aS)—N-(6-(phenylsulfinyl)pyridazin-3-yl)-2-((tetrahydro-2H-pyran-4-yl)methyl)octahydrocyclopenta[c]pyrrol-5-amine;
(3aR,5s,6aS)-2-(2,2-dimethyltetrahydro-2H-pyran-4-yl)-N-(6-(phenylsulfinyl)pyridazin-3-yl)octahydrocyclopenta[c]pyrrol-5-amine;
(3aR,5s,6aS)-2-(benzo[d][1,3]dioxol-5-ylmethyl)-N-(6-(phenylsulfinyl)pyridazin-3-yl)octahydrocyclopenta[c]pyrrol-5-amine;
(3aR,5s,6aS)-2-(4-fluoro-3-methylbenzyl)-N-(6-(phenylsulfonyl)pyridazin-3-yl)octahydrocyclopenta[c]pyrrol-5-amine;
(3aR,5s,6aS)-2-(4-fluoro-3-methylbenzyl)-N-(6-(phenylsulfinyl)pyridazin-3-yl)octahydrocyclopenta[c]pyrrol-5-amine;
(3aR,5s,6aS)—N-(6-(cyclohexylsulfonyl)pyridazin-3-yl)-2-(pyridin-2-ylmethyl)octahydrocyclopenta[c]pyrrol-5-amine;
(3aR,5s,6aS)—N-(6-(cyclohexylsulfonyl)pyridazin-3-yl)-2-((tetrahydro-2H-pyran-4-yl)methyl)octahydrocyclopenta[c]pyrrol-5-amine;
(3aR,5s,6aS)—N-(6-(cyclohexylsulfonyl)pyridazin-3-yl)-2-((1, 5-dimethyl-1H-pyrazol-3-yl)methyl)octahydrocyclopenta[c]pyrrol-5-amine;
(3aR,5s,6aS)—N-(6-(cyclohexylsulfonyl)pyridazin-3-yl)-2-(2,2-dimethyltetrahydro-2H-pyran-4-yl)octahydrocyclopenta[c]pyrrol-5-amine;
(3aR,5s,6aS)-2-(benzo[d][1,3]dioxol-5-ylmethyl)-N-(6-(cyclohexylsulfonyl)pyridazin-3-yl)octahydrocyclopenta[c]pyrrol-5-amine;
(3aR,5s,6aS)—N-(6-(cyclohexylsulfonyl)pyridazin-3-yl)-2-(4-fluoro-3-methylbenzyl)octahydrocyclopenta[c]pyrrol-5-amine;
N⁵-cyclopropyl-6-(phenylsulfonyl)-N³-((3aR,5s,6aS)-2-((tetrahydro-2H-pyran-4-yl)methyl)octahydrocyclopenta[c]pyrrol-5-yl)pyridazine-3,5-diamine;
(3aR,5s,6aS)-2-((3-methylpyridin-2-yl)methyl)-N-(6-(phenylsulfonyl)pyridazin-3-yl)octahydrocyclopenta[c]pyrrol-5-amine;
(3aR,5s,6aS)—N-(6-(phenylsulfonyl)pyridazin-3-yl)-2-((3-(trifluoromethyl)pyridin-2-yl)methyl)octahydrocyclopenta[c]pyrrol-5-amine;
(3aR,5s,6aS)-2-((6-methylbenzo[d][1,3]dioxol-5-yl)methyl)-N-(6-(phenylsulfonyl)pyridazin-3-yl)octahydrocyclopenta[c]pyrrol-5-amine;
(3aR,5s,6aS)—N-(6-(cyclohexylsulfonyl)pyridazin-3-yl)-2-((3-methylpyridin-2-yl)methyl)octahydrocyclopenta[c]pyrrol-5-amine;
(3aR,5s,6aS)—N-(6-(cyclohexylsulfonyl)pyridazin-3-yl)-2-((6-methylbenzo[d][1,3]dioxol-5-yl)methyl)octahydrocyclopenta[c]pyrrol-5-amine;
(3aR,5s,6aS)-2-((5-bromo-3-methylpyridin-2-yl)methyl)-N-(6-(cyclohexylsulfonyl)pyridazin-3-yl)octahydrocyclopenta[c]pyrrol-5-amine;
4-(((3aR,5s,6aS)-5-((6-(cyclohexylsulfonyl)pyridazin-3-yl)amino)hexahydrocyclopenta[c]pyrrol-2(1H)-yl)methyl)tetrahydro-2H-pyran-4-ol;
(3aR,5s,6aS)-2-((1H-indol-5-yl)methyl)-N-(6-(cyclohexylsulfonyl)pyridazin-3-yl)octahydrocyclopenta[c]pyrrol-5-amine;
(3aR,5s,6aS)-2-((1H-indol-6-yl)methyl)-N-(6-(cyclohexylsulfonyl)pyridazin-3-yl)octahydrocyclopenta[c]pyrrol-5-amine;
(3aR,5s,6aS)—N-(6-(cyclohexylsulfonyl)pyridazin-3-yl)-2-(3, 3-dimethylbutyl)octahydrocyclopenta[c]pyrrol-5-amine;
(3aR,5s,6aS)-2-(3,3-dimethylbutyl)-N-(6-(phenylsulfonyl)pyridazin-3-yl)octahydrocyclopenta[c]pyrrol-5-amine;
(3aR,5s,6aS)—N-(6-chloropyridazin-3-yl)-2-(2,2-dimethyltetrahydro-2H-pyran-4-yl)octahydrocyclopenta[c]pyrrol-5-amine;
(3aR,5s,6aS)-2-(2,2-dimethyltetrahydro-2H-pyran-4-yl)-N-(6-(2-fluorophenyl)pyridazin-3-yl)octahydrocyclopenta[c]pyrrol-5-amine;
(3aR,5s,6aS)-2-(2,2-dimethyltetrahydro-2H-pyran-4-yl)-N-(6-(3-fluorophenyl)pyridazin-3-yl)octahydrocyclopenta[c]pyrrol-5-amine;
(3aR,5s,6aS)-2-(2,2-dimethyltetrahydro-2H-pyran-4-yl)-N-(6-(4-fluorophenyl)pyridazin-3-yl)octahydrocyclopenta[c]pyrrol-5-amine;
(3aR,5s,6aS)—N-(6-(2,5-difluorophenyl)pyridazin-3-yl)-2-(2,2-dimethyltetrahydro-2H-pyran-4-yl)octahydrocyclopenta[c]pyrrol-5-amine;
(3aR,5s,6aS)-2-(2,2-dimethyltetrahydro-2H-pyran-4-yl)-N-(6-(pyridin-3-yl)pyridazin-3-yl)octahydrocyclopenta[c]pyrrol-5-amine;
(3aR,5s,6aS)—N-(6-(1,4-dimethyl-1H-pyrazol-5-yl)pyridazin-3-yl)-2-(2,2-dimethyltetrahydro-2H-pyran-4-yl)octahydrocyclopenta[c]pyrrol-5-amine;
(3aR,5s,6aS)—N-(6-(4,4-difluoropiperidin-1-yl)pyridazin-3-yl)-2-(2,2-dimethyltetrahydro-2H-pyran-4-yl)octahydrocyclopenta[c]pyrrol-5-amine;
N-(5-(6-(((3aR,5s,6aS)-2-(2,2-dimethyltetrahydro-2H-pyran-4-yl)octahydrocyclopenta[c]pyrrol-5-yl)amino)pyridazin-3-yl)pyridin-2-yl)acetamide;
(3aR,5s,6aS)-2-(2,2-dimethyltetrahydro-2H-pyran-4-yl)-N-(6-morpholinopyridazin-3-yl)octahydrocyclopenta[c]pyrrol-5-amine;
N-((3aR,5s,6aS)-2-(2,2-dimethyltetrahydro-2H-pyran-4-yl)octahydrocyclopenta[c]pyrrol-5-yl)-[1,2,4]triazolo[4,3-b]pyridazin-6-amine;
(3aR,5s,6aS)—N-(6-(2-methyl-2H-indazol-5-yl)pyridazin-3-yl)-2-(tetrahydro-2H-pyran-4-yl)octahydrocyclopenta[c]pyrrol-5-amine;
(3aR,5s,6aS)-2-(2,2-dimethyltetrahydro-2H-pyran-4-yl)-N-(6-(2-methyl-2H-indazol-5-yl)pyridazin-3-yl)octahydrocyclopenta[c]pyrrol-5-amine;
(3aR,5s,6aS)—N-(6-(2-chloro-5-fluorophenyl)pyridazin-3-yl)-2-(1-(tetrahydro-2H-pyran-4-yl)cyclopropyl)octahydrocyclopenta[c]pyrrol-5-amine;
(3aR,5s,6aS)-2-(2,2-dimethyltetrahydro-2H-pyran-4-yl)-N-(6-(phenylsulfinyl)pyridazin-3-yl)octahydrocyclopenta[c]pyrrol-5-amine;
(3aR,5s,6aS)—N-(6-(cyclohexylsulfonyl)pyridazin-3-yl)-2-(2,2-dimethyltetrahydro-2H-pyran-4-yl)octahydrocyclopenta[c]pyrrol-5-amine;
(3aR,5s,6aS)—N-(6-(2-chloro-5-fluorophenyl)pyridazin-3-yl)-2-(4,4-difluorocyclohexyl)octahydrocyclopenta[c]pyrrol-5-amine;
(3aR,5s,6aS)—N-(6-(2-chloro-5-fluorophenyl)pyridazin-3-yl)-2-(4,4-dimethylcyclohexyl)octahydrocyclopenta[c]pyrrol-5-amine;
(3aR,5s,6aS)—N-(6-(2-chloro-5-fluorophenyl)pyridazin-3-yl)-2-(2-oxaspiro[3.3]heptan-6-yl)octahydrocyclopenta[c]pyrrol-5-amine;
7-cyclopropyl-5,5-dimethyl-3-(((3aR,5s,6aS)-2-((tetrahydro-2H-pyran-4-yl)methyl)octahydrocyclopenta[c]pyrrol-5-yl)amino)-5,7-dihydro-6H-pyrrolo[2,3-c]pyridazin-6-one;
7-allyl-3-(((3aR,5s,6aS)-2-(3,3-dimethylbutyl)octahydrocyclopenta[c]pyrrol-5-yl)amino)-5,5-dimethyl-5,7-dihydro-6H-pyrrolo[2,3-c]pyridazin-6-one;
3-(((3aR,5s,6aS)-2-(3,3-dimethylbutyl)octahydrocyclopenta[c]pyrrol-5-yl)amino)-5,5-dimethyl-7-propyl-5,7-dihydro-6H-pyrrolo[2,3-c]pyridazin-6-one;
1-((3aR,5s,6aS)-5-((6-(2-Chloro-5-fluorophenyl)pyridazin-3-yl)amino)hexahydrocyclopenta[c]pyrrol-2(1H)-yl)-2-methylpropan-2-ol;
(3aR,5s,6aS)—N-(6-(2-chloro-5-fluorophenyl)pyridazin-3-yl)-2-cyclopentyloctahydrocyclopenta[c]pyrrol-5-amine;
(3aR,5s,6aS)—N-(6-(2-chloro-5-fluorophenyl)pyridazin-3-yl)-2-cycloheptyloctahydrocyclopenta[c]pyrrol-5-amine;
(3aR,5s,6aS)—N-(6-(2-chloro-5-fluorophenyl)pyridazin-3-yl)-2-(2,3-dihydro-1H-inden-2-yl)octahydrocyclopenta[c]pyrrol-5-amine;
(3aR,5s,6aS)—N-(6-(2-chloro-5-fluorophenyl)pyridazin-3-yl)-2-(spiro[5.5]undecan-3-yl)octahydrocyclopenta[c]pyrrol-5-amine;
(3aR,5s,6aS)—N-(6-(2-chloro-5-fluorophenyl)pyridazin-3-yl)-2-(3-oxaspiro[5.5]undecan-9-yl)octahydrocyclopenta[c]pyrrol-5-amine; and
4-((3aR,5s,6aS)-5-((6-(2-chloro-5-fluorophenyl)pyridazin-3-yl)amino)hexahydrocyclopenta[c]pyrrol-2(1H)-yl)tetrahydro-2H-thiopyran 1,1-dioxide;
or a pharmaceutically acceptable salt thereof.

40. A compound of any of claims 1-39, or a pharmaceutically acceptable salt thereof, for use in the treatment of a neurodegenerative disorder, a movement disorder, or a brain disorder.
